(19) United States
(12) Patent Application Publication
(10) Pub. No.: US 2005/0272677 A1
(43) Pub. Date: Dec. 8, 2005

(54) Delivery of a substance to a pre-determined site

(76) Inventors: Robert Heinz Edward Friesen, Houten (NL); Cornelis Johannes Leenhouts, Haren (NL); Harm Jan Hektor, Groningen (NL); Johannes Henricus van Esch, Groningen (NL); Andre Heeres, Groningen (NL); George Thomas Robillard, Zuidhorn (NL)

Correspondence Address: TRASK BRITT, P.O. BOX 2550, SALT LAKE CITY, UT, 84110, US

(21) Appl. No.: 10/957,887

(22) Filed: Oct. 4, 2004

Related U.S. Application Data
(63) Continuation of application No. PCT/NL03/00256, filed on Apr. 4, 2003.
(60) Provisional application No. 60/369,927, filed on Apr. 4, 2002.
(60) Provisional application No. 60/370,485, filed on Apr. 5, 2002.

(30) Foreign Application Priority Data
Apr. 4, 2002 (EP) 020763176.5
Dec. 20, 2002 (EP) 02080481.1

(51) Int. Cl. A61K 48/00; A61K 9/127

(52) U.S. Cl. 514/44; 424/450

(57) ABSTRACT

The invention is directed to delivery vehicles for delivering a substance of interest to a predetermined site. The delivery vehicle includes a substance and means for inducing availability of at least one compartment of the delivery vehicle toward the exterior, thus, allowing access of the substance to the exterior of the delivery vehicle at the predetermined site. The invention is further directed to uses of the delivery vehicle and methods for preparing the delivery vehicle.

### CROSS-REFERENCE TO RELATED APPLICATIONS

This application is a continuation of International Application No. PCT/NL03/00256, filed Apr. 4, 2003, designating the United States of America, corresponding to International Publication No. WO 03/084508 (published in English on Oct. 16, 2003), which claims priority to European Application No. 02076316.5 filed Apr. 4, 2002; U.S. Provisional Application No. 60/369,927 filed Apr. 4, 2002; U.S. Provisional Application No. 60/370,485 filed Apr. 5, 2002; and European Application No. 02080481.1 filed Dec. 20, 2002; the contents of the entirety of which are incorporated herein by this reference.

### TECHNICAL FIELD

The invention relates generally to the field of biotechnology and, more particularly, to the field of delivery of substances. The invention is useful in several areas including the fields of health, medicine, agriculture and cosmetics.

### BACKGROUND

Many substances are delivered to sites of interest to allow their action at the site. Applications of substance delivery vary widely. Examples of such applications range from pharmacological delivery of drugs, to the delivery of substances for pest control and plant growth, and for cosmetic reasons. Delivery of substances to sites of interest is so entangled in our daily practice that it is almost impossible to give a complete listing of all possible applications.

A lot of substances have properties that are far from ideal. Substances are therefore typically not delivered in an essentially pure form. Typically, substances are formulated in compositions. Compositions are formulated, for instance, to allow for appropriate dosage, delivery or application of the substance of interest.

Various problems with the use of substances can be overcome by appropriately formulating the composition containing the substance of interest. However, with the advent of technology, more and more sophisticated applications of drug delivery come into focus.

The present invention provides means and methods for broadening the use of substances even further. The present invention allows control over the availability of the substance at the site of interest. Through this control another level of accuracy and predictability is introduced which can be utilized to increase the utility of substances to be delivered and to increase the number of substances that can be used for a certain application. The problems and solutions provided by the present invention will be exemplified predominantly using medical and general health examples. However, the invention is by no means limited to these fields.

In the medical field, delivery is typically used to provide an individual with a drug or a precursor thereof. Many drugs, either in the clinic or in development, have properties which limit their applicability. They may have poor solubility, rapid metabolism, instability under physiological conditions or unfavorable biodistribution leading to, for instance, toxicities. One attempt at achieving a solution to these problems has been to associate the drugs with a variety of drug carriers such as liposomes. Liposomes, i.e., phospholipid membrane vesicles, have the ability to function as carriers for either water-soluble or lipid-soluble drugs. In a liposomal formulation, a drug is typically slowly released from the liposome over several hours to several days (Allen et al., Cancer Research 1992; 52:2431-9). In these cases, the formulation typically is used as a means for obtaining slow release of the substance. Though this may be of great help in some instances, it is still not optimal. Though slow release formulations may help to provide a more or less continuous source of substance to be made bioavailable, they do nothing with respect to making the substance available at the site and/or time where and when the action of the drug is desired.

On the other hand there are many methods for delivering drugs of interest to a predetermined site. A problem with current methods is that it is difficult to control the availability of a compound once delivered. Control over availability is desired for various reasons, for instance, in cases when the compound is toxic, degraded or otherwise rapidly removed once available. The present invention provides a solution to problems encountered with delivering substances in that it allows control of availability of the substance at the pre-determined site. To this end, the invention provides a delivery vehicle for delivering a substance of interest to a predetermined site, the vehicle comprising the substance and a means for inducing availability of at least one compartment of the vehicle toward the exterior, thereby allowing access of the substance to the exterior of the vehicle at the predetermined site. Through the capability of inducing availability of at least one compartment of the vehicle at the predetermined site, it is possible to control the availability of the substance at the site where activity of the substance is desired. With a method of the invention, it is possible to at least concentrate the substance in the area of the predetermined site. If the substances are also toxic, the toxic effects can be restricted to the predetermined site, thereby mitigating the systemic toxicity of the substance. A vehicle of the invention not only allows availability of the substance at the predetermined site, it also allows for control over the availability. Through the means for inducing availability of at least one compartment of the vehicle toward the exterior of the vehicle, it is possible to at least change the availability of the substance at the predetermined site after the vehicle has been delivered to the predetermined site. The substance may be transported from one predetermined site to another site, for instance, by a means of fluid transport in the body. Such transport will generally also encompass dilution of the substance in at least the transport fluid, thereby lowering the effective concentration of the substance and thereby rendering the substance less effective at the other sites where its presence is not desired.

### SUMMARY OF THE INVENTION

Induction of Availability

Induction of availability of at least one compartment at the predetermined site can be achieved in various ways, depending on the nature of the vehicle and the nature of the induction means. Various vehicle formulations are detailed below. Non-limiting examples of vehicles and induction means that can advantageously be used for the present invention are, for instance, various gel formulations, wherein the gel comprises a means for at least in part inducing fluidization of the gel at the predetermined site. Fluidization (gel-to-sol transition) can be induced, for instance, under the influence of a specific pH, salt concentration, temperature and/or radiation (e.g., light or sound) at the predetermined site. In this way, by formulating the substance to be delivered in a gel with particular properties, the availability of the substance can be controlled by internal and/or external environmental conditions and/or stimuli, which will be discussed in more detail below. Gels of a various nature can be used in this respect. Gelators or gelling agents are low molecular weight compounds that can gelate or thicken organic solvents or water. Gelation or thickening occurs by means of self-assembly of these gelator molecules through non-covalent interactions such as hydrophobic interactions, π-π interactions, electronic interactions, hydrogen bonding or combinations thereof (F. M. Menger, K. L. Caran, J. Am. Chem. Soc., 2000, 122, 11679; J. H. Jung, M. Amaike, K. Nakashima, S. Shinkai, J. Chem. Soc., Perkin Trans. 2, 2001, 1938; J. van Esch, F. Schoonbeek, M. de Loos, M. Veen, R. M. Kellogg, B. L. Feringa, NATO ASI Series, Kluwer Academic Publishers, 527 (1998) 223; P. Terech, R. G. Weiss, Chem. Rev., 97 (1997) 3133).

In a preferred embodiment the means for inducing availability of the compartment involves response to conditions at the site of interest. For instance, response to the specific pH, salt concentration, chemical substance, radiation (such as light, (ultra)sound, magnetic or nuclear radiation) or temperature at the site of interest allows one to control availability of the substance of interest. In another example, the inducing means comprises a light-sensitive compound that, upon exposure to light, undergoes a change in conformation, thereby allowing availability of the compartment, for instance, by enhancing fluidization of the gel. The control over the specific light at the predetermined site allows control over the availability of the substance at the site of interest. In yet another embodiment, the availability of the compartment is induced by means of the application of an electrical field at the predetermined site. The stimulus (or signal) can have a direct effect on the gelating compound, but also indirect effects are envisaged. These would consist of a signal/receptor system where the gel comprises a “receptor” that is stimulated by the signal and causes the gel to release the encompassed substance. Such a signal-receptor combination can comprise radiation as a signal in combination with a radiation-sensitive receptor. Examples of receptors in this embodiment are iron-oxide or cobalt alloys. These receptors are sensitive to various kinds of radiation and utilize the energy contained in the radiation to induce availability of the substance. Iron-oxide and cobalt alloys are particularly suited to raise the temperature in the vehicle of the invention as a result of the adsorption of radiation. Increase in heat can be used, for instance, to fluidize at least part of the vehicle at the predetermined site. Radiation can be provided to a predetermined site in a sufficiently specific fashion to allow preferential induction of availability of the compartment at or near the predetermined site. The mentioned examples are far from exhaustive and serve only to illustrate some preferred embodiments of vehicles of the invention.

It is also possible that (inducible) transition from sol to gel is used to take up compounds at a specific site. This phenomenon can, of course, be used in vitro to load a gel before it is used as a delivery vehicle, but it can also be used as main application of the vehicle in therapy or other fields in vivo or in vitro. Specific applications are the removal of unwanted (e.g., toxic) compounds from a solution, removal of skin compounds in topical applications, use in artificial kidneys, and the like. The advantage of such a system is that the compound which needs to be removed is caught in the gel that has formed and is not further released in the system from where it has been removed.

A particular embodiment in both the delivery and the catching gel systems is the use of reversible gels, which given the right stimuli, can switch from gel to sol and vice versa. This would enable reuse of the gelating compound and/or use in closed circuits in which the gelating compound acts as a transport vehicle to entrap substances at one side of the system and deliver these substances at another side of the system.

For the present invention, induction of availability of the compartment is alternatively achieved by inducing opening of at least one compartment towards the exterior of the vehicle. Inducing opening can be achieved using various means, for instance, by inducing the generation of a physical opening in a compartment of the vehicle. This embodiment will be discussed in more detail elsewhere in this document.

Induction of availability and/or opening of a compartment toward the exterior is useful for allowing the entrapped substance to passage out of the vehicle. However, it is also useful for allowing compounds to enter the vehicle and associate with the substance. In this way, vehicles of the invention can be used to take up a desired compound at the predetermined site and discontinue its availability at the predetermined site. Thus, vehicles of the invention may make the substance specifically available to the predetermined site by allowing passage of the substance outside the vehicle at that site or it may specifically allow uptake of compounds at the predetermined site by inducing availability of the substance at the site. Preferably, the induction allows passage of the substance to the exterior of the vehicle.

An inducing means of the invention comprises both an effector capable of making the compartment available and a signal with which the effector can be switched (activated) into a situation wherein the compartment is available to the exterior of the vehicle. It is clear that an effector must be able to respond directly or indirectly to the provision of a signal. The response can be any type of response that makes the compartment available. For instance, it may be that indeed a physical opening of a compartment of the vehicle is induced. However, it is also possible that the effector induces availability in a different way, for instance, by fluidization of (a compartment of) the vehicle.

Suitable Effectors

Suitable effectors allow for induction of availability of the compartment in response to a signal. In a preferred embodiment, an effector of the invention comprises a radiation-responsive molecule. Preferably, the radiation-sensitive molecule comprises a light-responsive molecule. A light-responsive molecule of the invention is a molecule that can assume a different conformation upon exposure to light. The difference in conformation is utilized to allow a physical change in the vehicle of the invention wherein the physical change induces the availability of at least one compartment of the vehicle toward the exterior. Preferred radiation-sensitive effectors are light-switchable gelling or thickening molecules and light-switchable molecules that are part of a film. Non-limiting examples of the latter are light-switchable lipids and light-switchable channel proteins.

In another embodiment of the invention, an effector comprises a binding molecule that, upon binding, induces availability of the compartment toward the exterior of the vehicle, preferably to induce release of the substance from the vehicle (for Instance, by fluidization). The signal in this case can be the binding event. A binding molecule as an effector may also induce the prolonged presence of the vehicle at the predetermined site thereby inducing availability by conditions at the predetermined site, for instance, a lower pH at the site of a solid tumor. A non-limiting example of such a binding molecule is a binding molecule that undergoes a change in conformation under the influence of conditions at the predetermined site wherein the conformation change allows preferential binding of the binding molecule to its binding partner at the predetermined site. An example of such a binding molecule is a pH-sensitive binding molecule. In a preferred embodiment, the pH-sensitive binding molecule comprises a pH-sensitive variant of the carbohydrate binding domains of the AcmA and/or AcmD protein of Lactococcus lactis. The design of a vehicle of the invention can be tailored to accommodate to a specific pH at which the vehicle is retained at the site of interest. One way of tailoring the vehicle is by manipulating the ratio of AcmA and AcmD in the vehicle. For examples of suitable AcmA and AcmD proteins, reference is made to the Examples, to Table A and to WO 99/25836 and WO 02/101026.

In a preferred embodiment of the invention, a compartment is induced to become available by inducing opening of at least one compartment in the vehicle, thereby allowing access of the substance to the exterior of the vehicle. In this preferred embodiment of the invention, the vehicle comprises a film wherein the continuity of the film (“open” or “closed” state) can be controlled by providing a signal. Induction of the open state is preferably achieved by inducing opening of a pore in the film. Therefore, in this preferred embodiment, the film comprises an effector molecule capable of forming a pore. To this end, it is preferred that the effector molecule comprises a proteinaceous channel that allows availability by forming a pore through which the compartment is made available towards the exterior of the vehicle. The proteinaceous channel can be any proteinaceous channel that allows induced opening of at least one compartment of the vehicle. Preferably, the proteinaceous channel comprises a solute channel. A solute channel is capable of allowing passage of ions and small molecules, preferably hydrophilic or amphipathic molecules. Preferably, the proteinaceous channel comprises an ion channel. More preferably, the proteinaceous channel comprises a mechanosensitive channel, preferably one of large conductance (MscL) or a functional equivalent thereof. In nature, MscL allows bacteria to rapidly adapt to a sudden change in environmental conditions such as osmolarity. The MscL channel opens in response to increases in membrane tension, which allows for the efflux of cytoplasmic constituents. By allowing passage of the constituents to the outside of the prokaryote, it is able to reduce the damage that the sudden change in environmental conditions would have otherwise inflicted. The genes encoding MscL homologues from various prokaryotes are cloned (P. C. Moe, P. Blount and C. Kung (1998) Mol. Microbiol. 28, 583-592). Nucleic acid and amino acid sequences are available and have been used to obtain heterologous (over)-expression of several MscL proteins (P. C. Moe, P. Blount and C. Kung (1998) Mol. Microbiol. 28, 583-592). Certain applications require MscL channels with specific characteristics. For this, it is possible to use mutants or chemically modified MscL channels of E. coli. Alternatively, homologues of mechanosensitive channels from other organisms could be used. A useful MscL homologue can be found in Lactococcus lactis. An additional advantage of this system is the significantly higher overexpression of the channel protein and the MscL channel protein originates and is overexpressed in a GRAS organism.

In the present invention, vehicles, preferably liposomes, comprising MscL or a functional equivalent thereof, are loaded with small molecules. These loaded small molecules can be released from the vehicle under activation or opening of the channel. Loading of the lipid vesicle can be accomplished in many ways as long as the small molecules are dissolved in a solvent which is separated from the surrounding solvent by a lipid bilayer. Activation of the MscL channel protein has been found to be controllable. It is possible to tune the type and relative amount of lipids in the vehicle such that the amount of membrane tension required to activate the channel is altered. Thus, depending on the circumstances near target cells of selected tissue, the lipid vehicle can be tuned to allow preferential activation of the channel and thus preferential release of the small molecule in the vicinity of the cells of the tissue.

Compositions comprising lipid vehicles have been used in vivo, for instance, to enable delivery of nucleic acid or anti-tumor drugs to cells. It has been observed that bloodstream administration of such vehicles often leads to uptake of vehicles by cells. Uptake by cells seems to correlate with the charge of the lipid in the vehicle. Uptake is particularly a problem with negatively charged lipid vehicles: these vehicles are very quickly removed from the bloodstream by the mononuclear phagocytic system in the liver and the spleen. Although the present invention may be used to facilitate uptake of small molecules by cells, it is preferred that the small molecules are delivered to the outside of cells. In the present invention, it has been found that MscL is also active in lipid vehicles that consist of positively and/or neutrally charged lipids. Lipid vehicles comprising positively and/or neutrally charged lipids are more resistant to uptake by cells of the mononuclear phagocytic system. Lipid vehicles of the invention, therefore, preferably comprise positively and/or neutrally charged lipids. Such vehicles exhibit improved half-lives in the bloodstream. Such vehicles also demonstrate improved targeting to non-mononuclear phagocytic system cells. The lipid part directed towards the exterior of a lipid vehicle of the invention preferably consists predominantly of positively and/or neutrally charged lipids, thereby postponing or nearly completely avoiding cellular uptake through negatively charged lipids and thereby further increasing the bloodstream half life of lipid vehicles of the invention. Apart from increasing the half-life of the vehicle in the bloodstream, positively and/or neutrally charged lipids can also be used to alter the amount of added pressure needed to activate the channel in the vehicle. This results from changes in the lateral pressure in the membrane due to changes in the attractive/repulsive forces among the lipid head groups.

The signal or event leading to activation of a channel of the invention can also be changed by altering the MscL in the vehicle. Besides the pH-sensitive mutants, other mutants are available that have a higher open probability as compared to the wild-type MscL from Escherichia coli (P. Blount, S. I. Sukharev, M. J. Schroeder, S. K. Nagle and C. Kung (1996) Proc. Natl. Acad. Sci. U.S.A. 93, 11652-11657; X. Ou, P. Blount, R. J. Hoffman and C. Kung (1998) Proc. Natl. Acad. Sci. U.S.A. 95, 11471-11475). This property can be used to tune the activation potential of the channel in a method or vehicle of the invention. For instance, it is known that in tumors the pH is very often considerably lower than in the normal tissue surrounding the tumor. Other areas in the body that have a lowered pH are the liver, areas of inflammation and ischemic areas. A lower pH can be used as a trigger for activation of the MscL in a vehicle of the invention. Mutant MscLs are available that activate (open) in response to a pH that is frequently encountered in tumors. One non-limiting example of such a pH-sensitive mutant is the G22H mutant. It was previously shown that substitution of a residue that resides within the channel pore constriction, Gly-22, with all other 19 amino acids affects channel gating according to the hydrophobicity of the substitution (K. Yoshimura, et al., 1999, Biophys. J. 77:1960-1972). One mutant (G22H, in which the glycine residue was replaced by a histidine residue) was of particular interest for clinical applications since it exhibited a significantly higher open probability at pH 6.0 compared to pH 7.5. This MscL-mutant G22H is an interesting candidate to deliver drugs at target sites with a lowered pH value, such as in solid tumors or at sites of inflammation. Supposedly, a decrease of the pH from 7.5 to 6.0 shifts the equilibrium from the unprotonated to the protonated state of the imidazole side chain of the MscL-mutant G22H. This protonation results in the introduction of a charge at amino acid position 22 and thereby affects the opening of the MscL channel.

In the Examples, it is shown that another MscL mutant, G22C, can be overexpressed and purified to high enough yield to be applicable in a drug delivery vehicle. Additionally, this mutant allows the specific attachment of an MTS (methanothiosulfonate) compound, thereby introducing a charge and consequently releasing the substance from the liposomes (FIG. 29). In the Examples, the chemical synthesis of compounds, reactive specifically with cysteine at amino acid position 22, which introduce chemical groups responsive to pH or light, thereby effecting the local hydrophobicity at the pore constriction and thus affect the gating of the channel protein will be shown.

The crystal structure of the closed form of MscL from Mycobacterium tuberculosis is used as a starting point for modeling the gating mechanism of E. coli MscL (S. Sukharev et al. (2001), Nature 409 (8):720-724). In this model, the gating consists of two steps. The first transmembrane domain forms the first gate from the closed to the closed-expanded conformation. The second gate, from the closed-expanded to the open conformation, is formed by the S1 bundle, which includes the amino acid residues 2 to 13 from each subunit. It is necessary to have the closed-expanded conformation of MscL as template for generating pH-sensitive mutants in the S1 region. Therefore, the G22S mutant of MscL, which exhibits a lower growth rate as well as a lower pressure required for gating compared to WT, was used as a template.

In a preferred embodiment of a method or composition of the invention, an MscL mutant allows preferred release of the small molecule in the target tissue. A small molecule can be any molecule small enough to pass through the pore of a channel of the invention. This molecule can be hydrophilic, amphiphilic or hydrophobic, whereas the use of hydrophilic compounds is preferable because these can easily be dissolved in an aqueous solution and will not stick to the lipid bilayer of the delivery vehicle. Preferably, the small molecule comprises a diameter of no more than 60 Å, more preferably no more than 50 Å and, still more preferably, no more than 40 Å. Particularly, peptides are a preferred substance of interest for the present invention. The present invention provides a method for obtaining controlled release of (hydrophilic) drugs from liposomes. For practical reasons, either calcein release or ion fluxes are monitored, as shown in the Examples, to functionally characterize the delivery system. It is further shown that the observed principles in these Examples also apply to therapeutically relevant hydrophilic molecules. Additionally, the applied filter-binding assay (in Example 1-I) can be used to test the controlled release of many different substances from these delivery vehicles.

Peptides typically have very poor pharmacodynamic properties when injected into the bloodstream. With the present invention, it is possible to significantly increase the half-life of peptides in the circulation. Moreover, by enabling controlled release of a small molecule with a vehicle of the invention it is also possible to have a relatively high bioavailability of the peptide at the predetermined site, whereas systemically, the bioavailability is low or even absent. This also allows for the therapeutic use of molecules that are otherwise too toxic when bioavailable systemically.

There are very likely many substances that can cause activation of the MscL channel. One example in this context is a group of compounds that are capable of associating with MscL-mutant G22C (K. Yoshimura, et al., 2001, Biophys. J. 80:2198-2206). Attachment of these reagents that are positively charged ((2-(Trimethylammonium)ethyl)methanethiosulfonate) (MTSET) and (2-aminoethyl methanethiosulfonate) (MTSEA) or negatively charged (sodium (2-sulfonatoethyl)methane thiosulfonate) (MTSES) to the cysteine under patch clamp causes MscL to gate spontaneously, even when no tension is applied (K. Yoshimura, et al., 2001, Biophys. J. 80:2198-2206). These results indicate that chemically charging the pore constriction at amino acid position 22 opens the MscL channel. In literature, experiments were performed on spheroplasts containing the MscL-mutant G22C in its natural environment, including a wide variety of lipidic and proteinaceous molecules. It is relevant to show that the methanethiosulfonate compounds attach specifically to the MscL mutant and this charged-induced gating occurs in artificial lipid membranes without the involvement of other cellular or membrane components.

Induction of availability of a small molecule by means of a proteinaceous channel in a vehicle of the invention can further be achieved in many ways. For instance, by tuning of the composition of the lipid vehicle and/or the use of a mutant MscL, it is possible to control how and where release of the small molecule will occur. In a preferred embodiment, activation of the channel is triggered upon the availability of a signal. The signal for activation can, for instance, be exposure of the vehicle to a certain pH, to light or to a certain temperature. Exposure to the signal can directly or indirectly (through an intermediary signal) lead to the activation of the channel. Preferably, the signal comprises light. Upon exposure to light, a photo-reactive lipid will alter its chain conformation, which induces a change in lateral pressure in the membrane to control the gating of the MscL channel.

The basic components of such a drug delivery vehicle are a lipid membrane and the MscL channel protein. Controlled release of a drug from these vehicles can either be achieved by directly effecting the gating mechanism of the channel protein or indirectly by effecting the physical properties of the lipid bilayer, which subsequently controls the gating of the channel. The Examples show the synthesis of photo-reactive lipids, that when incorporated in liposomes, can affect the lateral pressure in these membranes, thereby controlling the gating of the MscL channel protein. For example, there are hydrophobic compounds such as azobenzene phospholipids and related compounds available (X. Song, J. Perlstein and D. G. Whitten (1997) J. Am. Chem. Soc. 119, 9144-9159) that mix with the lipids in the vehicle and that upon exposure to light, undergo a structural change such that the gating of the MscL channel can be controlled.

In addition to activation via photo-active lipids, photo-reactive compounds can be designed to react with the MscL mutant, G22C, and respond to the absorption of light by changing the local charge or hydrophobicity. An example of such a photo-reactive molecule is 4-{2-(5-(2-Bromo-acetyl)-2-methyl-thiophen-3-yl)-cyclopent-1-enyl}-5-methyl-thiophene-2-carb oxylicacid (DTCP1), which was designed and synthesized to reversibly switch conformation after light absorption of specific wavelengths (FIG. 10). The experimental results show that we have synthesized this molecule and that it can be conjugated to a specific site in the MscL channel, known to alter the gating properties of the channel, while maintaining the desired photo-chemical properties. The DTCP1 molecule (Example I-B1) contains free carboxylic groups which modify the hydrophobicity of the pore of MscL. To enhance the hydrophilic properties of the synthesized molecule, a spiropyran derivative SP1 (FIG. 15) was prepared, which after UV irradiation, changes into a highly charged merocyanine form.

Activation through light is just one example of an embodiment wherein opening/activafion of the channel can be induced by a signal. An alteration in the redox-potential is another non-limiting example. MscL can be made sensitive to the local redox-potential after conjugation of a redox-sensitive molecule, such as a nicotinamide adenine dinucleotide derivative, to a specific site of the MscL protein. Such a redox-sensitive MscL can be (de)activated by changing the redox-potential of the environment.

Recognition of only the open conformation of MscL by a binding molecule is another non-limiting example of an embodiment that gating of the channel can be induced by another signal other than membrane tension. Such a binding molecule is preferably an antibody. The binding molecule can, for instance, be used to preferentially increase the open probability of the channel near target cells. A binding molecule capable of binding MscL in the open state is a preferred embodiment of an effector molecule that, upon binding, induces the vehicle to make the substance available for the exterior at the predetermined site.

Delivery of a substance from liposomes can also be accomplished through an enzymatically cleavable activating mechanism of the MscL. Trypsin as well as chymotrypsin are capable of cleaving MscL and thereby decreasing the gating threshold tension of MscL (B. Ajouz et al. (2000) J. Biol. Chem. 275 (2):1015-1022). Instead of trypsin or chymotrypsin, the tumor-associated protease plasmin can alternatively be used to induce MscL-mediated drug release specifically at the target site. The MscL protein does not naturally have a cleavage site for plasmin. To achieve a functional plasmin-cleavable MscL, such a plasmin site should be engineered into the MscL molecule, preferably in the extracellular part at or near the place of the natural trypsin cleavage site. Plasmin is a serine protease-like trypsin and is present in elevated levels at the tumor target site. The plasminogen activator urokinase-type (uPA) converts inactive plasminogen to active plasmin (E. A. Baker et al. (2000) J. Clin. Pathol. 53:307-312). Because plasmin is a broad-spectrum protease, it degrades most proteins within the extracellular matrix surrounding the tumor cells and thereby plays a central role in tumor cell migration and invasion. The second plasminogen activator, tissue-type (tPA), differs in biological function and distribution and is important in fibrinolysis. Plasmin has already been shown to be an interesting target for activating doxorubicin and paclitaxel prodrugs (F. M. H. de Groot et al. (2002) Mol. Cancer Therapeutics 1:901-911, and E. W. P. Damen et al. (2002) Bioorg. Med. Chem. 10:71-77).

For its application, it is important to establish a relatively simple procedure to produce and formulate the delivery vehicles. In the Examples, it is shown that mixing of synthetic lipids, detergent, detergent-solubilized MscL channel protein, and the substance that needs to be delivered, followed by detergent removal, results in a functional controllable drug delivery vehicle. Additionally, depending on the clinical application, specific lipid compositions of the liposomal drug delivery vehicle can be required. It is further shown that the MscL-mediated controlled release of a substance can be achieved in sterically stabilized liposomes.

Another example of a signal that triggers activation of an MscL is local anesthetics (B. Martinac, J. Adler and C. Kung (1990) Nature 348, 261-263). Local anesthetics most probably work to activate the channel through their incorporation in the lipid bilayer, which changes the bilayer properties. In a preferred embodiment, a vehicle of the invention comprises an asymmetrical bilayer. An asymmetrical bilayer is yet another example of a method to tune the lipid vehicle such that the activation of the channel is altered. It seems that the force gating MscL is from the lipid bilayer and amphipaths probably generate this force by differential insertion into the two leaflets (B. Martinac, J. Adler and C. Kung (1990) Nature 348, 261-263). In a preferred embodiment, a signal required for activation is provided through an intermediate. The intermediate is here capable of transforming the given signal into a pressure signal thereby allowing, if sufficient, the opening of the channel.

In one aspect, the invention provides a composition comprising a lipid vehicle comprising a proteinaceous channel and a small hydrophilic molecule, wherein the lipid vehicle and/or the proteinaceous channel is formulated such that the proteinaceous channel is in the open state in the vicinity of a target cell. Preferably, the proteinaceous channel comprises an MscL or functional part, derivative and/or analogue thereof. In a preferred aspect, the invention provides a composition comprising a lipid vehicle comprising an MscL or functional part, derivative and/or analogue thereof, wherein the composition is formulated and prepared for use in a human. Preferably, the lipid vehicle comprises a small hydrophilic molecule capable of passing through an activated MscL. Preferably, the composition is used in the preparation of a medicament. Preferably the small molecule is intended to be delivered to the outside of a cell in the tissue. A composition as described is, of course, ideally suited to be used in a method of the invention. Preferably, the MscL is a mutant MscL or a functional part, derivative and/or analogue thereof. A functional part of MscL comprises at least the region that in E. coli comprises residue 4 to 110 (P. Blount, S. I. Sukharev, M. J. Schroeder, S. K. Nagle and C. Kung (1996) Proc. Natl. Acad. Sci. U.S.A. 93, 11652-11657). It is possible to generate MscL proteins that comprise amino-acid substitution(s), insertion(s) and/or deletion(s) compared to the protein found in bacteria. Such derivatives can, of course, also be used for the present invention provided that the derivative is functional, i.e., it comprises the channel activity in kind, but not necessarily in amount. The channel activity may, as will be apparent from the description, be inducible by means other than pressure. With “activity in kind” is meant the capability of the channel protein to allow passage of a hydrophilic substance from one side of the lipid obstruction to the other. The amount of activity, both in the amount of small molecules that may pass per time unit or the size of the pore through which the small molecule can pass may differ. A derivative of MscL is also an MscL that comprises more or less or different (post-translational) modifications as compared to the native protein. Other options with mutant or derivative channels would be using MscL with genetically engineered changes in the outside loop, like receptor-recognizing domains (e.g., RGD) that, upon binding with the receptor, undergo conformation changes that induce opening of the channel. One may also add (part of) an antibody to that loop that induces channel opening after ligand binding. An MscL analogue is a molecule comprising the same activity in kind to allow passage of hydrophilic molecules through a lipid obstruction other than MscL itself, but not necessarily in amount.

In another aspect, the invention provides a method for generating a vehicle for delivery of a small hydrophilic molecule to a cell, the method comprising generating in an aqueous fluid a lipid vehicle comprising a proteinaceous channel, the vehicle formulated such that the proteinaceous channel is in the open state in the vicinity of the cell. Preferably, the proteinaceous channel assumes the open state upon the presence of a signal in the vicinity of the cell. More preferably, the lipid vehicle further comprises the small molecule. A method for the generation of a vehicle as described above can be advantageously used to generate a composition of the invention.

In another aspect, the invention provides the use of a lipid vehicle comprising an MscL for controlling delivery of a small hydrophilic molecule to a target tissue in a body. A lipid vehicle of the invention may be used to deliver a small molecule to any part of the body. However, it is preferably used for delivery to tissue with permeable endothelium such as the liver, the spleen, areas of inflammation, or tumor-bearing tissues.

A lipid vehicle of the invention can comprise lipids but may also comprise other molecules. Glycolipids or lipids modified in other ways that maintain the classical bipolarity of a lipid molecule in kind, not necessarily in amount, are also called lipids in the present invention. In a preferred embodiment of the invention, the lipid vehicle comprises a liposome, more preferably a long-circulating liposome. Long-circulating liposomes are typically small (150 nm or smaller). Preferably, the long-circulating liposome comprises neutral lipids. The long-ciculating liposome preferably comprises cholesterol with either phosphatidylcholine and PEG or sphingomyelin.

Considering that a vehicle of the invention may comprise a molecule that is regarded as foreign to the human body it is preferred in these cases that the vehicle further comprises a masking group. A masking group prevents, at least in part, the immune system of the individual to which the vehicle is administered to respond to the vehicle. MscL is typically a protein foreign to the human body. It is, therefore, conceivable that the immune system of a human administered with a vehicle comprising MscL responds to MscL, either upon first administration or upon repeated administration. To allow evasion of the immune system of the host, at least in part, so-called masking groups can be attached to the outside of the vehicle of the invention. Preferably, these masking groups comprise PEG.

In a preferred embodiment, a vehicle comprising MscL comprises a gel or thickening agent. In a preferred embodiment, the gel or thickening agent comprises an anionic polymer that decondensates under the influence of an electrical field and thus results in swelling of the polymer matrix. Preferably, the anionic polymer comprises derivatives of acrylic acid, haluronic acid and other physiologically relevant anionic polymers (Y. Qiu and K. Park, 2001, Advanced Drug Delivery Reviews 53:321-339). Swelling can be achieved at field strengths of 2.5 V across a 5 μm vehicle which corresponds to a field strength of 5000 Vcm⁻¹. This is a factor of ten less than the field strength needed to induce leakage of ions through the ion channels. In a confined volume of a vehicle of the invention, the swelling is translated into a membrane stress or internal pressure sufficient to induce opening of the MscL or functional equivalent thereof, thereby allowing availability of the compartment toward the exterior of the vehicle at the predetermined site.

Besides triggered drug release (by pH, osmotic pressure and light), MscL-containing liposomes can be used for sustained drug release. The rate of drug release can be controlled by the rate of channel gating, a property that can be manipulated by genetic or chemical modification. Not only is the rate of channel gating important, but also the mere number of channel proteins per surface area of the vehicle (or film) will influence the releasing properties. In these experiments, it was found that 10 to 10 channel protein molecules per liposome of 200 nm is preferred. It is also envisaged that vehicles are prepared that contain a mixture of (mutant) channel proteins with different properties to enhance the control over the release of the contents of the vehicle.

A vehicle comprising a proteinaceous channel can also in another way make the substance of interest available at the predetermined site. In one embodiment, the vehicle further comprises a targeting means. In combination with a targeting means, a vehicle of the invention can be used to make the substance of interest available at the predetermined site. The means for inducing availability can in these cases comprise the targeting means. Targeting is preferably achieved using a binding molecule capable of binding to a target cell at the predetermined site. The binding of the targeting molecule holds the vehicle in place so that with gradual availability of the substance, the substance is still induced to become preferably available at the predetermined site. The (target) cell surface is a landscape of macromolecules, specific to the function and state of the cell. Ligands for specific macromolecules can serve as targeting agents, assuming they have specificity for the cell type and affinity that permits binding under biological conditions. Past efforts in the field of targeted therapies have utilized monoclonal antibodies or known peptide hormones as homing moieties. These approaches have met with mixed success (Scally, 1999, Eur. J. Endocrinol. 141:1-14; Farah, 1998, Crit. Rev. Eukaryot. Gene Expr. 8:321-356). More recent approaches using phage display have exploited screening of peptide libraries to identify cell-targeting peptides. This method was exploited to select a peptide for αvβ₃ integrin, which is overexpressed on many tumor cells (Koivunen, 1995, Biotechnol. 13:265-270; Pasqualini, 1997, Nat. Biotechnol. 15:542-546). Another example is specific peptide ligands for matrix metalloprotease (Koivunen, 1999, Nat. Biotechnol. 17:768-774). This is an important enzyme involved in tissue remodeling and cell migration. Its level is elevated in cancer cells, enabling invasion of surrounding tissues.

It is important to note that the cell surface receptors need not necessarily be proteins: many cell surface markers are carbohydrates. The surface of mammalian cells and also microbial cells, mainly contain carbohydrates that are associated with membrane lipids, proteins or peptide glycans. This membrane-associated, carbohydrate-rich material is referred to as glycocalix. The glycocalix is specially involved in cell processes such as cell-cell recognition and adhesion and the binding of pathogens, bacteria and viruses to their target tissue. These cell surface oligo-saccharides can serve as targets for drug delivery vehicles. In one embodiment, the targeting means comprises the cell wall-spanning (CWS) domain of the Lactococcus lactis protein PrtP or a functional part, derivative and/or analogue thereof. Preferably, the targeting means is capable of binding preferably to the target at the predetermined site. Another preferred targeting means of the invention comprises AcmA or AcmD-type protein anchors of the AcmA (SEQ ID NOS:1-3) and AcmD-type (SEQ ID NOS:4-6) carbohydrate binding domains or homologs thereof. In general, all homolog AcmA-type protein anchors (whether they are already known or will be newly derived) contain the consensus sequence as given in Table A, with a minimum of 65% similarity. The consensus sequence was derived by using the Simple Modular Architecture Research Tool (SMART;_is available at the website smart.embl-heidelberg.de/; Schultz et al. 1998, Proc. Natl. Acad. Sci. U.S.A. 95, 5857-5864; Letunic et al. 2002, Nucl. Acids Res. 30, 242-244). The protein anchor may contain one or several repeats of the consensus sequences, each separated by a spacer. This type of protein anchors can be made to bind their target preferentially in a pH-dependent fashion. A vehicle of the invention provided with AcmA is capable of inducible binding as a result of the signal pH. For examples of suitable AcmA and AcmD proteins, reference is made to the Examples, Table A, WO 99/25836, and WO 02/101026. The natural function of the AcmA-type anchors is binding to carbohydrates in the bacterial cell wall. It is likely that protein anchor homologs of other (higher) organisms also bind to carbohydrates on other cells (Example 3-A) and that combinations of homologs result in other useful binding properties (Example 3-B). Therefore, AcmA-type anchor homologs were used in mutagenesis strategies to select new variants that bind to mammalian cells (Example 3-D).

Irrespective of the nature of the targeting molecule (whether it is an AcmA-type protein anchor, a homolog thereof, or a PrtP CWS domain) and of the target, a complicating factor with respect to selectivity is that there are few, if any, cell-specific markers for a particular cell type. More common is that expression of a certain marker is elevated in a specific cell type. To overcome or avoid/minimize side effects in drug-targeting strategies, one approach might be to make the binding to the target dependent on an external signal. An example of this is the lower pH in tumor tissues (pH 6.5) compared to that in surrounding healthy tissues and bodily fluids (pH 7.4). Therefore, AcmA-type anchor homologs and the PrtP CWS domain were mutagenized (see Examples) and variants were selected that showed pH-dependent binding.

In one embodiment, availability is preferably induced upon providing the binding molecule capable of binding MscL in the open state. Preferably, the means for inducing availability comprises both the AcmA protein and the binding molecule capable of binding MscL in the open state. In another embodiment, a bi-specific antibody comprising the above-mentioned specificity for the open state and specificity for a target cell can be used to accumulate open vehicles near target cells.

In one embodiment of the invention, the target cells can be cells from a microorganism. For this embodiment, the protein anchor is the cell wall binding domain (anchor) of the major cell wall hydrolase AcmA of Lactococcus lactis (SEQ ID NOS:1-3), a Generally Recognized As Safe (GRAS) gram-positive bacterium. This protein anchor consists of three homologous repeats of 45 amino acids that contain a specific consensus sequence (Table A; patent applications WO99/25836 and WO 02/101026), separated by intervening sequences of about 30 amino acids that are highly enriched for serine, threonine and asparagine residues. This protein anchor has the ability to attach from the outside to a wide variety of gram-positive (G+) bacteria and also when it is part of a chimeric fusion protein. This trait offers the possibility to use this protein anchor in therapeutic applications as a device to target pathogenic bacteria in order to inactivate them. Inactivation may be achieved by coupling antibodies, cytokines (signaling molecules for the immune system) or drugs to the protein anchor. In another approach and in the case of the unmodified AcmA Protein Anchor of mostly G+ bacteria, the drugs or other compounds may be incorporated in nano- or micro delivery vehicles to which the protein anchor is attached in order to direct these vehicles to a specific target.

The protein anchor has many homologs (domains in other proteins that resemble the protein anchor) in a wide variety of microbes and higher organisms (Table A and patent applications WO 99/25836 and WO 02/101026). These homologs have a different binding spectrum, which is directly applicable. An example of this is given in the experimental section. In addition, new protein anchors with different binding spectra have been obtained by random mutagenesis and/or by combining the traits of the homologs using in vitro recombination (see Examples) and this resulted in anchors that showed pH-dependent binding to the target. This is particularly relevant for targeting tumor cells. Tumors are known to have a lower pH (approximately pH 6.5) than healthy tissues and bodily fluids (approximately pH 7.4).

The native AcmA Protein Anchor can be used to target gram-positive bacteria. In the Examples, it is demonstrated that homologs of the AcmA anchor can be used to extend the range of microorganisms that can be targeted. In the Examples, a reporter molecule was fused to the anchor. In some applications, anchors are coupled to the proper delivery vehicles that have the ability to make the drugs available upon induction (e.g., liposomes with MscL). The Examples demonstrate that modified AcmA-type anchors can be made in order to obtain pH-dependent binding (induced availability) to the target. Also, in these Examples, a reporter molecule is fused to the anchor and a model target is used. In some applications, the modified anchors are coupled to delivery vehicles (e.g., liposomes, hydrophobin particles, etc.).

The cell wall binding domain or anchor of the lactococcal cell wall hydrolase, AcmA, consists of three repeats of 45 amino acids (SEQ ID NOS:1-3) that show a high degree of homology (Buist et al. 1995, J. Bacteriol. 177:1554-1563). These repeats belong to a family of domains that meet the consensus criteria (Table A) as defined in patent application WO 99/25836 and can be found in various surface-located proteins in a wide variety of organisms. Another feature that most of these domains have in common is that their calculated pI values are high: approximately eight or higher (Table A). At a pH lower than 8, these binding domains are positively charged. The AcmA protein anchor (cA) homolog of the lactococcal cell wall hydrolase AcmD (cD) consists also of three repeats with a calculated pI that is much lower (approximately pI 3.8) than that of the cA domain (Table B). Consequently, the cD anchor was negatively charged at pH 4 and higher. We have demonstrated that no binding of the MSA2::cD reporter protein occurred under these conditions. Therefore, we investigated here the influence of the pH during binding of a cD fusion protein (MSA2::cD). Furthermore, we constructed a hybrid protein anchor consisting of the three cD repeats and one cA repeat that has a calculated pI value that is higher than that of the cD repeats alone. The hybrid protein anchor showed better binding at pH values above the pI of the cD repeats alone, indicating that the pH binding range of AcmA-type protein anchors can be manipulated by making use of the pI values of the individual repeats in hybrids.

Alternatively, the cell to be targeted is a mammalian cell. The protein anchor of L. lactis AcmA has many homologs (domains in other proteins that resemble the Protein Anchor) in a wide variety of microbes and higher organisms (Table A and patent applications WO 99/25836 and WO 02/101026). These homologs may have a different binding spectrum, including eukaryotic cells. New protein anchors with altered binding specificities were obtained by random mutagenesis involving error-prone PCR and/or in vitro recombination and this resulted in an anchor that is able to attach to human intestine tumor cells.

Next to the AcmA anchor, other protein anchors can be used for the present invention. A comparative in silico analysis of the amino acid sequences of known cell wall-bound extracellular serine proteinase (CEPs) from different lactic acid bacteria showed that the lactococcal PrtP has a modular, multidomain structure (Siezen et al. 1999, Antonie van Leeuwenhoek 76:139-155). The N-terminal part, with the pre-pro domain-containing signals for secretion and activation, is followed by the subtilisin-like serine proteinase domain and two large central domains that are thought to have regulatory and stabilizing functions. The C-terminal part of PrtP consists of (i) a helical spacer followed by (ii) a hydrophobic Gly/Thr/Asp-rich putative cell wall spacer (CWS) domain that can span the peptidoglycan layer, and (iii) a cell wall-anchoring domain.

Some of the bacterial cell wall-anchored proteins are known to have adhesive properties (Navarre and Schneewind, 1999, Microbiol. Mol. Biol. Rev. 63:174-229). L. lactis cells can adhere to one another via the sex factor CluA in order to allow conjugal transfer of DNA. The cell-to-cell binding causes a cell aggregation phenotype (Godon et al. 1994, Mol. Microbiol. 12:655-663). Pathogenic gram-positive bacteria carry cell wall-anchored surface proteins that contribute to virulence (Foster and McDevitt, 1994, FEMS Microbiol. Lett. 118:199-205). They do so by promoting adherence to the host cells and/or tissue components and by binding a variety of serum proteins including albumin, collagen, complement-regulatory factors, soluble forms of fibronectin and fibrinogen, and the proinflammatory plasminogen and kininogen (Patti et al. 1994, Annu. Rev. Microbiol. 48:585-617). Some streptococcal M proteins as well as fibrinogen-binding protein (FGBP) from Streptococcus equi can also mediate bacterial auto-aggregation, a property shown to be crucial for adherence and resistance to phagocytosis (Frick et al. 2000, Mol. Microbiol. 37:1232-1247; Meehan et al. 2001, Microbiology 147:3311-3322). In the experimental section, the direct involvement of the proteinase PrtP-duplicated CWS domain in adherence to a human small intestine cancer cell line is reported.

Specifically, in light of the present invention, protein anchors are used as targeting elements of delivery vehicles. They can be used in combination with these delivery vehicles to target specific human or animal cells providing induced availability of the substance of interest. This substance of interest can, for instance, be a drug or a diagnostic. In the case of a diagnostic, the delivery vehicle is preferably filled with a contrast agent for visualization of tissue. However, also in absence of drug delivery vehicles, the protein anchors as described can be used for targeted drug delivery. It is possible to directly bind drugs or prodrugs to the protein anchor either through strong covalent bonds or by using labile connections, for instance, bonds that are sensitive to pH and will disconnect on a pH change (such as is encountered in the vicinity of cancer cells). Chelators can also be coupled to the protein anchor, which, in turn, can be used to deliver radionuclids (radioactivity emitting metal ions) to a target site.

In the Examples, the chemical coupling of the Protein Anchor to liposomes and sterically stabilized liposomes is described. The liposomes contain calcein as a reporter drug. The liposomes, with the coupled protein anchor displayed on the surface were then incubated with TCA-pretreated L. lactis cells (ghost cells). After washing the ghost cells to remove unbound liposomes, binding to the ghost cells was demonstrated by measuring an increase in fluorescence in a fluorometer and microscopically by using a fluorescence microscope. Further, the reconstitution of a protein anchor derivative into liposomes is described in the Examples. As its N-terminus, the protein anchor derivative contained hydrophobic peptide sequences that enabled the efficient incorporation of this part of the fusion into the lipid bilayer of the liposomes. The liposomes with the inserted protein anchor displayed on the surface were then incubated with TCA-pretreated L. lactis cells (ghost cells). After washing the ghost cells to remove unbound liposomes, binding to the ghost cells was demonstrated in Western blots. The reconstitution of a protein anchor derivative into liposomes is also described. The protein anchor derivative contained a modified secretion signal sequence that enabled the efficient coupling in vivo of the protein anchor to the lipid bilayer of the bacterial membrane. In this way, the protein anchor was produced as a lipoprotein that was isolated and reconstituted into liposomes with the protein anchor attached. The liposomes with the coupled protein anchor displayed on the surface were then incubated with TCA-pretreated L. lactis cells (ghost cells). After washing the ghost cells to remove unbound liposomes, binding to the ghost cells was demonstrated in Western blots. In the same way, the reconstitution of another protein anchor derivative into liposomes is described. Here, the protein anchor derivative contained a defective processing site for the bacterial leader peptidase and the signal sequence functioned in this way as an efficient transmembrane- (TM-) spanning domain. This enabled the efficient incorporation of this part of the fusion protein into the lipid bilayer of the liposomes. In a subsequent experiment, the coupling of the protein anchor to a polymer particle is described. The particles contain an organogel with a reporter drug (calcein). The protein anchor was displayed on the particle surface, which was then incubated with TCA-pretreated L. lactis cells (ghost cells). After washing the ghost cells to remove unbound polymer particles, binding of calcein-loaded polymer particles to the ghost cells was demonstrated.

This way of targeted delivery also works with other delivery vehicles and was demonstrated in an experiment in which the coupling of the protein anchor PA3 to SC3 vesicles loaded with calcein is described. The SC3 vesicles with the coupled PA3 displayed on the surface were then incubated with TCA-pretreated L. lactis cells (ghost cells). After washing the ghost cells to remove unbound vesicles, binding of calcein-loaded hydrophobin vesicles to the ghost cells was demonstrated. Thus, in a preferred embodiment, a lipid vehicle of the invention further comprises a non-channel protein. Preferably, the non-channel protein is a binding molecule capable of binding to a binding partner in the tissue, thereby enabling at least a prolonged stay of the vehicle in the tissue and/or near a target cell.

The Vehicle

The vehicle can be generated using various means. For instance, the vehicle may comprise a film that forms a barrier between the interior of the vehicle and the exterior. Alternatively, the vehicle can comprise a gel which more or less traps the substance of interest in the interior. Such gels may be continuous or discontinuous. In a preferred embodiment, a vehicle of the invention comprises a gel and a film. In this way unintended leakage of the substance from continuous and discontinuous gels can at least in part be reduced. In a preferred embodiment of the invention the film comprises a membrane. The membrane preferably generates at least one compartment in the vehicle. In this embodiment, inducing means comprises means to break up or dissolve or otherwise interrupt the membrane. Creating a discontinuity in the membrane makes the compartment comprising the substance in the interior available to the exterior of the vehicle. The membrane may consist of many different substances. Preferably the membrane comprises a lipid. Preferably, the membrane comprises a lipid bilayer.

Compositions for Films

Amphiphiles

In a preferred embodiment, the membrane comprises an amphiphile. Amphiphiles are capable of self-assembly to form vehicles in a predominantly polar or predominantly apolar environment. Amphiphiles are widely used for generation of membranes. In a preferred embodiment, the amphiphile comprises a lipid, in particular a phospholipid. Vehicles comprising such films are generically called liposomes. Thus, in a preferred embodiment, a vehicle of the invention comprises a liposome or a functional equivalent thereof. Liposomes typically have a size between 50 and 2000 nm. For human use, the size is preferably between 50 and 200 nm. A functional equivalent of a liposome comprises a film comprising a lipid, in particular, a phospholipid but with a smaller size, for instance, the so-called nanosomes. Nanosomes typically have a size not exceeding 100 nm and are considered to be a functional equivalent of a liposome in the present invention. Typical liposome formulations comprise DMPC, DOPC, cholesterol, etc. (R. J. Baneijee, 2001, Biomat. Appli. 16:3-21). In a preferred embodiment, part of the lipids are conjugated with polyethylene glycol (PEG), which causes a longer circulation time in vivo.

In one embodiment of the invention, the membrane comprises a cationic amphiphile. Since the introduction of the quaternary ammonium-containing amphiphile dioleoyloxypropyl trimethyl ammonium chloride by Felgner et al. (Proc. Natl. Acad. Sci. U.S.A., 1987, Vol. 84:7413-7417), which in combination with the phospholipid dioleoylphosphatidylethanolamine (DOPE) is commercially available as Lipofectamine™, many more cationic amphiphiles have been developed and marketed. For example, cationic amphiphiles having a pyridinium group, which is an aromatic ring comprising a nitrogen atom, as cationic part for introducing biologically active compounds into eukaryotic cells, have been developed and are disclosed in EP 0755924 and reviewed in A. D. Miller, 1998, Angew. Chem. Int. Ed. Engl. 37:1768-1785. A film comprising a cationic amphiphile is, therefore, also part of the invention. In a particularly preferred embodiment, the cationic amphiphile comprises a cationic amphiphile having an aromatic ring comprising a nitrogen atom according to the following formula (I): 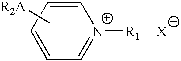
in which:
R¹ is selected from the group consisting of:
   a branched or linear (C6-C24) carbon chain optionally interrupted by one or more heteroatoms, optionally containing one or more functional groups, optionally containing one or more double or triple carbon-carbon bonds or combinations of double and triple carbon-carbon bonds, optionally being substituted or combinations thereof,
      and
R3-Ar-A-R2 in which R3 is a C2-C10 carbon chain,
   in which Ar is an aromatic or a heteroaromatic ring optionally to which relative to R3 in the ortho-, meta-, or para-position A-R2 is attached,
   A is attached to the aromatic ring in the ortho-, meta- or para-position relative to the nitrogen atom in the aromatic ring, and selected from the group consisting of CH₂, O, S, S—S, NH, OC═O, O═CO, SC═O, O═CS, NHC═O, O═CNH, CH═N, N═CH, phosphate, alkylphosphate and phosphonate,
R2 represents a branched or linear (C6-C24) carbon chain optionally interrupted by one or more heteroatoms, optionally containing one or more functional groups, optionally containing one or more double or triple carbon-carbon bonds or combinations of double and triple carbon-carbon bonds, optionally being substituted or combinations thereof, and
X⁻ is a physiologically acceptable anion.

For a more detailed description of these compounds, see WO 02/090329, which is incorporated herein by reference.

Cationic amphiphiles according to this embodiment and as specifically disclosed in WO 02/090329 are comprised of an aromatic ring to which two carbon chains (R1 and R2) are attached. The aromatic ring comprises a nitrogen atom. One of the two carbon chains (R1) is attached to the nitrogen atom in the ring. The second carbon chain (R2) is attached to the ortho-, meta- or para-position relative to this nitrogen. Both groups R1 and R2 in the formula can be identical but this is not necessary. Preferably, A is CH₂ and is attached in the para-position relative to the nitrogen atom in the aromatic ring. Preferably, A is OC═O and is attached in the para-position relative to the nitrogen atom in the aromatic ring. Also preferred is a cationic amphiphile wherein A is OC═O and is attached in the meta-position relative to the nitrogen atom in the aromatic ring. In a particularly preferred embodiment, R1 is a carbon chain selected from the group consisting of C16, C18, C20 and C22 carbon atoms, optionally containing one or more double or triple carbon-carbon bonds or combinations thereof and R2 is selected from the group consisting of C14, C16 and C18 carbon atoms, optionally containing one or more double or triple carbon-carbon bonds or combinations thereof. In yet another embodiment, A is CH₂ and is attached in the para-position relative to the nitrogen atom in the aromatic ring and R2 is R4C═OO in which R4 is a C5-C23 carbon chain. Preferably, R1 is a carbon chain selected from the group consisting of C16, C18, C20 and C22 carbon atoms, optionally containing one or more double or triple carbon-carbon bonds or combinations thereof and R2 is selected from the group consisting of C11, C13, C15 and C17 carbon atoms, optionally containing one or more double or triple carbon-carbon bonds or combinations thereof. In a particular preferred embodiment, R1 is longer than R2.

In their most elementary form, the carbon chains R1 and R2 are linear alkyl chains of 6 to 24 carbon atoms. Optionally, one of the two or both carbon chains comprise one or more unsaturations in the form of double or triple carbon-carbon bonds. Also, it is possible that the carbon chains comprise, optionally in combination with one or more unsaturations, one or more heteroatoms in the chain and/or one or more functional groups in the chain and/or substitutions on the chain. It is also possible that the carbon chain is branched. Preferably, such branching does not occur on the first six carbon atoms calculated starting from the aromatic ring. Such branching can occur in combination with the presence of unsaturated carbon-carbon bonds and also in combination with the presence of heteroatoms in the chain and/or with the presence of functional groups and/or in the presence of substitutions.

In one embodiment, the carbon chain R1 on the aromatic nitrogen atom comprises an aromatic group. In particular, the aromatic group can be a phenyl group. The aromatic group in R1, represented by Ar, is positioned near the nitrogen atom containing the aromatic ring. “Near” in this respect means that the carbon chain connecting the nitrogen-containing aromatic ring and the aromatic group in R1 is of such a length that “backfolding” of R1 towards the nitrogen atom-containing aromatic ring allows alignment of the aromatic group in R1 with the nitrogen atom-containing aromatic ring. With the aid of models, the skilled person will be able to determine an appropriate length of the chain connecting Ar and the nitrogen-containing aromatic ring for backfolding to occur. Backfolding resulting in sufficient alignment is achieved with R3 which is a C2-C10 carbon chain. Optionally attached to the aromatic group in R1 is A-R2 in the ortho-, meta- or para-position relative to R3, in which R2 is defined as above and A as will follow. Another particular embodiment is a cationic amphiphile in which Ar is a heteroaromatic ring. A heteroaromatic ring is an aromatic ring comprising one or more heteroatoms such as O, N and S. In particular, Ar is a heteroaromatic ring which comprises a nitrogen atom. R3 is attached to the nitrogen in this aromatic ring Ar. In the ortho-, meta- or para-position in the aromatic ring, A-R2 is attached.

R1 is directly attached to the nitrogen atom in the aromatic ring. R2 is attached to a carbon atom in the aromatic ring via group A. Various ways exist for attaching R2 to an aromatic carbon atom. The skilled person will be able to determine what groups are available for A. Particularly suited as group A for attachment is a group chosen from CH₂, O, S, S—S, NH, OC═O, O═CO, SC═O, O═CS, NHC═O, O═CNH, CH═N, N═CH, phosphate, alkylphosphate and phosphonate.

X⁻ represents a physiologically acceptable anion. The cationic amphiphiles of the invention can be used for in vitro as well as in vivo purposes. In this respect, it may vary what anions are physiologically acceptable. The skilled person will be able to determine for what purpose which anion may be suitable. Examples of suitable anions are Cl⁻, Br⁻, I⁻, HSO₄—, H₂PO₄—, ClO₄— and organic anions such as CH₃CO₂—, —O₂CCO₂— and the like.

As mentioned above, the R1 and R2 groups in preferred cationic amphiphiles may contain one or more unsaturated carbon-carbon bonds. Also, the R1 and R2 groups may contain, in any position, one or more heteroatoms such as O, N and S. Such a heteroatom can be part of a functional group. Thus, R1 and R2 may contain in any position, one or more functional groups, such as ethers, disulphides, esters, amides, phosphates, imines, amidines and the like. For research, diagnostic or therapeutic purposes, R1 or R2, or both, may also comprise fluorescent groups, such as fluorescein rhodamine, acridine, diphenylhexatrienepropionic acid and the like, in the chain or as substituent attached to the chain or R1 and/or R2 may comprise or be substituted with radioactive labels. Of particular interest are substituents that can be involved in targeting of cells. For instance, ligands for particular receptors on cells or antibodies or parts of antibodies comprising binding domains for a particular epitope at or in the neighborhood of the site where the incorporated biologically active compound has to exert its activity can be attached to R1 and/or R2. Such targeting substituents can be attached directly or, for instance, through a spacer. Also, functional groups and/or substituents that can be involved in the release from endosomes in cells, such as pH labile groups or substituents can be of interest.

Optionally, the carbon chains in the vicinity of the nitrogen-containing aromatic ring are substituted with groups introducing additional positive charge such as, for instance, amino groups that are protonated under physiological conditions and trialkylammonium groups. In this respect, substituents that can be involved in hydrogen bonding are also considered.

Cationic amphiphiles can be synthesized following known procedures such as described in EP 0755924 and more specifically in WO 02/090329. In short, 4-methylpyridine is treated with base and subsequently mono-alkylated on the 4-methyl group to introduce R2. Next, the nitrogen in the pyridine ring is quaternized with an alkyl halide to introduce R1 followed by ion-exchange to obtain the desired X⁻ as counter-ion. However, in order to obtain the desired compounds in acceptable yield and purity, the general known method described above is not always satisfactory. Surprisingly, it has been found that cationic amphiphiles that would not be available in an economically acceptable manner using known procedures can be synthesized by applying the microwave technique in the procedure. In particular, the attachment of R1 to the nitrogen in the aromatic ring is carried out under microwave conditions. In general, this means that the step of attaching a group on the nitrogen in the nitrogen atom-containing aromatic ring is carried out in a microwave oven. The attachment reaction is particularly a substitution of a halide in the group to be attached. Depending on the specific reaction that is carried out, the skilled person will be able to determine the optimal reaction time and settings for the microwave oven.

Preferred cationic amphiphiles for this invention are:

1-Benzyl-4-nonadecylpyridinium Chloride (SF-65):

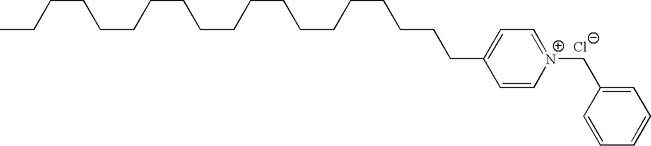

1-Benzyl-4-(cis-10′-nonadecenyl)pyridinium Chloride (SF-11):

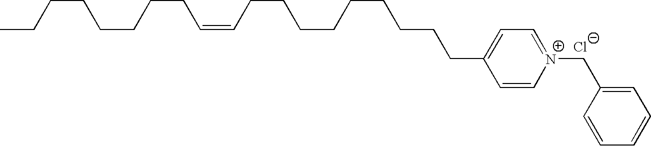

1-(6′-Phenylhexyl)-4-nonadecyl pyridinium Chloride (SF-5):

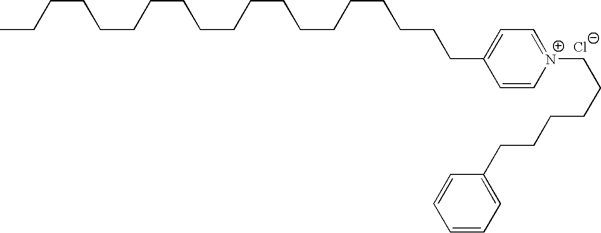

1-(6′-Phenylhexyl)-4-(cis-10″-nonadecenyl)pyridinium Bromide (SF-4):

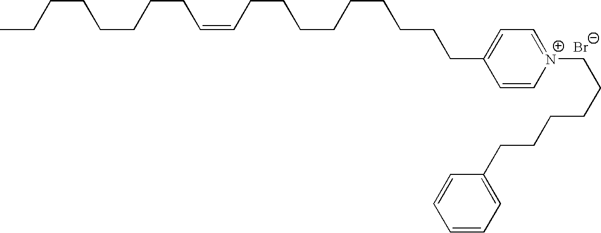

1-(4′-Phenylbutyl)-4-nonadecylpyridinium Chloride (SF-12):

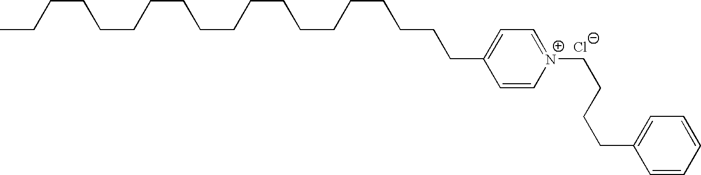

1-(4′-Phenylbutyl)-4-(cis-10″-nonadecenyl)pyridinium Chloride (SF-3):

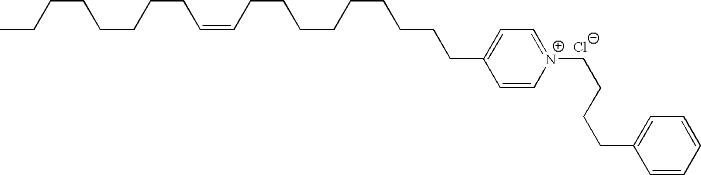

1,1′-bis-(1″,4″-butyl)-4-(cis-10-nonadecenyl)pyridinium Chloride (SF-8):

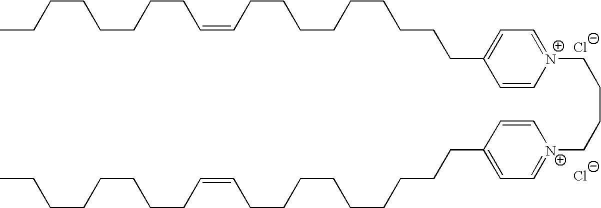

1,1′-bis-(1″,6″-hexyl)-4-(cis-10-nonadecenyl)pyridinium Chloride (SF-9):

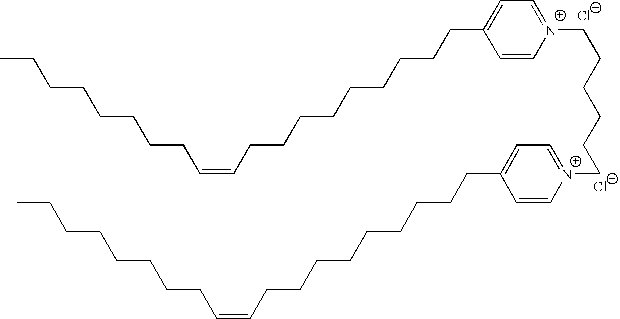

1-Methyl-4-(10′-cis-nonadecenyl)pyridinium Chloride (SF-7):

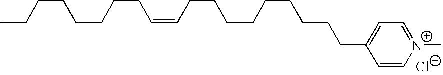

1-(9′-cis-octadecenyl)-4-(10″-cis-nonadecenyl)pyridinium Chloride (SF-6):

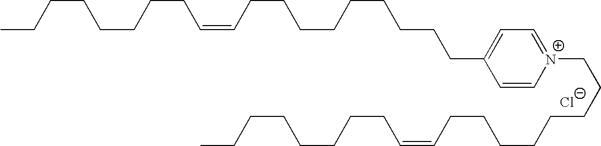

1-(9′-cis-octadecenyl)-4-heptadecyl-pyridinium Chloride (SF-45):

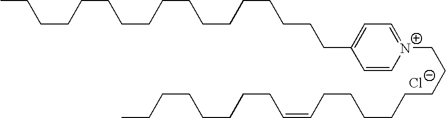

1-(9′-cis-octadecenyl)-4-propyl-pyridinium Chloride (SF-39):

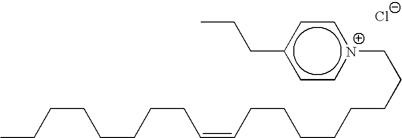

1-(9′-cis-octadecenyl)-4-pentyl-pyridinium Chloride (SF-40):

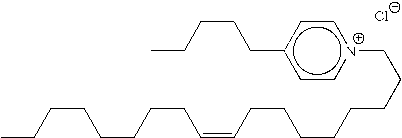

1-(9′-cis-octadecenyl)-4-heptyl-pyridinium Chloride (SF-41):

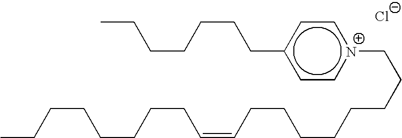

1-(9′-cis-octadecenyl)-4-nonyl-pyridinium Chloride (SF-42):

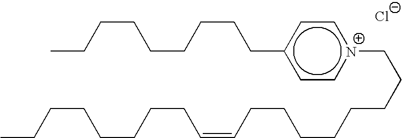

1-(9′-cis-octadecenyl)-4-undecyl-pyridinium Chloride (SF-43):

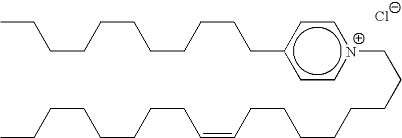

1-hexyl-4-tridecyl-pyridinium Chloride (SF-31):

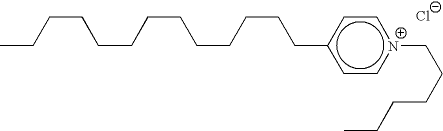

1-octyl-4-tridecyl-pyridinium Chloride (SF-32):

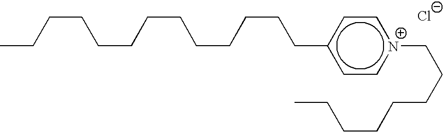

1-decyl-4-tridecyl-pyridinium Chloride (SF-33):

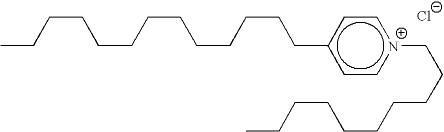

1-dodecyl-4-tridecyl-pyridinium Chloride (SF-34):

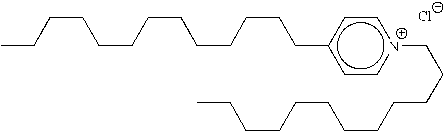

1-tetradecyl-4-tridecyl-pyridinium Chloride (SF-35):

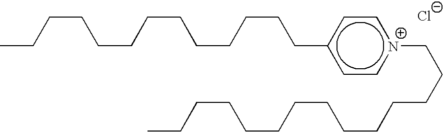

1-hexadecyl-4-tridecyl-pyridinium Chloride (SF-36):

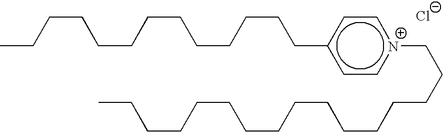

1-stearyl-4-tridecyl-pyridinium Chloride (SF-37):

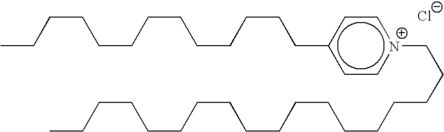

1-(9′-cis-octadecenyl)-4-tridecyl-pyridinium Chloride (SF-30):

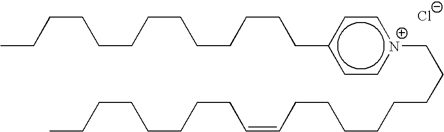

1-(9′-cis-octadecenyl)-4-pentadecyl-pyridinium Chloride (SF-44):

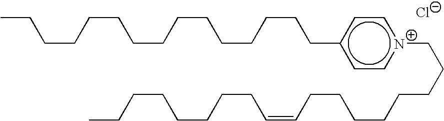

1-(9′-cis-octadecenyl)-4-heptadecyl-pyridinium Chloride (SF-45):

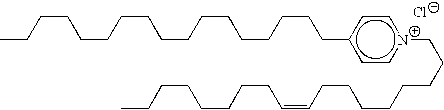

1-stearyl-4-(10′-cis-nonadecenyl)-pyridinium Chloride (SF-46):

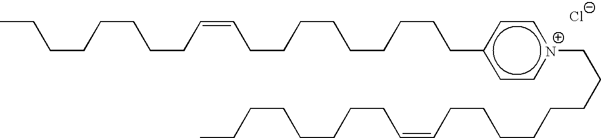

1-(9′-cis-octadecenyl)-4-nonadecyl-pyridinium Chloride (SF-14):

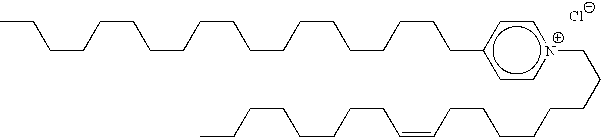

1-(9′-cis-octadecenyl)-4-methyl-pyridinium Chloride (SF-38):

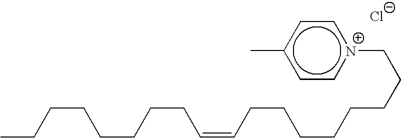

1-(9′-cis-octadecenyl)-4-(-carbo-(9″-cis-octadecenyloxy))pyridinium Chloride (SF-15):

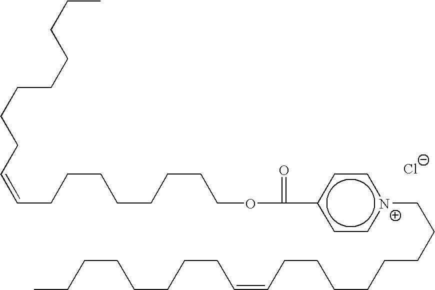

1-(9′-cis-octadecenyl)-4-(-carbo-(hexadecyloxy))pyridinium Chloride (SF-20):

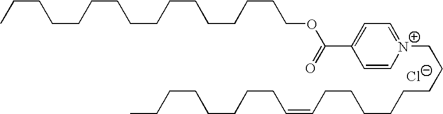

1-dodecyl-4-(-carbo-(dodecyloxy))pyridinium Chloride (SF-53):

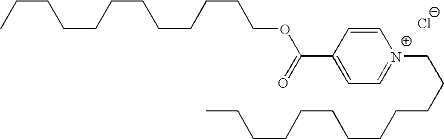

1-(9′-cis-octadecenyl)-4-(-carbo-(dodecyloxy))pyridinium Chloride (SF-55):

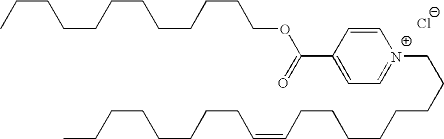

1-dodecyl-4-(-carbo-(hexadecyloxy))pyridinium Chloride (SF-47):

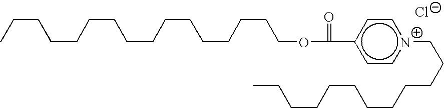

1-Methyl-4-(-carbo-(9′-cis-octadecenyloxy))pyridinium Chloride (SF-18):

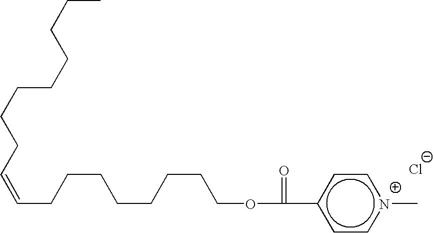

1-Methyl-4-(-carbo-(hexadecyloxy))pyridinium Chloride (SF-19):

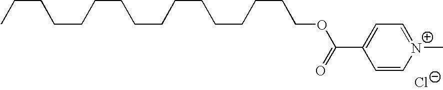

1-hexyl-4-dodecylpyridinium Chloride (SF-26):

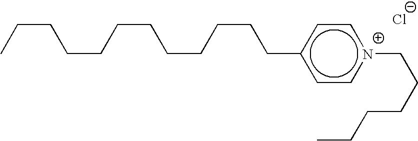

1-(9′-cis-octadecenyl)-3-(10″-cis-nonadecenyl)pyridinium Chloride (SF-63):

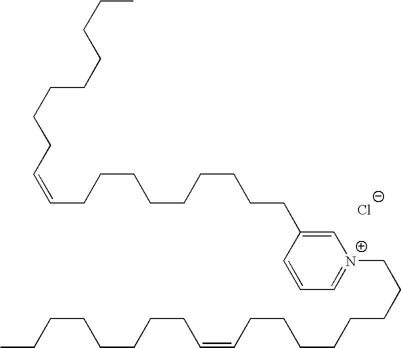

1-(polyethyleneglycol5000-ω-methylether)-4-(10′-cis-nonadecenyl)pyridinium Bromide (SF-79):

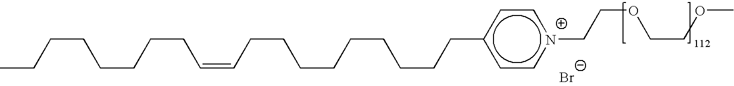

1-tetradecyl-4-pentadecylpyridinium chloride (SF-80):

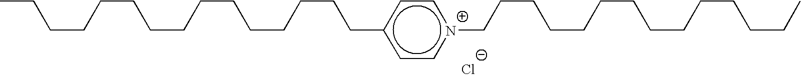

A description of the synthesis of the above-mentioned compounds can be found in WO 02/090329.

These compounds are effective molecules for in vitro delivery of nucleic acids. The possibilities for in vivo application are numerous, such as, for introducing nucleic acids into cells. Local administration can, in principle, be used for easily accessible cells, such as in the skin, eye, lung, joint and muscle. This can be used for the treatment of diseases, but, for instance, a muscle may be transformed into a bioreactor for production of secreted gene products (e.g., erythropoietin, insulin and for vaccination purposes).

For the treatment of more generalized disease, such as cancer metastases and autoimmune disease as well as for the treatment of diseased organs, such as liver, heart, spleen and kidney, invasive routes, for example, via the bloodstream, subcutaneous tissue, and peritoneum are required.

Example 5 shows in vivo gene expression after intravenous injection of DNA complexed to a cationic amphiphilic compound in the mouse.

Hydrophobin or Functional Equivalent Thereof

In another embodiment, the film comprises a hydrophobin. Hydrophobins are capable of forming tight membranous structures that are impermeable to fluid or dissolved molecules. The membranes can be very thin. In nature, this film is used, for example, to cover aerial structures of fungi, which results in a hydrophobic surface, with all the effects on the organism and its environment. The number of similar proteins is increasing rapidly and, therefore, also the diversity of specific characteristics. Hydrophobin membranes are particularly suited in vehicles that also comprise a viscous material and/or a lipid membrane. In these cases, undesired leakage of the substance from the vehicle can be prevented to a large extent. Particularly for vehicles comprising viscous material and/or lipid membranes leakage is a problem. By providing an extra layer comprising hydrophobin or a functional equivalent thereof (e.g., the rodlins), such vehicles can be made essentially non-leaky. In the present invention, hydrophobins are shown to be ideal supports for the lipids, which results in a more stable and/or less leaky liposome. Introducing hydrophobins to liposomes increases the stability and/or decreases leakage problems of liposomes. The diversity of hydrophobins and liposomes makes it possible to adjust the properties to virtually any specific application. In the Examples, the formation of liposomes stabilized with hydrophobins, the verification of the resulting compartment, and the assay used to measure the stability and leakage is described. Hydrophobins are also known to have low immunogenicity, which makes vesicles consisting of or coated with hydrophobins ideally suitable for drug delivery. In a preferred aspect of this embodiment, the film comprises an amphiphile and a hydrophobin or equivalent. Vehicles of this preferred embodiment are less prone to release of the substance of interest in the absence of induction of availability. These vehicles are less leaky, thereby allowing more control over the availability of the substance of interest at the predetermined site.

Hydrophobins are a well-defined class of proteins (described in Wessels, 1997, Adv. Microb. Physiol. 38, pp 1-45) capable of self-assembly at a hydrophobic-hydrophilic interface, and having a conserved sequence:

(SEQ ID NO:7)

X_{N}-C-X₅₋₉-C-C-X₁₁₋₃₉-C-X₈₋₂₃-C-X₅₋₉-C-C-X₆₋₁₈-C-Xₘ

wherein X represents any amino acid, and n and m represent an integer. In general, a hydrophobin has a length of up to 125 amino acids. The cysteine residues (C) in the conserved sequence are part of disulfide bridges. In the present invention, the term “hydrophobin” has a wider meaning to include functionally equivalent proteins and encompasses a group of proteins comprising the sequence or functional parts thereof

| | |
|---|---|
| Xₙ-C-X₁₋₅₀-C-X₀₋₅-C-X₁₋₁₀₀-C-X₁₋₁₀₀- | (SEQ ID NO:8) |
| C-X₁₋₅₀-C-X₀₋₅-C-X₁₋₅₀-C-Xₘ | |

still displaying the characteristics of self-assembly at a hydrophobic-hydrophilic interface resulting in a protein film. Means and methods for the manipulation, purification and the formation of films comprising hydrophobin are described or can be derived from L. G. Lugones, et al., 1998, Microbiology 144 (Pt 8):2345-2353; G. G. Martin, et al., 2000, Biomacromolecules 1:49-60; J. G. Wessels, 1997, Adv. Microb. Physiol. 38:1-45; H. A. Wösten, 2001, Annu. Rev. Microbiol. 55:625-646; H. A. B. Wösten, et al., 1993, The Plant Cell 5:1567-1574. In accordance with the definition of the present invention, self-assembly can be detected by adsorbing the protein to Teflon™ and using Circular Dichroism to establish the presence of secondary structures (in general, α-helix and β-sheet). By choosing the proper conditions, the film can be formed in different shapes and manipulated to have alternative characteristics. Examples of interfaces can be Teflon™—water, where the hydrophobic Teflon™ is coated with a hydrophilic layer, and water—oil. For a recent review, see Wösten 2001, Annu. Rev. Microbiol. 55:625-646. In the experimental part, the assembly of hydrophobins into small vesicles that can be used as delivery vehicles is described.

A possible functional equivalent of a hydrophobin is a rodlin. Rodlins are proteins with similar properties to hydrophobin but that do not share the same consensus sequence. A typical class of rodlins is described in WO 0174864.

Compositions for Gels

A vehicle of the invention may comprise a gel, either alone or in combination with a film. Any type of gel may be used for the vehicle of the present invention as long as the vehicle comprises a means for inducing availability of a compartment formed by the gel at the predetermined site. The gel may comprise low molecular weight compounds (gelators) that can gelate or thicken organic solvents or water. Preferred examples of such gels are organogels such as (a) amino acid type, b) carbohydrate derived, c) bis urea derivatives, d) bis amide derivatives, e) steroid derivatives, and f) fatty acid derivatives (J. van Esch, F. Schoonbeek, M. de Loos, M. Veen, R. M. Kellogg, B. L. Feringa, NATO ASI Series, Kluwer Academic Publishers, 527 (1998) 223; P. Terech, R. G. Weiss, Chem. Rev., 97 (1997) 3133; F. M. Menger, K. L. Caran, J. Am. Chem. Soc., 2000, 122, 11679; J. H. Jung, M. Amaike, K. Nakashima, S. Shinkai, J. Chem. Soc., Perkin Trans. 2, 2001, 1938). Preferably, the gel comprises a so-called thermally reversible gelling or thickening agent. In a preferred embodiment, a gel is formed from reversible gelling of low molecular weight compounds in solvents (typically water and/or organic solvents). These gelators are of particular interest for many technical applications. The self-assembly of these gelator molecules often occurs by means of non-covalent interactions, such as hydrophobic interaction, π-π interactions, electronic interactions, hydrogen bonding, or combinations thereof.

Amino Acid-Derived Gels

Surprisingly, it has been found that such gels can be excellently prepared from amino acids, oligopeptides or derivatives thereof. A gelling agent or thickener according to this embodiment comprises a rigid core which is functionalized with three amino acid ester or amide groups by means of an amide or urea linkage. These groups may be the same or different; however, it is preferred that these three groups are the same. Accordingly, the gelling agent has the general formula: 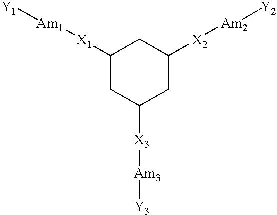
wherein
X₁=X₂=X₃=—NH—, —C(O)— or —NH—C(O)—, and preferably —C(O)—
Am₁=Am₂=Am₃=an alpha, beta or gamma amino acid, or an oligopeptide of from two to four alpha, beta or gamma amino acids, wherein each amino acid may be substituted with a substituent, wherein each substituent may be a substituted or unsubstituted, branched, cyclic or straight alkyl or alkenyl group which possibly contains an aromatic, ester or ether moiety or one or more other heteroatoms chosen from the group of N, S, O, P and B. Preferably, each substituent does not contain more than 12 carbon atoms. Preferably, each of Am₁, Am₂ and Am₃ contain zero or one substituent;
Y₁=Y₂=Y₃=—OH, —OR, or —NHR, if the corresponding X is —C(O)— or —NH—C(O)—, and Y₁, Y₂, and Y₃ are chosen from the group of —C(O)R, —C(O)—NHR, —C(O)—OR, and R, if the corresponding X is —NH—, wherein R is H, or a substituted or unsubstituted, branched, cyclic or straight alkyl, alkenyl or alkynyl group which possibly contains an aromatic, ester or ether moiety or one or more other heteroatoms and may have from 1 to 40 carbon atoms.

Preferably, Y₁=Y₂=Y₃=—OH, —OCH3, —OCH2CH3, —OCH₂CH₂OH, —NH2, —NHCH₂CH₂OCH₂CH₂OH, —OCH2CH2COH2CH3, —NHOH, —NHCH3, —NH—CH2-p-C6H4-B(OH)2, or —NHCH2CH2OH.

The amino acids may be chosen from all natural and unnatural (synthetic, e.g., β-amino acids or α-alkylated amino acids) amino acids. Preferably, the amino acids are α-amino acids, of which both the D and the L isomers are eligible. Suitable examples of amino acids are leucine, isoleucine, lysine, valine, proline, methionine, glycine, histidine, alanine, phenylalanine, tryptophan, serine, threonine, cysteine, tyrosine, asparagines, glutamine, aspartic acid, glutamic acid, and arginine. In the context of the invention, a derivative of an amino acid is defined as to include esters or amides (e.g., of aspartic acid, lysine or glutamic acid) and (thio)ethers (e.g., of serine, tyrosine or cysteine).

In another embodiment, R contains a terminal reactive group, such as an alkenyl group. By choosing an appropriate terminal reactive group, a gelling agent or thickener according to the invention may be used to form a gel which can be subjected to further reaction. For instance, a gelling agent or thickener with a terminal alkenyl group (C═C) can, after formation of a viscous solution in an aromatic solvent, be interconnected by a metathesis reaction following standard procedures as found in, e.g., J. Am. Chem. Soc. (1995) 117, 12364. The metathesis reaction transforms the viscous solution into a stiff gel, which can, for instance, be used in columns for chromatographic purposes (see also Sinner et al., Angew. Chem. Int. Ed. 39 (2000) 1433-1436 and Sinner et al., Macromolecules 33 (2000) 5777-5786).

The above-mentioned gelling agents or thickeners can be prepared by reaction of an appropriate substituted cyclohexane, such as 1,3,5-tri(chlorocarbonyl)cyclohexane or 1,3,5-triaminocyclohexane, with a pre-prepared, optionally activated amino acid or di-, tri-, or oligopeptide derivative, such as an amino acid alkyl ester, an amino acid alkyl amide, an amino acid glycol ester or an amino acid glycol amide. Feasible reactions and their conditions may be based on standard synthetic methods for amide and urea formation as described in M. B. Smith, J. March, March's Advanced Organic Chemistry, 2001, 5th edition, Wiley Interscience; and E. Muller, O. Bayer, Houben-Weyl, Methoden der Organischen Chemie, Synthesen von Peptiden, Band XV/1 and 2, 1974, George Thieme Verlag.

Preferred groups of compounds for use in this invention comprise:

CHex-Am-Met-OH

Gelates water and lower alcohols

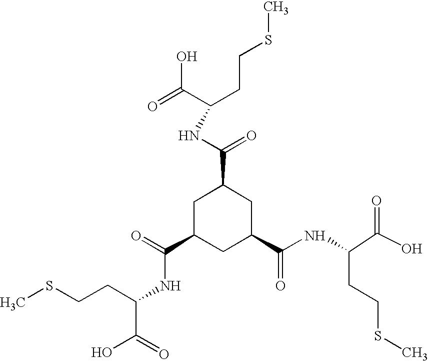

CHex-Am-Val-OH

Gelates water

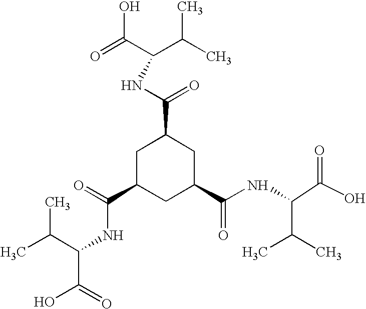

CHex-Am-Met-OMe

Gelates both organic solvents and water

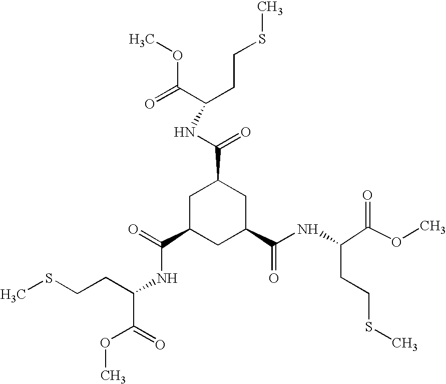

CHex-Am-Glu-OMe

Gelates both organic solvents and water

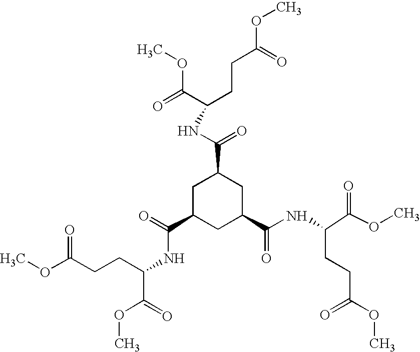

CHex-Am-Asp-OMe

Gelates both organic solvents and water

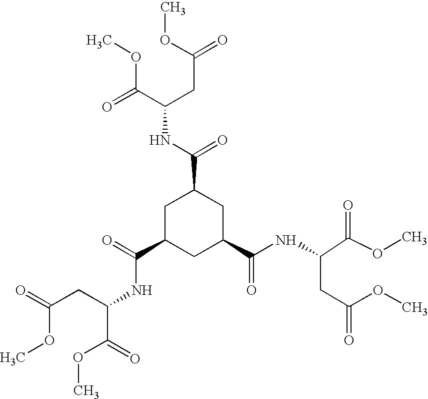

CHex-Am-Phe-Am-EtOEtOH

Gelates water and lower alcohols

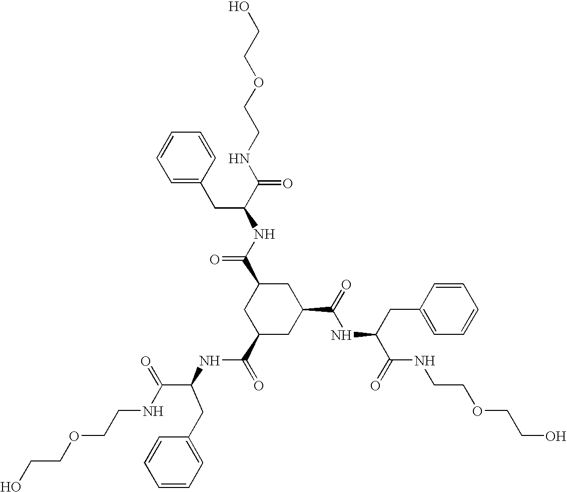

CHex-Am-Phe-OEtOH

Gelates water and lower alcohols

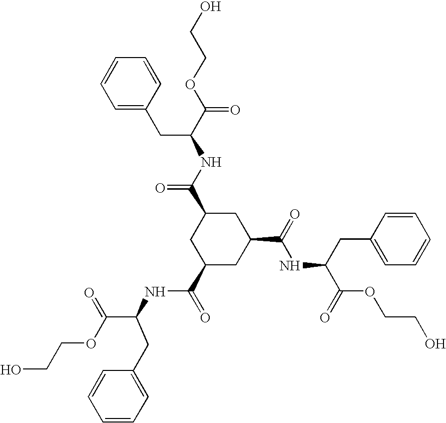

CHex-Am-(L)Phe-(D)Ala-OH

Gelates water and lower alcohols

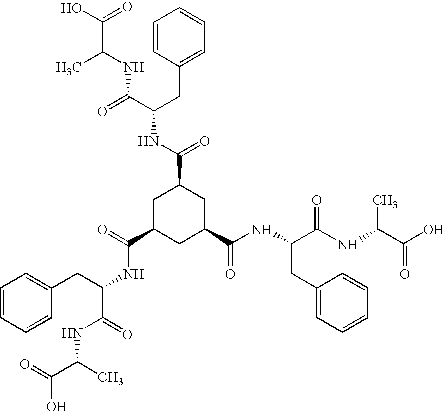

CHex-Am-(L)Phe-βAla-OH

Gelates water

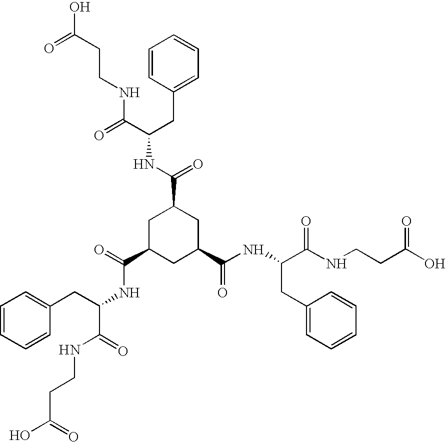

CHex-Am-Phe-Am-Glu-OH

Gelates water

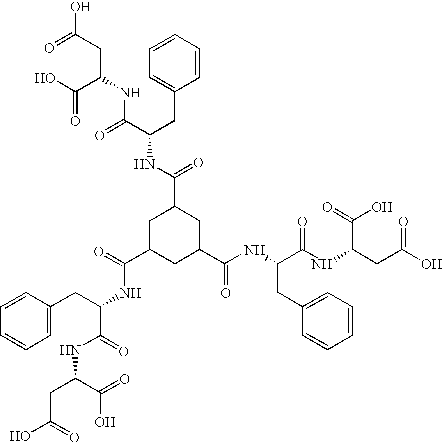

CHex-Am-Met-Hista

Gelates water and lower alcohols

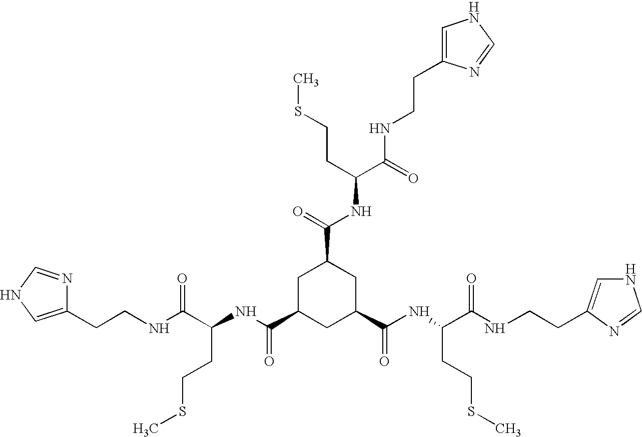

CHex-Am-Met-His-OMe

Gelates water

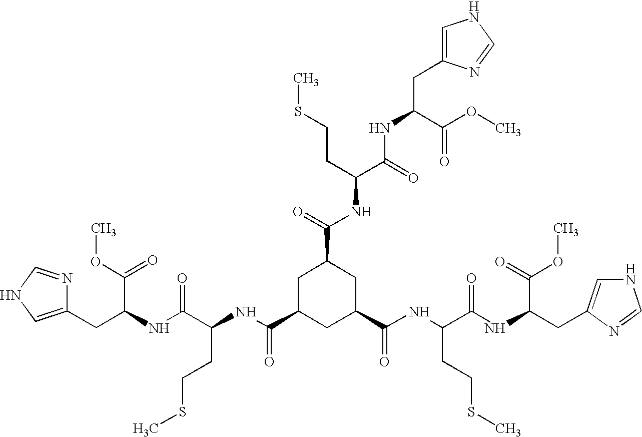

CHex-Am-Met-Am-Borate

Gelates water and lower alcohols

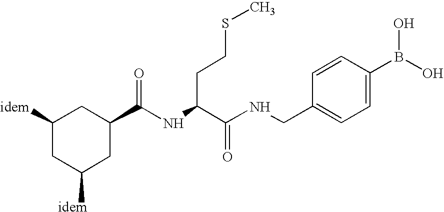

CHex-Am-Phe-Am-Borate

Gelates water and lower alcohols

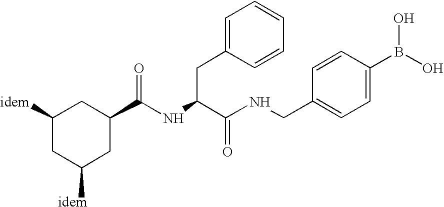

CHex-Am-Ser(Bzl)-Am-Borate

Gelates water and lower alcohols

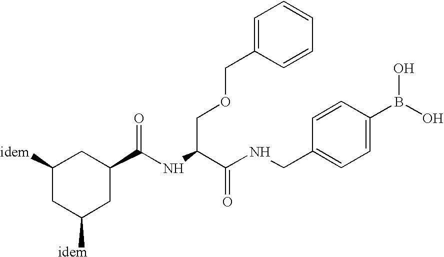

CHex-Am-Met-Am-EtOH

Gelates water

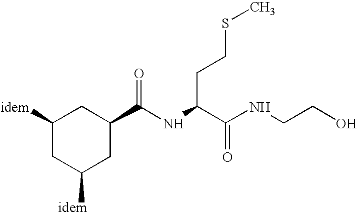

CHex-Am-Phe-OH (racemic)

Gelates water

This is a racemic compound mixture (i.e., mix of DDD, LLL, DDL, DLL), the LLL diasteroisomer only gelates lower alcohols.

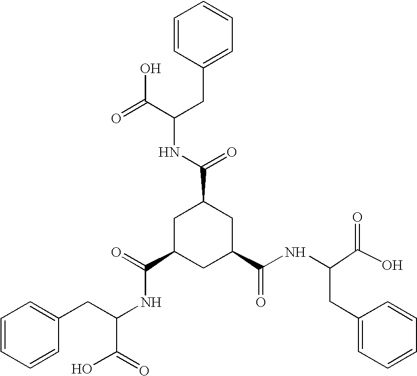

CHex-Am-Phe-OH (DDL)

Gelates water

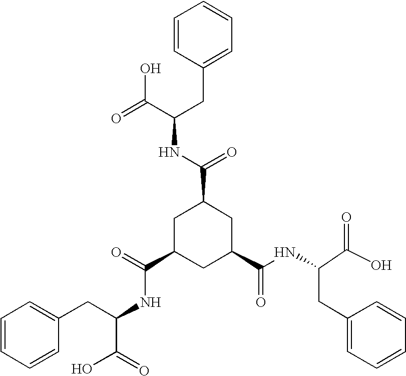

The above-identified compounds are particularly useful since they are able to gelate water. When used as or in a delivery vehicle according to the invention, gelators of water are especially preferable since water is an acceptable compound for pharmaceutical preparations, while many organic solvents are toxic and thus less useful for in vivo delivery of therapeutics or diagnostics.

To obtain a gel or to thicken the solvent, the compound is mixed with the required solvent or a mixture of solvents in an amount between 0.01 and 50 wt. %, based on the weight of the composition. Typically, the dissolution of the components will be performed by heating (in some cases it may be helpful to homogenize the components, e.g., vortex) them together at temperatures of 20° C. to 200° C., preferably 50° C. to 150° C. Cooling these hot solutions to a preferred temperature in the range of −20° C. to 100° C., preferably 4° C. to 100° C. affords the gel or thickened solvent. The gels obtained have been found to comprise thin, intertwining fibers. In an alternative embodiment, the gelling agent is first dissolved in a polar or apolar solvent followed by the addition of another solvent or solvent mixture, thereby causing gelation to take place.

Carbohydrate Gels

Surprisingly, it has been found that gelling agents or thickeners can also be prepared from low molecular weight carbohydrates. A preferred gelling or thickening agent of the invention, therefore, relates to a gelling agent in the form of N,N′-disubstituted aldaramides and N,N′-disubstituted pentaramides and derivatives thereof. Specifically, the gelling or thickening agent relates to a gelling agent having the following structure: 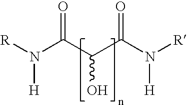
wherein n is 3 or 4, and wherein R and R′ represent the same or different substituents chosen from the group of substituted or unsubstituted, branched, possibly containing aromatic groups, cyclic or linear alkyl, alkenyl, alkynyl groups having from 1 to 40 carbon atoms. These compounds are the subject of WO 02/070463, which is herein incorporated by reference. In a preferred embodiment, R and R′ each independently represent a linear, branched, or cyclic alkyl group having 4 to 20 carbon atoms. More preferred is that R and R′ are each independently selected from the group of cyclo-alkyl groups having 4 to 16 carbon atoms. In a preferred embodiment, R and R′ represent the same substituent. One of the advantages of the present gelling agents or thickeners is that they can be based on naturally occurring products, such as carbohydrates. Thus, the starting materials for producing them are from a renewable source.

A gelling agent or thickener, according to this embodiment of the invention, may be prepared by converting an aldose or pentose to its corresponding aldaric or pentaric acid, or a salt thereof, such as an alkali metal salt or an (alkyl)ammonium salt. It is preferred to use an aldose or pentose chosen from the group of allose, altrose, glucose, mannose, gulose, idose, galactose, talose, ribose, arabinose, xylose, lyxose and derivatives thereof, as these lead to products having particularly favorable gelling and/or thickening properties. It is to be noted that both the L and the D isomers of the aldose or pentose, as well as mixtures thereof, can be used. Suitable derivatives of the mentioned aldoses and pentoses include deoxy aldoses or pentoses, ethers, esters and the like. In a more preferred embodiment, D-glucose is chosen as aldose.

The conversion of the aldose or pentose to its corresponding aldaric or pentaric acid is generally achieved by oxidation. The oxidation can suitably be carried out using Pt/O₂,TEMPO/NaOCl/(NaBr) or HNO₃/(NaNO₂) as an oxidizing agent. Further details for the manner in which the oxidation may be carried out can be found in U.S. Pat. Nos. 5,831,043, 5,599,977 and 6,049,004; in J. Org. Chem., 1977, 42, 3562-3567; and in J.-F. Thaburet et al., Carbohydr. Res. 330 (2001), 21-29, all of which are incorporated herein by reference. The resulting aldaric or pentaric acid or salt thereof is subsequently condensed with a primary amine to obtain the objective gelling agent or thickener. The aldaric or pentaric acid can be condensed with an amount of at least 200 mole %, with respect to the aldaric or pentaric acid, of a primary amine. It is preferred to activate the aldaric or pentaric acid first by means of lactonization and/or esterification, depending on the stereochemistry of the carbohydrate. Further details may be found in Kurtz et al., J. Biol. Chem., 1939, 693-699; Hoagland, Carbohydrate Res., 1981, 98, 203-208, and U.S. Pat. No. 5,312,967, all of which are incorporated herein by reference.

In an alternative embodiment, non-symmetrical N,N′-dialkylaldaramides or N,N′-dialkylpentaramides may be prepared, wherein R and R′ represent different substituents. In accordance with this embodiment, the aldaric or pentaric acid may be converted into an N-alkyl-1-aldar/pentaramid-6-ate or N-alkyl-6-aldar/pentaramid-1-ate (as disclosed in U.S. Pat. No. 5,239,044; L. Chen et al., J. Org. Chem. 61 (1996) 5847-5851; R. Lee et al., Carbohydr. Res. 64 (1978) 302-308; and K. Hashimoto et al., J. Polym. Sci. Part A, Polym. Chem. 37 (1999) 303-312), activated, and subsequently condensed, with preferably 100 mole % with respect to the N-alkyl aldar/pentar-ate of a second primary amine.

Carbohydrate gelling compounds preferably used for the present invention are dicyclohexyl glucaramide, dicyclododecyl glucaramide, dicitronellyl glucaramide and didodecyl galactaramide, which are described in more detail in WO 02/070463. In general, the resulting gelling agent or thickener precipitates from the reaction mixture in which it is formed and can be easily isolated by filtration. Further purification can be performed by conventional techniques like crystallization or, in the case of products based on galactaric acid derivatives, by thoroughly washing with ethanol, water, acetone or hexane.

It will be understood that the use of the present gelling agents or thickeners to prepare a gel or to thicken a composition is also encompassed by the invention. As is well-known, gelling behavior of compounds or compositions is highly unpredictable. In principle, a solution of a specific compound in a solvent, e.g., an organic solvent, is considered a gel when a homogeneous substance is obtained which exhibits essentially no gravitational flow. Preferably, the gelling phenomenon is thermoreversible. However, in as far as the present compounds do not provide a gel in a composition, they may be used as a thickener or rheology controlling agent as their addition to a composition may give rise to an increase in viscosity of the composition.

Compositions in which the present compounds have been found to produce a gel include compositions in numerous organic solvents. Preferred examples include aromatic and non-aromatic hydrocarbons, alcohols, ethers, esters, aldehydes, alkanoic acids, epoxides, amines, halogenated hydrocarbons, silicon oils, vegetable oils, phosphoric esters, sulfoxides and mixtures thereof. The compound also produces a gel in polar solvents such as water. The choice of composition for gelling can be tuned to the invented use. For instance, in situations where clinical application of a vehicle of the invention is intended, biocompatibility of the composition is preferred. In order to obtain a gel, the gelling agent or thickener is preferably mixed with the composition to be transformed to a gel in an amount preferably between 0.01 and 50 wt. %, based on the weight of the composition. In a preferred embodiment, the mixture of the gelling agent or thickener and the composition is heated to allow for an even better gel formation or thickening. Typically, the heating will involve raising the temperature of the mixture to about 30° C. to 175° C. until a clear solution is obtained. In an alternative embodiment, the gelling agent is first dissolved in a polar or apolar solvent and then added to or sprayed into a composition or solvent to be converted into a gel. Another method of producing a gel is by dissolving the gelling agent in a solution and evaporating the solvent.

Alternatively, some other methods to produce gels are dependent on an environmental stimulus, such as light, pH and/or chemical stimuli. Photo-controlled gelation (see below) and pH-controlled gelation (see below) are two mechanisms which can be used to induce the sol-to-gel transition, while in some cases, this process is reversible and thus can also be used for gel-to-sol transition. Chemical inducers for triggering gel-to-sol or sol-to-gel formation are disulfide-reducing enzymes and thiol-oxidizing enzymes, which in nature also occur in the human body. Tris-(2-carboxy ethyl)phosphine, mercaptoethanol, 1,4-dithiothreitol, glutathione and dimethyl sulfoxide (DMSO) can also be used for chemical triggering, as shown in the Examples.

One further way to form a gel is by mixing solutions of two different gelling agents that, at the reaction temperature and concentration, each independently remains in the sol phase, but when mixed, transit to the gel phase. An example for this (as shown in the experimental section) is a mixture of CHexAmPheOCH₂CH₂OH and CHexAmPheNCH₂CH₂OCH₂CH₂OH.

Preferably, the substance to be made available in an induced way at the predetermined site is incorporated in the gel at the time of gel formation. However, this need not always be true. Substances may also be allowed to enter a preformed gel under the appropriate conditions.

In a preferred embodiment, the substance to be made available, i.e. the substance of interest, is in some way prevented from unintended leakage out of the gel. This is preferably achieved by allowing for an interaction of the substance with the gel. Interaction can be achieved using a covalent bond of any type or a non-covalent bond (such as electrostatic or hydrophobic interactions, H-bonds). Release of the substance from the gel can be achieved in a number of ways known to the person skilled in the art and depending on the type of gel, substance and environment. Covalent bonds can also comprise labile links that can be broken under certain conditions such as pH, temperature, enzyme activity, light and the like. Using such labile linkers between the substance of interest and the gelling agent nearly totally prevents leakage and enables the immediate release of the substance of interest when the environmental conditions supersede the threshold for breaking the link. Preferably, the enzymatically labile linker is cleaved by an enzyme which is present in the neighborhood of the target cell. If a substance of interest is covalently linked via an enzymatically labile linker to either a gelling agent or a prodrug-gelling agent conjugate (which can be incorporated into the gel structure), enzymatic cleavage in the gel state should be strongly disfavored. The gel-to-sol transition, however, will make the prodrug available to the enzyme, resulting in cleavage and subsequent release of the drug.

Another embodiment in which leakage is minimized is when the substance of interest is comprised in the gel in relatively large particles and thus is more entrapped in the gel. The large particles can be agglomerates of a low molecular weight substance of interest or large (bio)molecules such as proteins (enzymes) (S. Kiyonaka, et al., 2003, Chem. Eur. J. 9(4), 976-983), polymers, DNA, RNA, or the like, but they can also comprise lipid vesicles, micelles, liposomes or even cells which contain a substance of interest. Obviously, the size of the particles, their “stickyness” to the gelling agent and the degree of viscosity of the gel influence the leakage properties of the combination.

Lastly, the gels are perfectly applicable in the present invention when they are comprised in a lipid vesicle such as a micelle or liposome, which is then called a lipogelosome. The encapsulation of these gels, either already containing a substance of interest or taking up the substance during encapsulation, can be done by standard methods, such as encapsulation by means of a pH or salt gradient or by means of a gelator concentration gradient. However, the inducible nature of sol-to-gel transition of the above-described gelling agents allows for encapsulation in the sol state and activating the transition via a signal, such as pH, light or chemical, which would induce the formation of the gel inside the lipid vesicle. This embodiment for a delivery vehicle is especially preferable when hydrophobic compounds need to be delivered. In normal lipid vesicles, hydrophobic compounds would be released with difficulty because of the hydrophobic interaction with the lipid. However, when entrapped in a gel, this interaction would not occur and the substance of interest will be released together with the gel or after the gel-to-sol transition at the predetermined site.

Next to temperature inducibility, some gelling agents can also be induced by other stimuli such as pH, light and chemicals. In a preferred aspect of the invention, the gelling and/or thickener agent comprises a light-switchable gelator. Photo-controlled gelation has been reported by Murata et al., J. Am. Chem. Soc. (1994), 116, 6664-6676. They disclose certain cholesterol-azobenzene derivatives which isomerize from the trans-state into the cis-state upon irradiation with light. Gelators comprising a light switch resulting in altered gelling behavior between the ground state and the light-activated state are very well suited for a vehicle of the invention. A further suitable light-switchable gelator is provided by the invention in the form of a light-switchable gelator having the formula: 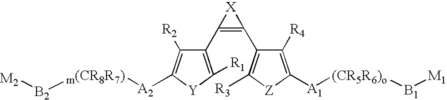
wherein
X is chosen from the group of the moieties —(CH₂)ₙ—, —(CF₂)ₙ—, —C(═O)—O—C(═O)— and —C(═O)—NR—C(═O)—, wherein n is 3 or 4 and wherein R is hydrogen, a (cyclo)alkyl group or an aryl group;
Y and Z each are nitrogen or sulfur;
R₁ and R₃ each are an alkyl group;
R₂ and R₄ each are hydrogen or an alkyl group;
A₁ and A₂ each are absent or are an aryl group;
R₅, R₆, R₇, and R₈ each are hydrogen, an alkyl group or an aryl group;
m and o each are integers chosen from the group of 0, 1, 2, 3, and 4;
B₁ and B₂ are hydrogen bonding moieties; and
M₁ and M₂ each are an aryl group, a (cyclo)alkyl group, or —CR₉R₁₀R₁₁, wherein R₉, R₁₀ and R₁₁ each are hydrogen, a (cyclo)alkyl group, an aralkyl group or an aryl group.
   It is to be noted that all symbols defined above may be chosen to have a meaning as defined, independent from the meaning of any of the other symbols, unless otherwise indicated herein.

Such a compound can be used to form a stable gel. The gelation phenomenon can be induced by light and has been found to be reversible. This opens a wide range of possible applications including the generation of a delivery vehicle for delivering a substance of interest to a predetermined site. An additional advantage of use of a light-switchable gelator with these characteristics is that induction of availability of the substance can be achieved by providing the correct light at the pre-determined site. In this embodiment of the invention, the means for inducing availability include the signal comprising the correct type of light and the receptor comprising the light-switchable gelator.

As used for the compounds of the above general formula, the alkyl group refers to a straight-chain or a branched-chain alkyl radical containing from one to ten, preferably from one to eight, carbon atoms. The term “(cyclo)alkyl group” refers to an alkyl group or a cyclic alkyl radical. The latter includes saturated or partially saturated monocyclic, bicyclic or tricyclic alkyl radicals wherein each cyclic moiety contains three to eight carbon atoms. Examples of such radicals include methyl, ethyl, n-propyl, isopropyl, n-butyl, isobutyl, sec-butyl, tert-butyl, pentyl, iso-amyl, hexyl, octyl, cyclopentyl, cyclopentenyl, cyclohexenyl and cyclohexyl.

The term aryl group for the compounds of the above general formula refers to an aromatic or hetero-aromatic ring system, such as a phenyl, naphtyl or anthracene group radical, preferably a phenyl, which optionally carries one or more substituents chosen from the group of alkyl, methoxy, halogen, hydroxy, amino, nitro, and cyano. Examples of such radicals include phenyl, p-tolyl, 4-methoxyphenyl, 4-tert-butoxy) phenyl, 4-chlorophenyl, 4-hydroxyphenyl, 1-naphtyl, and 2-naphtyl. It is to be noted that fused and connected rings, as well as 5, 6, 7 or 8-membered rings, such as cyclopentadienyl, imidazolyl, thiophenyl, thienyl, etc., are included.

The term “aralkyl group” means an alkyl radical as defined above in which one hydrogen atom is replaced by an aryl radical as defined above, such as benzyl, or 2-phenylethyl.

In a preferred embodiment, the invention relates to a light-switchable gelator as defined above having the general formula: 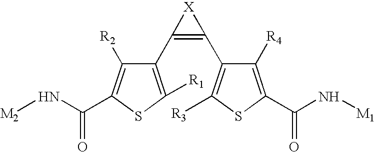
wherein R₁ and R₃ are both methyl, and the other symbols having the same meanings as defined above. It is further preferred that R₂ and R₄ each are hydrogen or a methyl group. Preferably, R₂ and R₄ have the same meaning.

In another preferred embodiment of the invention, M₁ and M₂ have the same meaning. Preferably, M₁ and M₂ are phenyl or —CH₉R₁₀R₁₁, wherein R₉ is hydrogen, R₁₀ is cyclohexyl, cyclopentyl, or an aryl group, and R₁₁ is an alkyl group. Even more preferred is an embodiment wherein M₁ and M₂ are phenyl, —CH(CH₃)(C₆H₅), or —CH(CH₃)(C₆H₁₁).

Specific compounds according to the above given formulas include 1,2-Bis(5′-formyl-2′-methylthien-3′-1)cyclopentene; 1,2-Bis(5′-carboxylic acid-2′-methylthien-3′-1)cyclopentene; 1,2-Bis(5′-anilinocarbonyl-2′-methylthien-3′-yl)cyclopentene; 1,2-Bis(2′-methyl-5′-{(((R)-1phenylethyl)amino)carbonyl}-thien-3′-yl)cyclopentene; 1,2-Bis(2′-methyl-5′-{(((R)-1cyclohexylethyl)amino)carbonyl}-thien-3′-yl)cyclopentene; 1,2-Bis(5′-boronyl-2′-methylthien-3′-yl)cyclopentene; 1,2-Bis(5′-(methyl-5-(2-thienyl)-acetic acid)-2′-methylthien-3′-1)cyclopentene; 1,2-Bis(5′-(5-(2-thienyl)-acetic acid)-2′-methylthien-3′-1)cyclopentene; 1,2-Bis(5′-{N-dodecyl-N′-(4-(2-thienyl)methyl)urea}-2′-methylthien-3′-1)cyclopentene and 1,2-Bis(5′-(4-bromophenyl)-2′-methylthien-3′-1)cyclopentene. These compounds and their preparation are described in more detail in international application no. PCT/NL02/00747.

The invention further relates to the use of a light-switchable gelator as described herein to prepare a gel. According to the invention, a gel may be prepared by dissolving the gelator in a suitable solvent by heating (if necessary), and subsequently inducing gel formation by cooling and/or irradiating it with light.

Suitable solvents may be chosen from the group of water, aliphatic hydrocarbons, aromatic hydrocarbons, halogenated hydrocarbons, non-aromatic hydrocarbons, aromatic solvents, alcohols, ethers, esters, aldehydes, alkanoic acids, epoxides, amines, silicon oils, vegetable oils, phosphoric esters, sulfoxides, ketones and mixtures thereof. Preferred solvents are water, hydrocarbons, aromatic hydrocarbons and other aromatic solvents.

A light-switchable gelator according to this invention will typically be present in the solution in a concentration of between 0.01 and 10 wt. %, based on the weight of the solution. The temperature needed in order to form a gel will depend on the solvent chosen, as well as on the exact structure of the gelator and its concentration. In a preferred embodiment, the mixture of the gelling agent and the solvent is heated to dissolve the gelling agent, and subsequent cooling allows the formation of a gel. Typically, the heating will involve raising the temperature of the mixture to about 30° C. to 175° C. Typically, the minimal temperature needed to achieve gelation will lie in the range of −10° C. to 100° C., preferably in the range of 30° C. to 80° C. The gelation process can monitored by rheology, microscopic methods, and spectroscopic methods. In the non-restrictive case that M₁ and/or M₂ are chiral, gelation results in a strong enhancement of the elipticity of the samples as measured by circular dichroism (CD) spectroscopy. An important property of the gelators according to the present invention is that they can exist as two thermally stable valence isomers, which can be converted into each other by irradiation with light in the range of 200 to 800 nm (FIG. 57). It will be understood that both valence isomers are encompassed by the invention.

Irradiation of the open form of the gelator with light of a lower wavelength (λ₁) causes conversion to a photo-stationary state (PSS) in which the ring closed form is predominant, and irradiation of the PSS with light of higher wavelength (λ₂) causes conversion to the open form of the gelator. Here, λ₁ is preferably in the range of 250 to 600 nm, and even more preferably, 300 to 450 nm, whereas λ₂ is preferably in the range of 350 to 900 nm, and even more preferably, 450 to 700 nm. The isomerization process can be monitored by spectroscopic methods, and especially UV-VIS spectroscopy, due to the presence of a strong absorption of the closed form with a maximum between 400 and 700 nm, which is absent for the open form.

Most remarkably, photo-switching between the two valence isomers of the gelator has a pronounced effect on thermal stability of the gels, as well as on the kinetics of gel formation. In a preferred embodiment, a gelator with the structure of formula IV (in PCT/NL02/00747) can be switched from the open form to the closed form by irradiation with light between 300 nm and 450 nm, which is accompanied by a change of the melting point of the gel by 5° C. to 50° C., the exact value depending on the structure of the gelator, the solvent used, and the concentration of the gelator. In an even more preferred embodiment, the melting point of the closed form of a gelator is by 5° C. to 50° C. higher than that of the open form, and gel formation in solutions of the closed form of the gelling agent is faster than gel formation in solutions of the open form.

In accordance with the present invention, the differences in thermal stability and kinetics of gelation between the open and closed form of the gelators may be exploited to induce gel formation by irradiation with light. When the melting point of a gel of the closed form is lower than that of the open form, photo-induced gelation can be achieved at a temperature between the melting point of the open and closed form, by irradiation of such a solution with light of wavelength λ₂ that causes isomerization from the closed to the open form. In another case, a gel of the open form has a lower melting temperature than that of the closed form, and gelation can be achieved at temperatures between the melting point of the open and closed form, by irradiation of such a solution with light of wavelength λ₁ that causes isomerization from the open to the closed form.

It is also possible to make use of the differences in kinetics of gelation between the open and closed form to induce gelation by irradiation with light. In general, gelation at a set temperature below the melting point of the gels is faster for gels of the valence isomer of the gelator having the higher melting point. Irradiation of a supercooled solution of an isomer of the gelator having the lower melting point with light of a wavelength causing isomerization to the other isomer of the gelator, results in a strong acceleration of the gelation process. In a preferred embodiment, a solution of a carbohydrate gelator as described above is cooled to 10° C. to 50° C. below the melting point, and irradiation of such a solution with light of wavelength λ₁ causing isomerization to the PSS (see above), together with gelation within ten minutes, whereas a similar non-irradiated solution does not turn into a gel within this period.

Another important aspect of the invention is that gelation by a gelator according to the invention often is reversible. This reversibility also holds for the photo-induced isomerization processes and all the photo-induced gelation processes described above can be reversed by performing the back-isomerization by irradiation with light as depicted in FIG. 57.

### DESCRIPTION OF THE FIGURES

FIG. 1. SDS-PAGE stained with Coomassie Brilliant Blue (molecular weight markers indicated in kDa on the left of lane A and purified detergent-solubilized MscL in lane B) and the Western blot (lane C).

FIG. 2. Electrospray ionization mass spectrometry of G22C-MscL-6His and its MTSES conjugate (solid line) Spectrum of G22C-MscL-6His. Not all peptides that are present in the sample are indicated in the spectrum (broken line) Spectrum of MTSES-conjugated G22C-MscL-6His. The masses are indicated at the peaks and show that all proteins are conjugated.

FIG. 3. Equilibrium centrifugation on sucrose gradients of proteoliposomes. 6His-MscL purified with Triton X-100 and incorporated in liposomes titrated with 4.0 mM Triton X-100 (Rsat; open squares) and liposomes titrated with 10.0 mM Triton X-100 (Rsol; closed squares). After centrifugation, the gradients were fractionated (0.5 mL) and assayed for the presence of lipids and protein. All protein, as determined by Western blotting as shown in the inset, is shown to be associated with the lipids, as determined by measuring fluorescence (AU) of R₁₈.

FIG. 4. Freeze-fracture image of proteoliposome showing the MscL channel protein as a transmembrane vesicle (white box).

FIG. 5. Patch-clamp recordings of channel activities at −20 mV from MscL reconstituted into liposomes of different lipid compositions as indicated in the figures. Pressure in the pipette, relative to atmospheric, is shown in the lower traces, and recording of the current through a patch of membrane excised from a blister is shown in the upper traces.

FIG. 6. Pressure dependence of the MscL channel reconstituted in liposomes of different lipid composition. Open probability in the patch of a membrane with a lipid composition of PC:PS, 90:10 m/m (A) and PC:PE, 70:30, m/m (B) versus the applied pressure. Smooth curves are Boltzman fits.

FIG. 7. Calcein efflux from liposomes with (closed circles) and without (closed squares) MscL as a function of a decrease in osmolality of the external medium. A small volume (typically 20 μL) containing (proteo)liposomes in iso-osmotic buffer is rapidly diluted with buffer of decreasing osmolality and calcein release was determined by dividing the fluorescence at 100 seconds after dilution by the total fluorescence obtained after Triton X-100 lysis.

FIG. 8. Calcein release under iso-osmotic condition mediated by conjugated G22C-MscL-6His. Calcein release of MTSES-conjugated G22C-MscL-6His channel protein reconstituted into liposomes (PC:Chol, 60:40, m/m) (closed circles). Liposomes with the same lipid composition and sample treatment as above but without MscL (closed squares).

FIG. 9. Effect of 5 mol % DGPE-PEG(2000) on the calcein release from liposomes (PC:Chol, 60:40, m/m). Calcein release from PC:Chol:DGPE-PEG(2000) liposomes in the presence of buffer (closed triangles), rat plasma (closed circles), human plasma (closed squares). Calcein release from liposomes without DGPE-PEG(2000) (closed diamond).

FIG. 10. Structure of DTCP1 in the open state (A) and in the closed state (B). The molecule can reversibly isomerize depending on the wavelength of the absorbed light.

FIG. 11. ESI-MS analysis of the DTCP1 conjugation to a single cysteine mutant of MscL at position 22. Unconjugated G22C-MscL with expected mass of 15697 Da (closed squares). DTCP1-conjugated G22C-MscL with a 344 Da mass increase (closed triangles).

FIG. 12. Absorption spectra of DTCP1. Open isomer A has a maximum at 260 nm and no absorbance at higher wavelengths than 400 nm; closed isomer B has very distinct peak with maximum at 535 nm. Gray line shows subtracted spectra of open and closed isomer.

FIG. 13. Substracted spectra of open and closed isomer of DTCP1 after conjugation to G22C-MscL and reconstitution in DOPC:DOPS (90:10, mol/mol) lipid bilayer.

FIG. 14. Four switching cycles of DTCP1 conjugated to MscL and reconstituted in lipid bilayer.

FIG. 15. Photo-chromic molecule SP1 in its spiropyran form (left) and merocyanine zwitterionic form (right).

FIG. 16. Absorption spectrum of SP1 conjugated to MscL in spiropyran form SP1 and after irradiation, in highly charged merocyanine form MC1.

FIG. 17. Reversible switching between spiropyran (SP) and merocyanine (MC) form by alternating irradiation with UV and visible light.

FIG. 18. Structure of sodium di(C4azo-O—C6)-phosphate in the trans- and cis-conformation.

FIG. 19. UV/Vis spectra of sodium di(C4azobenzene-O—C6)-phosphate (7) in the trans and cis state. The molar ratio of DSP to sodium di(C4azobenzene-O—C6)-phosphate is 95:5. Concentration of sodium di(C4azobenzene-O—C6)-phosphate is 12.5 μM.

FIG. 20. Repeated cycles of the isomerization of lipid 6 in a vesicle which is composed of 95% DOPC and 5% lipid 6. For the trans-configuration, the absorbance at 349 nm is given and for the cis-configuration, the absorbance at 313 nm is given.

FIG. 21. UV/Vis spectra of lipid 6 in a vesicle which composes 95% DOPC and 5% lipid 6. The times indicated are the irradiation times. The sample was irradiated with 365 nm light.

FIG. 22. DSC graphs of pure DSP and a mixture of DSP and sodium di(azobenzene-O—C9)-phosphate (molar ratio 95/5).

FIG. 23. ESI-MS analysis of the IMI conjugation to a single cysteine mutant of MscL at position 22. Unconjugated G22C-MscL with expected mass of 15697 Da (closed squares). IMI-conjugated G22C-MscL with a 156 Da mass increase (closed triangles).

FIG. 24. Patch-clamp recordings of IMI coupled and uncoupled MscL-mutant G22C channels as shown in FIGS. 24B and 24A, respectively. Five μl of G22C spheroplasts were incubated with IMI (2 mM final concentration) or with patch buffer overnight at 4° C. The next day, currents through cell attached patches held at +20 mV were recorded for unlabeled (FIG. 24A) and unlabeled (FIG. 24B) proteins and histograms showing the conductance states of each recording is given in FIG. 24C and FIG. 24D, respectively.

FIG. 25. Different pKas of substituents for MscL-mutant G22C labels.

FIG. 26. Patch-clamp recordings of patches excised from proteoliposomes containing BP coupled and uncoupled MscL-mutant G22C channels. FIG. 26A shows the labeled channel behavior at pH 7.2 and the histogram showing the conductance levels is given in FIG. 26B. FIGS. 26C and 26E show the behavior of unlabeled MscL channel at pH 5.2, respectively. FIGS. 26D and 26F show the labeled channel at pH 5.2 and their conductance histograms are given in FIG. 26G for the unlabeled and FIG. 26H for the BP-labeled MscL channel, respectively. Measurements were performed with +20 mV constant voltage.

FIG. 27. Dependence of the amount of membrane tension needed to open the MscL channel on the lipid composition of the membrane. Increase in DOPE content results in a decrease in the membrane tension needed to open the channel.

FIG. 28. Patch-clamp recordings of MscL channel activities at +20 mV in spheroplasts. Pressure in the pipette, relative to atmospheric, is shown in the lower traces (FIGS. 28A and 28B) and recordings of the current through a cell-attached patch of a spheroplast are shown in the upper traces (FIGS. 28A and 28B). FIG. 28A shows results of MscL-mutant G22C in spheroplast before MTSET attachment and FIG. 28B is the same as A but after MTSET attachment. Buffer: 200 mM KCl, 90 mM MgCl₂, 10 mM CaCl₂, 5 mM HEPES, pH 6.0. FIGS. 28C and 28D show the histograms of the conductivity preferences of FIGS. 28A and 28B, respectively.

FIG. 29. Calcein efflux from liposomes (DOPC:DOPS, 90:10, mol/mol) with MscL-mutant G22C (protein to lipid, 1:20 wt/wt). One mM MTSET, 2.5 mM MTSEA, and 10 mM MTSES was added at time indicated by the arrow.

FIG. 30. SDS-PAGE (12%) stained with Coomassie Brilliant Blue. Lane M: BioRad broad range protein molecular weight marker: 207,000; 120,000; 92,000; 55,900; 35,400; 29,000; 21,700; and 7,300. Lane 1: MscL isolated with Triton X-100. Lane 2: MscL isolated with n-octyl-β-D-glucopyranoside.

FIG. 31. Urinary excretion of MAG3 after subcutaneous or intravenous injection. Free MAG3 intravenously (open circle, right y-axis), free MAG3 subcutaneously (closed circle), MAG3 in “empty” liposomes subcutaneously (open squares), MAG3 in G22C-MscL-containing liposomes subcutaneously (closed squares).

FIG. 32. Subcutaneous pH reduction. MES buffer (0.5 ml, pH 6.1) of different molarities was injected subcutaneously in conscious rats.

FIG. 33. Urinary excretion of IOT after subcutaneous injection. Free IOT (open squares), IOT in DOPC/PS liposomes (closed squares), IOT in DOPC/PE liposomes (closed triangles).

FIG. 34. SDS-PAGE gel stained with Coomassie Brilliant Blue. Lane A: vesicles containing the overexpressed protein; lane B: molecular weight marker; lane C: purified protein.

FIG. 35. Freeze-fracture image of proteoliposome showing the MscL channel protein as a transmembrane particle (white box).

FIG. 36. A typical trace of channel activity of Msc^{Ll} in MscL^{Ec-}/MScS^{Ec+} E. coli cells. The upper trace shows the current across the membrane due to channel activity. Flow of current is shown upward in all traces. From left to right, in time, first two channels of small conductivity open (also shown in enlarged left panel) and later, the opening of a single MscL^{Ll} is shown (also shown in enlarged left panel). The lower trace indicates the pressure applied to the membrane. The panels show enlargements of the upper trace.

FIG. 37. Top panel shows the dependence of opening chance of the MScL^{Ec} channel on the applied pressure in the pipette. The sigmoidal curve shows that no channels open at 0 mmHg pressure and that all channels are open all the time at 90 mmHg. Center panel shows the time channels spend in the open state. The bars indicate the distribution of opening times of the L. lactis MscL (<0.1 ms and 0.7 ms). Unfortunately, resolution of the traces doesn't allow analysis on a shorter time scale. Bottom panel shows relationship between voltage and current through the channel. The slope of this graph is the conductivity of the channel, which is 2.5 nS.

FIG. 38. Electrophysiological analysis of MscL^{Ll} in left panel: PC:Cholesterol 8:2 mol/mol, right panel: PC:PS 9:1. Protein:lipid ratio in both cases is 1:1000.

FIG. 39. Calcein release from PC:PS (9:1) proteoliposomes containing MscL^{Ll} (protein:lipid 1:500) or not containing any protein after dilution of the iso-osmotic buffer, with dH₂O to indicated dilutions. It can be seen that the protein-containing liposomes release more calcein than the liposomes without protein. This is MscL^{Ll}-mediated efflux of the calcein.

FIG. 40. Efflux of FITC-insulin under different conditions from DOPC:DOPS (9:1, mol/mol) liposomes containing E. coli MscL-mutant G22C. Not filtered (200 μl proteoliposomes); not filtered after triton (200 μl proteoliposomes with Triton X-100); filtered (200 μl after filtration); five minutes or ten minutes+MTSET (200 μl proteoliposomes incubated with 1 mM MTSET for five minutes or ten minutes followed by filtration; ten minutes—MTSET (200 μl proteoliposomes incubated ten minutes without MTSET followed by filtration; Triton (200 μl proteoliposomes with Triton X-100 followed by filtration).

FIG. 41. Western blot that shows effect of pH supernatant on binding of MSA2::cD to TCA-pretreated L. lactis cells. As before, the Western blot shows the amount of MSA2::cD that was bound by the cells. In addition, the amount of MSA2::cD that was not bound and remained in the medium after binding is shown. The arrow indicates the expected position for pro-MSA2::cD and the asterisk, the position of mature MSA2::cD. Lane 1, pH during binding 6.2, cells; Lane 2, pH during binding 6.2, supernatant after binding; Lane 3, pH during binding 3.2, cells; Lane 4, pH during binding 3.2, supernatant after binding; and Lane 5, Positive control: L. lactis, TCA pretreated with bound MSA2::cA at pH 6.2. It is clearly visible that MSA2::cD binds better at pH 3.2 than at pH 6.2 (compare Lanes 1 and 3).

FIG. 42. Western blot of medium supernatant (S) after binding to ghost cells at the indicated pHs and ghost (G) with the bound protein anchor. Lanes 1 and 2, binding at pH 3; Lanes 3 and 4, binding at pH 5; Lanes 5 and 6, binding at pH 7. The figure shows that there is still considerable binding at pH 5. At this pH, the native cD anchor (D1D2D3) shows little binding. The addition of the A3 repeat, which has a high pI value, results in an increase of binding at pH 5.

FIG. 43. Schematic presentation of the CWS domain containing protein hybrids used for adherence. SS: signal sequence of L. lactis PrtP; PS: prosequence of L. lactis PrtP; MSA2: merozoite surface antigen 2 of P. falciparum (reporter protein); CWS: cell wall-spanning domain of L. lactis PrtP; CWA: cell wall anchor (covalent) of L. lactis PrtP.

FIG. 44. Adherence of L. lactis NZ9000 cells expressing (A) MSA2 fused to AcmA protein anchor and (B) MSA2 fused to three CWS domains (M::C3a) to human intestine 407 Henle cells.

FIG. 45. Reconstitution of PA38 into DOPC liposomes and orientation of the reconstituted PA38. About 200 μl liposomes reconstituted by sonication (Lanes 1-4) or by spontaneous integration (Lanes 5-8) were incubated for one hour at 37° C. in the presence (Lanes 1, 3, 5 and 7) or absence (Lanes 2, 4, 6 and 8) of 8 μg/ml trypsin. Liposomes were treated with Triton X-100 (3%) to release integrated and loaded proteins (Lanes 3, 4, 7 and 8). Lane 9 contains free full-length PA38 as a reference. Lanes 1, 3, 5 and 7 show smaller fragments of PA38 due to the proteolytic activity of trypsin. The proteins were visualized by the use of an antibody that specifically recognized the myc-epitope in PA38.

FIG. 46. PA38-liposomes incubated with (Lanes 1 and 2) and without ghost cells (Lanes 3 and 4). After incubation, ghost cells and supernatant were separated. The pellet fractions (P) and the supernatant fractions (S) were analyzed in the Western blot. The proteins were visualized by the use of an antibody that specifically recognized the myc-epitope in PA38. Ghost cells are present in the pellet fraction. PA38-liposomes were predominantly in the ghost fraction when ghosts were present in the incubation mixture (Lane 1), whereas these liposomes remained in the supernatant fraction (Lane 4) when ghosts were absent from the incubation mixture. Lane 5 contained free full-length PA38 as a reference.

FIG. 47. Chemical structures of gelators 1 to 4 and 7 to 15.

FIG. 48. Melting points of gels of various concentrations of gelators 1 to 4 (see FIG. 47) as determined by the dropping ball method.

FIG. 49. Melting points of mixed gels of 2 and 4 (see FIG. 47) as determined by the dropping ball method.

FIG. 50. Melting points of gels of 1 at increasing pH as determined by the dropping ball method.

FIG. 51. Chemical structure of Cyclohexane bis-ureidohexylamine (compound 5).

FIG. 52. Chemical structure of CHexAmMetAmCH2Pyr (compound 6).

FIG. 53. Chemical structure of DBC (dibenzoyl-L-cystine).

FIG. 54. Light-switchable gelators; preparation of compounds A, B, C, D, E, and Z.

FIG. 55. Light-switchable gelators; preparation of compounds Z, F, G, H, and I.

FIG. 56. Light-switchable gelators; preparation of compounds Z, F, and J.

FIG. 57. Photo-switching between the two valence isomers of the gelator.

FIG. 58. Photo-switching of a gel of compound D. (a) UV-VIS spectra of D in toluene of a solution (0.35 mM) (—) and of a gel (1.8 mM) ( - - - ). (b) UV-Vis spectra of D in toluene after irradiation at λ=313 nm of a solution of (0.35 mM) (—) and of a gel ( - - - ). (c) CD spectra at 15° C. of a toluene gel of D (1.8 mM) (—) and of a solution (0.35 mM) ( - - - ). (d) CD spectra at 15° C. of a toluene gel of D (1.8 mM) after irradiation at λ=313 nm (—), after heating and cooling ( - - - ), and of a diluted solution after irradiation, at λ=313 nm ( . . . ) (PSS).

FIG. 59. Reaction scheme for the gel-to-sol transition of a DBC gel using tris-(2-carboxy ethyl)phosphine as chemical trigger.

FIG. 60. Reaction scheme for the sol-to-gel transition of a benzoyl cysteine solution (from the reaction shown in FIG. 59) using DMSO as chemical trigger.

FIG. 61. Chemical structures of 8-aminoquinoline (left) and 2-hydroxyquinoline (right).

FIG. 62. Time-dependent release of 8-aminoquinoline (diamonds) and 2-hydroxyquinoline (squares) from 0.2 wt % DBC gels. The initial quinoline concentration in the gel was 0.001 M.

FIG. 63. Determination of the cmc of micelles (consisting of NC₂nC₁₀ lactose) within a gel network of 1 or DBC (probe: ANS).

FIG. 64. Chemical structure of compound 16.

FIG. 65. Luciferase activity in the lung. Mice are injected intravenously with luciferase-DNA complexed with Sunfishes (SF) or DOTAP and terminated after 24 hours (three mice per group). Values are given as mean+SD.

FIG. 66. Body distribution: ¹²⁵Iodine-TC accumulation, 30 minutes after injection. Mice (n=5) were injected intravenously with: free myoglobin (white), SF-30/DOPE (dark gray), SF-30/cholesterol (checked, dark), SF-26/DOPE (light gray), SF-26/cholesterol (checked, light). Values are given as mean+SEM.

FIG. 67. Catabolism in the liver: ¹³¹I versus ¹²⁵I-TC, 30 and 120 minutes after injection. Mice (n=5 (30 minutes) and n=4 (120 minutes) were injected intravenously with: SF-30/DOPE (dark gray), SF-30/cholesterol (checked, dark), SF-26/DOPE (light gray), SF-26/cholesterol (checked, light). Values are given as mean+SEM.

FIG. 68. Circulation time of ¹²⁵I-TC-myoglobin in blood versus RES, 120 minutes after injection. Mice (n=4) were injected intravenously with: SF-30/cholesterol complex without PEGylated sunfish, white bars; SF-30/cholesterol complexes containing 8% SF-79, light gray bars; and SF-30/cholesterol complexes containing 20% SF-79, dark gray bar.

FIG. 69. Neurospheres were immobilized on poly-ornithine (left; phase contrast) and transfected with SF-8/DOPE. Two days after transfection, GFP-positive cells could be visualized with fluorescence microscopy (right; fluorescence).

### DETAILED DESCRIPTION OF THE INVENITON

It is clear that both the induction of gel formation and the reverse process by switching between open or closed states can be used to work the present invention. Induction of gel formation in or of a vehicle of the invention can be used to limit availability at sites that are not the predetermined site, where the induction of fluidization of the gel is typically used to allow for availability of the entrapped substance at the predetermined site. However, the reverse is also true; for instance, when the substance is made available to bind another compound at the predetermined site, whereupon inadvertent release of the bound other compound or substance at further sites should be at least in part prevented. In this particular embodiment, it is preferred that the gel or components thereof are enclosed in a film. The film in this embodiment prevents, at least in part, leakage of the loose components and substrate in the fluidized state at the predetermined site. In this condition, the substrate is maximally available for binding another compound at the predetermined site. Inadvertent release of substrate or compound bound thereto can then be prevented, at least in part, by providing light of the wavelength suitable to induce gel formation, thereby effectively trapping the substance and bound compound in the gel at or subsequent to passage from the predetermined site.

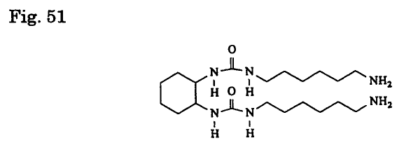

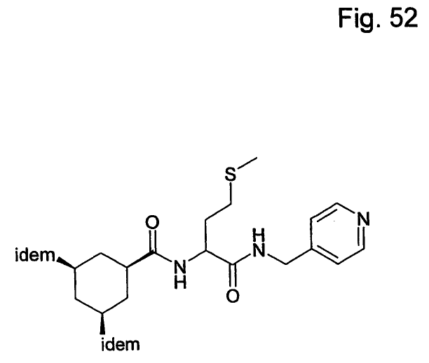

In a similar way, induction of gelation or solubilization can be achieved with changes in pH. The pH-induced gel-to-sol transition (and the reverse sol-to-gel transition) can be caused by either a decrease or an increase in pH depending on the gelator. A gelator where the gelation is induced by lowering the pH (an acidic gelator) is ChexAmMetOH, where addition of a basic compound leads to a decrease in melting temperature. Basic gelators are, for instance, cyclohexane bis-ureidohexylamine (FIG. 51) and the structure of FIG. 52. The sol-to-gel transition point of this last gelator lies around pH 4.47 (at room temperature) but gelation is also temperature dependent. Also, chemical compounds that can influence the pH can be used for inducing gelation. It has been found that DBC (dibenzoyl-L-cysteine) slowly gelates due to a decrease of the pH induced by the formation of D-gluconic acid in a reaction mixture containing DBC (in sol phase), D-glucose, glucose oxidase, catalase and NaHCO₃.

Gelation or solubilization of this gelating agent DBC has also been proven to be inducible by addition of a chemical substance: addition of the reducing compound tris-(2-carboxy ethyl)phosphine to a gel of DBC induces solubility because the DBC is cleaved into two benzoyl cysteine residues, while subsequent addition of the oxidizing agent DMSO to the solution of benzoyl cysteine causes formation of DBC and sol-to-gel transition. Addition of other compounds such as mercaptoethanol, 1,4-dithiothreitol or glutathione, also induces solubilization of a DBC gel.

The Predetermined Site

The predetermined site can be any site where the compartment should be made available toward the exterior of the vehicle. A predetermined site, preferably comprises a site in a mammalian body, preferably a human body the site can be on the outside of the body, for instance, the skin or eye. Preferably the site is an internal site. An internal site is preferably characterized by a certain molecule that is present at the predetermined site. Preferably, the characterizing molecule is not present at other sites in the body. In a preferred embodiment, the characterizing molecule is present on a cell. Preferably, the molecule is a target molecule used to target the vehicle of the invention to the predetermined site with the use of a vehicle comprising a targeting means. With “a targeting means” is meant a means for concentrating the vehicle at the predetermined site. A targeting means is typically provided to the vehicle, though this is not necessarily so. Suitable targeting means have been discussed above. Concentration at the predetermined site can, in these situations, be achieved by providing a targeting means for a target that is specifically present at the predetermined site. It is possible that the target is also present at “a limited number” of other sites. In these cases, it is preferred that the means for inducing availability of the compartment are either less or unresponsive to conditions at these “limited number” of other sites. However, in cases where the inducing means are responsive and thus induce availability of the compartment at a number of these other sites, it is still possible to achieve advantageous effects with a delivery vehicle of the invention, depending on the nature of the other sites where the compartment is made available. It can be that at the mentioned other sites, the substance is less toxic or that limited loss of substance at a non-relevant site can be tolerated without affecting the effectivity of a delivery vehicle of the invention. Similarly, it is within the scope of the invention that the inducing means is also active at a limited number of other sites, independent of the presence or absence of a targeting means. Such activation of the inducing means at other than relevant sites can be tolerated to some extent as long as the reasons for which a delivery vehicle of the invention was used are not negated. This is so the toxic effects and/or stability problems or accelerated loss of activity can be tolerated. In a preferred embodiment, the targeting means acts in synergy with an inducing means of the invention to preferentially make at least one compartment of the vehicle available toward the exterior at the predetermined site.

A preferred internal predetermined site is the bloodstream, where a compound should be made available without suffering from rapid clearance or deactivation problems typical for some (e.g., peptidic) substances. Other preferred internal sites are the lymph, the gastro-intestinal tract, the urogenital system, the central nervous system, the respiratory system, the peritoneum, organs and tumors. Other preferred sites are sites comprising invading organisms such as bacteria, fungi, yeasts and viruses. Such sites can, for instance, comprise a certain tissue or cell type that is otherwise distributed throughout or over more places in the body.

The Substance of Interest

In principle, any type of substance can be made available using a vehicle of the invention. Substances can range from herbicides, insecticides and cosmetics to drugs. Where the vehicle is used in a mammalian body or for mammalian cells, the substance preferably comprises a biologically active substance. A biologically active substance can be any substance which is able to exert an effect upon a biological system such as a biosynthesis pathway, a cell, an organ or an organism. Examples of suitable substances include:
Interleukins: peptides and proteins that modulate the immune response
Diphtheria toxin (fragment): potent inhibitor of protein synthesis in human cells
Muramyl dipeptide: activator of immune system; macrophage-mediated destruction of tumor cells
Cis-4-hydroxyproline: potential treatment for lung fibrosis
Cisplatin (derivatives): cancer treatment
Cytosine arabinose: cancer treatment
Phosphonopeptides: antibacterial agent
b-Glucuronidase: activator of prodrugs (e.g., epirubicin-glucuronide)
Cytostatic drugs (doxorubicin, ciplatin, etc.) and radionuclids
Small therapeutic proteins/peptides (interleukins, growth factors, chemokines)
diagnostic tools (antibodies, contrast fluid, radionuclids)
prodrugs, which can be enzymatically cleaved at the target site

### EXAMPLES

### Example 1

### Use of Channel-Containing Liposomes as Delivery Vehicles for the Controlled Release of Drugs

### Example 1-A

### MscL-Containing Liposomes as Drug Delivery Vehicles

Material and Methods

MscL Expression and Purification

E. coli PB104 cells containing the plasmid pB104 carrying the MscL-6His construct was grown to mid-logarithmic phase in Luria Bertani medium (10 L fermentor) and induced for four hours with 0.8 mM IPTG (P. Blount, et al., 1996, EMBO J. 15:4798-4805). Cells were French-pressed and membranes were isolated by differential centrifugation, as previously described (I. T. Arkin, et al., 1998, Biochim. Biophys. Acta. 1369:131-140). The membrane pellet (5-8 g wet weight) was solubilized in 100 mL of buffer A (50 mM Na₂HPO₄.NaH₂PO₄, 300 mM NaCl, 10 mM imidazole) containing 3% n-octyl-β-glucoside. The extract was cleared by centrifugation at 120,000×g for 35 minutes, mixed with 4 mL (bed volume) Ni²⁺-NTA agarose beads (Qiagen, Chatsworth, Calif.) equilibrated with buffer A and gently rotated for 15 minutes (batch loading). The column material was poured into a Bio-Spin column (Bio-Rad) and washed with ten-column volumes of buffer B (as buffer A, except 1% n-octyl-p-glucoside) followed by five-column volumes of the buffer B but with 100 mM imidazole. The protein was eluted with buffer B but with 300 mM imidazole. Eluted protein samples were analyzed by fractionation on an SDS-15% polyacrylamide gel followed by staining with Coomassie Blue or transferring the fractionated proteins to PVDF membranes by semi-dry electrophoretic blotting for immunodetection with an anti-His antibody (Amersham Pharmacia Biotech). Immunodetection was performed with an alkaline phosphatase-conjugated secondary antibody as recommended by the manufacturer (Sigma).

Electrospray Ionization Mass Spectrometry of Detergent-Solubilized MscL Proteins

Purified detergent-solubilized G22C-MscL-6His was heated to 60° C. for 15 minutes and precipitated protein was spun down at 14,000 rpm in a tabletop centrifuge (Eppendorf) for five minutes. The pellet was dissolved in 50% formic acid and 50% acetonitrile just before electrospray ionization mass spectrometry (ESI-MS) analysis. The average molecular masses of the proteins were calculated from the m/z peaks in the charge distribution profiles of the multiple charged ions. Spectral deconvolution was performed on the peaks over the mass range from 800 to 1700 using the computer program MacSpec (Sciex). All molecular masses quoted in this paper are average, chemical atomic masses.

2-Sulfonatoethyl Methanethiosulfonate Labeling of G22C-MscL-6His

The single cysteine mutant, G22C-MscL-6His, was labeled with (2-sulfonatoethyl)methanethiosulfonate (MTSES). A suspension of 20 to 30 μM of G22C-MscL-6His in buffer B with 300 mM imidazole (0.5 mL final volume), was incubated with 0.6 mM MTSES at 4° C. for 30 minutes. Conjugation was monitored employing ESI-MS.

Membrane Reconstitution of 6His-MscL

Dry lipid mixtures were prepared by co-dissolving lipids (Avanti Polar Lipids, Alabaster, Ala.) in chloroform, in weight-fractions as indicated in the experiments, and removing the chloroform by evaporation under vacuum for four hours. All acyl chains of the synthetic lipids were of the dioleoyl type unless indicated otherwise. The dried lipid film was dissolved (20 mg/mL) in 50 mM potassium phosphate, pH 7.0, followed by three freeze/thaw cycles. An aliquot, 200 μL of the rehydrated liposomes and 5% n-octyl-β-glucoside, was added to 200 μL purified 6His-MscL. Final protein-to-lipid molar ratio was as indicated in the experiments. Subsequent membrane reconstitution was achieved by exhaustive dialysis into a buffer containing 0.1 mM Na₂HPO₄.NaH₂PO₄ pH 6.8 containing detergent-absorbing Bio-Beads SM-2 (Bio-Rad, Inc.).

Sucrose Gradient Centrifugation

Discontinuous sucrose gradients were employed to analyze membrane reconstituted 6His-MscL as described elsewhere (J. Knol, et al., 1998, Biochemistry 37:16410-16415).

Freeze-Fracture Electron Microscopy

Freeze-fracture electron microscopy of membrane-reconstituted 6His-MscL were performed as described elsewhere (R. H. E. Friesen, et al., 2000, J. Biol. Chem. 275:33527-33535, and 40658).

Electrophysiologic Characterization of Membrane-Reconstituted MscL

MscL was reconstituted into liposomes of different lipid composition and aliquots of 200 μL were centrifuged at 48,000 rpm in a tabletop ultracentrifuge (Beckmann). Pelleted proteoliposomes were resuspended into 40 μL buffer C (10 mM 4-morpholinepropanesulfonic acid (MOPS)-buffer, 5% ethylene glycol, pH 7.2), and 20 μl droplets were subjected to dehydration-rehydration cycle on glass slides (A. H. Delcour, et al., 1989, Biophys. J. 56:631-636). Rehydrated proteoliposomes were analyzed employing patch-clamp experiments as described previously (P. Blount, et al., 1996, EMBO. J. 15:4798-4805).

In Vitro Release Profiles of a Model Drug from Proteoliposomes

The percentage release of a fluorescent model drug, calcein, from MscL-containing liposomes was calculated from the dequenching of calcein fluorescence according to the following equation:

$$\% Release = \frac{{F}_{x} - {F}_{0}}{{F}_{t} - {F}_{0}} \times 100$$

Where F₀ is the fluorescence intensity at zero time incubation, Fₓ is the fluorescence at the given incubation time points and Fₜ is the total fluorescence, obtained after Triton X-100 lysis. Fluorescence was monitored with an SLM 500 spectrofluorimeter in a thermostatted cuvette (1 mL) at 37° C., under constant stirring. Excitation and emission wavelengths were, respectively, 490 (slit 2 nm) and 520 nm (slit 4 nm). The experiments were performed at lipid concentrations of approximately 50 μM. Control and MscL-containing liposomes were prepared as described above, followed by mixing with an equal volume of 200 mM calcein in PBS buffer. Then a freeze-thaw cycle was repeated three times followed by extrusion through a 100 nm polycarbonate membrane (L. D. Mayer, et al., 1986, Biochim. Biophys. Acta. 858:161-168). The liposomes were separated from free calcein by using Sephadex 50 column chromatography equilibrated with PBS (160 mM NaCl, 3.2 mM KCl, 1.8 mM KH₂PO₄, 0.12 mM Na₂HPO₄, 1.2 mM EGTA, pH 8.0), which was isotonic to the calcein-containing buffer.
Results
Overexpression and Purification of the MscL Channel Protein

Since the expression level of 6His-MscL in E. coli was relatively low, based on the absence of a significant IPTG-inducible band on an SDS-PAGE, attention was focused on obtaining a high biomass during fermentation and a high yield after protein purification.

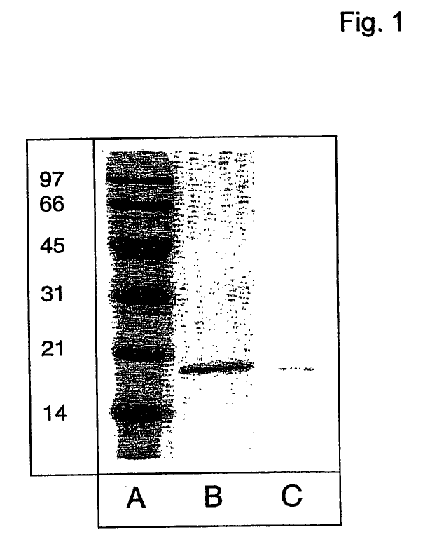

The 6His-tagged MscL could be purified to apparent homogeneity in a single step using nickel chelate-affinity chromatography as shown by SDS-PAGE (FIG. 1, lane B). The yield of this eluted His-tagged MscL was ±2 mg per Liter of cell culture with an estimated purity of >98% based on analysis using SDS-PAGE and Coomassie Brilliant Blue staining.

The rate of excretion via MscL of small molecules is >10,000 nmol/second×mg of cell protein, i.e., when the protein is in the open state. Since the expression level of MscL in wild-type bacteria is four to ten functional units per cell and the MscL channel is a homopentamer of 15,000 Da, it can be concluded that the flux via a functional MscL channel is >10⁶×s⁻¹. This activity of MscL is such that, on average, five molecules of pentameric MscL per liposome with a diameter of 400 nm should suffice. Such a liposome contains approximately 1.67×10⁶ molecules of lipid; the molar ratio of lipid over MscL will thus be 0.67×10⁵. Consequently, 2 mg of MscL will yield 6 g of proteoliposomes.

Electrospray Ionization Mass Spectrometry of Detergent-Solubilized MscL Proteins

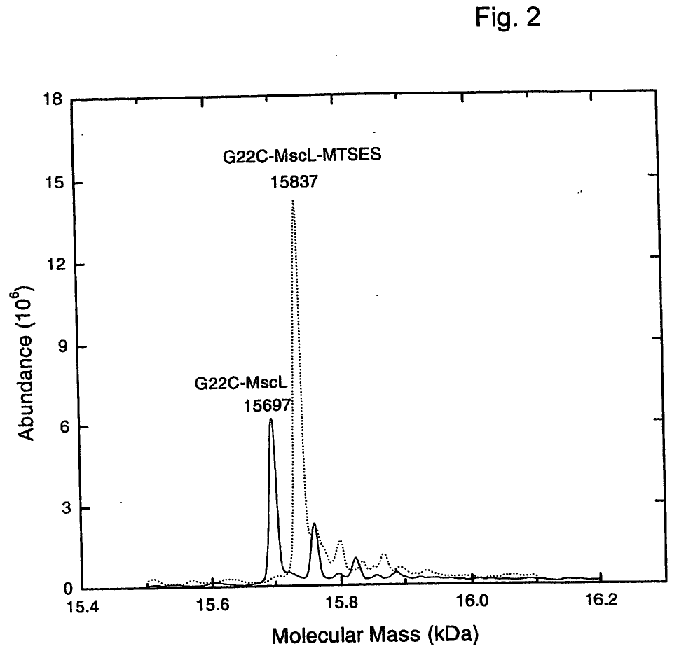

ESI-MS is an accurate and effective method to verify primary sequences of the 6His-MscL protein and the stoichiometry of conjugation reactions. FIG. 2 shows the ESI-MS spectra of the G22C-MscL-6His and the MTSES-conjugated G22C-MscL-6His samples.

Based on the deduced amino acids, the average molecular weight of G22C-MscL-6His is 15,826 Da. ESI-MS analysis of G22C-MscL-6His resulted in a molecular weight of 15,697 Da, which corresponds to the deduced molecular weight minus a methionine. This observation would be consistent with an excision of the N-terminal methionine as reported for many proteins expressed in E. coli (P.-H. Hirel, et al., 1989, Proc. Natl. Acad. Sci. U.S.A. 86:8247-8251). ESI-MS analysis of the MTSES-conjugated G22C-MscL-6His resulted in a molecular weight of 15,837 Da, which corresponds exactly with the calculated mass increase of the MTSES conjugation. ESI-MS analysis is routinely used to verify the average masses of MscL mutants and the products of conjugation reactions.

Membrane Reconstitution into Liposomes of Different Lipid Compositions

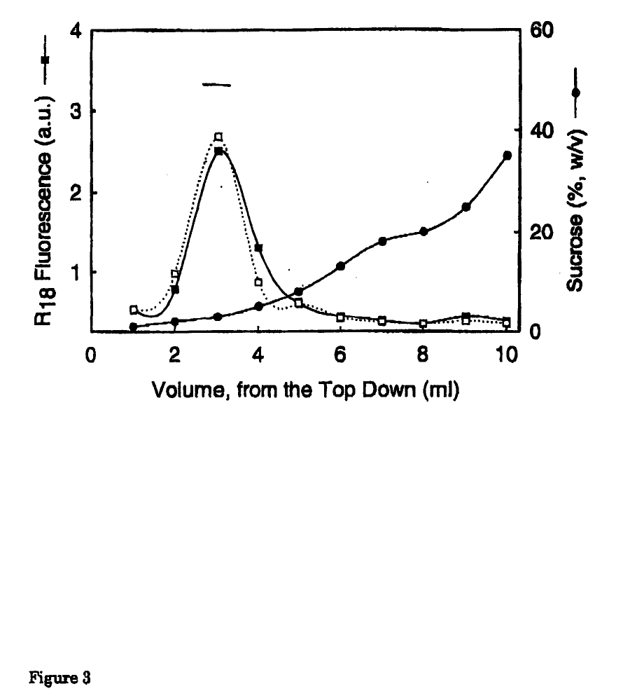

Purified detergent-solubilized MscL was reconstituted into preformed liposomes, which were titrated with low amounts of detergent. After removal of the detergent by adsorption onto polystyrene beads, proteoliposomes were formed. The proteoliposomes were characterized by equilibrium sedimentation on a sucrose gradient as shown in FIG. 3. All 6His-MscL protein detected by the Western blot (inset in FIG. 3) was associated with the lipid bilayer as detected by octadecylrhodamine-β-chloride (R₁₈) fluorescence.

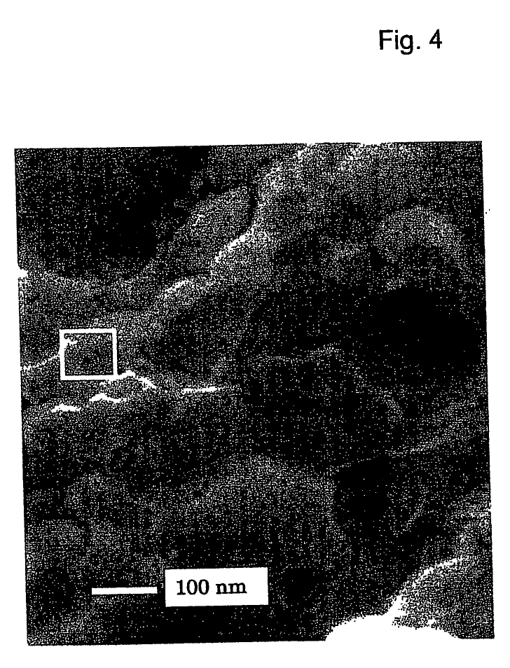

Association of the 6His-MscL protein with the liposomes does not necessarily mean the protein is inserted correctly into the lipid bilayer. Correctly inserted MscL protein should be transmembrane and shows up as an intra-membrane vesicle (IMP) in a freeze-fracture image as shown in the white boxed area of FIG. 4.

The equilibrium sedimentation and freeze-fracture electron microscopy experiments provided structural evidence for the correct reconstitution of the 6His-MscL protein into lipid bilayers.

Electrophysiologic Characterization of Membrane-Reconstituted MscL Activity

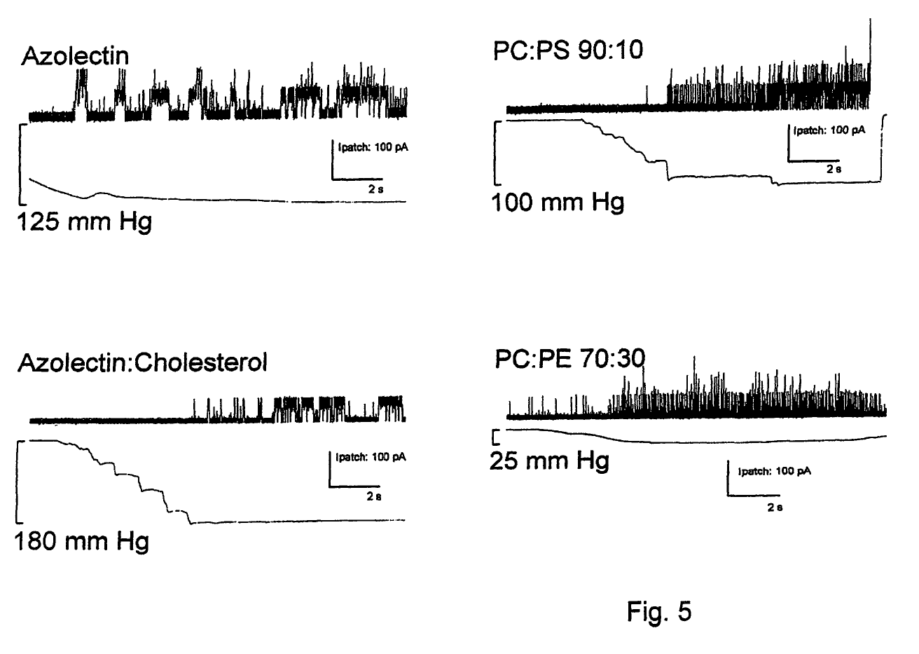

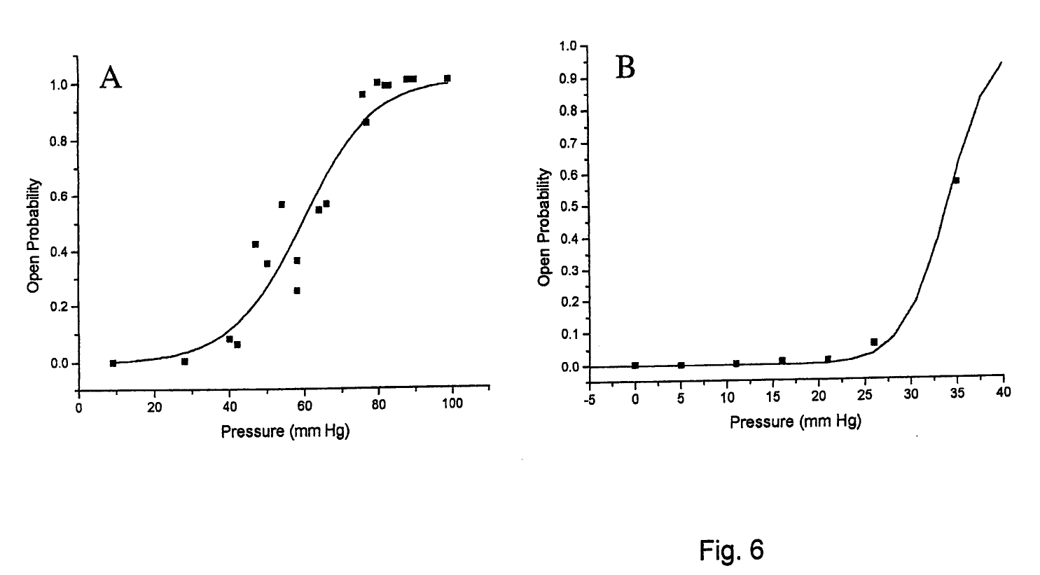

The purified protein reconstituted into phospholipid liposomes forms functional mechanosensitive channels, as seen from the traces in FIG. 5 at different pipette pressures (mechanical activation). The MscL open probability plotted against pressure can be fitted with a Boltzmann distribution (FIG. 6).

Interestingly, reconstituted MscL is active in the absence of negatively charged lipid head groups (FIG. 5, PC:PE 70:30). This is a very important finding since negatively charged head groups prevent targeting to most target sites in the human body. Additionally, these experiments have shown for the first time that the pressure threshold is significantly affected by the lipid composition of the membrane reconstituted MscL channels (FIG. 6). This allows tailor making of the drug release profiles to the specific needs.

We have constructed several 6His-MscL mutants with altered gating properties, i.e., mutants that are hypersensitive to membrane tension and mutants with increased open probability at lower pH values.

In Vitro Release Profiles of a Model Drug

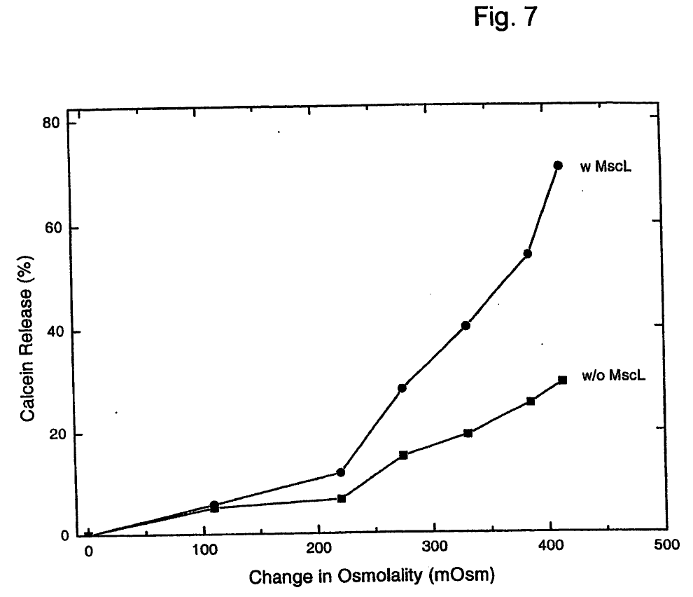

The fluorescence efflux assay was developed to monitor the MscL-mediated release profiles. Liposomes (DOPC:Chol, 60:40, m/m) with and without MscL-6His were subjected to an osmotic downshock, thereby effectively increasing the membrane tension, to monitor the calcein release. As shown in FIG. 7, less calcein remained in the liposomes containing MscL (closed circles) relative to the liposomes without MscL (closed squares) when change in osmolality was larger than 200 mOsm. These data demonstrate that, upon osmotic downshock, liposomes containing reconstituted MscL exhibit a greater efflux of calcein than liposomes without MscL. This MscL-mediated efflux is consistent with the electro-physiologic analysis, showing that the MscL is reconstituted into membranes of synthetic lipids while retaining its functional properties.

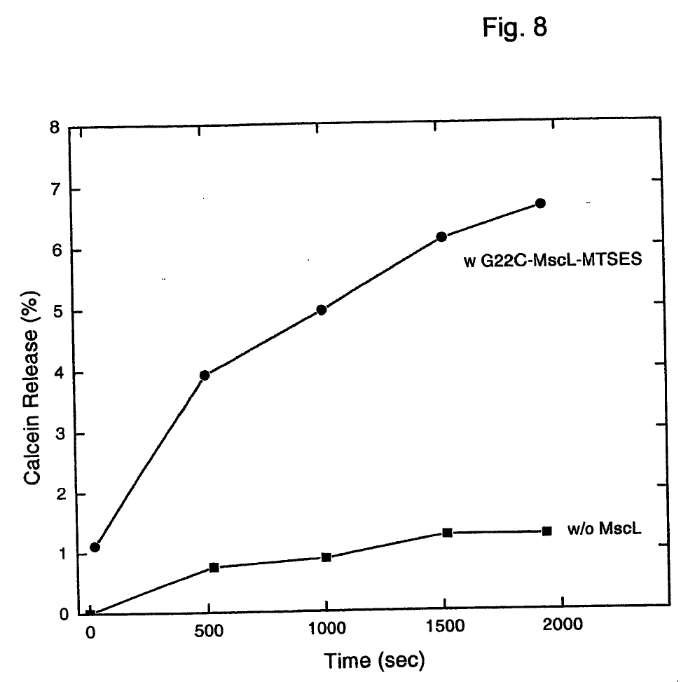

For controlled release of drugs at the target site, membrane tension may not be the most promising stimulus since little is known about osmotic differences in the human body. Several alternatives to activate the MscL channel at the target site are described in this patent. Introducing a charge through conjugation of MTSES to cysteine at position 22 serves as an example for channel activation under iso-osmotic conditions. ESI-MS analysis of the MTSES-conjugated G22C-MscL-6His protein showed that all MscL monomers were conjugated to a stoichiometry of 1:1. The conjugated G22C-MscL-6His samples were subsequently reconstituted into liposomes as described above and calcein release was measured as shown in FIG. 8. These data demonstrate that, in the absence of an increased membrane tension, MscL exhibits drug release from drug-laden synthetic liposomes. This patent includes MscL conjugates that will release drugs at the target site as a function of pH, light activation and specific interactions with target-associated molecules.

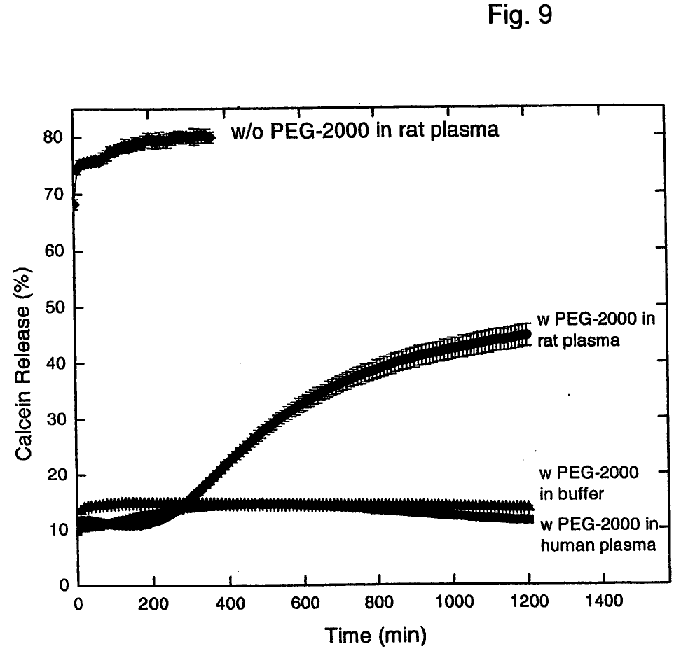

The integrity of liposomes consisting of phosphatidylcholine is seriously affected at first contact with a biological milieu following intravenous injection (J. Damen, et al., 1981, Biochim. Biophys. Acta. 665:538-545). The integrity of liposomes (PC:Chol:DGPE-PEG, 55:40:5, m/m/m) was studied as a function of the molecular mass of the PEG group attached to the DGPE lipid in the presence of rat and human plasma at 37° C. Calcein release from only the liposomes without DGPE-PEG and with 5 mol % DGPE-PEG(2000) are shown in FIG. 9. These data show that addition of 5 mol % of DGPE-PEG(2000) significantly increase liposomal integrity in rat and human plasma up to several hours and will serve to prevent drug leakage during the traveling time to the target cells.

### Example 1-B

### Light-Switchable Opening of the MscL Channel; Conjugation with DTCP1

Photo-reactive compounds can be designed to react with MscL-mutant G22C and respond to the absorption of light by changing the local charge or hydrophobicity. An example of such a photo-reactive molecule is 4-{2-(5-(2-Bromo-acetyl)-2-methyl-thiophen-3-yl)-cyclopent-1-enyl}-5-methyl-thiophene-2-carboxylic acid (DTCP 1).

Materials and Methods

MscL-mutant G22C was overexpressed, purified, labeled, and membrane reconstituted as described in Example 1-C and E. 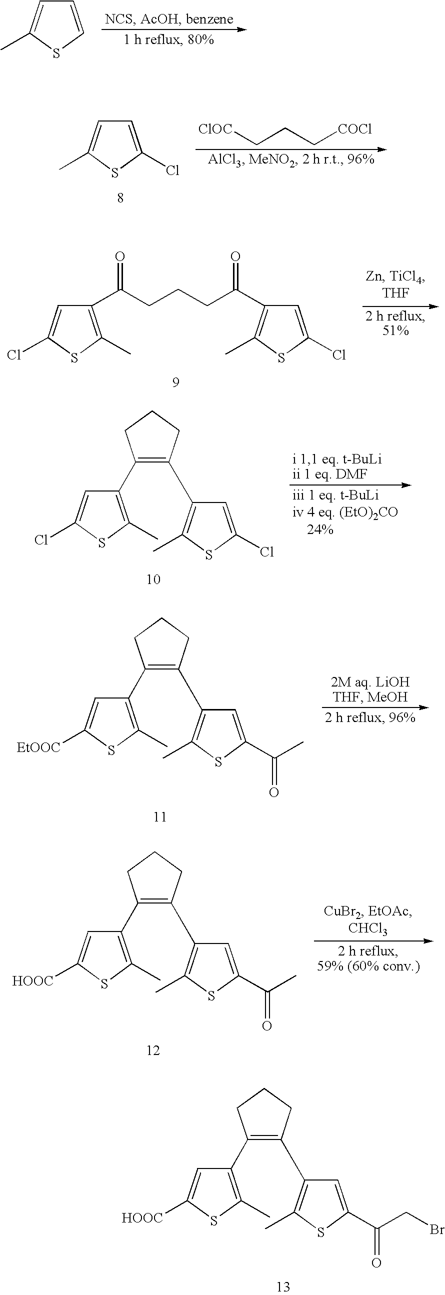
2-chloro-5-methylthiophene (8) (L. N. Lucas, et al., 1998, Chem. Commun. 2313-2314)

Suspension of N-chlorosuccinimide (75.9 g, 0.568 mol) and 2-methylthiophene (50 ml, 50.7 g, 0.516 mol) in a mixture of benzene (200 ml) and acetic acid (200 ml) was stirred 30 minutes at room temperature and then one hour at reflux temperature. Cooled mixture was poured into aq. NaOH (3 M, 150 ml), organic phase washed with NaOH (3M, 3×150 ml), dried over Na₂SO₄ and evaporated in vacuo. Slightly yellow liquid product was further purified by vacuum distillation (19 mm, 55° C.) to give colorless liquid of 2-chloro-5-methylthiophene 8 (55 g, 80.3%).

1,5-bis(5′-chloro-2′-methylthien-3′-yl)pentadione (9) (L. N. Lucas, et al., 1998, Chem. Commun. 2313-2314).

To a solution of 2-chloro-5-methylthiophene 8 (32.3 ml, 39.8 g, 0.3 mol) and glutaryl dichloride (19.2 ml, 25.4 g, 0.15 mol) in nitromethane (300 ml) was added at 0° C. under vigorous stirring AlCl₃ (48 g, 0.36 mol) in several portions. After two hours of stirring at room temperature, ice-cold water (150 ml) was added carefully and extracted with diethyl ether (3×150 ml). Combined ether extracts were washed with water (100 ml), dried over Na₂SO₄ and evaporated in vacuo to yield a brown tar (52 g, 96%). This crude 1,5-bis(5′-chloro-2′-methylthien-3′-yl)pentadione 9 was used further without purification.

1,2-bis(5′-chloro-2′-methylthien-3′-yl)cyclopentene (10) (L. N. Lucas, et al., 1998, Chem. Commun. 2313-2314).

To a Zn dust (10 g, 0.153 mol) suspension in dry THF (200 ml) in a three-neck flask under nitrogen, TiCl₄ (24.8 ml, 42.9 g, 0.226 mol) was slowly added through a glass syringe and resulting mixture was refluxed for 45 minutes. Then, the flask was cooled in an ice bath and crude 1,5-bis(5′-chloro-2′-methylthien-3′-yl)pentadione 9 (27.4 g, 75.9 mmol) was added. After refluxing for two hours, reaction was quenched with aq. K₂CO₃ (10%, 200 ml) and extracted with diethyl ether (4×80 ml). Combined organic extracts were washed with water (100 ml), dried over Na₂SO₄ and evaporated in vacuo. After column chromatography on silica gel (petroleum ether 40-60), 1,2-bis(5′-chloro-2′-methylthien-3′-yl)cyclopentene 10 was obtained as a white solid (12.6 g, 50%).

ethyl 4-(2-(5-acetyl-2-methyl-3-thienyl)-1-cyclolpenten-1-yl)-5-methyl-2-thiophenecarboxylate (11).

To a 1,2-bis(5′-chloro-2′-methylthien-3′-yl)cyclopentene 10 (700 mg, 2.13 mmol) in diethyl ether (50 ml), t-BuLi (1.5M in pentane, 1.7 ml) was added at 0° C. After ten minutes, the cooling bath was removed and reaction mixture stirred for the next 50 minutes at room temperature. Then, N,N-dimethylacetamide (0.2 ml, 195 mg, 2.23 mmol) was added at 0° C., stirred at this temperature for ten minutes and 50 minutes at room temperature. Again, t-BuLi (1.5 M in pentane, 1.4 ml) was added at 0° C., stirred at this temperature for ten minutes and at room temperature for 50 minutes and finally, diethyl carbonate (1 ml, 957 mg, 8.25 mmol) was added at 0° C., stirred at this temperature for ten minutes and at room temperature for 50 minutes. Reaction was then quenched with aq. HCl (1M, 20 ml), the organic layer separated, and the water layer extracted with diethyl ether (3×20 ml). Combined organic layers were washed with saturated aq. NaHCO₃ (10 ml), dried over Na₂SO₄ and evaporated in vacuo. After column chromatography on silica gel (hexane:ethyl acetate/9:1) ethyl 4-(2-(5-acetyl-2-methyl-3-thienyl)-1-cyclopenten-1-yl)-5-methyl-2-thiophenecarboxylate 11 is obtained (192 mg, 24%)

4-(2-(5-acetyl-2-methyl-3-thienyl)-1-cyclopenten-1-yl)-5-methyl-2-thiophenecarboxylic acid (12).

To a solution of ethyl 4-(2-(5-acetyl-2-methyl-3-thienyl)-1-cyclopenten-1-yl)-5-methyl-2-thiophenecarboxylate 11 (114 mg, 0.315 mmol) in mixture of THF (3 ml) and methanol (1 ml), aq. LiOH (2 M, 0.6 ml) was added and the mixture was refluxed for 24 hours. Then aq. HCl (1 M, 10 ml) was added and extracted with ethyl acetate (3×10 ml). Organic extracts were washed with water (5 ml), dried over Na₂SO₄ and evaporated in vacuo. After column chromatography on silica gel (hexane:ethyl acetate/1:1, then CH₂Cl₂:methanol/9:1), 4-(2-(5-acetyl-2-methyl-3-thienyl)-1-cyclopenten-1-yl)-5-methyl-2-thiophenecarboxylic acid 12 was obtained (101 mg, 92%).

4-{2-(5-(2-bromoacetyl)-2-methyl-3-thienyl)-1-cyclopenten-1-yl}-5-methyl-2-thiophenecarboxylic acid (13).

To a boiling suspension of finely ground CuBr₂ (130 mg, 0.581 mmol) in ethyl acetate (2 ml), a solution of 4-(2-(5-acetyl-2-methyl-3-thienyl)-1-cyclopenten-1-yl)-5-methyl-2-thiophenecarboxylic acid 12 (101 mg, 0.292 mmol) in chloroform (2 ml) was added while vigorously stirring. After two hours of reflus, the mixture was filtered and evaporated. After column chromatography on silica gel (CH₂Cl₂:methanol/99:1), 4-{2-(5-(2-bromoacetyl)-2-methyl-3-thienyl)-1-cyclopenten-1-yl}-5-methyl-2-thiophenecarboxylic acid 13 was isolated (73 mg, 59%), together with starting material (40 mg, 40%).

Starting materials were commercially available (Aldrich, Acros Chimica, Fluka) and were used without further purification. Diethyl ether and THF were distilled from Na. For column chromatography Aldrich silica gel Merck grade 9385 (230-400 mesh) was used.

Compounds 10 through 13 are light sensitive and were handled in dark, respectively, using brown glassware.

All compounds were characterized using ¹H NMR (Varian VXR-300 at 300 MHz, or Varian Gemini-200 at 200 MHz), 13C NMR (Varian VXR-300 at 75.4 MHz, or Varian Gemini-200 at 50.3 MHz), and mass analysis (MS-Jeol mass spectrometer).

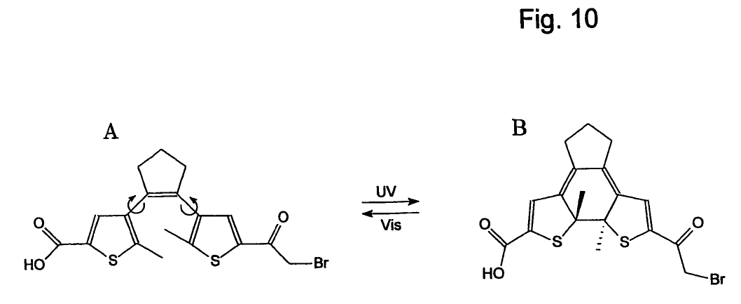

Nuclear Magnetic Resonance and spectroscopic analysis (data not shown) indicated DTCP1 was chemically and functionally correct as shown in FIG. 10.

Results

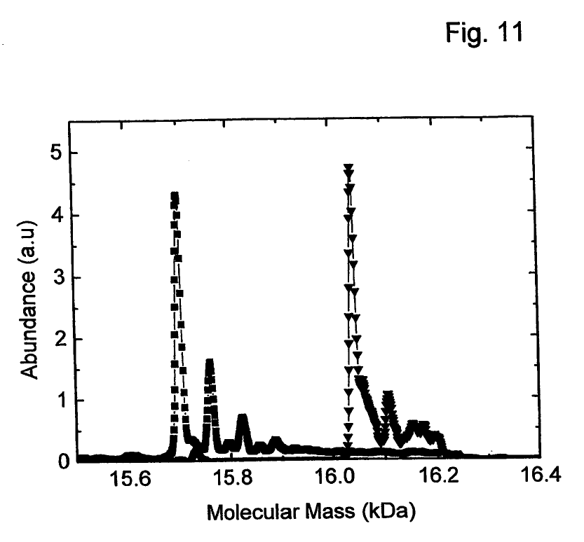

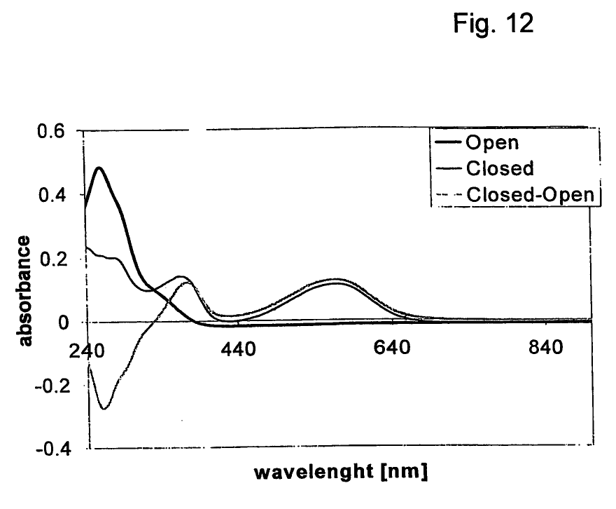

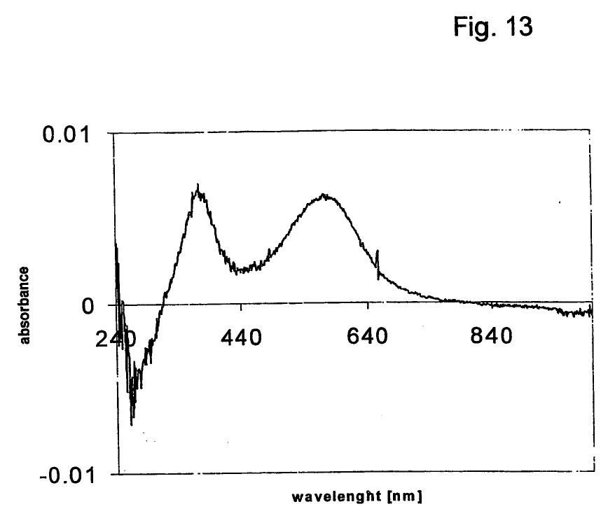

DTCP1 was designed to specifically react with the free sulfhydryl group of a single cysteine at position 22 of MscL (G22C-MscL). Position 22 in the MscL channel was chosen for its involvement in the gating mechanism of the channel. A conjugation protocol was developed and the products were analyzed employing electrospray ionization mass spectrometry (ESI-MS) and absorption spectroscopy. ESI-MS indicated that the mass of all MscL subunits increased with 344 Da, a mass increase expected for a conjugation of DTCP1 to a sulfhydryl group of MscL as shown in FIG. 11. The two photo-isomers of DTCP1 exhibit different absorption spectra in the UV region (FIG. 12). This difference was used to monitor the switching of DTCP1 after conjugation to MscL and reconstitution of the detergent-solubilized G22C-MscL-DTCP1 conjugate into DOPC:DOPS-(90:10, mol/mol) containing lipid bilayer (FIG. 13; due to light scattering by liposomes, only substracted spectra before and after irradiation can be shown).

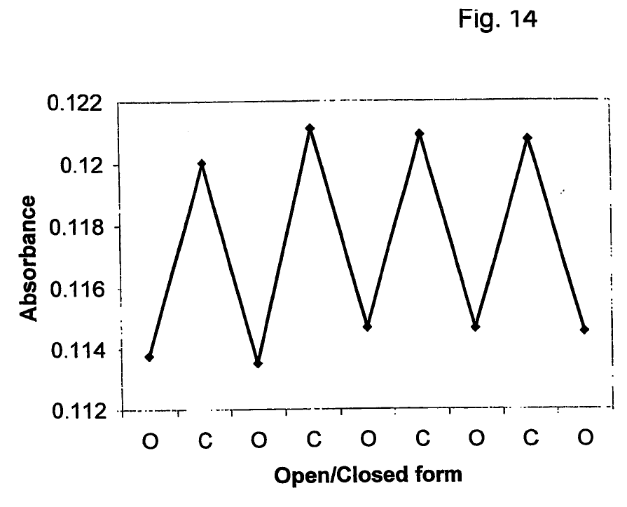

As can be seen from FIG. 13, conjugation and reconstitution into lipid bilayer has no effect on switching of DTCP1. To prove reversibility and reproducibility of switching, this system was repeatedly irradiated with 313 nm UV light to achieve closed form and with light with a wavelength longer than 460 nm to return back to open form, while monitoring at 535 nm (absoprtion maximum of closed form). The results are shown in FIG. 14.

These data show that we have synthesized an organic molecule (DTCP 1) and that this molecule can be conjugated to a specific site in the MscL channel, known to alter the gating properties of the channel, while maintaining the desired photo-chemical properties.

### Example 1-B2

### Light-Switchable Opening of the MscL Channel; Conjugation with SP1

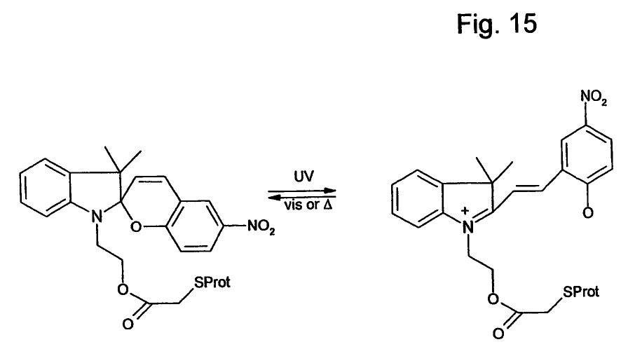

To enhance the hydrophilic properties of the synthesized molecule, we prepared spiropyran derivative SP1 (FIG. 15), that, after UV irradiation, changes into a highly charged merocyanine form.

Materials and Methods

MscL-mutant G22C was overexpressed, purified, labeled, and membrane reconstituted as described in Example 1-C and E, except that labeling on column was 30 minutes instead of three days. 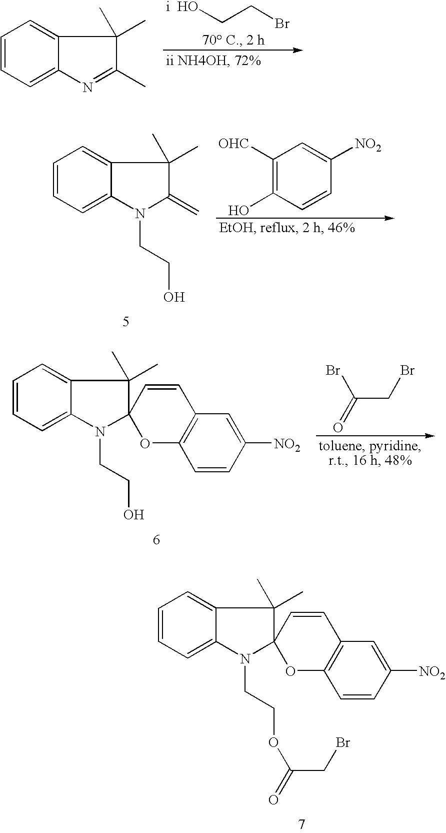
2-(3,3-dimethyl-2-methylene-2,3-dihydro-1H-indol-1-yl)-1-ethanol (5) (M. Sakuragi, et al., 1990, Bull. Chem. Soc. Jpn. 63:74-79).

The mixture of 2,3,3-trimethyl-3H-indole (5 g, 31.4 mmol) and 2-bromoethanol (2.22 ml, 3.92 g, 31.4 mmol) was heated with stirring at 70° C. for two hours, then cooled to room temperature and washed with aq. ammonia (25%, 25 ml). Separated yellow oil was extracted with diethyl ether, dried over Na₂SO₄ and evaporated to give 2-(3,3-dimethyl-2-methylene-2,3-dihydro-1H-indol-1-yl)-1-ethanol 5 as an oil (4.57 g, 72%).

2-(3,3-dimethyl-6-nitrospiro(2H-1-benzopyran-2,2′indoline))-1-ethanol (6) (M. Sakuragi, et al., 1990, Bull. Chem. Soc. Jpn. 63:74-79).

The solution of 2-(3,3-dimethyl-2-methylene-2,3-dihydro-1H-indol-1-yl)-1-ethanol 5 (2 g, 9.8 mmol) and 2-hydroxy-5-nitrobenzaldehyde (1.64 g, 9.8 mmol) in ethanol (50 ml) was refluxed for Two hours. After filtration, the product was recrystalized from ethanol. Yield of pure 2-(3,3-dimethyl-6-nitrospiro(2H-1-benzopyran-2,2′indoline))-1-ethanol 6 was obtained (1.6 g, 46%).

2-(3,3-dimethyl-6-nitrospiro(2H-1-benzopyran-2,2′indoline))ethyl 2-bromoacetate (7).

The solution of 2-(3,3-dimethyl-6-nitrospiro(2H-1-benzopyran-2,2′indoline))-1-ethanol 6 (1 g, 2.84 mmol), bromoacetyl bromide (0.37 ml, 0.86 g, 4.26 mmol) and pyridine (0.35 ml, 0.34 g, 4.26 mmol) in toluene (10 ml) was stirred at room temperature for 16 hours. Then, water (10 ml) was added and extracted with diethyl ether (3×10 ml). Organic extracts were dried over Na₂SO₄, filtered and evaporated in vacuo. After chromatography on silica gel (hexane:ethyl acetate/5:1), oily product 7 (0.65 g, 48%) was obtained.

Results

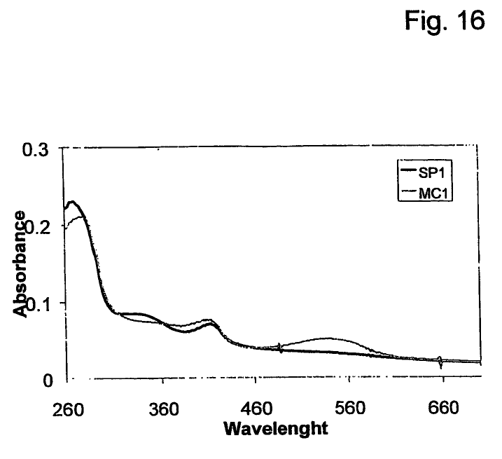

SP1 reacts specifically with the free sulfhydryl group of cysteine at position 22 of MscL, allowing us to specifically modify the channel protein. FIG. 16 shows the UV change of the SP1 conjugated to MscL after irradiation with 313 nm UV light. The new peak at 550 nm belongs to the merocyanine form of the molecule.

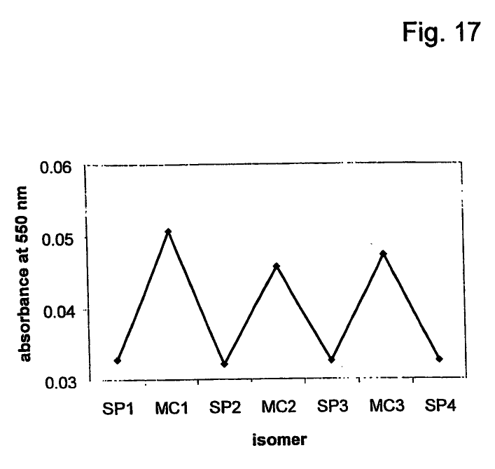

To show reversibility and reproducibility of switching, we repeatedly irradiated SP1 conjugated to protein at 313 nm UV light to achieve merocyanine form and with light with a wavelength longer than 460 nm to return back to spiropyran form, while monitoring at 550 nm (absorption maximum of closed form). The result is shown in FIG. 17.

### Example 1 -B3

### Light-Switchable Opening of the MscL Channel; Use of Photo-Reactive Lipids

This Example shows that photo-reactive lipids can be used to affect the lateral pressure in liposome membranes, thereby controlling the gating of the MscL channel protein.

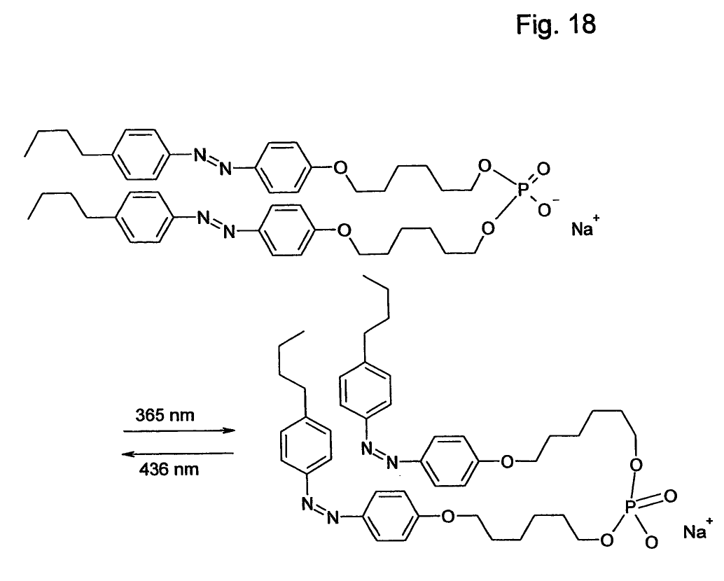

Photo-reactive lipids were designed and synthesized to reversibly switch conformation upon radiation with light of an appropriate wavelength (FIG. 18). Three different lipids with an azobenzene unit have been synthesized (structures shown below; J. M. Kuiper and J. B. F. N. Engberts, to be published). The synthesis of lipid 6 is described in the experimental section. The synthesis of 7 and 8 is similar. 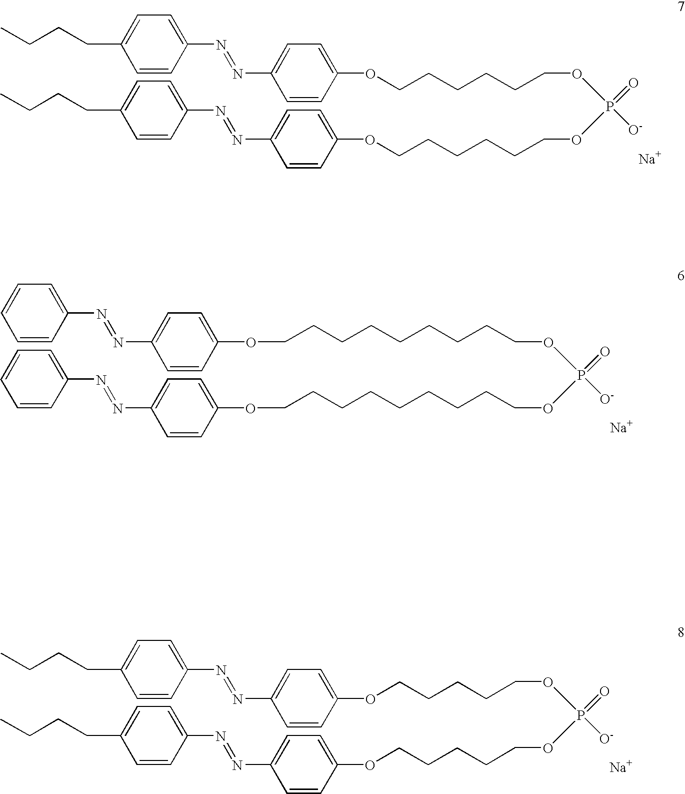
Materials and Methods

Synthesis of 1. To 4.0 g (20.16 mmol) of 4-phenylazophenol in 150 ml of acetone, 4.52 g (20.16 mmol) 9-bromonona-1-ol, 5.56 g (40.32 mmol) of K₂CO₃ and a catalytic amount of KI were added. The mixture was refluxed for five days. The acetone was removed by evaporation under reduced pressure. Dichloromethane (500 ml) was added and the organic layer was washed three times with a fresh layer of water. The organic layer was dried over NaSO₄, filtrated and evaporated under reduced pressure. The resulting yellow solid material was purified by crystallization from ethyl acetate (120 ml). Yellow crystals were obtained in 75% yield and the product was characterized by ¹H and ¹³C NMR.

Synthesis of 2. To 1.5 g (4.41 mmol) of 1 in 15 ml of dry dichloromethane under a nitrogen atmosphere, 238 μl (2.94 mmol) of pyridine and 128 μl (1.47 mmol) of PCl₃ were slowly added. The reaction was monitored by TLC (silica, ether) and additional portions of pyridine and PCl₃ (ratio 2:1) were added when the alcohol was still present. After the reaction was completed, the dichloromethane was washed twice with a saturated aqueous solution of NaCl. In the case of very difficult separations, the addition of some acid sometimes brought some relief. The organic layer was dried over NaSO₄, filtered and evaporated under reduced pressure. The resulting material was stirred overnight in hexane and the crystals were removed by filtration. These crystals were further purified by crystallization from ethanol. The hot solution of product in ethanol was filtrated. The crystallization took place at room temperature. The crystallization was repeated and pure yellow crystals were obtained in a 53% yield. The product was characterized by ¹H, ³¹P and ¹³C NMR.

Synthesis of 5. To 0.305 mg (0.42 mmol) of 2 in 15 ml of tetrachloromethane, 234 μl (1.68 mmol) of triethylamine and 8 μl (±0.1 eq.) of di-isopropylethylamine were added. The mixture was stirred at room temperature for 19 days. The reaction time is much shorter (two to three days); the conversion can easily be followed by ³¹P NMR. The volatile compounds were removed by evaporation under reduced pressure. To the resulting material (3), 0.6 ml acetic acid and 3 ml triethylamine was added. After two days of stirring at room temperature, the reaction was completed. Again, the volatile compounds were removed by evaporation under reduced pressure. The obtained crude product (4) was hydrolyzed by stirring in acidic water (pH=4-5, 100 ml) for 30 minutes. The resulting mixture was subjected to water/dichloroethane (100 ml) extraction. The organic layer was washed twice with a saturated aqueous solution of NaCl. With difficult separations, the addition of some acid is advantageous. The organic layer was dried over NaSO₄, filtrated and evaporated under reduced pressure. The solid material was further purified by crystallization from ethanol. The hot solution of product in ethanol was filtrated. The solution was put away in a refrigerator overnight. The crystals were washed with cold ethanol. Yellow crystals were obtained in a 66% yield and the product (5) was characterized by ¹H, ³¹P and ¹³C NMR.

Synthesis of 6. To 0.1773 g (0.245 mmol) of 5, 1.80 g of a sodium ethoxide solution in ethanol (0.136 mmol/g) was added. Extra dry ethanol was added and the solution was slowly warmed up until it became clear. After cooling down, crystals were formed and the solution was put in the refrigerator. The crystals were washed with cold ethanol. Yellow crystals were obtained in an 89% yield and the product (6) was characterized by ¹H, ³¹P and ¹³C NMR.

Preparation of the Vesicles

DSP/lipid 7 (95:5, mol/mol): The appropriate amounts of the lipids were solubilized in methanol. A thin film was created by evaporating the methanol under reduced pressure. Subsequently, the film was kept under a high vacuum for at least one hour. Water was added and the mixture was stirred firmly for one hour at 85° C. At the end, tripsonication was applied (three times for 30 seconds). A clear solution was obtained.

DOPC/lipid 6 (95:5, mol/mol): The appropriate amounts of the lipids were solubilized in methanol. A thin film was created by evaporating the methanol under reduced pressure. Subsequently, the film was kept under a high vacuum for at least one hour.

Water was added and the mixture was firmly stirred. The mixture was kept at 95° C. for 15 minutes and after that, the mixture was sonicated in a water bath for a few minutes. A clear solution was obtained.

Results

Synthesis: A new synthetic route was used to synthesize the sodium phosphates (see reaction scheme). Particularly, the combination of the third, fourth, and fifth steps is new. These are very mild steps and they can be followed easily by ³¹P NMR. The third, fourth, and fifth steps take place with a complete conversion. First, with the use of PCl₃ and pyridine, the phosphonate can be synthesized. Then, with the use of a base and tetrachloromethane compound, 3a is obtained. This is called the Atherton Openshaw Todd reaction. 3a can react further to 3b. 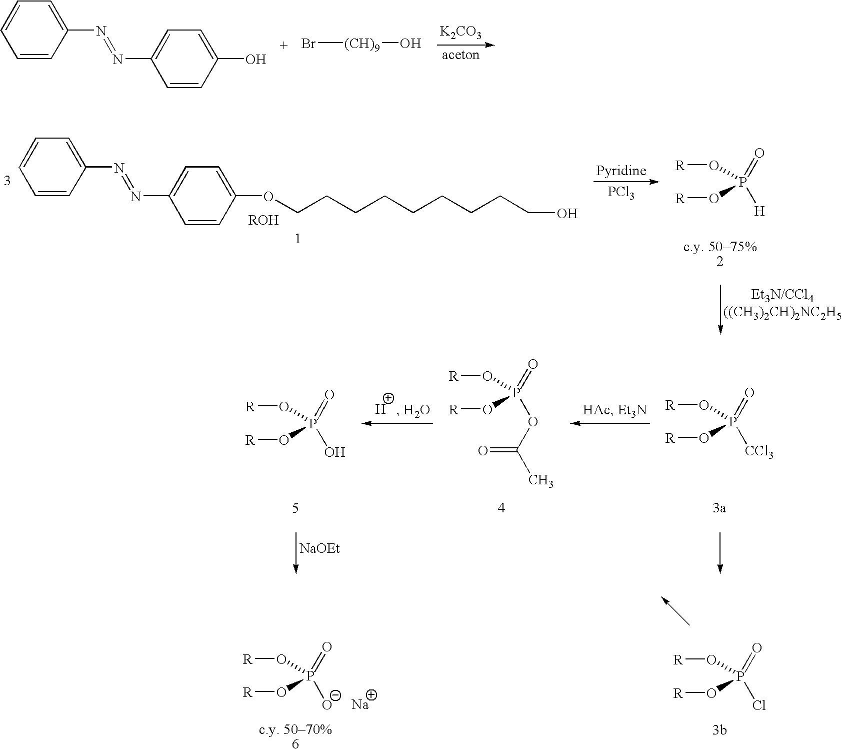
Reaction scheme: Synthesis of lipid 6

After addition of acetic acid and base, a nucleophilic attack of acetic acid takes place at the phosphorus atom. After acid-catalyzed hydrolysis, the phosphate acid is obtained, which can be converted into the sodium salt with the use of sodium ethoxide.

Vesicle Formation

It was found that lipids 6 to 8 are not vesicle forming. This was confirmed by EM (electron microscopy, data not shown). Therefore, the lipids were mixed with vesicle-forming lipids (e.g., DOPC, DOP (sodium dioleyl phosphate) and DSP (sodium distearyl phosphate)). With a ratio of 95:5 for a vesicle-forming lipid and an azobenzene-containing lipid, stable vesicle solutions could be prepared. All mixtures were examined by EM.

UV/Vis Spectroscopy and Irradiation Experiments

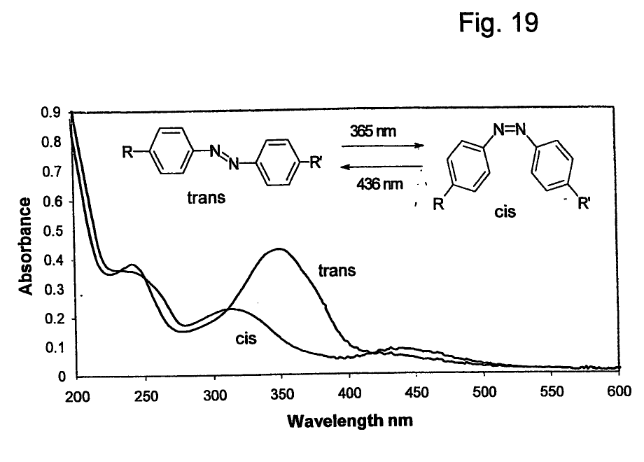

In FIG. 19 the UV/Vis absorption spectra of a mixture of DSP and 7 are shown. The trans isomer was easily switched into the cis isomer upon irradiation with light of 365 nm. Also the back isomerization went smoothly.

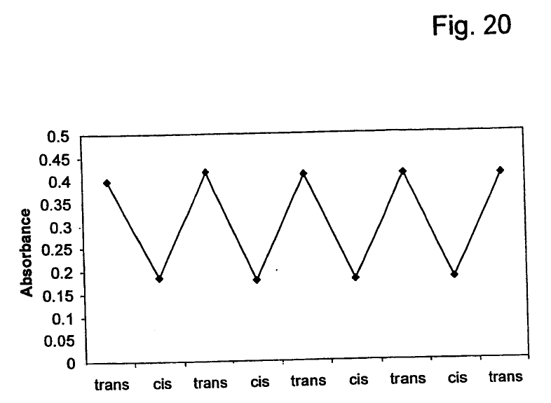

For a mixture of 95% DOPC and 5% lipid 6, the irradiation cycle was repeated several times (FIG. 20).

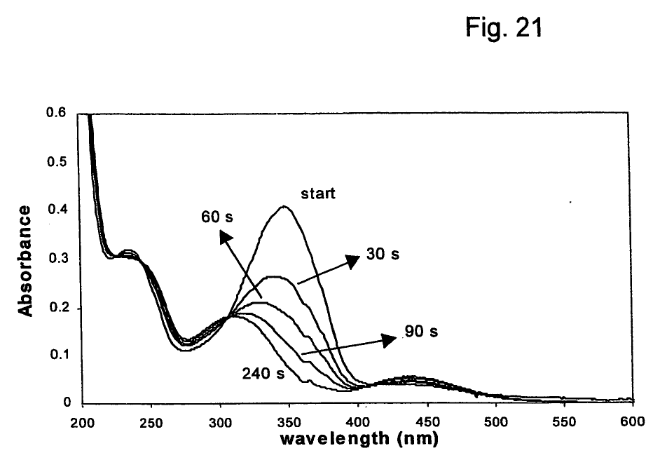

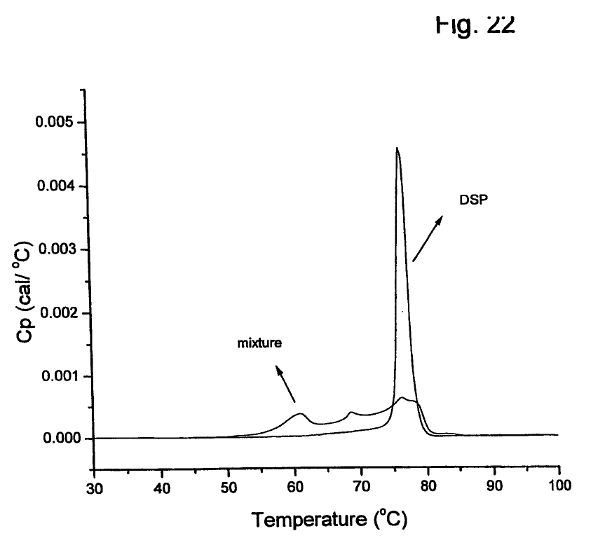

From the experiments, it can be concluded that the isomerization cycle can be repeated several times without decomposition of the material. The trans azobenzene was subjected to irradiation (at 365 nm) for 30 second intervals and the UV/Vis spectrum of the sample was taken between each irradiation cycle (FIG. 21). After four minutes of irradiation, the UV/Vis spectrum did not change any more, which points to a maximal isomerization to the cis isomer. As can be seen from FIG. 21, isobestic points are observed. This means that there is only a transition from the trans isomer to the cis isomer and that there are no side reactions.

DSC Experiments

The DSC graphs show that the phase transition temperature of the vesicles of DSP is changed if 5% of lipid 6 is added (FIG. 22). This indicates that the azobenzene-containing lipids are incorporated into the vesicles. The broad transition indicates that a variety of domains of different lipid compositions are present.

The photo-reactive lipids described above, in combination with other lipids, form liposomes and the physical properties of these liposomes can be altered upon irradiation. MscL channel or derivatives thereof can be reconstituted into these lipid membranes and become responsive to the cis trans switching of the photo-reactive lipids, resulting in controlled drug release.

### Example 1-C1

### pH-Dependent Opening of the MscL Channel; Conjugation with IMI

This example shows the chemical synthesis of a compound that is reactive specifically with cysteine at amino acid position 22 and contains an imidazole group, effectively mimicking the MscL-mutant G22H and circumventing the low production yield of the channel protein.

Materials and Methods

MscL-mutant G22C was overexpressed, purified and membrane reconstituted as described. For labeling of MscL-mutant G22C, protein is isolated as described in Example 1-E, but before elution, column is washed with 10 ml of the wash buffer without imidazole. The label is dissolved to a 1 mg/ml final concentration in the same buffer. The wash buffer in the column is allowed to equilibrate over the column matrix. An equal volume of the buffer containing the label is applied to the column matrix. The top of the column is closed after equilibration with nitrogen gas. The column is incubated at 4° C. for three days and then the elution procedure is followed as described. 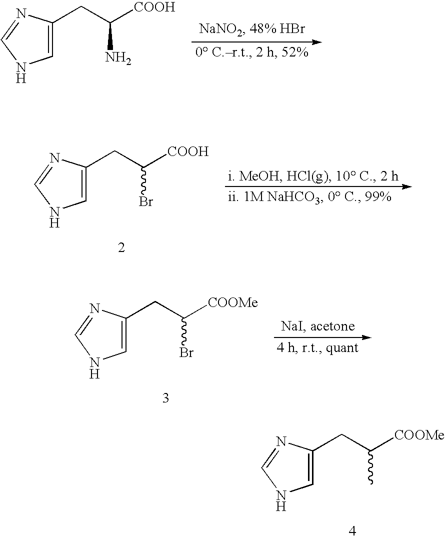
Reaction scheme: Synthesis of BI (2) and IMI (4)
2-bromo-3-(5-imidazolyl)propionic acid monohydrate (2) (J. A. Yankeelov and C. J. Jolley, 1972, Biochemistry 11:159-163).

To a vigorously stirred suspension of L-histidine (7.76 g, 50 mmol) in HBr (48%, 110 ml) kept at −5 to 0° C., a solution of NaNO₂ (10.4 g, 150 mmol) in water (20 ml) was added dropwise. After the addition, the solution was stirred one hour at 0° C., one hour at room temperature, and concentrated in vacuo below 50° C., leaving oil with precipitate. This residue was extracted with acetone (4×15 ml). Acetone extracts were evaporated in vacuo. Water (20 ml) was added and evaporated in vacuo below 50° C. The residue was dissolved in water (30 ml) and pH was adjusted to 4.6 by aq. ammonia (2 M) at 0° C. The solution was evaporated to dryness in vacuo below 50° C. and the solid residue was triturated with ice-cold water (2×30 ml). After drying in vacuo (24 hours, room temperature), the yield of 2-bromo-3-(5-imidazolyl)propionic acid monohydrate 2 was 6.20 g, 52%.

Methyl 2-bromo-3-(5-imidazolyl)propanoate (3) (L. Maat, et al., 1979, Tetrahedron 35:273-275).

Through a stirred solution of 2-bromo-3-(5-imidazolyl)propionic acid, monohydrate 2 (5 g, 21.1 mmol) in methanol (75 ml), kept at 10° C., was bubbled dry HCl during two hours. Then the solution was evaporated in vacuo below 50° C., the resulting oil was dissolved in aq. NaHCO₃ (1 M, 75 ml) and extracted with chloroform (3×50 ml). The extracts were dried over Na₂SO₄, filtered and evaporated in vacuo below 50° C. to yield methyl 2-bromo-3-(5-imidazolyl)propanoate 3 as a slightly yellow oil (4.87 g, 99%).

### Methyl 2-iodo-3-(5-imidazolyl)propanoate (4)

A solution of NaI (3 g, 20 mmol) in acetone (10 ml) was added to a solution of methyl 2-bromo-3-(5-imidazolyl)propanoate 3 (2.33 g, 10 mmol) in acetone (10 ml). Reaction mixture was well stirred and protected from light. After four hours, reaction mixture was evaporated in vacuo to dryness, residue dissolved in water (15 ml) and extracted with ethyl acetate (3×15 ml). Combined extracts were washed with aq. Na₂S₂O₃ (1 M, 5 ml), dried over Na₂SO₄, and evaporated in vacuo at room temperature to give methyl 2-iodo-3-(5-imidazolyl)propanoate 4 as slightly yellow oil (2.80 g, 100%).

Results

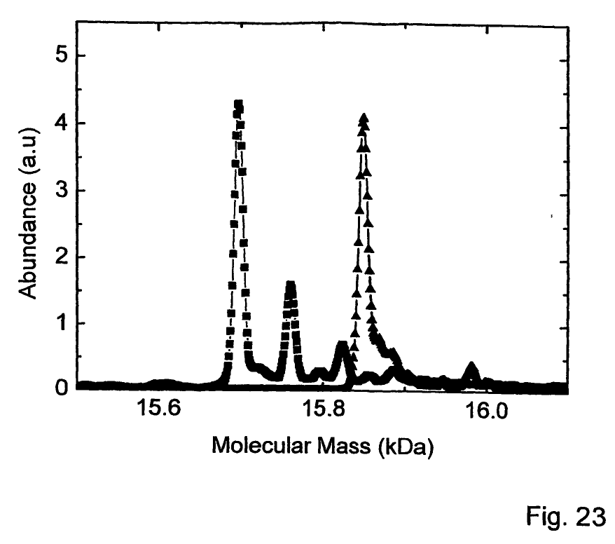

MscL-mutant G22C was labeled with 2-bromo-3-(5-imidazolyl)propionic acid monohydrate (BI) or methyl 2-iodo-3-(5-imidazolyl)propanoate (IMI) for three days and products were analyzed using ESI-MS. The product of the incubation with BI showed a mass identical to the calculated mass of unlabeled MscL-mutant G22C (data not shown). The product of the incubation with IMI showed a mass calculated for the MscL-mutant G22C with an expected additional mass of 153 Da, indicative of proper labeling (FIG. 23). Additionally, no unlabeled protein was observed after labeling with IMI under the described conditions and no doubly labeled subunits were observed, indicating that labeling conditions are optimal for IMI.

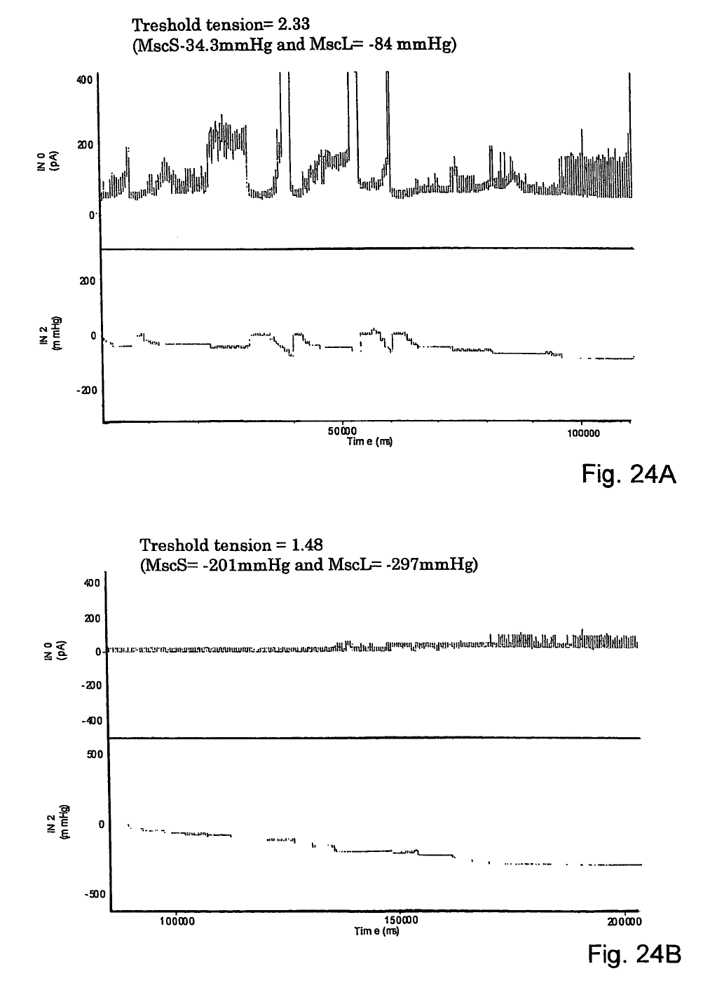

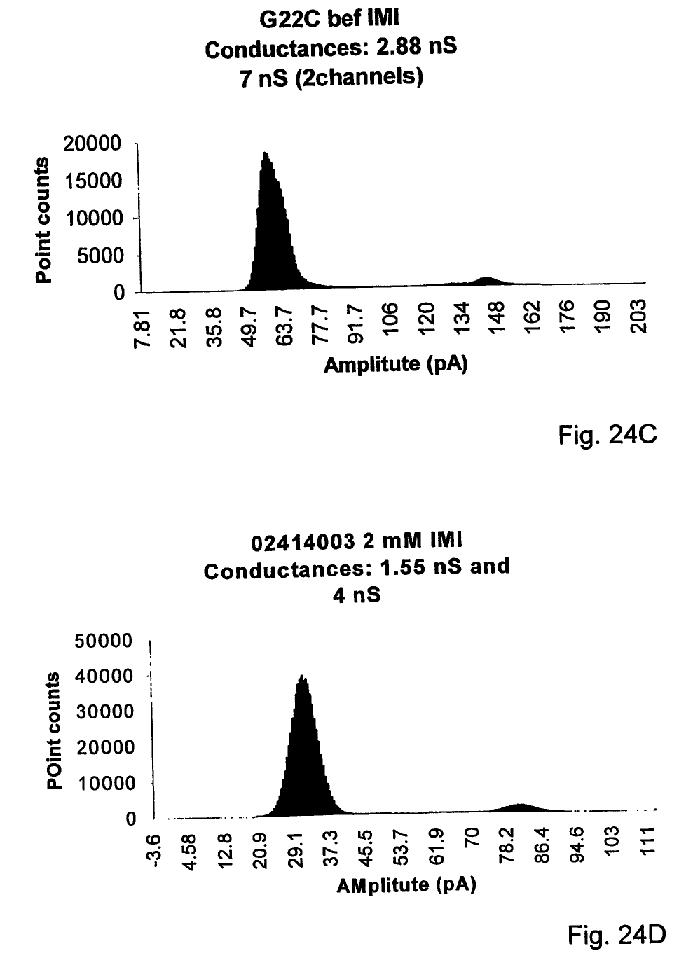

IMI-labeled MscL-mutant G22C in spheroplast was analyzed using patch clamp to characterize the channel properties as shown in FIG. 24.

Patch-clamp experiments on spheroplast allow quantitation of the tension sensitivity because of the presence of an internal control, which is the mechanosensitive channel of small conductance (MscS). By labeling the MscL-mutant G22C with IMI, the ratio of tension sensitivity of MscL over MscS is significantly decreasing from 2.33 to 1.48. This result indicates that the IMI labeling effectively makes the channel protein open at a lower membrane tension at pH 6. Additionally, calcein efflux assays showed that the channel still has a tendency to stay open at pH 7.0 and 8.0 (data not shown). For most clinical applications, it is important that the channel remains closed at pH values of 7.4. Therefore, another compound with a lower pKa was designed and synthesized as described below.

### Example 1-C2

### pH-Dependent Opening of the MscL Channel; Conjugation with pH-Sensitive Compounds Other than IMI

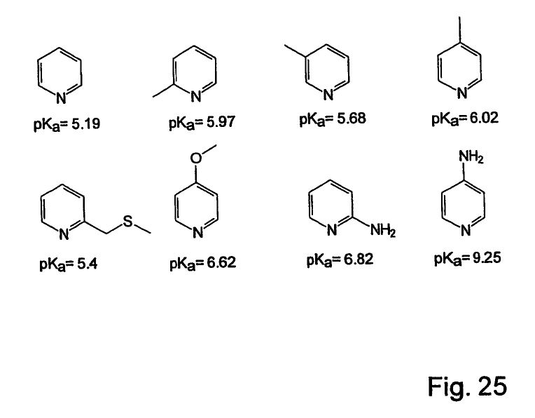

To manipulate the pH sensitivity of the drug delivery vehicle, several other pH-sensitive compounds were designed (FIG. 25). The different pKas of these compounds allow fine tuning of the drug release profile to the specific clinical application.

Materials and Methods

MscL-mutant G22C was overexpressed, purified, labeled, and membrane reconstituted as described in Examples 1-C and E. Synthesis of one of the substituents described in FIG. 25 is described below. 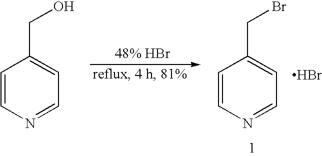
4-(bromomethyl)pyridine hydrobromide (1) (R. L. Bixler and C. Niemann, 1958, J. Org. Chem. 23:575-584).

4-pyridinylmethanol (2 g, 18.3 mmol) was dissolved in aq. HBr (48%, 20 ml), solution was refluxed for four hours and concentrated in vacuo. The semisolid material was triturated with absolute ethanol (10 ml), cooled to 0° C., filtered and washed with another portion of ice cooled absolute ethanol (10 ml). After drying in vacuo, the yield of 4-(bromomethyl)pyridine hydrobromide 1 was (3.67, 81%).

Results

Labeling was optimized for this pyridine compound, 4-(bromomethyl)pyridine hydrobromide (BP), to MscL-mutant G22C, and ESI-MS showed that all channel proteins were labeled (data not shown). Patch clamp was used to characterize the effect of this label on the channel-gating properties.

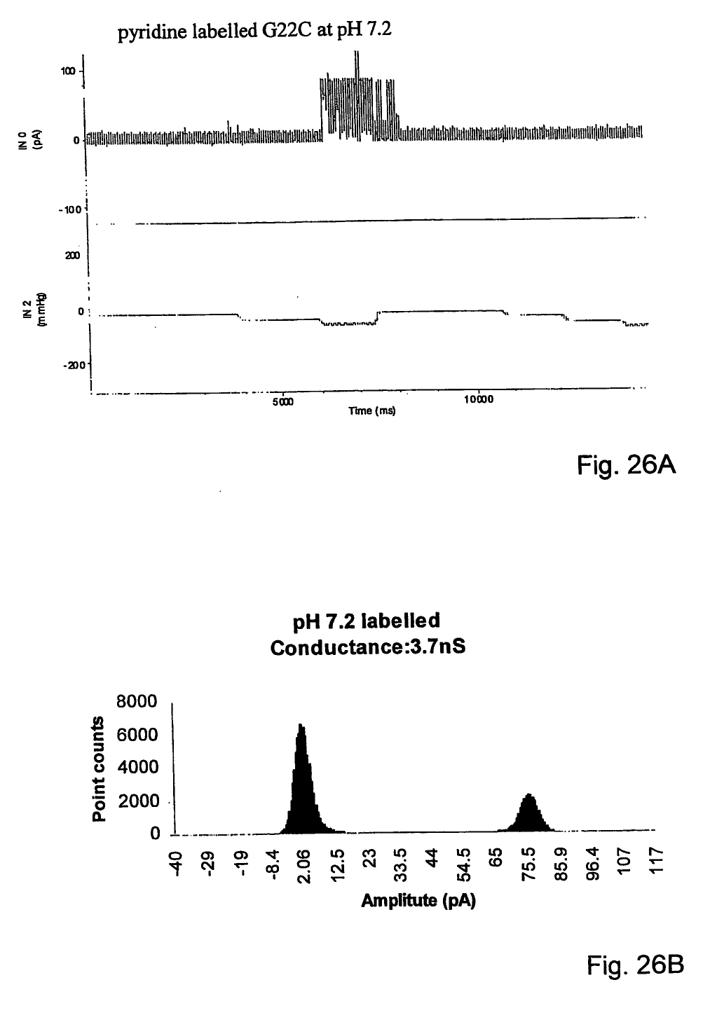

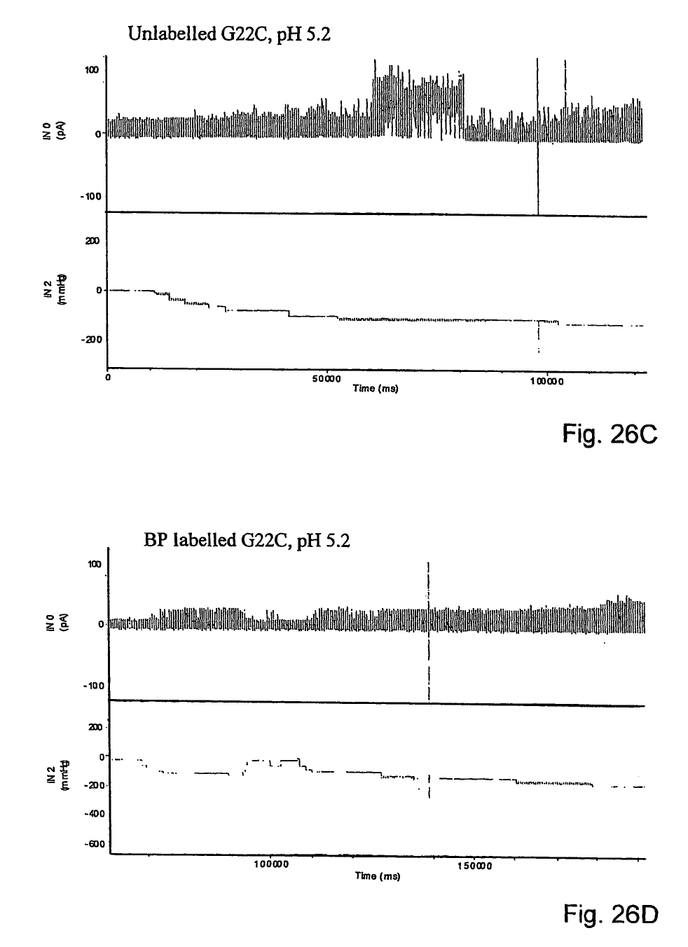

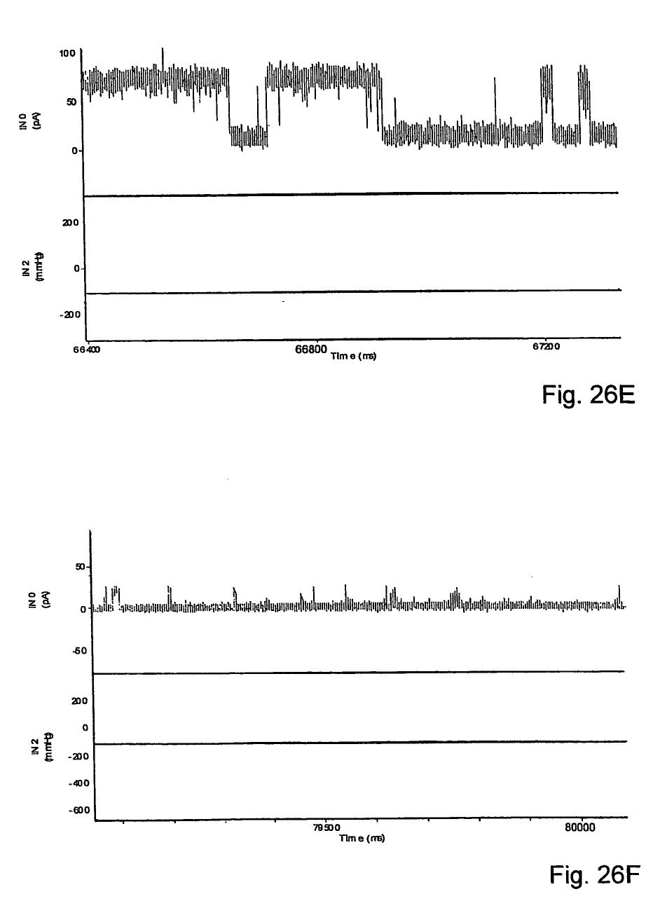

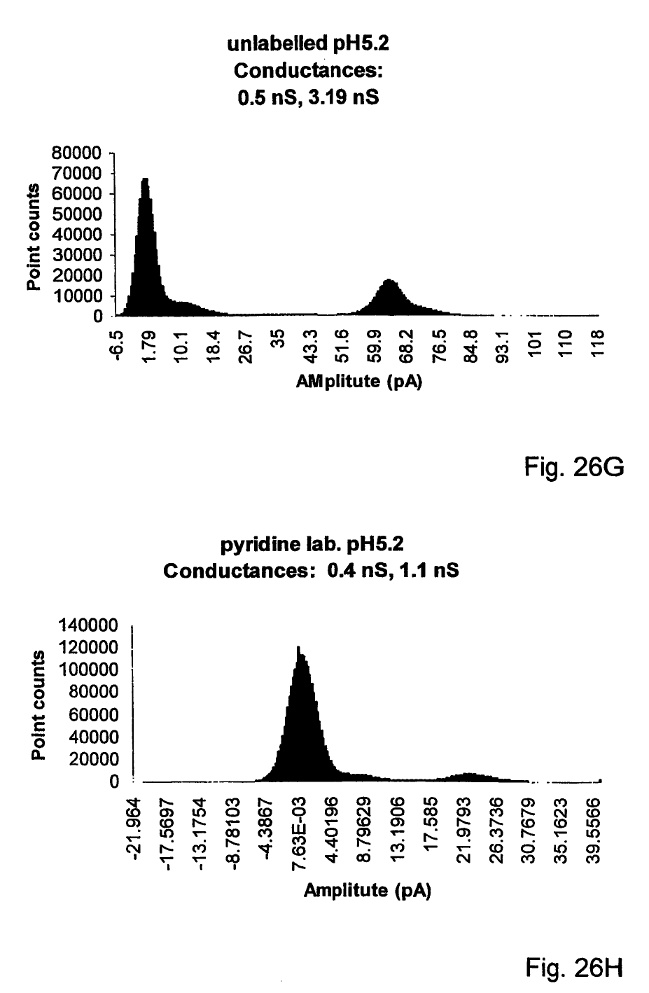

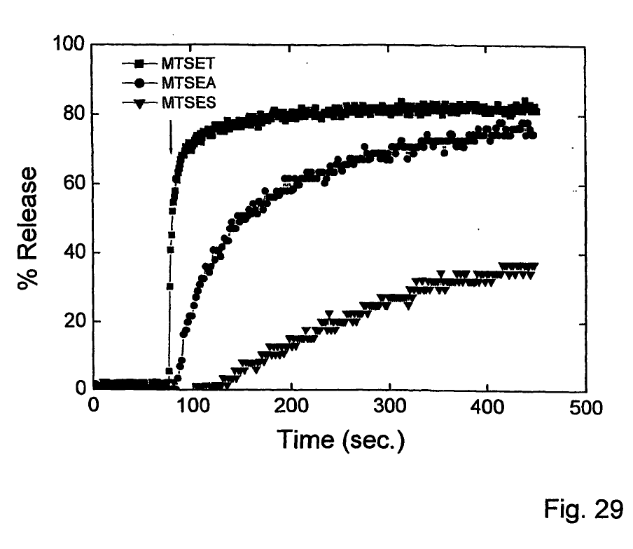

Upon labeling with BP, the MscL-mutant G22C channel shows a pH-dependent change (FIG. 26). At pH 7.2, the BP-labeled channel behaves as an unlabeled channel by exhibiting the same type of conductance preference and dwell times. However, when the channel is analyzed at pH 5.2, it prefers not to open completely but only to subconducting states. The dwell times get shorter. Comparing the behavior of BP-labeled protein at pH 5.2 to both unlabeled protein at pH 5.2 and labeled protein at pH 7.2, indicates that the channel opens normally at high pH values but at lower pH values, starts to open more readily with shorter dwell times. The behavior of the BP-labeled channel at low pH is very similar to the MTSET-labeled channel (FIG. 29), whereas at higher pH values, the BP-labeled channel behaves as unlabeled channel protein. Therefore, it can be concluded that this BP-labeled MscL-mutant G22C will release drugs comparable to the MTSET-induced release of calceine or insulin (see other Examples) at low pH, whereas at higher pH values, the channel is tightly closed, ensuring little or no release of these substances.

### Example 1-C3

### pH-Dependent Opening of the MscL Channel; Use of MscL Mutants

Materials and Methods

Growth Assay

The constructs of the MscL-6His mutants were constructed using standard molecular biology techniques. The mutants were tested in the pB104 strain (MscL null E. coli) by a growth assay on agar plates with or without IPTG (K. Yoshimura et al., 1999, Biophys. J. 77:1960-1972).

Patch Clamp

The G22S mutants with histidine replacements at the S1 region of the MscL were analyzed with the patch clamp technique. Giant spheroplasts were prepared as described before and electrophysiological measurements were done by using symmetrical buffers, both in the bath and in the pipette. The gating threshold of MscL (or its mutant) is given as the ratio of the suction required to open MscL to that at which MscS opens.

Results

Growth Assay

Growth of pB104 harboring Lac-inducible plasmid with mutated MscL inserts on agar plates without (left) or with (right) IPTG is presented in Table E. The results show that the single histidine mutations in the S1 region, except for the R13H mutant, grow after IPTG induction-like WT. However, the histidine mutants in the S1 region, combined with G22S mutation, showed four different phenotypes after IPTG induction. The first phenotype (++) grows like the single G22S mutant. The second phenotype (+++) shows a faster growth rate (K05H/G22S and E06H/G22S). The third phenotype (+) showed a slower growth rate (I04H/G22S and F10H/G22S), and in the fourth phenotype (−), the growth was completely absent (E09H/G22S and R13H/G22S). The K05H/G22S double mutant was analyzed by patch clamp at pH 5.85 and 7.5, respectively.

Patch Clamp

The wild-type MscL (WT), G22S and G22S-K05H mutants were analyzed for pH sensitivity. The results are presented in Table F. K05H/G22S mutant opened at a lower gating threshold at pH 5.85, as compared to wild-type MscL, and opened at a similar gating threshold as wild-type at pH 7.5. However, the K05H/G22S mutant opened at a higher gating threshold at pH 7.5, as compared to G22S MscL, and opened at a similar gating threshold as G22S at pH 5.85. These data show that the K05H/G22S double mutant exhibits a higher open probability at a lower pH value and makes it a suitable MscL mutant for pH-induced drug delivery.

### Example 1-C4

### pH-Dependent Opening of the MscL Channel; Effect of Lipid Composition

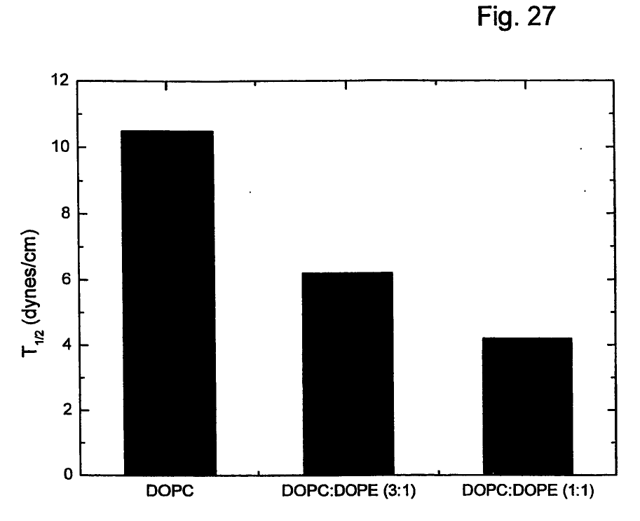

Electrophysiological characterization was performed on MscL and reconstituted in membranes of different lipid compositions. Lipid compositions were chosen to significantly effect lateral pressure profiles of the lipid membranes to gain insight in the gating of the channel. FIG. 27 shows that when the percentage of DOPE increases, the membrane tension necessary to open the channel decreases.

These results provide a rationale for designing a drug-delivery vehicle with appropriate characteristics for specific applications. For instance, the system allows combinations of a specific MscL mutant and lipid composition resulting in specific drug release profiles, tunable to specific needs. For example: N-Citraconyl-dioleoylphosphatidyl-ethanolamine (C-DOPE) was used in a lipid bilayer with DOPC (1:3, m %). The C-DOPE undergoes a proton-catalyzed elimination reaction at pH 5.0, resulting in an increased fraction of DOPE in the bilayer. This increase in DOPE in the bilayer results in a higher open probability of MscL (see FIG. 27). This pH-induced activation of MscL is used to effectively release drugs at the target site.

Another example of manipulation of the physical properties of the liposomal membrane, and thereby controlling MscL-mediated drug release, is modification by phospholipase A2.

### Example 1-D

### Induced Opening of the MscL Channel by Specific Recognition

Three peptides, spanning the portion of the channel accessible at the exterior of the liposomes, were synthesized and used to raise antibodies in rabbits. The bleeds from these rabbits all contained antibodies specific for the synthesized peptides and consequently the full length MscL. Single channel electrophysiologic characterization showed that these bleeds contain antibodies that specifically recognize MscL in the open conformation. These antibodies were used to shift the conformational equilibrium to the open state of the channel.

### Example 1-E1

### Delivery of a Substance from Liposomes through a Charge-Induced Channel Opening; Electrophysiological Characterization

This Example shows the effect of MTSET conjugation to MscL-mutant G22C on the pressure sensitivity of the channel and the change in preference for specific conductance states under patch-clamp conditions.

Materials and Methods

MscL-mutant G22C containing six C-terminal histidine residues was constructed using standard molecular biology techniques. Expression, purification, membrane reconstitution, and patch-clamp analysis were performed as described elsewhere in this application.

MscL Expression and Purification.

E. coli PB104 cells containing the plasmid pB104 carrying the MscL-6His construct were grown to early-logarithmic phase in enriched medium (yeast extract 150 g/l, Bactotrypton 100 g/l, NaCl 50 g/l, K₂HPO₄ 25 g/l, KH₂PO₄ 25 g/l, Antifoam A 2 ml. After sterilization, add 1.5 g Amp 10 ml 1000×Fe⁺² stock (Fe⁺² stock: 0.278 gr FeSO₄, 7H₂O in 100 ml 1N HCl) and 10 ml 1000× spore elements stock (spore elements per 100 ml: EDTA 1 gr, ZnSO₄, 7H₂O 29 mg, MnCl₂, 4H₂O 98 mg, CoCl₂, 6H₂O 254 mg, CuCl₂ 13.4 mg, CaCl₂ 147 mg, pH 4 with NaOH) (15 L fermentor), and induced for four hours with 1.0 mM IPTG (P. Blount, et al., 1996, EMBO. J. 15:4798-4805). Cells were French-pressed and membranes were isolated by differential centrifugation, as previously described (I. T. Arkin, et al., 1998, Biochim. Biophys. Acta. 1369:131-140). The membrane pellet (2.4 g wet weight) was solubilized in 30 ml of buffer A (50 mM K₂HPO₄.KH₂PO₄ pH 8.0, 300 mM NaCl, 35 mM imidazole, pH 8.0, 3% n-octyl-β-glucoside). The extract was cleared by centrifugation at 120,000×g for 35 minutes, mixed with 4 ml (bed volume) Ni²⁺-NTA agarose beads (Qiagen, Chatsworth, Calif.) equilibrated with wash buffer (300 mM NaCl, 50 mM K₂HPO₄.KH₂PO₄ pH 8.0, 35 mM imidazole pH 8.0, 1% n-octyl-p-glucoside) and gently rotated for 30 minutes at 4° C. (batch loading). The column material was poured into a Bio-Spin column (Bio-Rad) and washed with 25 ml of wash buffer, with 0.5 mL/minute flow rate. The protein was eluted with wash buffer containing 235 mM histidine. Eluted protein samples were analyzed by fractionation on a SDS-15% polyacrylamide gel followed by staining with Coomassie Brilliant Blue or transferring the fractionated proteins to PVDF membranes by semi-dry electrophoretic blotting for immunodetection with an anti-His antibody (Amersham Pharmacia Biotech). Immunodetection was performed with an alkaline phosphatase-conjugated secondary antibody as recommended by the manufacturer (Sigma).

Electrophysiologic Characterization of Membrane-Reconstituted MscL

MscL was reconstituted into liposomes of different lipid composition and aliquots of 200 μL were centrifuged at 70,000 rpm in a tabletop ultracentrifuge (Beckmann). Pelleted proteoliposomes were resuspended into 30 μL buffer C (10 mM 4-morpholinepropanesulfonic acid (MOPS) buffer, 5% ethylene glycol, pH 7.2), and 15 μL droplets were subjected to a dehydration-rehydration cycle on glass slides (A. H. Delcour, et al., 1989, Biophys. J. 56:631-636). Rehydrated proteoliposomes were analyzed employing patch-clamp experiments as described previously (P. Blount, et al., 1996, EMBO. J. 15:4798-4805).

Giant spheroplasts were prepared as described in P. Blount, et al., 1999, Methods Enzymol. 294:458-482.

Results

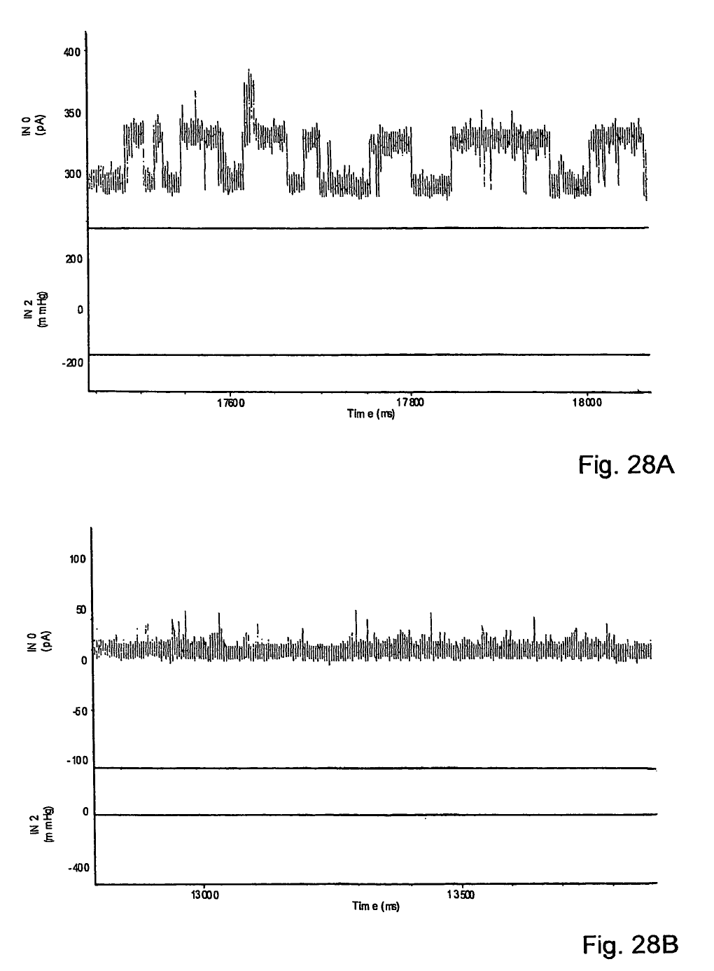

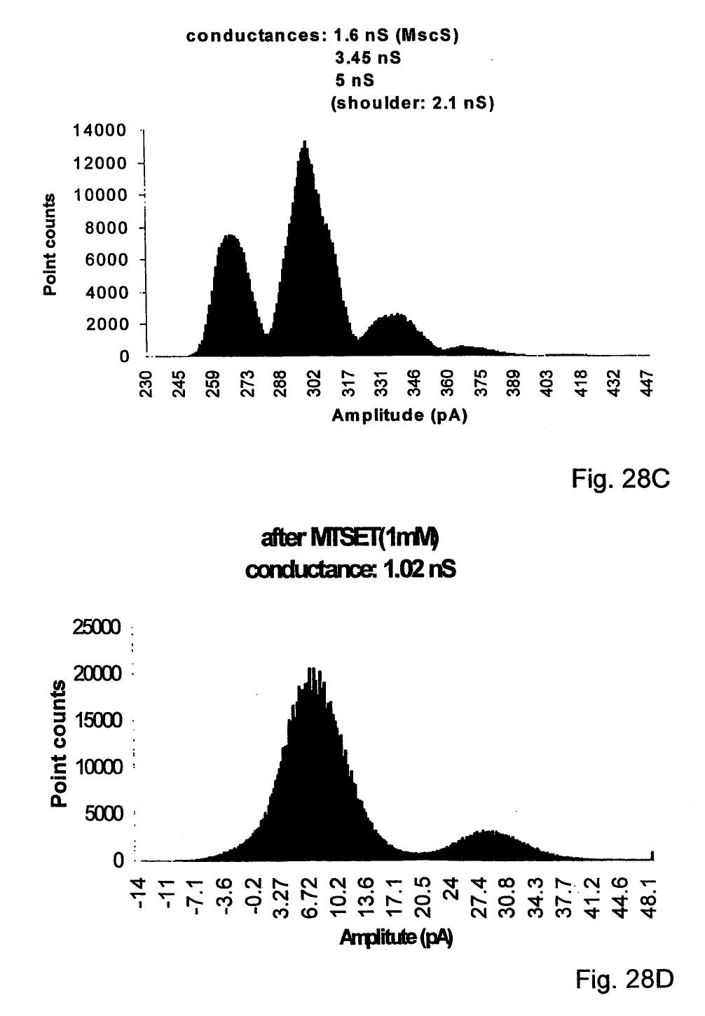

The electrophysiologic characterization of the MscL-mutant G22C as shown in FIGS. 28A and 28C resulted in similar channel properties as described in literature with respect to pressure sensitivity and a conductance of 3.42 nS. The other observed conductances are from MscS (1.6 nS), another mechanosensitive channel in spheroplast, and most probably a simultaneous opening of the MscL-mutant G22C and MscS (5.0 nS). However, when MTSET is attached to MscL-mutant G22C, channel properties change significantly as shown in FIGS. 28B and 28D. First, pressure sensitivity decreases significantly, resulting in spontaneous gating of the channel. Second, when the MscL channel opens, it closes much faster, with smaller dwell times. It appears as if the introduced charge at amino acid position 22 destabilizes both the closed and open state. As a consequence, the channel rapidly switches from the closed to open state, resulting in this “flickery” appearance even in the absence of membrane tension.

### Example 1-E2

### Delivery of a Substance from Liposomes through a Charge-Induced Channel Opening; Drug Release Profiles

This Example shows that after MscL purification and membrane reconstitution into an artificial lipid membrane, attachment of MTSET, MTSEA, or MTSES to MscL-mutant G22C results in spontaneous gating. Additionally, we show that the charge-induced channel opening can result in the release of a membrane-impermeable hydrophilic molecule from artificial liposomes containing MscL-mutant G22C upon the introduction of a charge by means of an MTS compound.

Materials and Methods

Samples were prepared and calcein efflux was monitored as described elsewhere in Example 1.

Results

Increase in MscL-mediated calcein release upon attachment of MTSET, MTSEA or MTSES to the MscL-mutant G22C is shown in FIG. 29. MscL-mutant G22C reconstituted into DOPC:DOPS (90:10, mol/mol) liposomes showed no calcein release at the time scale of this experiment as indicated by the stable fluorescence in the first 85 seconds of the experiment. At 85 seconds, 1 mM MTSET was added to the sample and calcein was rapidly released. In control liposomes, having the same lipid composition but without MscL, no calcein release was observed (data not shown). This experiment shows that when formulated as described, 80 percent of the encapsulated calcein is effectively released from these liposomes and half of the MscL-mediated efflux occurs within seven seconds. This experiment was repeated using another positively charged compound MTSEA, and a negatively charged compound, MTSES. Calcein is released with both these compounds, however, the kinetics of release are significantly slower. The release of calcein upon the addition of these compounds is a composite of the reactivity of the MTS compounds with cysteine at position 22, opening of the MscL channel in response to the introduced charge, and the efflux of calcein through the MscL channel.

These results establish the correlation between the gating properties as determined with patch clamp and the release profile of a hydrophilic molecule from liposomes. It can be concluded that even when a channel exhibits short dwell times, hydrophilic molecules of approximately 600 Da can be effectively and rapidly released under the control of a charging amino acid at position 22 of the MscL channel.

These results show that liposomes with MscL-mutant G22C can be used in a two-component system. In this, the first component, liposomes with MscL-mutant G22C with encapsulated drugs are administered. After these liposomes have accumulated at a target site, a second component is administered. This second component, the MTS compound or one similar in that it attaches specifically to the cysteine at position 22, will then effectively cause the release of the encapsulated drugs. By using second components of different hydrophobicities, tailor making of the drug release profiles is allowed as is shown in FIG. 29.

### Example 1-F

### Formulation of Sterically Stabilized Liposomes Containing MscL with Encapsulated Compounds

Materials and Methods

MscL-mutant G22C containing six C-terminal histidine residues was constructed using standard molecular biology techniques. Expression, purification, membrane reconstitution, and patch-clamp analysis were performed as described elsewhere or as described below.

MscL Expression and Purification

Membrane vesicles were prepared as described in Example 1-E1. MscL has previously been isolated by using the detergent n-octyl-β-D-glucopyranoside. In an improved method, the membrane pellet (2.4 g wet weight) was solubilized with a buffer (50 mM K₂HPO₄.KH₂PO₄ pH 8.0, 300 mM NaCl, 35 mM imidazole, pH 8.0, 1% Triton X-100) containing 1% Triton X-100 instead of 3% n-octyl-β-D-glucopyranoside. The extract was cleared by centrifugation at 120,000×g for 35 minutes, and the supernatant was mixed with Ni²⁺-NTA agarose beads (Qiagen, Chatsworth, Calif.), which were pre-equilibrated with 10 ml of water, followed by 25 ml of wash buffer (300 mM NaCl, 50 mM K₂HPO₄.KH₂PO₄ pH 8.0, 35 mM imidazole pH 8.0, 1% Triton X-100). The mixture of the solubilized membrane fraction and the Ni²⁺-NTA matrix slurry was gently rotated for 30 minutes at 4° C. It was poured into a column and washed with 30 ml of wash buffer containing 0.5% Triton X-100 instead of 1%, with a flow rate of 0.5 ml/minute. The protein was eluted with elution buffer (300 mM NaCl, 50 mM K₂HPO₄.KH₂PO₄ pH 8.0, 235 mM histidine and 0.2% Triton X-100). Eluted protein samples were analyzed by fractionation on a SDS-12% polyacrylamide gel followed by staining with Coomassie Brilliant Blue.

Membrane Reconstitution of MscL into Liposome-Containing PEG 2000

For the membrane reconstitution of MscL-mutant G22C in liposomes composed of DOPC/cholesterol/DSPE-PEG 2000=70:20:10 (mol %), preformed liposomes (4 mg/ml) were partially solubilized with 0.2% Triton X-100. 300 μl of detergent-treated liposomes were gently mixed with 240 μl of 0.25 mg/ml purified MscL-mutant G22C in elution buffer containing 0.2% Triton X-100. The mixture was incubated at 60° C. for 45 minutes, followed by the addition of an equal volume (540 μl) of calcein-loading buffer, containing 200 mM calcein, 25 mM Tris, 1 mM EDTA, pH 8.0. After addition of 20 mg of Triton X-100-absorbing Bio-Beads SM-2, the mixture was gently agitated for one hour at room temperature. Subsequently, 80 mg Bio-Beads were added and incubated at 4° C. overnight under mild agitation. Samples were prepared for calcein efflux assay as described elsewhere.

Results

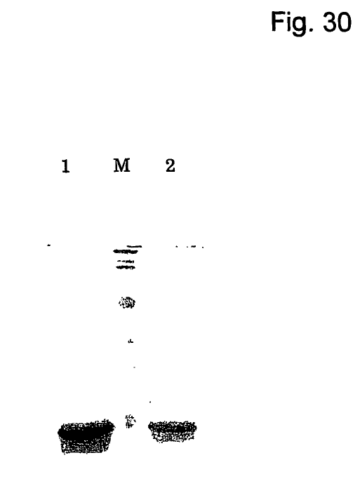

The isolation of MscL employing n-octyl-β-D-glucopyranoside or Triton X-100 resulted in purified MscL protein as shown in FIG. 30. The Triton X-100 was more effective in extracting MscL from the membrane vesicles (0.5 mg/ml as compared to 0.4 mg/ml) and resulted in a higher yield (4.5 mg/l of culture).

The isolated protein was reconstituted into DOPC liposomes and analyzed with the patch-clamp technique as described elsewhere. Detergent was removed by using Bio-Beads instead of dialysis. Both samples, isolated with n-octyl-β-D-glucopyranoside and Triton X-100, exhibited the same electrophysiologic channel characteristics as described in the literature.

MscL-mediated release from DOPC:DSPE-PEG (94:6, mol %) liposomes was examined by monitoring the increase in fluorescence of the self-quenching fluorescent dye, calceine. Upon addition of MTSET to these liposomal formulations, MscL-mediated calceine release of 66.2%+/−2.19 (n=5) were observed.

### Example 1-G1

### MscL-Mediated Drug Release In Vivo; MAG3 Release from Liposomes

The effect of drug formulation on the rate of drug release in vivo was tested in the rat by external counting of radioactivity. The model is based on liposomal formulations that remain at the subcutaneous site of administration with an encapsulated model drug which, in case released, is rapidly removed from the subcutaneous site of injection and excreted into the urinary bladder.

Materials and Methods

Material

DOPC/DOPS (90:10, mol/mol) liposomes with or without the MscL-mutant G22C were used (protein-to-lipid ratio of 1:20, wt/wt). Radio-labeled mertiatide (^{99m}Technetium-MAG3) was chosen as the model drug because of its rapid and exclusive excretion from the circulation into the urine (via active tubular secretion, 600 mL/minute in humans).

Sample Preparation

Encapsulation of MAG3 in liposomes was performed by freezing/thawing three times followed by extrusion through a 400 nm filter. The free fraction of the compound was removed by G50 Sephadex column separation.

The normal liposomes were loaded with MAG3 in 0.9% NaCl and eluted on the G50 column with 25 mM HEPES pH 8, 150 mM NaCl. The G22C-MscL liposomes were loaded with the model drug in 150 mM sucrose and 145 mM NaCl and eluted on the G50 column with 25 mM HEPES pH 8, 150 mM sucrose, 145 mM NaCl.

Kinetics of Encapsulated Model Drugs Administered Subcutaneously

Male Wistar rats were anaesthetized using iso-flurane O₂/NO throughout the study. Liposome-encapsulated MAG3 in a 0.5 ml volume was injected subcutaneously (in the neck) and accumulation of radioactivity in the urinary bladder constantly monitored by external counting using a gamma-camera (window: 140 keV, 250 keV width, one minute time resolution). At the end of the study (after 40 minutes), a ten times higher dose of free MAG3 was administered subcutaneously or intravenously to measure the urinary excretion rates of those formulations.

Results

Kinetics of Free and Encapsulated Model Drugs

Compared to intravenous administration of the free compound, the urinary excretion of MAG3 was slower after subcutaneous injection (50% in 3 and >12 minutes, respectively; FIG. 31).

Encapsulation in liposomes reduced the rate of urinary MAG3 excretion with a significant difference between the liposomes tested. Compared to the normal DOPC/DOPS liposomes, the G22C-MscL-containing liposomes released significantly more MAG3 (15% and 45% urinary MAG3 excretion in the first 30 minutes after injection).

These results show that liposomes containing MscL-mutant G22C exhibit release of the hydrophilic molecules MAG3. This MscL-mediated release is significantly faster than MAG3 release from liposomes without MscL but slower compared to free MAG3 injected subcutaneously. Therefore, it can be concluded that the MscL channel modulates the transport of hydrophilic molecules in vivo, and can be used as a drug delivery vehicle for sustained release.

### Example 1-G2 MscL-Mediated Drug Release in vivo; pH-Induced Drug Release

This Example describes a method to induce a temporary pH reduction subcutaneously for the testing of pH-sensitive MscL-mediated drug release.

Materials and Methods

Male Wistar rats remained conscious throughout the study. The subcutaneous pH was constantly recorded using a micro-glass electrode. After stabilization of the pH, 0.5 ml MES buffer (pH 6.1) was injected approximately 3 mm from the pH electrode. The effect of different molarities (50, 100 and 250 mM) of the MES buffer on the time-course of pH reduction was tested.

Results

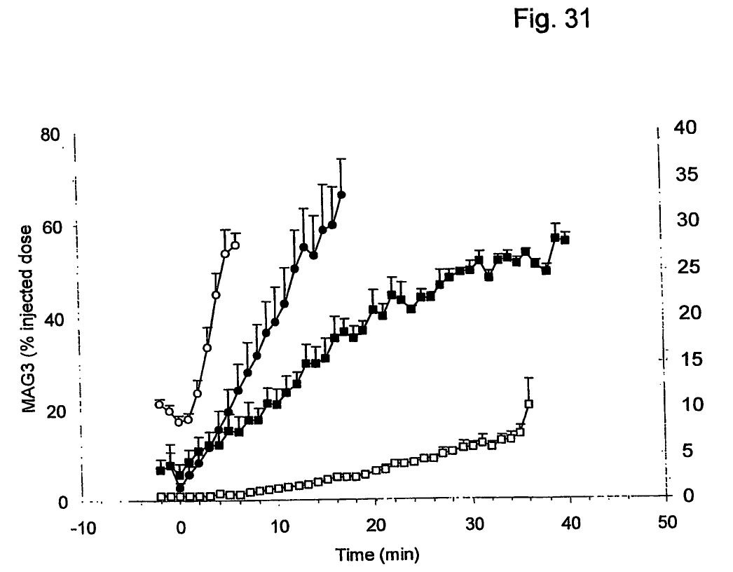

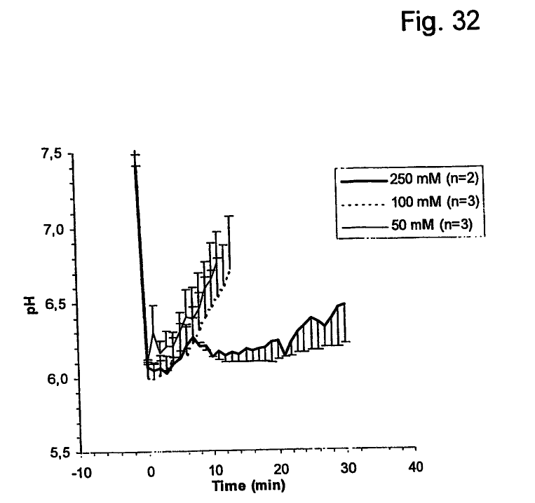

MES buffer is suitable to lower the pH in the subcutaneous tissue. The duration of pH reduction appeared to depend on the molarity of the buffer (FIG. 32). With 50 and 100 mM MES, the pH returned steadily to physiological pH (pH 7.4) within ten minutes. By using 250 mM MES, pH remained below pH 6.5 for more than 30 minutes.

The subcutaneous tissue can temporarily be acidified by an MES buffer with the molarity of the buffer determining the duration of pH reduction. Short-lasting pH reductions allow measurement of the effect of repeated gating and closing of the channel.

### Example 1-G3

### MscL-Mediated Drug Release In Vivo; Slow Release

The radioactive method, described in Example 1-G1, is suitable to determine the rate of subcutaneous-released drug administered in different formulations. Drawbacks are the unphysiological state of anesthesia and the limited period of time that can be measured (due to the short half-life of the radioactive label, the instability of the compound and the required anesthesia). Therefore, an alternative was developed. In this method, the release of a drug from different formulations subcutaneously can be determined in conscious rats for a long period of time (days).

Materials and Methods

Material

DOPC/DOPS (90:10, mol/mol) and DOPC/DOPE liposomes (70:30, mol/mol) liposomes were tested. Iodo-thalamate (IOT) was chosen as the model drug because of its rapid and exclusive excretion (via glomerular filtration, 1 ml/minute⁻¹ 100 g rat) from the circulation into the urine.

Sample Preparation

Encapsulation of IOT in liposomes was performed by freezing/thawing three times followed by extrusion through a 400 nm filter. The free fraction of the compound was removed by G50 Sephadex column separation.

The liposomes were loaded with IOT in an iso-osmotic solution (25 mM HEPES pH 7.4 and 145 mM NaCl) and eluted on the G50 column using the same buffer as eluens.

Kinetics of Encapsulated Model Drugs Administered Subcutaneously

Male Wistar rats remained conscious throughout the study. To increase the time-resolution, the diuretic furosemide (10 mg/kg dose s.c) was given in the morning, seven hours before administration of the sample. Urine was collected automatically with a one hour resolution. The concentration of IOT in the urine was measured by HPLC.

Results

Kinetics of Free and Encapsulated Model Drugs

After subcutaneous injection of free IOT, the first phase urinary excretion of the model drug was completed in the first couple of hours after injection (FIG. 33). In contrast, the rate of excretion was strongly reduced by liposomal encapsulation.

Both the radioactive (MAG3) method and the present method are suitable to measure the stability of subcutaneous liposomal drug formulations. The radioactive method is more suitable for relatively fast releasing formulations, whereas the last-described method is more suitable for the slower releasing formulations. These animal models can be used to monitor the controlled release of drugs from liposomal formulations containing MscL channels or derivatives thereof that respond to changes in pH, light of a specific wavelength, changes in osmolality, or the addition of an activator such as MTSET or reduced glutathione (described in previous Examples).

### Example 1-H1

### Use of MscL Homologues from Organisms Other than E. coli; Preparation and Functional Characterization of MscL from L. lactis

Material and Methods

MscL^{Ll} Expression, Purification and Reconstitution

The gene of MscL^{Ll} was taken from the GRAS organism Lactococcus lactis IL1403 (NCBI: 12725155) and cloned with a 6-histidine tag into an overexpression vector. Lactococcus lactis NZ9000 cells containing the plamid pNZ8020MscL^{Ll}6H carrying the MscL-6Histidine construct was grown to OD₆₀₀ of approximately one in 3L M17 (Difco) medium supplemented with 10 mM argenine and 0.5% galactose and induced with 0.5 ng/ml (final concentration) Nisin for three hours. The cells were harvested and washed by centrifugation (10 minutes 6,000×g) in 50 mM Tris-HCl pH 7.3 buffer. After incubation for 30 minutes at 30° C. with 10 mg/ml lysozyme, MgSO₄ was added to the cell suspension to a final concentration of 10 mM and DNase and Rnase were added to 0.1 mg/ml ruptured by two-fold passage through a French Pressure cel (15,000 Psi L. lactis). The cell debris and cell membranes were separated by centrifugation (10 minutes 11,000×g) after addition of 15 mM Na-EDTA pH 7.0. The membranes contained in the supernatant were collected by ultra-centrifugation (one hour 150,000×g) and resuspended in 3 ml (total protein content: 20 mg/ml) in 50 mM Tris-HCl pH 7.3 and stored at −80° C. until further use.

Before purification, one volume of membranes was solubilized with nine volumes of 50 mM Na₂HPO₄.NaHPO₄, 300 mM NaCl, 10 mM imidazole at pH 8.0 (buffer A) containing 3% n-octyl-β-glucoside. The extract was cleared by ultra-centrifugation (20 minutes 150,000×g) and mixed with 1 (bed) volume Ni2+-NTA agarose beads (pre-equilibrated with buffer A+1% n-octyl-β-glucoside) and gently rotated for 30 minutes at 4° C. The mixed column material was then poured into a Bio-spin column (Bio-Rad) and washed with 20 volumes buffer A+1% n-octyl-β-glucoside. The protein was eluted with buffer A+1% n-octyl-β-glucoside and increasing amounts of L-Histidine (one volume 50 mM, one volume 100 mM, 2× one volume 200 mM). Protein concentration was determined according to Schaffner and Weissmann (Anal. Biochem. 1973, 56:502-514). Further analysis was done on a SDS-15% polyacrylamide gel followed by staining with Coomassie Brilliant Blue or transferral to PVDF membranes by semi-dry electroforetic blotting for immunodetection with anti-His antibodies (Amersham Pharmacia Biotech). Immunodetection was performed with an alkaline phosphatase-conjugated secondary antibody as recommended by the manufacturer (Sigma).

The purified protein was reconstituted in a mixture of the following lipids: 1,2-Dioleoyl-sn-Glycero-3-Phosphocholine (avanti 850375) and 1,2-Dioleoyl-sn-Glycero-3-Phospho-L-serine (avanti 810225) 9:1 w/w or Dioleoyl-sn-Glycero-3-Phosphocholine and Cholesterol (avanti 700000) 8:2 mol:mol. Before reconstitution, the lipids were washed and mixed in chloroform (20 mg/ml) and dried under N₂ gas. The dried lipids were then resuspended in 50 mM Kpi buffer pH 7.0 to a final concentration of 20 mg/ml. The suspension was then sonicated using a tip sonicator (eight cycles 15 seconds on, 45 seconds off, intensity of 4 μm (peak to peak)). The formed liposome solution was then completely solubilized using n-octyl-β-glucoside and the purified protein was added (1:1000, 1:500 or 1:50 w/w protein/lipid). Proteoliposomes were then formed by dialyzing the lipid-protein mixture for three days at 4° C. against 500 volumes 50 mM Kpi pH 7.0 without any detergent, using a 3,500 Da MWCO Spectrum spectrapor dialysis-membrane. After the first night of incubation, 0.5 g of polystyrene beads (Bio-Beads SM2™) were added for extra detergent removal.

Freeze Fracture Electron Microscopy

Freeze fracture electron microscopy of reconstituted MscL^{Ll} was performed as described elsewere (R. H. Friesen, et al., 2000, J. Biol. Chem. 275:33527-33535).

Electrophysiological Characterization of MscL^{Ll}

Electrophysiological characterization was essentially performed as described (P. Blount, et al., 1999, Methods Enzymol. 294:458-482). Giant spheroplasts of E. coli PB104 (MscL negative) containing the plasmid pB10bMscL^{Ll}6H (for overexpression of MscL^{Ll}) were generated: cells were grown to OD₆₀₀ of 0.5, diluted ten-fold, and grown in the presence of 60 μg/ml cephalexin (preventing septation, but not cell growth) and 1.3 mM IPTG. When the cells had formed, non-septated filamentous snakes of 50 to 150 μm were harvested at 5,000×g. The pellet was resuspended in 1/10th of the original volume of 0.8 M sucrose. Cell outer membranes (peptidoglycan) were digested with lysozyme (200 μg/ml) in the presence of DNase (50 μg/ml) in 50 mM Tris-HCl, 6 mM Na-EDTA at pH 7.2 for two to five minutes. The reaction is stopped when sufficient giant spheroplasts have formed by the addition of 8 mM MgCl₂ (final concentration). Spheroplasts were enriched by spinning on a 0.8 M sucrose cushion.

Alternatively, patches were studied from liposomes with reconstituted MscL^{Ll}. Proteoliposomes were centrifuged at 200,000×g, resuspended to 100 mg/ml in 10 mM MOPS, pH 7.2, 5% ethylene glycol, and dried overnight for four hours on glass slides in a desiccator at 4° C. Rehydration of the lipids to 100 mg/ml was performed in liposome patch buffer (200 mM KCl, 0.1 M EDTA, 10⁻² mM CaCl₂ and 5 mM HEPES pH 7.2). The proteoliposomes were then loaded into the sample well containing patch buffer with 20 mM MgCl₂ and caused the lipid sample to form large unilammelar blisters, which were patched.

Patches were examined at room temperature, with symmetrical solutions for pipette and bath. The buffer is composed of 200 mM KCl, 0.1 M EDTA, 10⁻² mM CaCl₂ and 5 mM HEPES pH 7.2. For spheroplasts, 90 mM of MgCl₂ was added; for proteoliposomes, 20 mM. Recordings of currents through open channels were performed at ±20 mV. Data on pressure and current were acquired at a sampling rate of 30 kHz with a 10 kHz filtration and analyzed using PCLAMP8 software. Pressure was measured using a piezoelectric pressure transducer (Micro-switch) (World Precision Instruments PM01).

In Vitro Release Profiles of a Model Drug from Proteoliposomes

As described in previous Examples.

Results

MscL^{Ll} Expression, Purification, and Reconstitution

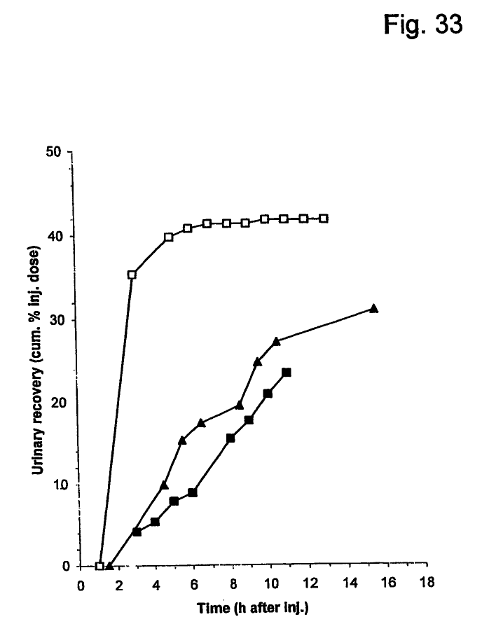

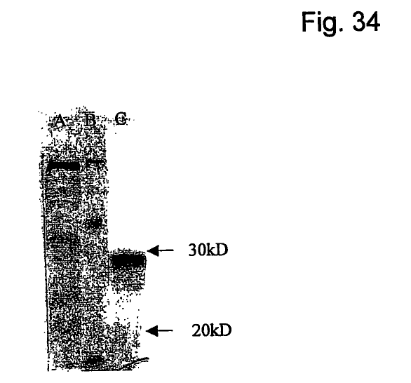

The purification of MscL^{Ll} was to obtain apparent homogeneity in a single step using nickel chelate-affinity chromatography as shown in FIG. 34 lane c. Per ml of vesicles, about 1 mg of protein could be obtained at an estimated purity of 95% based on SDS-PAGE and Coomassie Brilliant Blue staining. This means that expression levels in the membrane were around 5% of total membrane protein. The band also showed up very clearly after immunological detection on a PVDF membrane (data not shown). The amount of protein for reconstitution was determined experimentally. For patch-clamp experiments, a 1:1000 w/w protein/lipid ratio was found to be useful, whereas for the calcein release assay, a 1:500 ratio seemed to give the clearest results.

Freeze Fracture Electron Microscopy

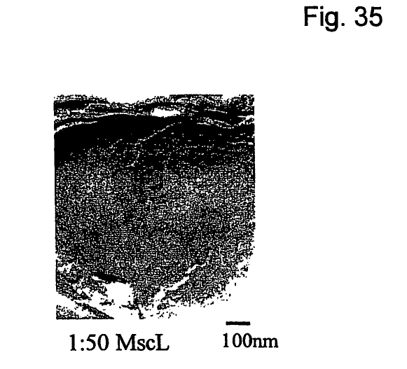

FIG. 35 shows an electron micrograph of freeze-fractured proteoliposomes. As can be seen, the MscL^{Ll} protein was indeed inserted into the lipid bilayer.

Electrophysiological Characterization of MscL^{Ll}

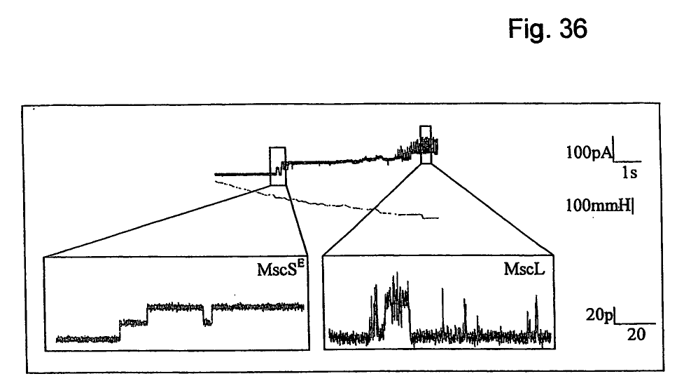

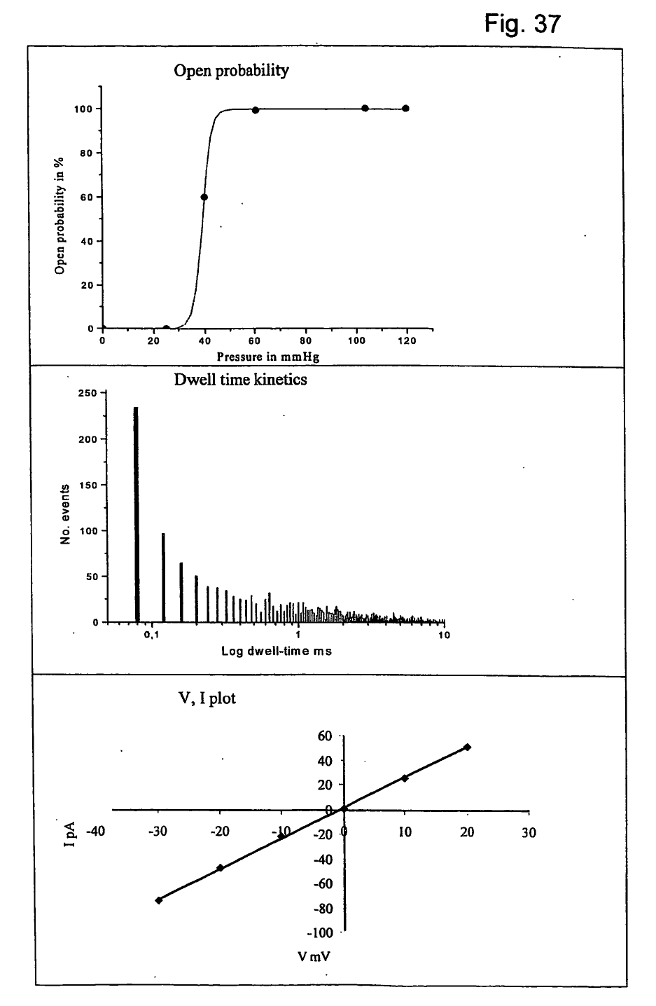

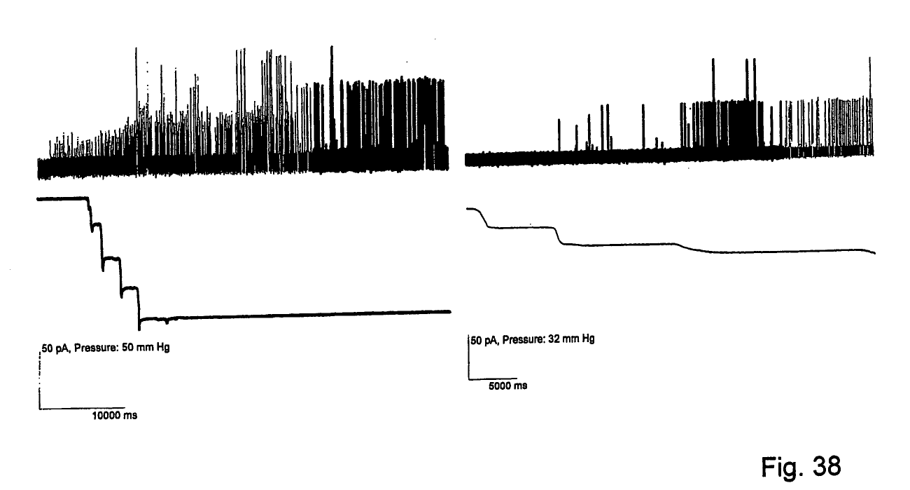

FIG. 36 shows a typical trace of the MscL^{Ll} in E. coli spheroplasts. The channel openings are indicated as an upward current as a result of the applied pressure. Both MscS^{Ec} and MscL^{Ll} channels are visible in this patch enabling a sensitivity comparison to MscL^{Ec}. This showed that MscL^{Ll} in E. coli cells opens at higher pressures than MscL^{Ec}. The ratio of pressures for opening MscL/MscS are 2.4 for MscL^{Ec} and 2.8 for MscL^{Ll}. FIG. 37 gives information on pressure sensitivity, open dwell time and conductance of MscL^{Ll}, which are all comparable to the values found for MscL^{Ec}. FIG. 38 shows traces of MscL^{Ll} reconstituted into different lipid compositions. It can be seen that the initial full openings occur at different pressures in the different liposome compositions.

In Vitro Release Profiles of a Model Drug from Proteoliposomes

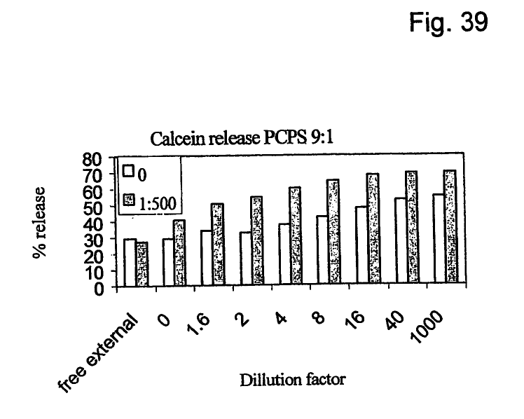

FIG. 39 shows the release of calcein in response to an osmotic shock in proteoliposomes containing MscL^{Ll}. The results of patch clamp and the calcein release assay show that this MscL homologue can be used to deliver substances from liposomes as described for MscL from E. coli and derivatives thereof.

### Example 1-H2

### Use of MscL Homologues from Organisms Other than E. coli; Charge-Induced Channel Opening of the L. lactis G20C and V21C Mutants

Material and Methods

The constructs of the G20C^{Ll}, V21C^{Ll} and G22C^{Ec} MscL-6His mutants were constructed using standard molecular biology techniques. The mutants were tested in pB104 strain (MscL null E. coli) by a MTSET shock assay on agar plates with IPTG (A. F. Batiza, et al., 2002, PNAS, 99:5643-5648).

Results

The results shown in Table G show an MTSET-dependent opening of the G20C^{Ll}, V21C^{Ll} and G22C^{Ec} MscL channels, resulting in the absence of growth on agar plates with IPTG. Since charge-induced channel gating is a mechanism to introduce pH-controlled drug release, these results indicate that the G20C^{Ll} or V21C^{Ll} could be an alternative for G22C^{Ec} MscL. This means that we can use chemically modified G20C^{Ll} and V21C^{Ll}, or G20H^{Ll} MscL, as pH-sensitive channels in liposomes.

### Example 1-I

### Controlled Release of Insulin from Liposomes Containing MscL-Mutant G22C

This Example shows the release of a therapeutically relevant hydrophilic molecule from MscL-containing liposomes using a filter-binding assay.

Materials and Methods

DOPC:DOPS (9:1 mol/mol) liposomes containing the G22C MscL were prepared as described previously. Insulin and FITC were obtained from Sigma (St. Louis, Mo., USA). Insulin (23 mg) was reacted with a four-fold molar ratio of FITC in 0.1 N borate buffer pH 9.0 for 60 minutes. The pH was lowered to 7.5 with 0.1 N boric acid and the solution was extensively dialyzed, using dialysis membrane with molecular weight cutoff of 2,000 Da, for 96 hours against water at 4° C. with frequent water changes. Absorption spectra of the dialyzed sample was used to quantify the protein concentration and the stoichiometry of labeling. Concentrations of fluoresceine and insulin were both 0.1 mM. The labeled insulin was encapsulated by three freeze-thaw cycles, followed by extrusion through a 400 nm polycarbonate membrane. The proteoliposomes containing labeled insulin were separated from free-labeled insulin by using sephadex G-50 column chromatography equilibrated with 145 mM NaCl, 300 mM Sucrose, 25 mM Tris.HCl and 1 mM EDTA pH 8.0.

Proteoliposomes were prepared as described in Example 1-E and MTSET is used for opening of the MscL-mutant G22C channels. Samples were taken at different time points and triton was added as a control for maximum fluorescence (100%). Samples were filtered over a 450 nm Cellulose Nitrate filter (Schleicher & Schuell BA85). The filtrate of 2 ml was retained and the fluorescence of 200 μl of each filtrate was monitored in a f1600 platereader (Bio-Tek). All experiments were performed in triplo.

Results

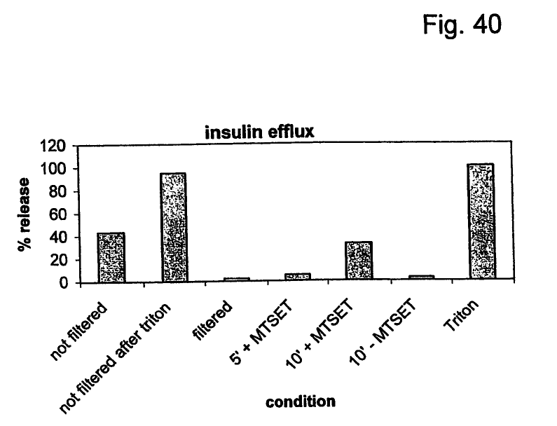

FIG. 40 shows the release of FITC insulin through MscL-mutant G22C upon activation with 1 mM MTSET. The difference between filtered and unfiltered conditions is the amount of FITC-insulin encapsulated in the proteoliposomes. The fluorescence of the unfiltered condition with and without Triton X-100 indicates that the concentration of FITC-insulin in the proteoliposomes exhibit self-quenching. With and without MTSET, are control conditions for the effect of MTSET on the FITC insulin efflux, to show that FITC insulin efflux is indeed MscL mediated.

The mass of FITC insulin is approximately 6,100 Da and considerably higher compared to calcein. Therefore, this Example shows the applicability of this delivery system for therapeutic macromolecules. The filter-binding assay can also be used for monitoring the controlled release of other labeled drug molecules from proteoliposomes.

### Example 2

### Method for the Rapid Release of Molecules at a Target Site with the Help of an Applied Electric Field

Decondensation of anionic polymers under the influence of an electric field results in a swelling of the polymer matrix. Swelling can be achieved at field strengths of 2.5 V across a 5 μm vesicle that corresponds to a field strength of 5000 Vcm⁻¹. This is a factor of 10 less than the field strength needed to induce leakage of ions through the ion channels. In the confined volume of a liposome, the swelling is translated into a membrane stress or internal pressure of approximately 10 bar. This is sufficient to induce the opening of the MscL-osmo-regulated channel and the release of soluble small molecular components.

Material and Methods

In vitro release profiles of a model drug were determined. Liposomes, as in Example 1, with and without MscL-6His, were prepared in the presence of the dye, calcein and heparin sulfate, a natural polymer found in secretory granules. A liposome was inserted into the tip of a glass pipette with the application of suction. The pipette voltage relative to the bath was controlled by a current-to-voltage converter. The solution in both the bath and the pipette was buffered to 6.5 with histamine dichloride and phosphoric acid. An Axopatch 200B was used to apply voltages and to sample the currents at 2.5 ms intervals. Swelling was induced by the application of a negative potential. To measure the swelling, the isolated liposomes were imaged and analyzed as described (J. M. Fernandez, et al., 1991, Biophys. J. 59:1022-1027, and J. R. Monck, et al., 1991; Biophys. J. 59:39-47).

Results

The heparin sulfate matrix swelled instantaneously and predictably with the application of a negative voltage as reported (C. Nanavati and J. M. Fernandez, 1993, Science 259:963-965). The swelling was accompanied by an instantaneous current and a visible diffusion of the calcein dye into the bath. When the voltage was turned off, the current and swelling returned to control levels but the calcein remained in the bath.

Variations on this theme are:
The gel network was covalently cross-linked to different extents, resulting in different drug release profiles.
The gelation process is fully reversible, depending on the type of gel, and can be modulated by several signals, e.g., temperature, light, pH, magnetic fields, electric currents, ultrasound and chemical or biological compounds.

### Example 3

### Use of the Protein Anchor as a Targeting Means

### Example 3-A

### Targeting of a Reporter Molecule through the Use of a Protein Anchor Homolog to a Microorganism Other than a Gram-Positive Bacterium

The cloning of a Protein Anchor homolog, the anchor of MltD of the gram-negative (G−) bacterium E. coli that has the consensus sequence as described in Table A and patent applications W099/25836 and EP 01202239.8, is described. The MltD anchor was fused to a reporter and binding of the reporter-anchor fusion to E. coli was demonstrated. This Example describes, therefore, the feasibility of targeting G-bacteria.

Materials and Methods

Plasmid pPA9 was used for cloning the MltD anchor (cMD). This plasmid contains the acmA gene (cell wall hydrolase) devoid of its native Protein Anchor (acmA′). This truncated AcmA has little or no cell wall hydrolase activity. The idea is that the cell wall hydrolase activity can be restored by cloning Protein Anchor homologs behind the truncated acmA. The cell wall hydrolase activity can be easily detected in plate and gel assays. For immunological detection of the new fusion protein, the c-myc epitope is present in pPA9 in-frame downstream from the truncated acmA. Downstream from the c-myc epitope, several unique restriction enzyme-recognition sites were present that allow in frame cloning of Protein Anchor homologs. The Polymerase Chain Reaction (PCR) method was used to amplify cMD using the primers MltDrepeat.fw (5′-GGAAGACTCAATTGCTGCTGTACAGTCGACG; SEQ ID NO:9) and MltDrepeat2.rev (5′-TAATAAGCTTAAAGGTCTCCAATTCCCAACGTCAGCTTATCGCCTGGTTTGC; SEQ ID NO:10) with E. coli chromosomal DNA as template. The cMD PCR fragment was digested with BglII and HindIII (underlined sequences in the primers) and was cloned into the BamHI and HindIII sites of pPA9, resulting in plasmid pPA10 (produces protein: AcmA′::myc::cMD).

Expression of AcmA′::myc::cMD using pPA10 in L. lactis NZ9000ΔacmA is directed from the acmA promoter. Detection of AcmA activity in plate- and SDS-polyacrylamide gel assays was described by Buist et al. (1995, J. Bacteriol. 177:1554-1563) for use of peptidoglycan from L. lactis. Peptidoglycan from E. coli was isolated according to the method described by Rosenthal and Dziarski (1994, Methods in Enzymology 235:261-263). Western blots and immunological detection using anti-c-myc-conjugated horseradish peroxidase (Roche) were performed according to the instructions of the supplier. E. coli cells were treated for ten minutes at 20° C. with 0.5 mM EDTA in TES buffer (200 mM Tris pH 8.0; 0.5 mM EDTA; 0.5 M sucrose) to destabilize the outer membrane. Prior to binding of the Protein Anchor chimera, the cells were washed with TSM buffer (200 mM Tris pH 8.0; 0.5 M sucrose; 10 mM MgCl₂) to remove the EDTA and to stabilize the spheroplasts. The L. lactis fusion protein AcmA′::myc::cMD was then incubated with the E. coli cells. The cells were subsequently washed with buffer TSM in order to remove unbound AcmA′::myc::cMD.

Results

Culture supernatants of L. lactis NZ9000ΔacmA(pPA10) producing AcmA′::myc::cMD were analyzed on SDS-PAA gels containing either peptidoglycan of L. lactis or of E. coli. The proteins in the gels were renatured and activity was only observed in the gels containing the E. coli peptidoglycan. From this result, we concluded that the MltD anchor binds to the gram-negative E. coli peptidoglycan, but not to that of the gram-positive L. lactis peptidoglycan and that binding is a prerequisite for activity of the AcmA enzyme. This is in agreement with the observations of Buist et al. (1995, J. Bacteriol. 177:1554-1563). Binding of AcmA′::myc::cMD to whole cells of L. lactis and E. coli was also studied by Western blot analysis. No binding was observed to L. lactis, whereas some binding to E. coli cells could be detected. The binding to E. coli could be further improved by pretreating the cells in order to disrupt the outer membrane. Taken together, these results showed that for targeting purposes based on the Protein Anchor principle, the range of microorganisms can be broadened by using AcmA Anchor homologs.

### Example 3-B

### pH-Dependent Binding of AcmA Protein Anchor Homologs and Hybrids

The lactococcal cell wall hydrolase AcmD (cD), a homolog of AcmA protein anchor (cA), consists also of three repeats with a calculated pI that is much lower (approximately pI 3.8) than that of the cA domain (Table B). This Example shows the influence of pH during binding of a cD fusion protein (MSA2::cD). In addition, it is shown that the pH binding range of AcmA-type protein anchors can be manipulated by making use of the pI values of the individual repeats in hybrids.

Materials and Methods

Bacterial strains, growth and induction conditions, TCA pretreatment of L. lactis cells, incubation of the MSA2 protein anchor fusion proteins to TCA-pretreated cells, washing conditions, protein gel electrophoresis, Western blotting and immunodetection were the same as described in patent application EP01202239.8. Under the conditions used, the cell-free culture supernatants with MSA2::cA, MSA2::cD or A3D1D2D3 have a pH of approximately 6.2. To examine the influence of a pH, the pH of the cultures was adjusted either by the additional HCl or NaOH in order to obtain the required pH.

Plasmid constructions. The plasmid that expresses the MSA2::cD (pPA43) is based on the nisin-inducible expression system (Kuipers et al. 1997, TibTech 15:135-140). Plasmid pPA43 furthermore contains an in-frame fusion of the lactococcal signal sequence of Usp45 (ssUsp; van Asseldonk et al. 1990, Gene 95:155-160) that drives secretion of the fusion protein, the c-myc epitope for detection purposes, the A3 cA repeat and repeats D1, D2 and D3 of cD. Primers that were used for cloning A3 were cArepeat3.fw (CCG TCT CCA ATT CAA TCT GCT GCT GCT TCA AAT CC; SEQ ID NO:11) and cA repeat3.rev (TAA TAA GCT TAA AGG TCT CCA ATT CCT TTT ATT CGT AGA TAC TGA CCA ATT AAA ATA G; SEQ ID NO:12) (in bold are the A3-specific sequences). The primers used for cloning the three cD repeats were cDrepeat1.fw (CCGTCTCCAATTTCAGGAGGAACTGCTGTTACAACTAG; SEQ ID NO:13) and cDrepeat3.rev (TAATAAGCTTAAAGGTCTCCAATTCCAGCAACTTGCAAAACTTCTCCTAC; SEQ ID NO:14) (in bold are the cD-specific sequences).

Results

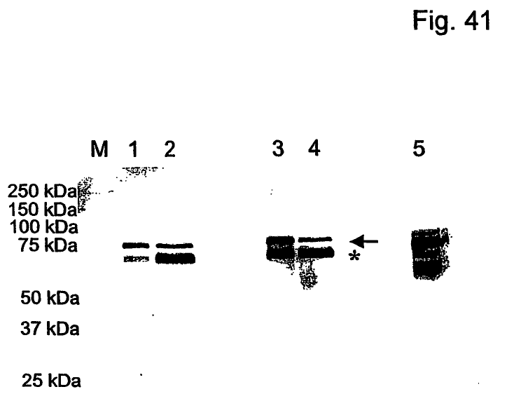

Binding of MSA2::cD at low pH. Since binding of MSA2::cD was not observed at a pH (the pH of the culture medium after growth and induction is about 6.2) higher than the calculated pI for the cD domain (pI 3.85), the binding was studied when the pH of the medium was adjusted to pH 3.2. TCA-pretreated L. lactis cells were used as the binding substrate and the relative amounts of bound MSA2::cD were analyzed in Western blots. The amounts of unbound reporter protein left behind in the culture supernatant after binding were also analyzed. FIG. 41 shows that there is a clear increase in bound MSA2::cD when binding is performed at pH 3.2 (compare lanes 1 and 3). At the same time, less unbound reporter protein remained in the supernatant (compare lanes 2 and 4). This result indicates that positive charges are important for binding of cA-type anchoring domains.

Binding of cAcD hybrid anchors. Analysis of the pI values of the cA homologs in Table A learns that two classes of repeats can be distinguished: a majority (99 out of 148) of homologs that have a high pI value (>8) and a group (33 out 148), of which cD is a representative, that has pI values lower than 6. Based on the experimental results, it is likely to assume that these types of anchoring domains only bind to bacterial cell walls at a pH that is lower than its pI. Notably, most cell wall binding domain homologs consist only of repeats with a pI that are representatives of one of the two groups, i.e., only repeats with a high or low pI. Interestingly, some proteins with putative cell wall binding domains, e.g., those of DniR of Trepanoma pallidum and an amidase of Borrelia burgdorferi, consist of repeats with high and low pI. We assumed that the binding pH of such “natural hybrid” cell wall binding domains is below the intermediate pI value of the total number of repeats present in the domain. Therefore, using the cA and cD repeats that are available, a hybrid cell wall protein anchor can be constructed that has an intermediate pI value. Table C lists both the native AcmA and AcmD anchors and a number of examples of cA/cD hybrids. The hybrid protein anchor constructed (A3D1D2D3) has a calculated pI value of approximately 5.1. A protein anchor consisting of only D1D2D3 shows little binding at a pH above its calculated pI (see above). The A3 (pI 10) domain shows similar binding at pH 5 and pH 7.

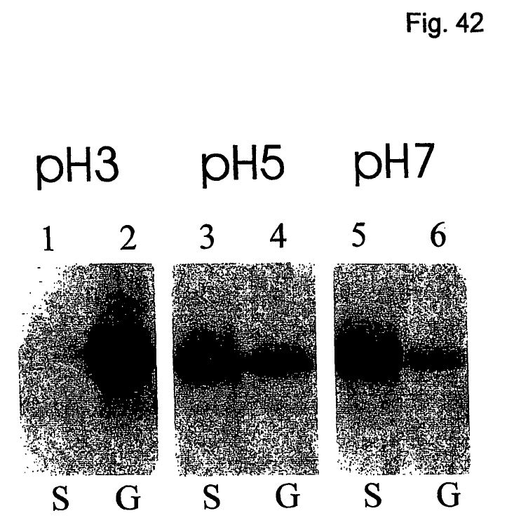

The binding of the hybrid anchor A3D1D2D3 was tested at pH 3, pH 5 and pH 7. At pH 3, almost all protein had been bound to the ghost cells (FIG. 42). At pH 5, there was still considerable binding (+/−40%), whereas there was only minimal binding at pH 7 (+/−20%). This result shows that the pH range of binding for cD repeats was shifted to higher pH values by the addition of one cA repeat (A3) that caused a shift in calculated pI values of 3.8 to 5.1. The increase of binding at pH 5 for the A3D1D2D3 hybrid cannot be attributed to binding of the A3 repeat alone. If this was the case, then the same level of binding should occur at pH 7 since the A repeats show the same binding at these pH values. In addition, the increased binding at pH 5 is not an additive effect in the sense that an extra binding domain results in increased binding. It was observed before that addition of one repeat to the cA anchor did not result in increased binding. The binding at higher pH values of the A3D1D2D3 repeats, as compared to D1D2D3 repeats alone, has, therefore, to be attributed to the increase in the calculated pI value of the hybrid cA/cD anchor.

The results clearly demonstrate that pH binding properties of these types of protein anchors can be manipulated on the basis of the pI values of individual repeats present in the hybrid anchor. This is particularly relevant for targeting to cells that are in an environment with another pH (e.g., tumor tissues have a pH of approximately 6.5) other than the surrounding tissues (normal body fluids and tissues have a pH of 7.4).

This Example demonstrates the increased availability (by pH) of a hybrid Protein Anchor for binding to a target. A model reporter molecule (MSA2) is used and a model target is used (TCA-pretreated L. lactis). In some applications, modified Anchors are coupled to delivery vehicles (e.g., liposomes, hydrophobin particles, etc.) and targeted to microorganisms or specific animal or human organs or tumors.

### Example 3-C

### pH-Dependent Binding of a Mutagenized Protein Anchor

Mutagenesis of the AcmA/AcmD Protein Anchors by error-prone PCR and/or in vitro recombination and the selection of a variant that binds to a model target (pretreated L. lactis “ghosts”) at pH 6, but does not bind at pH 7.4.

Materials and Methods

Strains, vectors and Protein Anchor gene fragments. L. lactis ghost cells were prepared as described in patent application EP 01202239.8. The phagemid pCANTAB5E (based on M13) and E. coli TG1 and HB2151 were used for cloning of the mutagenized Protein Anchor gene fragments according to the instructions of the supplier (Pharmacia). Gene fragments of the AcmA and AcmD Protein Anchor homologs of L. lactis (Table A; Genbank accession numbers U17696, QGC125) were used for random mutagenesis. Recombinant phages were immunologically detected using mouse monoclonal antibodies raised against the minor coat protein pIII of phage M13. Detection of the antibody was performed with alkaline phosphatase-conjugated rabbit anti-mouse IgG.

Mutagenesis. Random point mutations were introduced by error-prone PCR. The 100-μl reaction mixture contained 10 μl of 10× reaction buffer (10 mM Tris-HCl, 10 mM KCl, 1.5 mM (NH₄)SO₄, 0.1% (v/v) Triton X-100) with 2.5 mM MgCl₂, 0.2 mM MnCl₂, 200 μM dATP and dGTP, 1 mM dTTP and dCTP, 30 pmol each primer, and 5 units of Taq polymerase. The PCR schedule was two minutes at 94° C., followed by 25 cycles of 94° C. for 30 seconds, 50° C. for 30 seconds, and 72° C. for 30 seconds. The product was purified and, after digestion, ligated into an expression vector.

In vitro recombination. The Protein Anchor gene fragments of acmA and acmD were amplified by the polymerase chain reaction (PCR). After removal of the free primers, about 2 to 4 μg of the DNA substrates were digested with 0.15 units of DNAseI in 100 μl of Tris-Cl pH 7.5, 1 mM MgCl₂, for 5 to 10 minutes at room temperature. Fragments of 70 to 200 bp were extracted from 2% low melting point agarose gel. The purified DNA fragments (10 to 30 ng/μl) were resuspended in PCR mix and reassembled in a primerless PCR reaction using Taq DNA polymerase (2.5 U) and a program of 94° C. for 60 seconds, 40 cycles of 30 seconds at 94° C., 30 secon 30 seconds at 72° C., and a final extension of five minutes at 72° C. The reassembly mixture was 40-fold diluted into fresh PCR mix with primers. After 15 cycles of PCR, consisting of (30 seconds at 94° C., 30 seconds at 50° C., 30 seconds at 72° C.) single amplification, products of the corre were obtained. The resulting full-length amplification product was digested and ligated into the expression vector.

Library construction and screening. Construction, amplification and storage of the phage library were done according to the instructions of the supplier (Pharmacia). Screening for binding variants to L. lactis ghost cells was done by incubation of the phage library in phosphate-buffered saline (PBS) of pH 6. After binding, the ghost cells were extensively washed to remove unbound phages. The phages were released from the ghost cells by incubation with mutanolysin (Sigma) or by proteolytic cleavage. The released phages were amplified and the binding, washing and amplification procedure was repeated two times. Finally single phages were amplified and individual phage lysates were incubated with ghost cells seeded in microtiter plates in duplo. In one set of microtiter plates, the binding was done at pH 6 and in the other set of plates, the binding was done at pH 7.4 After multiple washings, an immunological detection was done using mouse monoclonal antibodies against pIII of the filamentous phage M13 and phosphatase-conjugated anti-mouse IgG. Phages that showed binding at pH 6 but not at pH 7.4 were identified using a microtiter plate reader.

Results

The biopanning of the recombinant phages resulted in a phage population that bound to ghost cells at pH 6. The counter screening at pH 7.4 identified several putative phages that did not show binding at this specific pH. One of these, containing the mutagenized protein anchor DNA insert X1, was isolated and subsequently cloned into a lactococcal expression vector that allows secretion of c-myc reporter fusions of protein anchors. Expression of the fusion protein, designated c-myc::X1, resulted in its secretion. Binding of c-myc::X1 to lactococcal ghost cells at pH 6 and pH 7.4 was analyzed by Western blotting. The results clearly showed that the X1 protein anchor binds to ghost cells at pH 6 but not at pH 7.4. Therefore, it was demonstrated that pH-dependent targeting proteins can be obtained using AcmA-type Protein Anchor homologs.

This Example demonstrates the induced availability (by pH) of a modified Protein Anchor for binding to a target. In the Example, the mutagenized Anchors are used to target a model substrate (TCA-pretreated L. lactis “ghosts”). In some applications, modified Anchors are coupled to delivery vehicles (e.g. liposomes, hydrophobin particles, etc.) and are targeted to microorganisms or specific animal or human organs or tumors.

### Example 3-D

### Selection of Mutagenized Protein Anchors to a Preselected Human Tumor Cell Line

Random mutagenesis of Protein Anchors and the selection of a variant that binds to a preselected model tumor cell line (human intestine cancer cells).

Materials and Methods

Strains, cell lines, vectors and Protein Anchor gene fragments. L. lactis ghost cells were prepared as described in patent application EP 01202239.8. The M13-based phage display system pCANTAB5E (Pharmacia) as described under Example 3-C was used for cloning of the mutagenized Protein Anchor gene fragments according to the instructions of the supplier. Gene fragments of the Protein Anchor homologs selected from Table A were used for random mutagenesis. The immunological detection of the recombinant phages was as described under Example 3-C. Human intestine cancer cells (Intestine 407/ATCC CCL6 also designated as Henle cells) were cultivated to confluence in RPMI 1640 medium (Gibco) supplemented with 10% fetal calf serum (Gibco), 1% L-glutamine, 1% non-essential amino acids, and penicillin-streptomycin followed by cultivation in microtiter plates.

Mutagenesis. Random point mutations were introduced by error-prone PCR. The 100-μl reaction mixture contained 10 μl of 10× reaction buffer (10 mM Tris-HCl, 10 mM KCl, 1.5 mM (NH₄)SO₄, 0.1% (v/v) Triton X-100) with 2.5 mM MgCl₂, 0.2 mM MnCl₂, 200 μM dATP and dGTP, 1 mM dTTP and dCTP, 30 pmol each primer, and 5 units of Taq polymerase. The PCR schedule was two minutes at 94° C., followed by 25 cycles of 94° C. for 30 seconds, 50° C. for 30 seconds, and 72° C. for 30 seconds. The product was purified and, after digestion, ligated into an expression vector.

In vitro recombination. The Protein Anchor gene fragments of the selected homologs were amplified by the polymerase chain reaction (PCR). After removal of the free primers, about 2 to 4 μg of the DNA substrates were digested with 0.15 units of DNAseI in 100 μl of Tris-HCl pH 7.5, 1 mM MgCl₂, for 5 to 10 minutes at room temperature. Fragments of 70 to 200 bp were extracted from 2% low melting point agarose gel. The purified DNA fragments (10 to 30 ng/μl) were resuspended in PCR mix and reassembled in a primeness PCR reaction using Taq DNA polymerase (2.5 U) and a program of 94° C. for 60 seconds, 40 cycles of 30 seconds at 94° C., 30 seconds at 50° C., 30 seconds at 72° C., and a final extension of five minutes at 72° C. The reassembly mixture was 40-fold diluted into fresh PCR mix with primers. After 15 cycles of PCR consisting of (30 seconds at 94° C., 30 seconds at 50° C., 30 seconds at 72° C.) single amplification, pro correct size were obtained. The resulting full-length amplification product was digested and ligated into the expression vector.

Library construction and screening. Construction, amplification and storage of the phage library was done according to the instructions of the supplier (Pharmacia). Screening for binding variants was done by incubation of the phage library in phosphate-buffered saline (PBS) with Henle cells immobilized on tissue culture plates. After binding, the Henle cells were extensively washed to remove unbound phages. The phages were released from the Henle cells by using release buffers or proteolytic enzymes as recommended by the supplier of the display kit (Pharmacia). The released phages were amplified and the binding, washing and amplification procedure was repeated three times. Finally, single phages were amplified and individual phage lysates were incubated with Henle cells seeded in microtiter plates. After multiple washings, an immunological detection was done as described under Example 3-C.

Results

The biopanning of the recombinant phages resulted in a phage population that bound to Henle cells. One of these, containing the mutagenized protein anchor DNA insert X2, was isolated and subsequently cloned into a lactococcal expression vector that allows secretion of c-myc reporter fusions of protein anchors. Expression of the fusion protein, designated c-myc::X2, resulted in its secretion. Binding of c-myc::X2 to Henle cells was analyzed by Western blotting. The results clearly showed that the X2 protein anchor binds to human intestine cancer cells. Therefore, we demonstrated that by using in vitro mutagenesis with AcmA-type Protein Anchor homologs as templates, targeting proteins can be obtained that are able to bind to eukaryotic cells.

This Example demonstrates that mutagenesis on Protein Anchor homologs can be used to obtain new Anchors that bind to tumor cells. In some applications, these Anchors are coupled to the proper delivery vehicles that have the ability to make the drugs available upon induction (e.g., liposomes with MscL).

### Example 3-E

### Use of Protein Domains Other than AcmA-type Protein Anchors for Targeting to Eukaryotic Cells; CWS Domains of Lactococcal PrtP

Material and Methods

Bacterial strains and growth conditions. L. lactis was grown at 30° C. in M17 (Difco, West Molesey, United Kingdom) or ½ M17 broth (containing 0.95% β-glycerophosphate, Sigma Chemicals Co., St. Louis, Mo.) as standing cultures or on ½ M17 agar plates containing 1.5% (wt/vol) agar. All media were supplemented with 0.5% (wt/vol) glucose, while 5 μg/ml chloramphenicol (Sigma Chemicals Co.), or 5 μg/ml erythromycin (Roche Molecular Biochemicals, Mannheim) were added when appropriate.

DNA techniques and transformation. Restriction enzymes, T4 DNA ligase and Expand™ High Fidelity DNA polymerase were obtained from Roche Molecular Biochemicals and used according to the instructions of the supplier. Synthetic oligo deoxynucleotides were obtained from Life Technologies B.V. (Breda, The Netherlands). PCR products were purified using the High pure PCR product purification kit (Roche Molecular Biochemicals). L. lactis NZ9000 was transformed by electroporation using a gene pulser (BioRad Laboratories, Richmond, Calif). Analytical grade chemicals were obtained from Merck (Darmstad, Germany), or BDH (Poole, United Kingdom).

Overexpression of fusion proteins. The DNA fragment encoding the CWS domain and cell wall anchor domain of the proteinase of L. lactis Wg2 was amplified from plasmid pGKV552 by PCR with oligonucleotides CWSI (5′-ATATAAAGCTTGCAAAGTCTGAA AACGAAGG; SEQ ID NO:15), and CWS2 (5′-CCGTCTCAAGCTCACTATTCTTCACGT TGTTTCCG SEQ ID NO:16). The purified 402-bp PCR product was digested with HindIII/Esp3I (underlined) and ligated into the HindIII site of pNG300, resulting in pNG301. The merozoite surface antigen of Plasmodium falciparum strain 3D7 (MSA2) was chosen as localization reporter protein. As this protein contains a eukaryotic GPI membrane anchor, which could obstruct secretion of the fusion proteins, a truncated version of MSA2 (lacking this membrane-spanning domain) was used. It was amplified using the oligonucleotides MSA2-1 (5′-ACCATGGCAAAAAATGAAAGT AAATATAGC; SEQ ID NO: 17) and MSA2-4 (5′-CGGTCTCTAGCTTATAAGCTTAGAATTCGGGATG TTGCTGCTCCACAG; SEQ ID NO:18). The PCR product was digested with NcoI/HindIII (underlined) and ligated into the corresponding restriction enzyme sites of pNG301, resulting in pCWS1a. One or two copies of the 264-bp PCR product obtained with oligonucleotides CWS1 and CWS3 (5′-ATTTAAGCTTTTACCGGATGTAAGTTGACCATTACG; SEQ ID NO:19), encoding the CWS domain, was/were introduced in the HindIII site of pCWS1a, resulting in pCWS2a and pCWS3a, respectively. Anchor-less variants of the CWS-containing fusion proteins were obtained using oligonucleotides MSA2-1 and Prt.Myc2 (5′-AAGATCTTCTTTGAAATAAG TTTTTGTTCCGTGCT; SEQ ID NO:20) with pCWS1a, pCWS2a or pCWS3a as templates, respectively. The PCR products were digested with NcoI and XhoI and ligated into these sites of pNG300, resulting in pCWS1, pCWS2 and pCWS3, respectively. Nisin induction of the nisA promoter upstream of the fusion protein-encoding fragments was performed as described by de Ruyter et al. (1996, Appl. Environ. Microbiol. 62:3662-3667).

Adherence to eukaryotic cells. Human Henle cells (Intestine 407/ATCC CCL6) were cultivated to confluence in RPMI 1640 medium (Gibco) supplemented with 10% fetal calf serum (Gibco), 1% L-glutamine, 1% non-essential amino acids, and penicillin-streptomycin followed by cultivation for two days on diagnostic glass slides (Danlab Oy, Helsinki, Finland). Before adhesion assays, the cells were washed once with PBS. L. lactis NZ9000 carrying either pNG304, pNG3041, pCWS1a, pCWS2a or pCWS3a was induced with nisin and grown overnight. After harvesting the cells by centrifugation (five minutes, 12,000×g) and washing with PBS, the cells were resuspended in PBS. Equal amounts of bacteria (10 μl, 2×10⁸ cells/ml) from each suspension were added to the epithelial cells followed by incubation for one hour at 37° C. in a moist chamber. After five washings with PBS at room temperature, the slides were fixed for ten minutes with methanol and the cells were stained for five minutes with 10 (v/v) Giemsa stain (Oy Reagena Ltd, Kuopio, Finland) and analyzed by light microscopy. The number of lactococcal cells that were in contact with the epithelial cells was counted from ten randomly chosen fields of view, taking into account only whole cells. The mean number of adhering bacteria was quantified.

Results

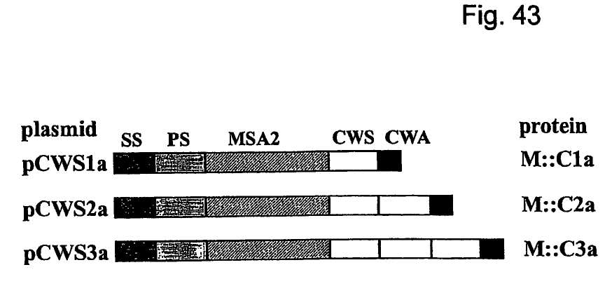

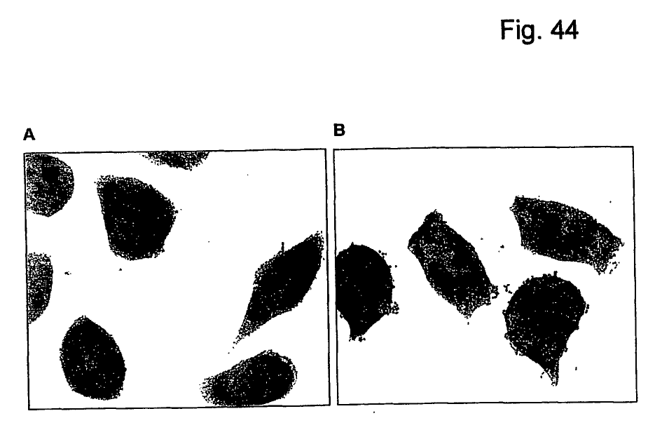

Adherence to human intestine 407 cells via multiple CWS domains. Surface-located bacterial proteins can be involved in adherence to eukaryotic cells. Some of these adherence proteins show autoaggregation properties. Since it was anticipated that the lactococcal proteinase CWS domain has autoaggregation properties,it was investigated whether the expression of the CWS domain on the surface of the lactococcal cells can result in adherence to eukaryotic cells. Cells of the human small intestine cancer cell line 407 (Henle) were used in this study. The adhering ability was tested of L. lactis NZ9000 cells expressing MSA2 fusions with one, two or three CWS domains, M::C1a, M::C2a or M::C3a, respectively (FIG. 43), covalently anchored to the bacterial cell wall via the LPXTG motif of the PrtP cell wall anchor. As controls, two types of NZ9000 strains were used: (i) one secreting MSA2 and, (ii) one that attaches MSA2 to the bacterial cell wall in a non-covalent way through the AcmA repeats (Leenhouts et al. 1999, Antonie van Leeuwenhoek 76:367-376, patent applications WO99/25836 and EP01202239.8). All cell types were incubated with the Henle cells and only those L. lactis NZ9000 cells expressing the covalent-anchored fusion proteins with one, two or three CWS domains showed adherence to these epithelial cancer cells of the human small intestine (FIG. 44). The degree of adherence with Henle cells was positively correlated to the number of CWS domain expressed on the lactococcal cells (Table D). MSA2 does not play any role in this binding since the two control strains did not show any adherence to the Henle cells.

This experiment shows that the CWS domain of the lactococcal PrtP can be used for targeting of bacteria and most likely proteins, drugs, hormones, immune-modulating signal, and delivery vehicles to eukaryotic tumor cells.

### Example 3-F

### Use of Protein Domains Other than AcmA-type Protein Anchors for Targeting to Eukaryotic Cells; pH-Dependent Binding of Mutagenized PrtP

Random mutagenesis of the 60 aa cell wall-spanning (CWS) domain of the lactococcal PrtP by error-prone PCR and the selection of a variant that binds in a pH-dependent manner to a preselected model tumor cell line (human intestine cancer cells).

Materials and Methods

Strains and vectors. Human intestine cancer cells (Intestine 407/ATCC CCL6 also designated as Henle cells) were cultivated to confluence in RPMI 1640 medium (Gibco) supplemented with 10% fetal calf serum (Gibco), 1% L-glutamine, 1% non-essential amino acids, and penicillin-streptomycin followed by cultivation in microtiter plates. The phage display vector pCANTAB5E as described under Example 3-C was used for cloning of the mutagenized PrtP CWS domain gene fragments according to the instructions of the supplier. The immunological detection of the recombinant phages was also performed as described under Example 3-C.

Mutagenesis. Random point mutations were introduced by error-prone PCR. The 100-μl reaction mixture contained 10 μl of 10× reaction buffer (10 mM Tris-HCl, 10 mM KCl, 1.5 mM (NH₄)SO₄, 0.1% (v/v) Triton X-100) with 2.5 mM MgCl₂, 0.2 mM MnCl₂, 200 μM dATP and dGTP, 1 mM dTTP and dCTP, 30 pmol each primer, and 5 units of Taq polymerase. The PCR schedule was two minutes at 94° C., followed by 25 cycles of 94° C. for 30 seconds, 50° C. for 30 seconds, and 72° C. for 30 seconds. The product was purified using Qiaquick (QIAGEN) and after digestion, ligated into an expression vector.

Library construction and screening. Construction, amplification and storage of the phage library was done according to the instructions of the supplier (Pharmacia). Screening for binding variants was done by incubation of the phage library in phosphate-buffered saline (PBS) of pH 6 with Henle cells immobilized on culture plates. After binding, the Henle cells were extensively washed to remove unbound phages. The phages were released from the Henle cells by using lysis buffer or proteolytic enzymes recommended by the suppliers of the phage display kit (Pharmacia). The released phages were amplified and the binding, washing and amplification procedure was repeated three times. Finally, single phages were amplified and individual phage lysates were incubated with Henle cells seeded in microtiter plates in duplo. In one set of microtiter plates, the binding was done at pH 6 and in the other set of plates, the binding was done at pH 7.4 After multiple washings, phages that showed binding at pH 6 but not at pH 7.4 were identified fusing a microtiter plate reader.

Results

The biopanning of the recombinant phages resulted in a phage population that bound to Henle cells at pH 6. The counter screening at pH 7.4 identified several putative phages that did not show binding at this specific pH. One of these, containing the mutagenized protein anchor DNA insert X3, was isolated and subsequently cloned into a lactococcal expression vector that allows secretion of c-myc fusions of protein anchors. Expression of the fusion protein, designated c-myc::X3, resulted in its secretion. Binding of c-myc::X3 to Henle cells at pH 6 and pH 7.4 was analyzed by Western blotting. The results clearly showed that the X3 protein anchor binds to Henle cells at pH 6 but not at pH 7.4. Therefore, we demonstrated that pH-dependent targeting proteins can be obtained using the lactococcal PrtP CWS domain.

This Example also demonstrates the induced availability (by PH) of a mutagenized PrtP CWS domain for binding to a target. In the Example, the mutagenized Anchor is used to target a model substrate (human small intestine cancer cells). In some applications, the mutagenized Anchors are coupled to delivery vehicles (e.g., liposomes, hydrophobin particles, etc.) and are targeted to microorganisms or specific animal or human organs or tumors.

### Example 4

### Use of Hydrophobin Vesicles as Delivery Vehicles

Methods

Hydrophobin SC3 was purified from the culture medium of strain 4-40 of Schizophylum commune (CBS 340.81) as described by Wessels and Wosten et al. (J. G. Wessels, 1997, Adv. Microb. Physiol. 38:1-45; H. A. B. Wösten, et al., 1993, The Plant Cell 5:1567-1574). Before use, the freeze-dried SC3 was disassembled with pure TFA and dried in a stream of nitrogen. The monomeric protein was then dissolved in 50 mM phosphate buffer or water.

Hydrophobin vesicles can be made with the hydrophobic site inwards, by coating oil droplets, or with the hydrophilic site inwards, by coating water droplets (H. A. Wösten, et al., 1994, EMBO. J. 13:5848-5854).

Coating of oil droplets was achieved by emulsifying 10 μl of an oil (kitchen grade olive oil, mineral oil or organic solvents that do not mix with water) in 300 μl water by sonication and adding 300 μl of an aqueous solution of hydrophobin (200 μg ml⁻¹). Alternatively, the oil was directly emulsified in the hydrophobin solution and 300 μl water was added.

After overnight incubation at room temperature or 60° C. in the presence of 0.02% NaN₃, the emulsions were centrifuged at 10,000 g for 30 minutes and the oil droplets washed four times with water by centrifugation to remove monomeric hydrophobin. Oil droplets coated with assembled hydrophobin were then resuspended in a small volume of water to study stability, size and leakage of encapsulated substances.

For assembly of hydrophobins on oil droplets of a uniform size, a micro filtration membrane was used. At the hydrophilic side of the membrane, an aqueous solution of hydrophobin (100 μg ml⁻¹) was applied and, at the hydrophobic side of the membrane, the oil with the substance of interest was present. The oil was forced through the filter into the aqueous solution by pressure, generating an emulsion of precisely sized droplets of as small as 0.1 μm. After overnight incubation at room temperature or 60° C., the emulsion was washed with water and concentrated as described above. Substances to be included in the vesicles are added to the water phase while generating these vesicles.

For assembly at the water/oil interface of an emulsion of water in oil, 10 μl of an aqueous solution of hydrophobin (1 mg ml⁻¹) was emulsified in 600 μl of oil by sonication. After overnight incubation, the mixture was centrifuged at 10,000 g for 30 minutes in order to collect the aqueous phase. The emulsion was washed with oil by centrifugation and was further characterized by electron microscopy.

Alternatively, 100 μg of freeze-dried monomeric hydrophobin was dispersed in 600 μl oil by sonication or agitation. Ten μl of water was emulsified in the oil/hydrophobin suspension by sonication. After overnight incubation, the emulsion was centrifuged at 10,000 g for 30 minutes and washed four times with oil by centrifugation to remove excess hydrophobin. The concentrated emulsion was studied for stability, size and leakage.

Furthermore, the water in oil emulsions of uniform droplet size were made with the micro-filtration membrane as described above. Rodlet structures or the so-called β-sheet form of hydrophobins are spontaneously formed at the oil/water interface. Stability of the emulsion and the size of the coated droplets were assessed by electron microscopy and atomic force microscopy according to standard techniques.

Leakage of the vesicles was studied in one of two ways, either by following the partitioning of the fluorescent model drug, pyrene, between the inner and outer compartments by monitoring the change in the intensity of pyrene excimer fluorescence or by following the efflux of the fluorescent model drug calcein out of the vesicle. The percentage release of calcein from the vesicles was calculated from the dequenching of calcein fluorescence according to the following equation:
% Release=(Fₓ−F₀)/(Fₜ−F₀)×100

Where F₀ is the fluorescence intensity at zero time incubation, Fₓ is the fluorescence at the given incubation time points and Fₜ is the total fluorescence, obtained after lysis by shearing of the vesicles. Fluorescence was monitored with an SLM 500 spectrofluorimeter in a thermostatted cuvette (1 mL) at 37° C. under constant stirring. Excitation and emission wavelengths were, respectively, 490 (slit 2 nm) and 520 nm (slit 4 nm). The experiments were performed at hydrophobin concentrations of approximately 2.5 μM. The hydrophobin vesicles were separated from free calcein by using Sephadex 50-column chromatography equilibrated with PBS (160 mM NaCl, 3.2 mM KCl, 1.8 mM KH₂PO₄, 0.12 mM Na₂HPO₄, 1.2 mM EGTA, pH 8.0), which was isotonic to the calcein-containing buffer.

### Example 5

### Use of Sunfish Amphiphiles as Delivery Vehicles

### Example 5-A

### In Vivo Delivery of DNA

This Example shows in vivo gene expression after intravenous injection of Sunfish-complexed DNA in the mouse.

Methods

Sunfish (SF) amphiphiles were tested and compared to DOTAP, a commercially available amphiphile (Avanti Polar Lipids). The used plasmid DNA codes for Luciferase, which is expressed upon transfection. Luciferase activity represents the efficiency of transfection.

Liposomes were prepared by drying the Sunfish amphiphiles or DOTAP in glass tubes under a nitrogen flow followed by being under vacuum for one hour. The lipid film was resuspended in 20% sucrose by mixing and warming up for five minutes to 60° C. in a water bath. The liposomal suspension was then sonicated for three minutes. For lipoplexes formation, plasmid DNA (30 μg/mouse) diluted in 20% sucrose was added to the liposomes (450 nmol/mouse) under mixing. The lipoplexes were allowed to rest at room temperature for minutes and were intravenously injected (150 μl) in male BALB/c mice.

Organs (lungs, heart, liver, spleen and kidneys) were collected 24 hours after injection for analysis. Luciferase activity was measured in homogenates of the different organs and related to a standard curve obtained with purified recombinant Luciferase.

Results

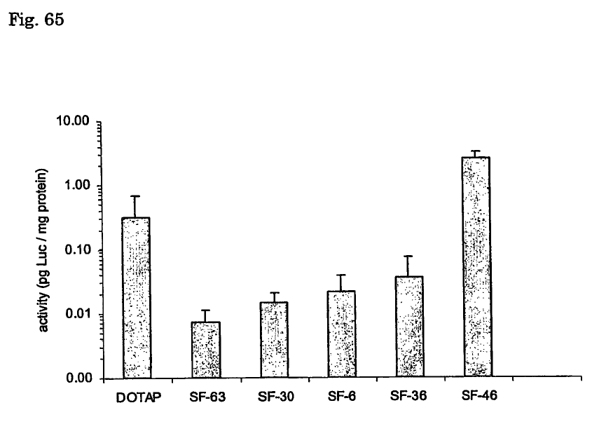

Like with DOTAP, intravenous injection of DNA complexed with Sunfish amphiphiles SF-6, SF-30, SF-36, SF-46 and SF-63 resulted in gene expression in the lungs (FIG. 65). This demonstrates that several Sunfish amphiphiles are active transfectants in vivo, with varying efficiencies in transfection: SF-46>SF-36>SF-6>SF-30>SF-63). Probably, the same transfection mechanism as with DOTAP plays a role, i.e., aggregation of the lipoplexes with blood components, leading to accumulation in the lungs and transfection in this organ. Especially with SF-46, the transfection was very good. Compared to DOTAP, complexation with SF-46 resulted in a five times higher Luciferase activity in the lungs.

### Example 5-B

### In Vivo Delivery of Protein

This Example shows (1) that complexation of protein with Sunfish/co-lipid systems has a clear effect on the body distribution and cellular degradation of a protein administered intravenously into a healthy mouse, and (2) that a prolonged circulation time of the protein-lipid complex can be obtained by using PEGylated Sunfishes.

Methods

The low molecular-weight protein myoglobin was chosen as the model protein, the molecular weight is 17.8 kDa and iso-electric point 7.3. To enable quantification of organ uptake of myoglobin, myoglobin was radio-labeled with ¹²⁵Iodine-tyramine-cellobiose that is retained within the cell in which it is internalized. In order to determine the rate of degradation in the target organs, the protein was radio-labeled with ¹³¹Iodine, a label that leaves the cell during degradation of the protein.

Study 1: For complexation, SF-30 and SF-26, in combination with the co-lipids DOPE and cholesterol, were tested. The complexation was performed by mixing 3.5 μg of myoglobin in Tricine buffer (pH 8.5) with 75 nmol liposomes consisting of Sunfish and co-lipid in a molar ratio of 1:1 in 5% glucose solution. This resulted in positively charged complexes with a size of approximately 80 to 85 nm for DOPE and 350 to 360 nm for cholesterol-containing complexes.

Study 2: For complexation, SF-30, in combination with cholesterol in a molar ratio of 1:1, was used. Part of SF-30 was replaced by PEGylated Sunfish SF-79 (1-(polyethyleneglycol5000-ω-methylether)-4-(10′-cis-nonadecenyl)pyridinium bromide). In other PEGylated Sunfishes, the polyethyleneglycol5000-ω-methylether can be replaced by polyethyleneglycol2000-ω-methylether and/or the nonadecenyl tail can be replaced by tridecyl. Either 8% or 20% of SF-79 was included in the complex.

In Vivo Procedure

Study 1: BALB/c mice were injected intravenously with ¹²⁵Iodine-tyramine-cellobiose and ¹³¹I-labeled myoglobin in the free form or complexed with different lipid formulations. The mice were terminated after 30 or 120 minutes following injection and the distribution of both ¹²⁵Iodine and ¹³¹Iodine determined. Each group consisted of four (120 minutes) or five (30 minutes) mice.

Study 2: BALB/c mice were injected intravenously with ¹²⁵Iodine-tyramine-cellobiose-labeled myoglobin complexed with different PEGylated lipid formulations. The mice were sacrificed after 10, 30 or 120 minutes following injection and the body distribution of ¹²⁵Iodine was determined. In the experiment comparing the body distributions between the different PEGylated Sunfishes, two mice per group were tested. In the time curves, four mice per group were included.

Data Analyses

The organ uptake is expressed as the percentage of injected dose ¹²⁵Iodine (tyramine-cellobiose) radioactivity. Percentage of degradation is estimated by dividing the ¹³¹Iodine by ¹²⁵Iodine radioactivity times 100%. The total percentage of radioactivity in the blood was determined by multiplying the percentage per milliliter blood by 0.073 times the body weight in grams, being the total blood volume of the mouse. To allow an accurate comparison, the ratio between radioactivity in total blood and reticuloendothelial system (RES=liver+spleen) was expressed. All data are expressed as mean±standard error of the mean (SEM); comparisons were made using the student T-test.

Results

Body Distribution and Catabolism (Study 1)

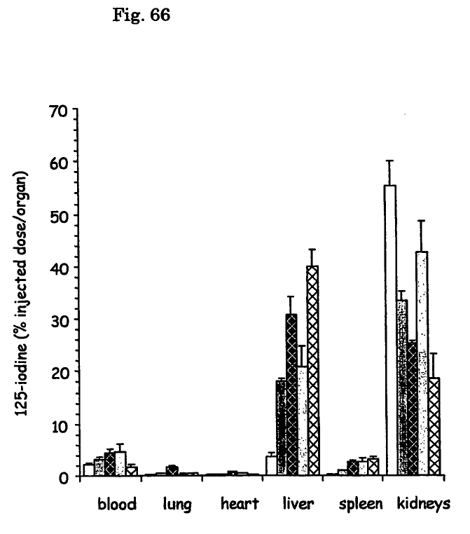

As shown in FIG. 66, injection of free myoglobin resulted in a primarily uptake of radioactivity in the kidney. After complexation with the Sunfish/co-lipid system, the distribution of myoglobin was clearly affected. Except for the kidney, a significant amount of the ¹²⁵Iodine accumulated in the liver. It is concluded that the protein-lipid complex is stable in the circulation and, hence, able to redistribute the protein. Comparison between the different complexes showed a significantly higher ¹²⁵Iodine uptake in the liver with co-lipid cholesterol as compared to DOPE (p<0.005). This suggests a preferential use of cholesterol for in vivo protein delivery.

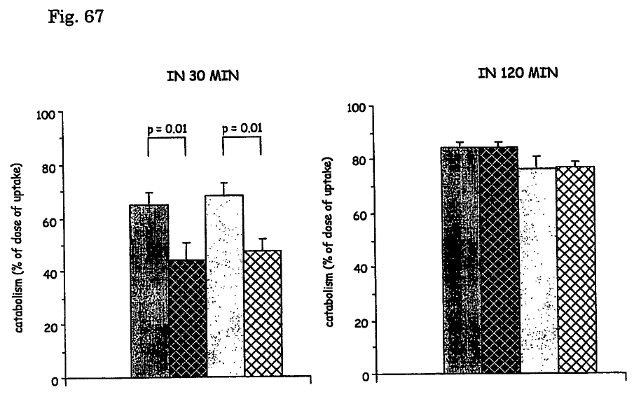

In the kidney, the rate of myoglobin degradation was not significantly different between the five groups tested. This is in agreement with the assumption that only free myoglobin reaches this side of degradation by glomerular filtration. In the liver, the Sunfish/co-lipid systems delivered the myoglobin intracellularly as proven by the finding that myoglobin was degraded in the liver (FIG. 67).

In all four groups of complexes, the liver degradation of myoglobin was completed in 120 minutes. However, the rate of degradation was lower with cholesterol than with DOPE being used as co-lipid (p=0.01). Tentatively, it might be suggested that cholesterol as co-lipid seems favorable for protein delivery since it prolongs the lifetime of the protein in the target cell.

Effect of PEGylation (Study 2)

Inclusion of 8% PEGylated Sunfish in the protein complex resulted in a prolonged circulation time (p<0.005). Of the different PEGylated lipids tested, the complex containing SF-79 appeared the most promising with the highest amount of radioactivity in the blood, 120 minutes after injection (data not shown). Therefore, SF-79 was studied in more detail.

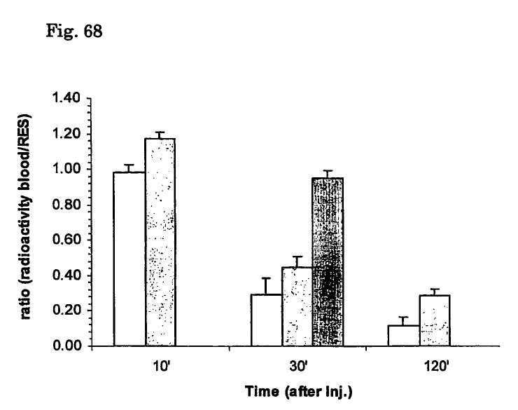

In FIG. 68, the time course of the radioactivity in blood (versus the RES) is shown, comparing complexes that do not contain PEG and complexes containing the PEGylated Sunfish, SF-79. A prolonged circulation time is obtained by inclusion of 8% PEGylated Sunfish into the complex. Especially promising is the blood/RES ratio of almost one 30 minutes after administration of complex containing 20% SF-79.

### Example 5-C

### Ex Vivo Delivery of DNA

This Example shows that Sunfish (SF) Amphiphiles can be used to deliver DNA to cells outside of the body (ex vivo), i.c. neural stem cells.

Methods

Neural stem cells were isolated from the forebrain of mice embryos and cultured in the presence of epidermal growth factor (EGF) and fibroblast growth factor (FGF). In the presence of these growth factors, neural stem cells form neurospheres, clusters of cells that grow in suspension.

Prior to transfection of neurospheres, the spheres were immobilized on Labtek culture plates that were coated with poly-ornithine. Subsequently, neurospheres were incubated for 4 hours with SF-6/DOPE (1:1 molar ratio), SF-63, SF-80/DOPE (1:1 molar ratio), or Lipofectamine 2000 lipoplexes, containing plasmid DNA encoding Enhanced Green Fluorescent Protein (pEGFP-N1). After two days, the transfection efficiency was determined with fluorescence microscopy.

Results

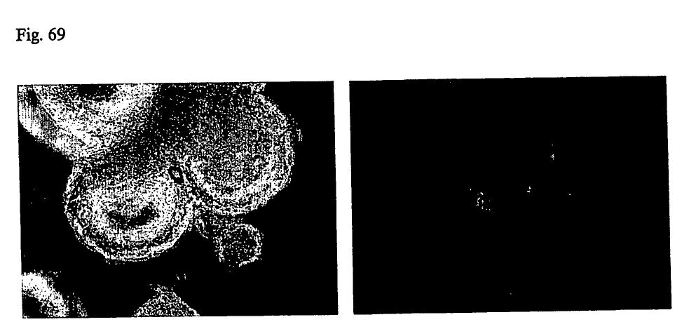

In the neurospheres, cells positive for green fluorescent protein could be observed indicating that pEGFP-N1 DNA was succesfully delivered and expressed. The transfection efficiency with SF-6/DOPE varies between experiments. The transfection efficiency with SF-80/DOPE and SF63 is constant, whilst SF-80/DOPE (FIG. 69) is more effective than SF-63 as well as Lipofectamine 2000.

### Example 6

### Use of the Protein Anchor to Target Liposomes/Polymer Particles/Hydrophobin Vesicles Complexes to Gram-Positive Cells

### Example 6-A1

### Targeting of Liposomes through the Protein Anchor to Gram-Positive Bacteria by Using Chemical Coupling of the Protein Anchor to Liposomes

The chemical coupling of the Protein Anchor to liposomes and sterically stabilized liposomes is described. The liposomes contain calcein as the reporter drug. The liposomes with the coupled Protein Anchor displayed on the surface were then incubated with TCA-pretreated L. lactis cells (ghost cells). After washing the ghost cells to remove unbound liposomes, binding to the ghost cells was demonstrated by measuring an increase in fluorescence in a fluorometer and microscopically by using a fluorescence microscope.

Materials and Methods

The Protein Anchor (cA) fusion used contained in the N-terminal domain a unique cysteine (cys) that is used as the functional element for the coupling to lipids in the liposome. In addition, it contained an epitope than can be used for detection (myc-epitope). This Protein Anchor fusion (cys::myc::cA) was designated PA3. Protein PA3 was produced in L. lactis using vector pPA3. Plasmid pPA3 is based on the nisin-inducible expression vector pNZ8048 (Kuipers et al. 1997, TibTech 15:135-140) and contains a modified multiple cloning site in which the cysteine and c-myc reporter epitope was cloned. An in-frame fusion of this reporter was made at the 5′-end the lactococcal Usp45 signal sequence and, at the 3′-end, the AcmA protein anchor sequence. Growth of L. lactis and induction for expression was as described before (Kuipers et al. 1997, TibTech 15:135-140). Isolation of PA3 from the culture medium was done by using a Sepharose SP cation exchange column (Pharmacia). The culture supernatant was loaded on the column at pH 5.8. The column was washed with phosphate buffer pH 5.8. Elution of the protein was obtained with a NaCl gradient from 0 to 0.5 M in the same buffer. Construction of liposomes loaded with calcein and detection of calcein was as described in Example 1. Coupling of PA3 to liposomes containing an activated lipid (1,2 dioleoyl-sn-glycero-3-phosphoethanolamine-N-(4-(p-maleimidophenyl) butyramide (MPB-PE)) was done as described before (Papahajopoulos et al. 1987, Ann. N.Y. Acad. Sci. 507:64-74; Maruyama et al. 1990, Proc. Natl. Acad. Sci. U.S.A. 87:5744-5748). In short, liposomes were made of DOPC and MPB-PE lipids (molar ratio 9:1) in Tris buffer pH 7 to 8. PA3 in water was added and incubated at 4 to 20° C. for 12 to 48 hours. Liposomes with coupled PA3 were isolated using a Sephadex G-50 column. Alternatively, coupling of PA3 to liposomes sterically stabilized with PEG-PE was to the polyethylene glycol moieties according to the method described by Ahmad et al. (1993, Cancer Res. 53:1484-1488). Preparation of the ghost cells and binding of the liposomes with coupled PA3 on the surface to ghost cells was done as described in patent application EP 01202239.8. Fluorescence was measured using a SpectroFluorometer (SLM500) and fluorescence was visualized using a Zeiss fluorescence microscope.

Results

The three types of calcein-loaded liposomes, one in which the PA3 anchor was coupled to the lipid, one that has PA3 coupled to the PEG-PE, and one that controls liposomes (with or without PEG-PE), were used to analyze targeting to lactococcal ghost cells. After binding, the ghost cells were extensively washed and analyzed in a spectrofluorometer. Fluorescence, which is an indication for the presence of liposomes, was only observed in the case that PA3-coupled liposomes were used. The type of coupling, to the lipid or to the PEG-PE, had no influence on the binding. The presence of the targeted liposomes on the surface of the ghost cells was confirmed using fluorescence microscopy. These results show that the AcmA-type Protein Anchor can be used to target liposomal delivery vehicles to bacteria. The liposomes may either provide slow release or can be induced to release the drugs upon an external signal (see Example 1). This may have applications in combating pathogenic bacteria. Furthermore, it is likely that mutagenized AcmA-type Protein Anchors similar to those described in Example 3 can be used to target specific human or animal cells. Alternatively, variants of the PrtP CWS domain, also described in Example 3, can be used for this purpose.

### Example 6-A2

### Targeting of Liposomes through the Protein Anchor to Gram-Positive Bacteria by Using a Protein Anchor Derivative with a Hydrophobic N-terminal Domain

The reconstitution of a Protein Anchor derivative into liposomes is described. The Protein Anchor derivative contained at its N-terminus hydrophobic peptide sequences that enabled the efficient incorporation of this part of the fusion into the lipid bilayer of the liposomes. The liposomes with the inserted Protein Anchor displayed on the surface were then incubated with TCA-pretreated L. lactis cells (ghost cells). After washing the ghost cells to remove unbound liposomes, binding to the ghost cells was demonstrated in Western blots.

Materials and Methods

The Protein Anchor (cA) fusion used contained a hydrophobic N-terminal domain used as the functional element for insertion into the lipid bilayer of the liposome. In addition, it contained an epitope than can be used for detection (myc-epitope). The hydrophobic domain (NH₂-KETWWETWWTEWSQPKKKRKV-COOH; SEQ ID NO:21) was based on translocation domains of the proteins Antennapedia (the C-terminal α-helix) of Drosophila, VP22 of Herpes simplex virus (C-terminal domain) and TAT of Human immunodeficiency virus. This Protein Anchor fusion (PEP::myc::cA) was designated PA38. Protein PA38 was produced in L. lactis using vector pPA38. Plasmid pPA38 is based on the nisin-inducible expression vector pNZ8048 (Kuipers et al. 1997, TibTech 15:135-140) and contains a modified multiple cloning site in which the PEP sequence and c-myc reporter epitope were cloned. An in-frame fusion of this reporter was made at the 5′-end with the lactococcal Usp45 signal sequence and at the 3′-end with the AcmA protein anchor sequence. Growth of L. lactis and induction for expression was as described before (Kuipers et al. 1997, TibTech 15:135-140). Isolation of PA38 from the culture medium was done by using a Sepharose SP cation exchange column (Pharmacia). The culture supernatant was loaded on the column at pH 5.8. The column was washed with phosphate buffer pH 5.8. Elution of the protein was obtained with a NaCl gradient from 0 to 0.5 M in the same buffer. DOPC liposomes were prepared as described in Example 1. PA38 was inserted into DOPC liposomes by sonication or by spontaneous insertion. Proteoliposomes were separated from free protein by gel filtration on a G-50 Sepharose column. Orientation of the inserted PA38 was studied by determining the degree of protection against proteolytic degradation by Trypsin. Preparation of the ghost cells, binding of the PA38 proteoliposomes with Protein Anchor moiety exposed on the surface to ghost cells, and detection by Western blots were done as described in patent application EP 01202239.8.

Results

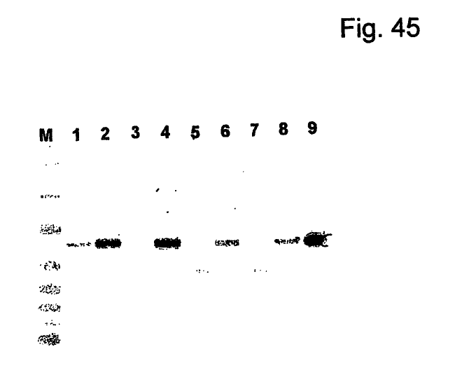

PA38 used in the preparation of liposomes was found in the column fractions containing phospholipids, meaning that it integrated into liposomes. Integration and the orientation of the integrated PA38 were studied by trypsin digestion (FIG. 45). Trypsin degrades free protein and cannot degrade those parts of the protein that are incorporated in the lipid bilayer or that are present in the lumen of the liposomes. The Western blot analysis showed that more PA38 was reconstituted into liposomes by sonication (lanes 1-4) compared to the spontaneous insertion of PA38 (lanes 5-8). The spontaneous integration of PA38 into lipidic membranes is directed by the function of PEP, which is translocation across a lipidic membrane. Furthermore, the analysis showed that approximately similar amounts of PA38 were inserted in the lipidic membrane using the two reconstitution methods. However, sonication resulted in higher loading of the protein into the lumen of liposomes. This can be concluded from the presence of full-length protein after incubation with trypsin (lane 1) as compared to the non-sonicated equivalent sample in lane 5.

The lower size fragments of PA38 were still protected against trypsin in the presence of 3% triton X-100, whereas free PA38 (result not shown) and full length PA38 in the lumen of liposomes (compare lane 1 and lane 3) were completely degraded under such conditions. This indicated that the conformation of the integrated PA38 is probably changed into a conformation, which is more resistant to trypsin degradation.

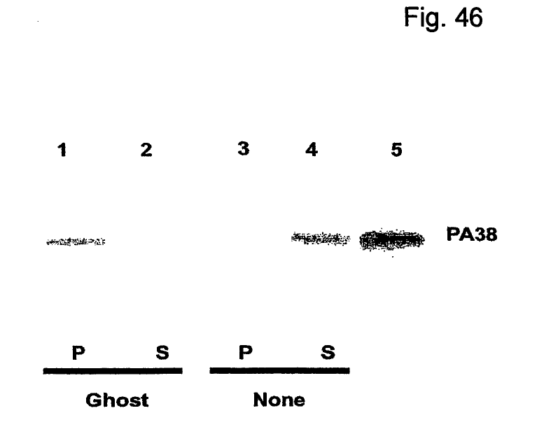

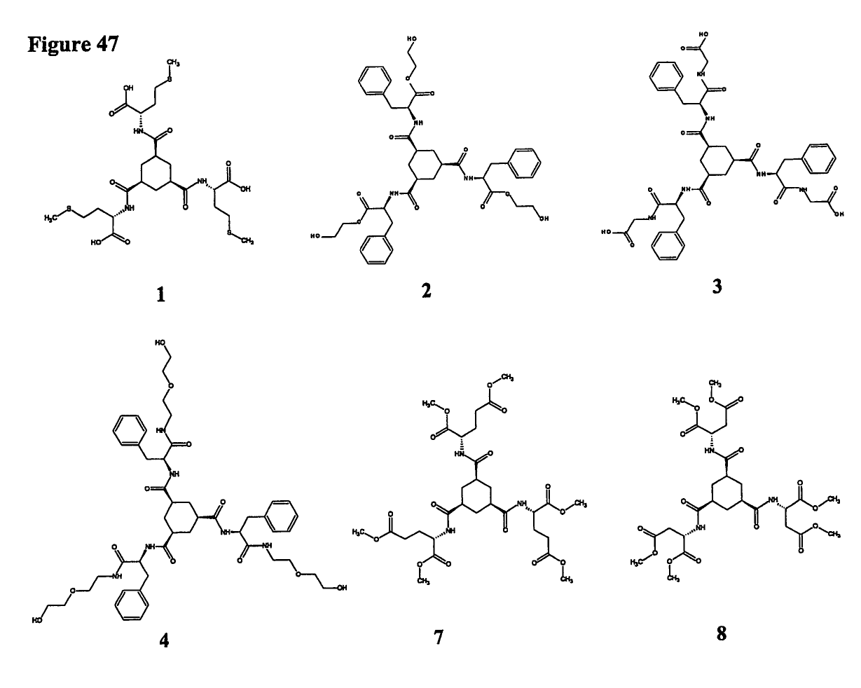

Liposomes with reconstituted PA38 generated without sonication were used to study the binding of these liposomes to lactococcal ghost cells in order to demonstrate the functionality of the Protein Anchor and its ability to target liposomes to bacterial cells. PA38-liposomes were incubated with or without ghost cells. After incubation, the samples were centrifuged at 3,000 g for five minutes and the samples were washed once with PBS to remove loosely associated liposomes. The samples were analyzed in Western blots and FIG. 46 clearly shows that the liposomes could be sedimented only in the presence of ghost cells (compare lanes 1 and 3), which demonstrated that they bind to the ghost cells through the Protein Anchor.

### Example 6-A3

### Targeting of Liposomes through the Protein Anchor to Gram-Positive Bacteria by Using a Lipo-Protein-Anchor Derivative

The Protein Anchor derivative contained a modified secretion signal sequence that enabled the efficient coupling in vivo of the Protein Anchor to the lipid bilayer of the bacterial membrane. In this way, the Protein Anchor was produced as a lipoprotein that was isolated and reconstituted into liposomes with attached Protein Anchor. The liposomes with the coupled Protein Anchor displayed on the surface were then incubated with TCA-pretreated L. lactis cells (ghost cells). After washing the ghost cells to remove unbound liposomes, binding to the ghost cells was demonstrated in Western blots.

Materials and Methods

The Protein Anchor (cA) fusion used contained a modified secretion signal by changing the processing site at Val −3 into Leu, Tyr −2 into Ser, Asp +1 into Cys and Thr +2 into Ser, which resulted in the in vivo production of the Protein Anchor as a lipoprotein. The exported and processed lipoprotein also contained an epitope that can be used for detection (myc-epitope). This Protein Anchor fusion (Lipo::myc::cA) was designated PA93. Protein PA93 was produced in L. lactis using vector pPA93. Plasmid pPA93 is based on pPA3 (Example 6-A1). Growth of L. lactis and induction for expression was as described before (Kuipers et al. 1997, TibTech 15:135-140). L. lactis cells were treated with lysozyme to remove the cell walls. Cell membranes containing the PA93 derivative were isolated by high-speed centrifugation. Resuspended membranes were mixed with DOPC lipids and liposomes were prepared essentially as described in Example 1. Proteoliposomes were purified by gel filtration on a G-50 Sepharose column. Preparation of the ghost cells and binding of the PA-proteoliposomes with Protein Anchor moiety exposed on the surface to ghost cells and detection by Western blots was done as described in patent application EP 01202239.8.

Results

Liposomes with reconstituted PA93 were used to study the binding of these liposomes to lactococcal ghost cells in order to demonstrate the functionality of the Protein Anchor and its ability to target liposomes to bacterial cells. PA93-liposomes were incubated with or without ghost cells. After incubation, the samples were centrifuged at 3,000 g for five minutes and the samples were washed once with PBS to remove loosely associated liposomes. The samples were analyzed in Western blots and showed that the liposomes could be sedimented only in the presence of ghost cells, which demonstrated that they bind to the ghost cells through the Protein Anchor.

### Example 6-A4

### Targeting of Liposomes through the Protein Anchor to Gram-Positive Bacteria by Using a Protein Anchor Derivative with a Transmembrane-Spanning Domain

The Protein Anchor derivative contained a defective processing site for the bacterial leader peptidase and the signal sequence functioned in this way as an efficient transmembrane-(TM-) spanning domain. This enabled the efficient incorporation of this part of the fusion into the lipid bilayer of the liposomes. The liposomes with the inserted Protein Anchor displayed on the surface were then incubated with TCA-pretreated L. lactis cells (ghost cells). After washing the ghost cells to remove unbound liposomes, binding to the ghost cells was demonstrated in Western blots.

Materials and Methods

The Protein Anchor (cA) fusion used contained a hydrophobic N-terminal domain that was derived from the lactococcal Usp45 secretion signal. The modified secretion signal functioned in this way as a transmembrane (TM) sequence rather than a secretion signal. As a result, the exported Protein Anchor moiety remained attached in vivo to the lipid bilayer. The following modifications were made in the Usp45 secretion signal: Ala −1 was changed into Phe and Asp +1 was changed into Tyr. The exported and processed transmembrane protein also contained an epitope that can be used for detection (myc-epitope). This Protein Anchor fusion (TM::myc::cA) was designated PA94. Protein PA94 was produced in L. lactis using vector pPA94. Plasmid pPA94 is based on pPA3 (Example 6-A1). Growth of L. lactis and induction for expression was as described before (Kuipers et al. 1997, TibTech 15:135-140). L. lactis cells were treated with lysozyme to remove the cell walls. Cell membranes containing PA94 were isolated by high-speed centrifugation. Resuspended membranes were mixed with DOPC lipids and liposomes were prepared, essentially as described in Example 1. Proteoliposomes were purified by gel filtration on a G-50 Sepharose column. Preparation of the ghost cells and binding of the PA-proteoliposomes with Protein Anchor moiety exposed on the surface to ghost cells and detection by Western blots was done as described in patent application EP 01202239.8.

Results

Liposomes with reconstituted PA94 were used to study the binding of these liposomes to lactococcal ghost cells in order to demonstrate the functionality of the Protein Anchor and its ability to target liposomes to bacterial cells. PA94 liposomes were incubated with or without ghost cells. After incubation, the samples were centrifuged at 3,000 g for five minutes and the samples were washed once with PBS to remove loosely associated liposomes. The samples were analyzed in Western blots and showed that the liposomes could be sedimented only in the presence of ghost cells, which demonstrated that they bind to the ghost cells through the Protein Anchor.

### Example 6-B

### Targeting of Polymer Particles through the Use of the Protein Anchor to Gram-Positive Bacteria

The coupling of the Protein Anchor to a polymer particle is described. The particles contain a gel (consisting of low molecular weight compounds) and a reporter drug (calcein). The Protein Anchor was displayed on the particle surface, which was then incubated with TCA-pretreated L. lactis cells (ghost cells). After washing the ghost cells to remove unbound polymer particles, binding of calcein-loaded polymer particles to the ghost cells was demonstrated.

Materials and Methods

Production and isolation of PA3 as described under Example 6-A. Preparation of ghost cells and binding conditions was as described before (patent application EP 01202239.8). Preparation of polymer particles consisting of methyl methacrylate or diethyleneglycol dimethacrylate was described before (N. B. Graham and A. Cameron, 1998, Pure Appl. Chem. 70:1271-1275). Cross-linking reagents were: N-(p-Maleimidophenyl)isocyanate PMPI (couples sulfhydryl groups to hydroxyl groups); N-(β-Maleimidopropionic acid)hydrazide BMPH (couples sulfhydryl groups to aldehyde groups). Coupling conditions of PA3 to the polymer particles were according to the specifications of the supplier (Pierce, Ill., USA). Basically, PMPI was used to react with an —OH group of the polymer to form a carbamate link at pH 8.5 and the maleimide moiety was used to react with the —SH group of PA3 at pH 6.5 to 7.5. BMPH was used to react with aldehyde groups in the polymer at pH 6 to 9 and the maleimide moiety was used to react with the —SH group of PA3 at pH 6.5 to 7.5. The particles were then loaded with a gel containing calcein as described in the other Examples. Specific binding to the bacteria was demonstrated by detection of calcein and by Western blotting using an anti-myc-specific antibody for detection.

Results

Two types of polymer particles loaded with organogel and calcein, one to which the PA3 anchor was coupled and one control to which no PA3 had been coupled, were used to analyze targeting to lactococcal ghost cells. After binding, the ghost cells were extensively washed and analyzed in a Spectrofluorometer and in Western blots. Fluorescence, which is an indication for the presence of the polymer particles, was only observed in the case that PA3-coupled polymer particles were used. The presence of the targeted polymer particles on the surface of the ghost cells was confirmed using fluorescence microscopy and in the Western blots. These results showed that the AcmA-type Protein Anchor can be used to target polymeric delivery vehicles filled with gels to bacteria. The gels can provide slow release or can be induced to release the drugs upon an external signal (see Example 7). This has applications in combating pathogenic bacteria. Furthermore, mutagenized AcmA-type Protein Anchors similar to those described in Example 3 can be used to target specific human or animal cells. Alternatively, variants of the PrtP CWS domain, also described in Example 3, can be used for this purpose.

### Example 6-C

### Targeting of Hydrophobin Vesicles through the Use of the Protein Anchor to Gram-Positive Bacteria

The coupling of the Protein Anchor PA3 to SC3 vesicles loaded with calcein is described. The SC3 vesicles with the coupled PA3 displayed on the surface were then incubated with TCA-pretreated L. lactis cells (ghost cells). After washing the ghost cells to remove unbound vesicles, binding of calcein-loaded hydrophobin vesicles to the ghost cells was demonstrated.

Materials and Methods

Production and isolation of PA3 as described in Example 6-A. Preparation of ghost cells was as described before (patent application EP 01202239.8). Preparation of SC3 vesicles loaded with calcein as reporter drug was according to the method as described in Example 4. Coupling of PA3 to the monomeric form of SC3 or to the SC3 vesicles was done by coupling the carbohydrates of SC3 to the unique cysteine in PA3 using the cross-linking reagent N-β-Maleimidopropionic acid (BMPH), which was used according to the instructions of the supplier (Pierce, Ill., USA). In short, the carbohydrates of SC3 were activated by treatment with 2.5% periodate. The oxidized carbohydrates (aldehydes) were reacted with hydrazide group in BMPH to form a stable hydrazone linkage. The SC3 with coupled cross-linker was then reacted through its maleimide moiety with the sulphydryl group in PA3 at pH 6.5 to 7.5. Alternatively, amino groups in PA3 were directly reacted with the activated sugar moieties in SC3 at pH 5.5 to 8.5 in order to obtain coupling. PA3 coupled by either method proved to be functional. The hydrophobin vesicles with coupled PA3 were then incubated with ghost cells. Specific binding to the bacteria was demonstrated by detection of calcein as described in Example 6-A1 and by Western blotting using a specific anti-myc antibody.

Results

The calcein-loaded hydrophobin vesicles to which the PA3 anchor had been coupled and the control to which no PA3 had been coupled, were used to analyze targeting to lactococcal ghost cells. After binding, the ghost cells were extensively washed and analyzed in a Spectrofluorometer. Fluorescence, which is an indication for the presence of the hydrophobin vesicles, was only observed in the case that PA3-coupled hydrophobin vesicles were used. The presence of the targeted hydrophobin vesicles on the surface of the ghost cells was confirmed using fluorescence microscopy and in Western blots. These results show that the AcmA-type Protein Anchor can be used to target hydrophobin vesicles to bacteria. The hydrophobin vesicles either provide slow release or can be induced to release the drugs upon an external signal when they are filled with a responsive organogel (see Example 7). This has applications in combating pathogenic bacteria. Furthermore, mutagenized AcmA-type Protein Anchors similar to those described in Example 3 can be used to target specific human or animal cells. Alternatively, variants of the PrtP CWS domain, also described in Example 3, can be used for this purpose.

### Example 7

### Use of Responsive Gels of Low Molecular Weight Compounds as Delivery Vehicles

### Example 7-A

### Temperature as a Stimulus for Gel-to-Sol and Sol-to-Gel Transitions

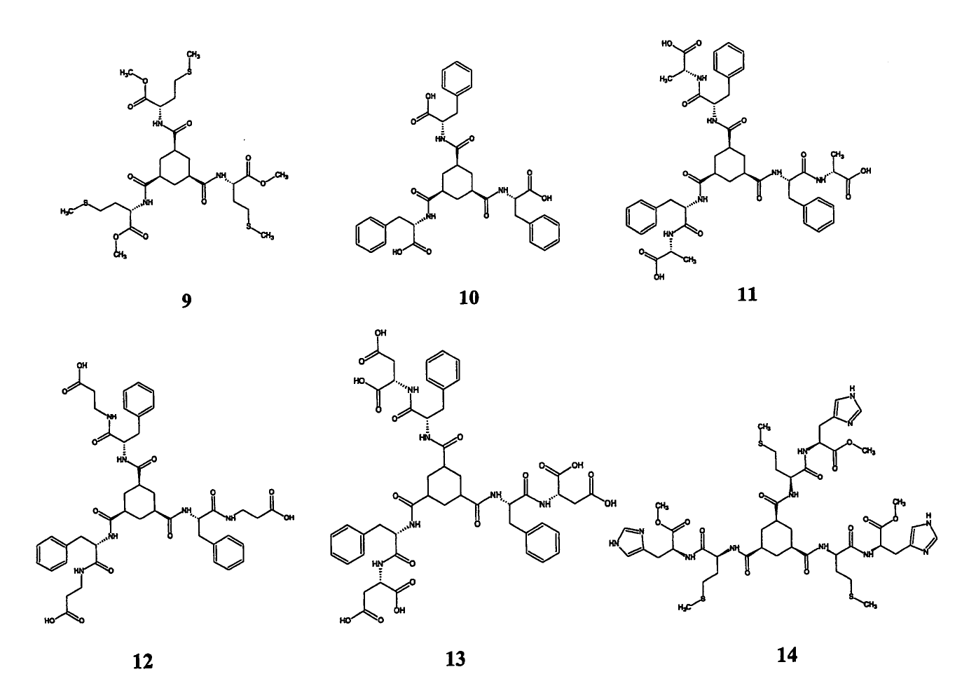

Synthesis of Gelators 1-4 and 7-13 (Chemical Structures in FIG. 47)

Synthesis of CHexAmMetOH (1)

a) Synthesis of CHexAmMetOMe (9a)

Cis, cis-1,3,5-cyclohexanetricarbonyl trichloride (1.40 g, 5.2 mmol) in dry CH₂Cl₂ (15 ml) was added to a solution of HCl.L-Met-OMe (3.20 g, 16.1 mmol) and Et₃N (4.5 ml, 32.8 mmol) in dry CH₂Cl₂ (200 ml, T=0° C.). The solution was slowly brought back to room temperature and left stirring overnight. The precipitate formed was filtered and washed with ethanol. The product dried in the vacuum oven. Yield: 2.96 g (4.54 mmol, 87%).

b) Synthesis of CHexAmMetOMe (Racemic) (9b)

9b was synthesized similarly to 9a, starting from the racemic HCl.L-Met-OMe. Yield: 1.33 g (2.0 mmol; 47%).

c) Synthesis of CHexAmMetOH (1)

To a solution (T=0° C.) of CHexAmMetOMe (9a) (1.5 g, 2.3 mmol) in MeOH (30 ml) was added 2 M NaOH (15 ml). The mixture was slowly brought back to room temperature and stirred for 20 hours. The solution was diluted with water (50 ml) and 2 M HCl was added until pH<3. The precipitate formed was filtered and finally dried in the vacuum oven. Yield 1.27 g (2.10 mmol, 91%).

Synthesis of CHexA mPheOCH₂CH₂OH (2)

a) Synthesis of CHexAmPheOMe

Cis, cis-1,3,5-cyclohexanetricarbonyl trichloride (1.01 g, 3.7 mmol) in dry CH₂Cl₂ (5 ml) was added to HCl.L-Phe-OMe (1.90 g, 11.1 mmol) and Et₃N (3.0 ml, 22.2 mmol) in dry CH₂Cl₂ (50 ml, T=0° C.). The solution was slowly brought back to room temperature and left stirring overnight. The precipitate formed was collected by filtration and washed with ethanol and finally crystallized from DMSO/ethanol. Yield: 2.12 g (3.30 mmol, 82%).

b) Synthesis of CHexAmPheOH

CHexAmPheOMe (0.50 g, 0.71 mmol) was added to MeOH (10 ml) and 2 M NaOH (5 ml, T=0° C.). The mixture was slowly brought back to room temperature and stirred for 20 hours. The solution was diluted with water (25 ml) and 2 M HCl was added until pH<3. The precipitate formed was filtered off and dried in the vacuum oven. Yield: 0.41 g (0.60 mmol, 84%).

c) Synthesis of CHexAmPheOCH₂CH₂OH (2)

CHexAmPheOH (0.49 g, 0.74 mmol) was dissolved in ethylene glycol (60 ml). After addition of concentrated HCl (three drops), the solution was heated slowly=until T=135° C. After three hours, the reaction mixture was cooled to T=−20° C. The precipitate formed was filtrated and washed with acetone. Yield: 0.36 g (0.46 mmol, 59%).

Synthesis of CHexAmPheGlyOH (3)

a) Synthesis of Boc-L-Phe-Gly-OMe

Boc-L-Phe-Suc (1.81 g, 5.0 mmol), HCl.Gly-OMe (0.63 g, 5.0 mmol) and Et₃N (0.70 ml, 5.0 mmol) were dissolved in ethyl actetate (60 ml). After stirring for 20 hours, the organic layer was washed with H₂O (60 ml), saturated NaHCO₃ (60 ml) and brine (60 ml) and dried with MgSO₄. After filtration and evaporation of the solvent, the residue was purified by column chromatography (Silica, CH₂Cl₂—CH₂Cl₂/MeOH 100:5). Yield: 1.55 g (4.61 mmol, 92%).

b) Synthesis of TFA.L-Phe-Gly-OMe

Boc-L-Phe-Gly-OMe (1.55 g, 4.61 mmol) was dissolved in 2M TFA/CH₂Cl₂ (30 ml). After four hours stirring, the solvents were evaporated and the residue was dried under high vacuum (0.1 mm Hg). Yield: 2.13 g (contaminated with free TFA, about 4.56 mmol).

c) Synthesis of CHexAmPheGlyOMe

Cis, cis-1,3,5-cyclohexanetricarbonyl trichloride (0.40 g, 1.47 mmol) in dry CH₂Cl₂ (15 ml) was added to TFA.L-Phe-Gly-OMe (about 4.61 mmol, contaminated with approximately 4.56 mmol TFA) and Et₃N (1.91 ml, 13.8 mmol) in dry CH₂Cl₂ (30 ml, T=0° C.). The solution was slowly brought back to room temperature and left stirring overnight. The precipitate formed was collected by filtration and washed with ethanol. Yield: 0.80 g (0.92 mmol, 62%).

d) Synthesis of CHexAmPheGlyOH (3)

CHexAmPheGlyOMe (0.42 g, 0.48 mmol) was added to MeOH (15 ml) and 2 M NaOH (7.5 ml, T=0° C.). The mixture was slowly brought back to room temperature and stirred for 72 hours. The solution was diluted with water (25 ml) and 2 M HCl was added until pH<3. The precipitate formed was filtered off and dried in the vacuum oven. Yield: 0.34 g (0.41 mmol, 85%).

Synthesis of CHeXAmPheNHCH₂CH₂OCH₂CH₂OH (4)

a) Synthesis of Boc-L-PheNHCH₂CH₂OCH₂CH₂OH

2(-2-aminoethoxy)-1-ethanol (0.72 g, 6.9 mmol) and Et₃N (0.96 ml, 6.9 mmol) were dissolved in ethyl acetate (60 ml). Subsequently, BOC-L-Phe-Suc (2.50 g, 6.9 mmol) in ethyl acetate (50 ml) was added to the reaction mixture. After stirring for 20 hours, the organic solvent was extracted with H₂O, 10% NaHCO₃, H₂O, brine and dried over MgSO₄. After filtration, ethyl acetate was evaporated in vacuo. Yield: 1.55 g (4.40 mmol; 64%).

b) Synthesis of TFA.L-PheNHCH₂CH₂OCH₂CH₂OH

Boc-L-PheNHCH₂CH₂OCH₂CH₂OH (1.55 g, 4.4 mmol) was dissolved in 2M TFA/CH₂Cl₂ (115 ml). After four hours stirring, the solvents were evaporated and the residue was dried under high vacuum (0.1 mm Hg). Yield: 3.01 g (contaminated with free TFA, about 12.2 mmol).

c) Synthesis of CHexAmPheNHCH₂CH₂OCH₂CH₂OH (4)

Cis, cis-1,3,5-cyclohexanetricarbonyl trichloride (0.40 g, 1.47 mmol) in dry CH₂Cl₂ (5 ml) was added to TFA.L-PheNHCH₂CH₂OCH₂CH₂OH (3.01 g, about 4.40 mmol, contaminated with approximately 12.2 mmol TFA) and Et₃N (2.9 ml, 20.8 mmol) in dry CH₂Cl₂ (100 ml, T=0° C.). The solution was slowly brought back to room temperature and left stirring overnight. The precipitate formed was collected by filtration, washed with ethanol and recrystallized from water. Yield: 0.78 g (0.85 mmol, 57%).

Synthesis of CHexAmGluOMe (7)

L-glutamic acid methyl ester hydrochloride (4.70 g; 22.1 mmol; 3.0 eq) in 200 ml dry CH₂Cl₂ was cooled to 0° C. and Et₃N (6.2 ml; 44.2 mmol; 6.0 eq) was added. Cis, cis-1,3,5-cyclohexanetricarbonyl trichloride (2.00 g; 7.4 mmol; 1.0 eq) in 15 ml dry CH₂Cl₂ was added to the cooled solution and the mixture was slowly brought back to room temperature and left stirring overnight. When the reaction was stopped, a “gel-like” precipitate was formed. This solid was collected by vacuum filtration. The precipitate formed a gel with MeOH, EtOH, H₂O and CH₂Cl₂. The precipitate was recrystallized in ether, filtered and dried in the oven for at least one week. Yield: 1.50 g (2.2 mmol, 30%).

Synthesis of CHexAmAspOMe (8)

L-aspartic acid methyl ester hydrochloride (1.70 g; 8.4 mmol; 3.0 eq) in 200 ml dry CH₂Cl₂ was cooled to 0° C. and Et₃N (2.3 ml; 16.8 mmol; 6.0 eq) was added. Cis, cis-1,3,5-cyclohexanetricarbonyl trichloride (0.76 g; 2.8 mmol; 1.0 eq) in 20 ml dry CH₂Cl₂ was added to the cooled solution and the mixture was slowly brought back to room temperature and left stirring for forty hours. Acetone was added and the precipitate was filtered and dried in the oven. The precipitate was recrystallized in ethanol, filtered and dried in the oven. Yield: 0.92 g (1.4 mmol; 51%).

Synthesis of CHexAmPheOH (racemic) (10a)

Racemic CHexAmPheOMe (0.65 g) was added to 20 ml MeOH and stirred. The mixture was cooled and NaOH (15 ml; 2 M) was added. The mixture was slowly brought back to room temperature and stirred for 20 hours. The solution was diluted with water (75 ml) and 2 M HCl was added until the pH was lower than 3. The precipitate was filtered and dried in the vacuum oven. Yield: 0.42 g (0.6 mmol; 69%).

Synthesis of CHexAmPheOH (DDL) (10b)

a) Synthesis of CHexAmPheAm-(1,4)-ArNO₂ (DDL)

CHexAm(L)PheAm-(1,4)-ArNO₂ (0.50 g; 1.0 mmol; 1.0 eq), D-phenylalanine methyl ester hydrochloride (0.46 g; 2.1 mmol; 2.1 eq), DMT-MM (0.61 g; 2.2 mmol; 2.2 eq) and Et₃N (0.29 ml; 0.21 g; 2.1 mmol; 2.1 eq) were stirred in 50 ml MeOH overnight. The next morning, approximately 20 ml ethanol was added and the mixture was stirred for a further 15 minutes. The remaining precipitate was filtered and dried in the vacuum oven. Yield: 0.50 g (0.6 mmol; 59%).

b) Synthesis of CHexAmPheOH (DDL) (10b)

CHexAm-(2×D)-PheOMe-(1×L)-PheAm-(1,4)-ArNO₂ (0.50 g; 0.6 mmol; 1.0 eq) was stirred in 15 ml MeOH and NaOH (10 ml; 2 M) was added. The reaction was stirred for two days. At the end of the reaction, the mixture had turned bright yellow. Two M HCl was added until the pH was 2 and a precipitate formed. The precipitate was filtered and washed with water until the precipitate and the filtrate were no longer yellow. The product was dried in the oven. Yield: 0.42 g (0.6 mmol; 97%).

Synthesis of CHexAm(L)Phe(D,)AlaOH (11)

a) Synthesis of TFA⁻H₃N⁺-(L)Phe(D)AlaOMe

BOC-(L)Phe(D)AlaOMe (2.69 g; 7.7 mmol; 1.0 eq) was dissolved in 25 ml CH₂Cl₂ and 15 ml TFA in 75 ml CH₂Cl₂ were added. The mixture was stirred for three hours. Solvent and excess TFA were evaporated in vacuo, yielding 4.83 g TFA⁻H₃N⁺-(L)Phe(D)AlaOMe and excess TFA, which could not be evaporated. The product was used in the next step without further purification.

b) Synthesis of CHexAm(L)Phe(D)AlaOMe

TFA⁻H₃N⁺-(L)Phe(D)AlaOMe (4.83 g) in 100 ml dry CH₂Cl₂ was cooled and Et₃N (4.70 ml; 3.42 g; 33.8 mmol) was added. Cis, cis-1,3,5-cyclo-hexanetricarbonyl trichloride (0.7 g; 2.6 mmol; 1.0 eq) in 5 ml dry CH₂Cl₂ was added to the reaction mixture. The solution was slowly brought back to room temperature and left stirring overnight. The next morning, ethanol was added to the mixture and the remaining precipitate was filtered and dried in the oven. The product was used in the next step without further drying or purification. Yield: 4.5 g (wet).

c) Synthesis of CHexAm(L)Phe(D)AlaOH (11)

4.5 g (wet) CHexAm(L)Phe(D)AlaOMe was stirred in 50 ml MeOH. 2M NaOH (15 ml) was added and the reaction was stirred overnight. The next day, 50 ml H₂O and 50 ml MeOH were added and the pH was brought to 2 with 2M HCl. The formed precipitate was filtered, washed with MeOH and dried in the vacuum oven. The obtained product was dissolved in approximately 3 ml NaOH (2M). EtOH (30 ml) was added after which a gel formed. The gel was filtered and the solid was dissolved in 1 ml 2M NaOH. To this solution, approximately 4 ml MEOH and 5 ml H₂O was added, then approximately 2 ml 2M HCl was added until pH 4. The precipitate was filtered and dried in the vacuum oven. Yield: 0.54 g (0.6 mmol; 24%).

Synthesis of cHexAm(L)Phe(β)AlaOH (12)

a) Synthesis of BOC-(L)Phe(P)AlaOMe

BOC-Phe-Suc (2.51 g; 6.9 mmol; 1.0 eq) and β-alanine methyl ester hydrochloride (0.96 g; 6.9 mmol; 1.0 eq) were stirred at room temperature in 50 ml ethyl acetate and Et₃N (1.92 ml; 1.40 g; 13.8 mmol; 2.0 eq) overnight. The next day, the formed precipitate (Et₃N*HCl) was filtered off. The organic solvent was extracted with H₂O, 10% NaHCO₃, H₂O, Brine and dried over MgSO₄. Ethyl acetate was evaporated in vacuo, giving BOC-(L)Phe(β)AlaOMe. The product was used in the next step without further purification. Yield: 2.10 g (6.0 mmol; 87%).

b) Synthesis of TFA⁻H₃N⁺-(L)Phe(P)AlaOMe

BOC-(L)Phe(O)AlaOMe (2.10 g; 6.0 mmol; 1.0 eq) was stirred for three hours in 100 ml CH₂Cl₂ and 15 ml TFA. Solvent and excess TFA were evaporated in vacuo, yielding 4.63 g TFA⁺H₃N⁻-(L)Phe(O)AlaOMe and excess TFA, which could not be evaporated. The product was used without further purification for the next reaction step.

c) Synthesis of CHexAm(L)Phe(β)AlaOMe

TFA⁻H₃N⁺-(L)Phe(O)AlaOMe (4.63 g) in 100 ml dry CH₂Cl₂ was cooled and Et₃N (7.00 ml; 5.09 g; 50.3 nmol; excess) was added. Cis, cis-1,3,5-cyclo-hexanetricarbonyl trichloride (0.45 g; 1.7 mmol; 1.0 eq) in 10 ml dry CH₂Cl₂ was added to the reaction mixture. The solution was slowly brought back to room temperature and left stirring overnight. The next morning, ethanol was added to the mixture and the remaining precipitate was filtered and dried in the oven. The product was CHexAm(L)Phe(β)AlaOMe and the yield was not determined. The product was used in the next step without further drying or purification.

d) Synthesis of CHexAm(L)Phe(P)AlaOH (12)

All the product made in the previous step (CHexAm(L)Phe(β)AlaOMe) was stirred in 5 ml MeOH and NaOH (10 ml; 2M) overnight. The next day, 15 ml MEOH and NaOH (10 ml; 2M) were added and the reaction was again stirred overnight. The next day, the mixture was filtered, after which water (approximately 50 ml) was added. The pH was brought to 2 with 2M HCl. The formed precipitate was filtered and dried in the vacuum oven to give pure CHexAm(L)Phe(β)AlaOH. Yield: 1.18 g (1.4 mmol; 80%).

Synthesis of CHexAmPheAmGluOH (13)

a) Synthesis of BOC-PheAmGluOMe

L-BOC-Phe-Suc (2.50 g; 6.9 mmol; 1.0 eq) and L-Glutamic acid dimethyl ester hydrochloride (1.5 g; 7.1 mmol; 1.1 eq) were stirred at room temperature in 50 ml ethyl acetate and Et₃N (1.92 ml; 1.40 g; 13.8 mmol; 2.0 eq) overnight. The next day, the formed precipitate (Et₃N*HCl) was filtered off. The organic solvent was extracted with H₂O, 10% NaHCO₃, H₂O, Brine and dried over MgSO₄. Ethyl acetate was evaporated in vacuo, BOC-PheAmGluOMe. The product was used in the next step without further purification. Yield: 2.53 g (6.4 mmol; 93%).

b) Synthesis of TFA⁻H₃N⁺-PheAmGluOMe

BOC-PheAmnGluOMe (2.53 g; mmol; 1.0 eq) was stirred for three hours in 60 ml CH₂Cl₂ and 10 ml TFA. Solvent and excess TFA were evaporated in vacuo, yielding 4.91 g TFA⁺H₃N⁻-PheAmGluOMe and excess TFA, which could not be evaporated.

c) Synthesis of CHexAmPheAmGluOMe

TFA⁻H₃N⁺-PheAmGluOMe (4.91 g) in 60 ml dry CH₂Cl₂ was cooled and Et₃N (5.00 ml; 3.64 g; 35.9 mmol; excess) was slowly added. Cis, cis-1,3,5-cyclo-hexanetricarbonyl trichloride (0.48 g; 1.8 mmol; 1.0 eq) in 10 ml dry CH₂Cl₂ was slowly added to the reaction mixture. The solution was slowly brought back to room temperature and left stirring overnight. The next morning, ethanol was added to the mixture. After 15 minutes of stirring, the remaining precipitate was filtered and dried in the oven. The product was CHexAmPheAmGluOMe and the yield was not determined, but 0.32 g (0.3 mmol) CHexAmPheAmGluOMe was obtained. The product was used in the next step.

d) Synthesis of CHexAmPheAmGluOH (13)

All the product minus 0.32 g made in the previous step was stirred in 50 ml MeOH and NaOH (20 ml; 2 M) overnight. The next day, 5 ml EtOH and water (approximately 50 ml) was added. The pH was brought to 2 with 2 M HCl. The formed precipitate was filtered and dried in the vacuum oven. The product was recrystalized twice by dissolving it in NaOH and precipitating it by acidification with HCl. The precipitate was filtered off and dried to give pure CHexAmPheAmGluOH. Yield: 0.47 g (0.47 mmol; 31%).

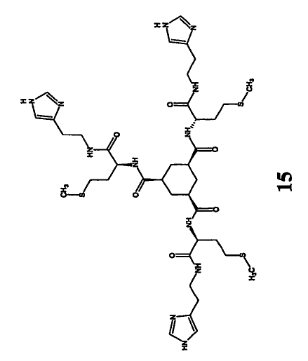

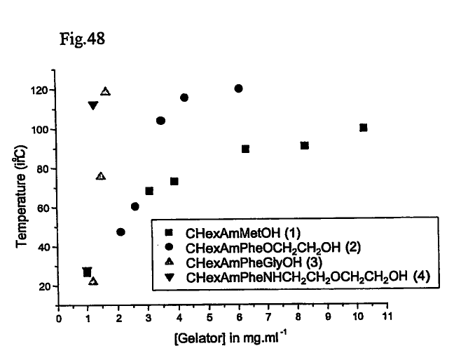

Melting temperatures of the gels (T_{gel}) were determined by the dropping ball method (H. M. Tan, A. Moet, A. Hiltner, E. Baer, Macromolecules 1983, 16, 28). As shown in FIG. 48, the melting temperature of the gels of 1-4 depends on the concentration and the chemical structure of the gelator.

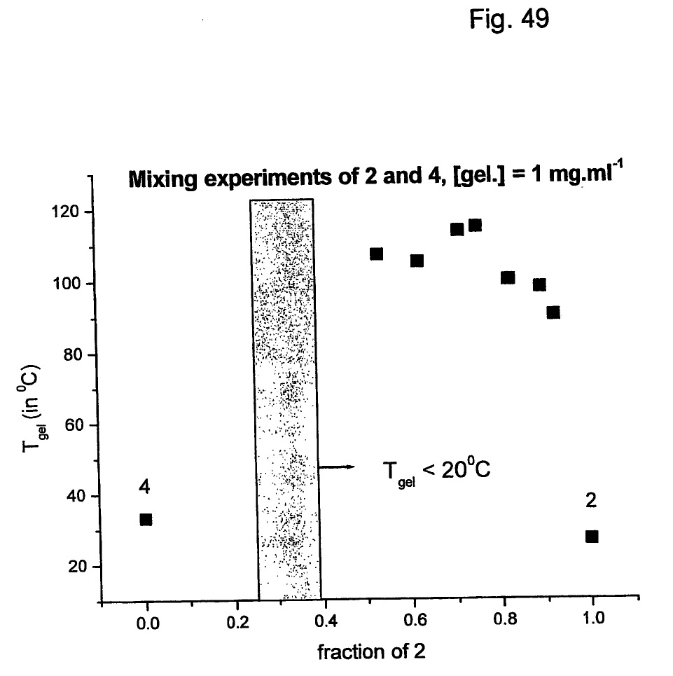

In a special case, mixing of two gelators leads to an increase in the melting temperature of the gel (FIG. 49).

### Example 7-B

### pH as a Stimulus for Gel-to-Sol and Sol-to-Gel Transitions

### Example 7-B1

### Acidic Gelators

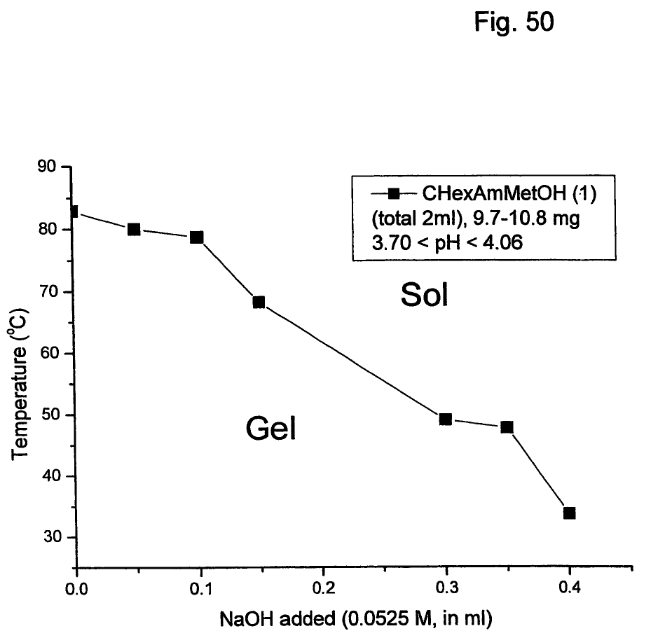

Addition of base to a gel of 1 led to a decrease in melting temperature of the gel. Above pH>4.06, gelation was no longer observed (reversible process; see FIG. 50). Similarly, addition of sufficient amounts of base to gels of 3, 8 and 9 also causes dissolution of these gels.

### Example 7-B2

### Basic Gelators

Cyclohexane bis-ureidohexylamine (Chemical Structure 5 in FIG. 51); Synthesis of 5a Representing the (1S,2S) Diastereomer

a) Synthesis of Phenyl (1S,2S)-2-((phenoxycarbonyl)amino) cyclohexylcarbamate or trans-(1S,2S) 1,2 bis-phenoxycarbonylamino cyclohexane

To a cooled (T=0° C.) solution of phenyl chloroformate (3.0 g, 19 mmol) in CH₂Cl₂ (16 ml), was added a solution of trans-(1S,2S)-1,2-diaminocyclohexane (1.0 g, 8.8 mmol and (iPr)₂EtN (3.0 ml) in CH₂Cl₂ (20 ml). The suspension was stirred at room temperature for 18 hours and subsequently placed in the cooling cell for 2 hours. The obtained precipitate was filtered off and washed with cold CH₂Cl₂ and hexane. The white solid was dried in a vacuum oven. Yield 2.23 g (6.3 mmol, 72%).

b) Synthesis of (1S,2S) chex-bisurea dihexyl BOC amine

To a solution of phenyl (1S,2S)-2-((phenoxycarbonyl)amino) cyclohexylcarbamate or trans-(1S, 2S)-1,2-bis-phenoxycarbonylamino cyclohexane (1.0 g, 2.82 mmol) and N-BOC-1,6-diaminohexane (1.34 g, 6.2 mmol) in DMSO (12 ml), was added Et₃N (0.6 ml). The obtained mixture was stirred at 40° C. for 48 hours yielding a white precipitate. After cooling to room temperature, water (15 ml) was added yielding a thick white precipitate which was filtered off and washed with water and ether. The obtained paste was dried in the vacuum oven. Yield: 0.99 g (1.65 mmol, 59%).

c) Synthesis of (1S,2S) CHex-bisurea dihexylamine (5a)

To a solution of TFA (8 ml) in CH₂Cl₂ (50 ml) was added (1S,2S) CHex-bisurea dihexyl BOC amine (0.4 g, 0.67 mmol). The mixture was stirred for two hours at room temperature, after which the solvents and excess TFA was evaporated in vacuo. A small amount of demi water (3 ml) and aqueous NaOH (2N; ˜50 ml) was added to the oily remains while stirring the mixture. A gel-like mixture was obtained which was filtered off on a suntered glass funnel (P 4) and washed with water. The obtained gel-like residue still contained a considerable amount of water, which was removed in the vacuum oven. Yield: 0.21 g (0.53 mmol, 79%).

Cyclohexane Bis-ureidohexylamine (Chemical Structure 5 in FIG. 51); Synthesis of 5b Representing the Racemic Compound

a) Synthesis of Phenyl-2-((phenoxycarbonyl)amino)cyclohexylcarbamate or trans-1,2-bis-phenoxycarbonylamino cyclohexane

To a cooled (T=0° C.) solution of phenyl chloroformate (15.1 g, 87.6 mmol) in CH₂Cl₂ (80 ml), was slowly added a solution of racemic trans-1,2-diaminocyclohexane (5.0 g, 43.8 mmol) and (iPr)₂EtN (15.2 ml, 87.6 mmol) in CH₂Cl₂ (100 ml). The suspension was stirred at room temperature for 18 hours and subsequently placed in the cooling cell for 2 hours. The obtained precipitate was filtered off and washed with cold CH₂Cl₂ and hexane. The white solid was dried in a vacuum oven. Yield 11.7 g (33.0 mmol, 75%).

b) Synthesis of racemic CHex-bisurea dihexyl BOCamine

To a solution of racemic phenyl-2-((phenoxycarbonyl)amino) cyclohexylcarbamate or trans-1,2-bis-phenoxycarbonylamino cyclohexane (1.0 g, 2.82 mmol) and N-BOC-1,6-diaminohexane (1.34 g, 6.2 mmol) in DMSO (12 ml), was added Et₃N (0.6 ml). The obtained mixture was stirred at 40° C. for 48 hours yielding a white precipitate. After cooling to room temperature, water (15 ml) was added yielding a thick white precipitate, which was filtered off and washed with water and ether. The obtained paste was dried in the vacuum oven, yield: 1.23 g (2.06 mmol, 73%).

c) Synthesis of racemic CHex-bisurea dihexylamine (5b)

To a solution of TFA (8 ml) in CH₂Cl₂ (50 ml) was added racemic CHex-bisurea dihexyl BOCamine (0.4 g, 0.67 mmol). The mixture was stirred for two hours at room temperature, after which the solvents and excess TFA was evaporated in vacuo. A small amount of demi water (3 ml) and aqueous NaOH (2N; 50 ml) was added to the oily remains while stirring the mixture. A gel-like mixture was obtained which was filtered off on a sintered glass funnel (P 4) and washed with water. The obtained gel-like residue still contained a considerable amount of water, which was removed in the vacuum oven. Yield: 0.23 g (0.58 mmol, 87%).

Synthesis of CHexAmMetAmCH2Pyr (6) (Chemical Structure in FIG. 52)

a) A solution of Boc-L-Met (3.0 g, 12.04 mmol), 4-aminomethyl pyridine (1.4 g, 13.24 mmol), and DMT-MM (3.7 g, 13.24 mmol) in methanol (50 mL) was stirred overnight at room temperature, after which the solvent was evaporated. The resulting mixture was dissolved in ethyl acetate and water (150 mL each) and brine (100 mL) was added to improve the separation of the layers. The ethyl acetate layer was washed with brine (2×150 mL), water (2×150 mL), and brine (150 mL), after which it was dried with Na₂SO₄ and evaporated to dryness. The resultant solid was purified by column chromatography (SiO₂, CH₂Cl₂:hexanes=1:1, going to CH₂Cl₂, going to CH₂Cl₂:methanol=97:3. Yield: 1.3 g (32%).

b) The product synthesized under a) (1.3 g, 3.84 mmol) was dissolved in CH₂Cl₂ (100 mL) to which trifluoroacetic acid (10 mL) and DMF (1 drop) were added. After stirring at room temperature for three hours, the solution was evaporated to dryness and the resultant blue oil was used for the next reaction without any further purification. Yield: 2.2 g.

c) To a cooled (0° C.) solution of the product synthesized under b) (2.2 g, 3.84 mmol) and Et₃N (3 mL, excess) in CH₂Cl₂ (100 mL), was added dropwise a solution of cis, cis-1,3,5-cyclohexanetriacid chloride (0.27 g, 1.0 mmol). The solution was stirred overnight while being allowed to come to room temperature. Meanwhile, an orange gel-like substance had formed, which was filtered off, washed with CH₂Cl₂, MeOH, H₂O/MeOH, and Et₂O (ca. 20 mL each) and subsequently dried. The resultant solid was dissolved in 1 N HCl and reprecipitated/regelled by addition of 2 N NaOH. Filtration of the precipitate/gel, followed by drying, gave the desired product. Yield: 0.2 g (23.7% based on the cis, cis-1,3,5-cyclohexanetriacid chloride).

Synthesis of CHexAmMetHisOMe (14) (Chemical Structure in FIG. 47) (MetHisOMe was Synthesized Following Standard Peptide Chemistry Protocols)

MetHisOMe (2.76 g; 9.23 mmol) containing a calculated amount of 14 mmol (1.6 g) TFA was dissolved in 100 ml CH₂Cl₂ and cooled to 0° C. Et₃N (2.8 ml=20 mmol) was added to neutralize the traces of TFA. Cis, cis-1,3,5-cyclohexane tricarbonyl trichloride (0.35 g; 1.30 mmol) was added, after which the temperature was slowly brought back to room temperature. After reacting for one night at room temperature, the formed precipitate was collected by filtration and dried in vacuo. Yield: 43.2%.

Synthesis of CHexAmMetHista (15) (Chemical Structure in FIG. 47)

A mixture of CHexAmMetOH (1) (1.0 g; 1.64 mmol), carbodiimidazole (0.82 g; 5.1 mmol) and Et₃N (0.82 ml; 5.9 mmol) was stirred at room temperature for one hour. Histamine dihydrochloride (0.94 g; 5.1 mmol) in 20 ml of DMSO was added dropwise. After reacting for one night at room temperature, an excess of H₂O was added and the formed precipitate was collected by filtration and dried in vacuo. Yield: 660 mg; 45%.

The gelation properties of 5a and 5b are given in Table H, and the gelation properties of 6 are given in Table I.

Compound 6 can gelate water:alcohol mixtures (1:1) in about 1.0-0.5 wt % and undergoes a sol-to-gel transition around pH=4.47 at room temperature (it is a solution below pH 4.47 and a gel above pH 4.47; 0.5 wt % in MeOH:H₂O=1:1). The gel-to-sol transition can also be achieved by raising the temperature. Compound 15 can gelate water and undergoes a sol-to-gel transition around pH=4.93 at room temperature (for a 0.5 wt % gel).

### Example 7-B3

### Chemical Substances that Cause a pH Change

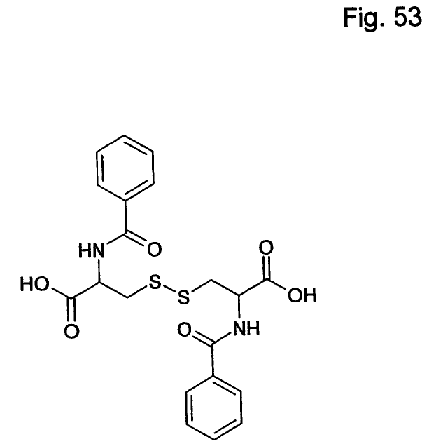

D-Glucose (42 mg, 0.23 mmol) was added to a mixture of DBC (chemical structure in FIG. 53) (42.4 mg, 0.095 mmol), NaHCO₃ (15.6 mg, 0.186 mmol), glucose oxidase (0.9 mg), catalase (4.0 mg) in H₂O (4 ml). The pH decreased owing to formation of D-gluconic acid. After three days, the reaction mixture was slightly turbid and completely gelified.

### Example 7-C

### Light as a Stimulus for Gel-to-Sol and Sol-to-Gel Transitions

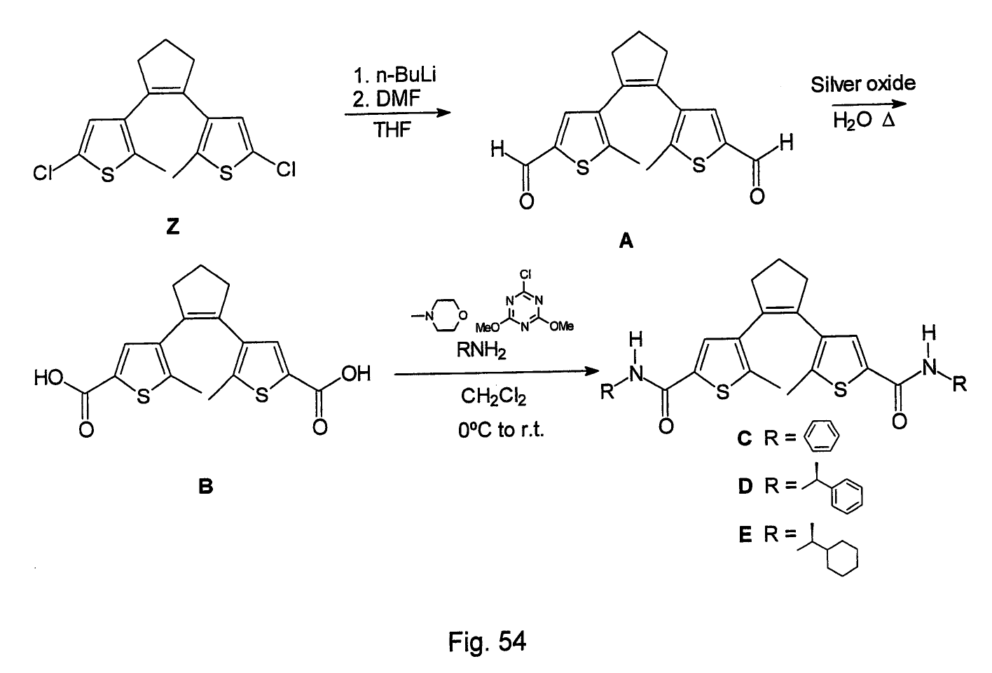

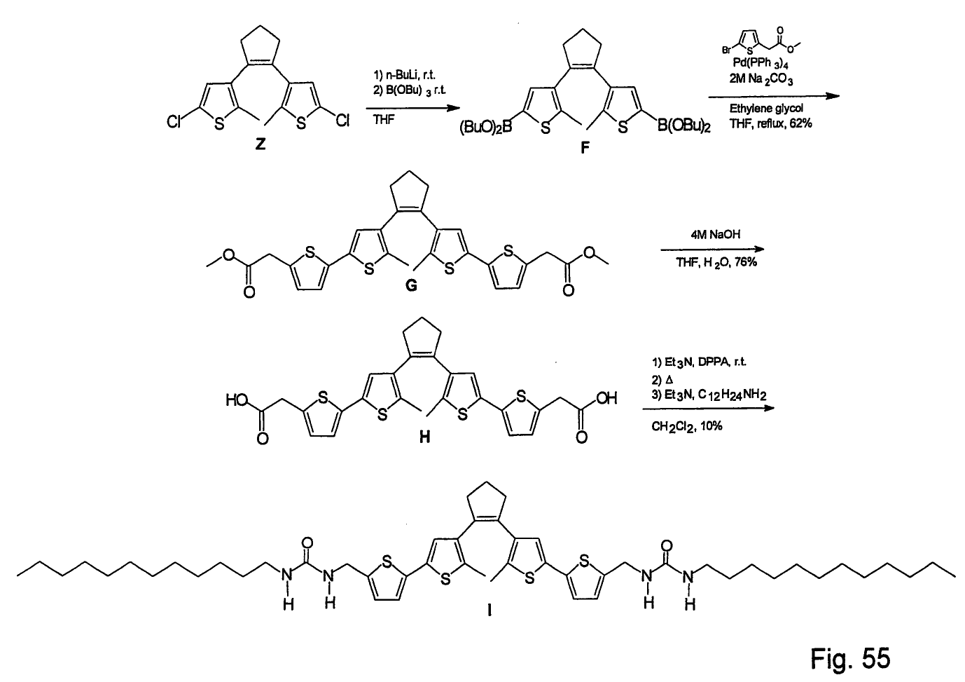

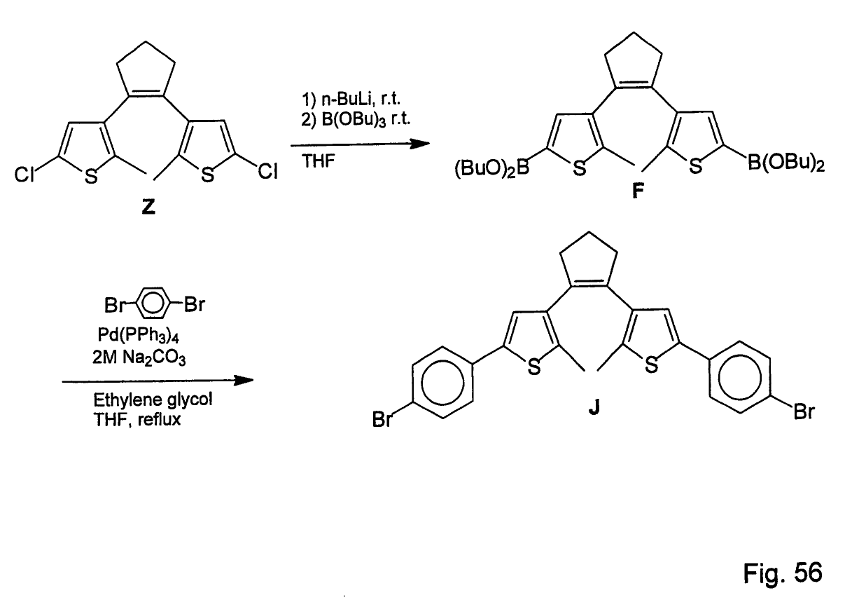

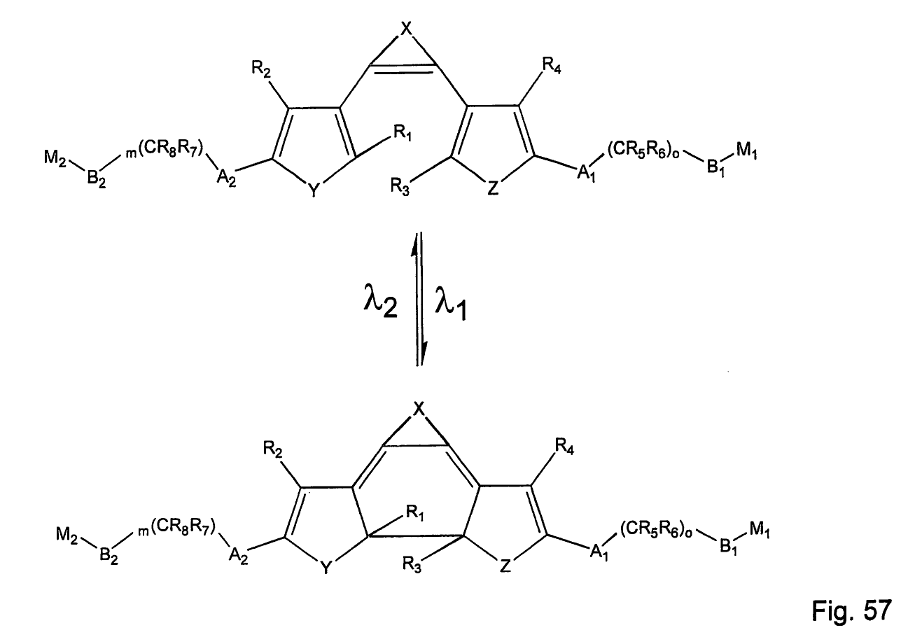

This Example describes suitable light-switchable gelators and a method of preparation thereof. The reaction schemes for the preparation of compounds A-E, F-I and J are given in FIGS. 54, 55 and 56, respectively. Photo-switching between the two valence isomers of the gelator is illustrated in FIG. 57.

1:1,2-Bis(5′-formyl-2′-methylthien-3′-l)cyclopentene (A)

n-Butyllithium (7.85 ml of 1.6 M solution in hexane, 12.56 mmol) was added to a stirred solution of 1,2-bis(5′-chloro-2′-methylthien-3′-l)cyclopentene (compound Z in FIGS. 54 to 56) (1.97 g, 5.98 mmol) in anhydrous THF (20 ml) under nitrogen at room temperature. One hour after the addition, the reaction mixture was quenched with anhydrous dimethylformamide (0.97 ml, 12.56 mmol). The mixture was stirred then for an additional hour at room temperature, before it was poured into HCl (2N, 50 ml). The mixture was extracted with diethyl ether (3×25 ml). The combined organic layers were washed with saturated sodium bicarbonate solution (2×25 ml) and H₂O (1×25 ml), and dried (Na₂SO₄), filtered and evaporated in vacuo to yield a brown solid (1.89 g, 90%). Chromatography of the solid over silica gel (hexane/ethyl acetate=9/1) afforded the compound as a brown/orange solid (0.98 g, 52%). In the first step, other lithiating agents can also be used. As a solvent, all ethers can be used but, preferably, THF and diethyl ether are used. The temperature at which the lithiation reaction can be performed ranges from −80° C. to 50° C., but preferably is 0° C.

1,2-bis(5′-carboxylic acid-2′-methylthien-3′-l)cyclopentene (B)

Silver oxide was used to oxidize dithienylcyclopentene bisaldehyde (compound A) that was prepared as described under compound A. This was done in situ by adding AgNO₃ (1.64 g, 9.6 mmol) to a solution of NaOH (0.75 g, 18.7 mmol) in H₂O (15 ml). Silver oxide immediately precipitated. This suspension was then added to compound A (0.74 g, 2.34 mmol) and refluxed for one hour, subsequently filtered over a glass filter and rinsed with hot water. The filtrate was cooled and acidified with 2M HCl in an ice bath. The compound precipitated and was filtered over a glass filter (G4). The residual water was azeotropically removed with toluene to yield an off-white solid (0.51 g, 62%).

1,2-Bis(5′-(anilinocarbonyl)-2′-methyl-thien-3′-yl) cyclopentene (C)

Dicarboxylic acid-thienylcyclopentene derivative (Compound B, 0.5 g, 1.44 mmol), which was prepared as described under compound B, was suspended in CH₂Cl₂ (5 ml) and placed in an ice bath. Subsequently, N-methylmorpholine (0.31 ml, 2.9 mmol) was added and the suspension became a solution. Then 2-chloro-4,6-dimethoxytriazine (0.48 g, 2.9 mmol) was added and a white precipitate was formed immediately after this addition. The reaction mixture was stirred for two hours at 0° C., then another two equivalents of N-methylmorpholine (0.31 ml, 2.9 mmol) were added followed by aniline (0.28 ml, 2.9 mmol). Stirring was continued for one hour at 0° C., and the reaction mixture was then stirred overnight at room temperature. CH₂Cl₂ (50 ml) was added and the solution was washed with, respectively, 1 M HCl (2×20 ml), brine (1×20 ml), saturated aqueous bicarbonate solution (1×20 ml) and H₂O (1×20 ml). The organic phase was dried (Na₂SO₄) and, after evaporation of the solvent, gave a solid product. After purification, refluxing in CH₂Cl₂/diethylether (excess), filtration (G4-glass filter), and drying under vacuum at 50° C., a white solid was obtained (0.27 g, 37%).

1,2-Bis(2′-methyl-5′-{(((R)-1-phenylethyl)amino)carbonyl}thien-3′-yl)cyclopentene (D)

This compound was prepared as described above for C, starting from diacid B (0.5 g, 1.44 mmol) and (R)-phenylethylamine (0.37 ml, 2.9 mmol). After purification by (CH₂Cl₂/MeOH=60:1), stirring in MeOH/ diethyl ether (excess) and filtration (G4-glass filter), a white solid was obtained (0.28 g, 35%) molecular formula C₃₃H₃₄N₂O₂S₂.

1,2-Bis(2′-methyl-5′-{(((R)-1-cyclohexylethyl)amino)carbonyl}thien-3′-yl)cyclopentene (E)

This compound was prepared as described above for C, starting from diacid B (1.34 g, 3.85 mmol) and (R)-cyclohexylamine (1.1 ml, 7.7 mmol). After purification by stirring in CH₂Cl₂/MeOH (60/1), filtration (G4-glass filter), and drying under vacuum at 50° C., a white solid was obtained (0.59 g, 44%). Molecular formula C₃₃H₄₆N₂O₂S₂.

1,2-Bis(5′-boronyl-2′-methylthien-3′-yl)cyclopentene (F)

Compound Z (1.0 g, 3.04 mmol) was dissolved in anhydrous THF (12 ml) under a nitrogen atmosphere, and n-BuLi (5.0 ml of 1.6 M solution in hexane, 8 mmol) was added at once using a syringe. This solution was stirred for 30 minutes at room temperature, and B(n-OBu)₃ (2.25 ml, 8.3 mmol) was added at once. The resulting solution was stirred for one hour at room temperature and was used directly in the Suzuki cross-coupling reaction.

1,2-Bis(5′-(methyl 5-(2-thienyl,)-acetic acid)-2′-methylthien-3′-yl)cyclopentene (G)

Methyl 2-(5-bromo-2-thienyl)acetic acid (2.94 g, 12.4 mmol) was dissolved in THF (75 ml) and Pd(PPh₃)₄ (0.42 g, 6 mol %) was added. This solution was stirred for 15 minutes at room temperature. Then, aqueous Na₂CO₃ (36 ml, 2 M) and six drops of ethylene glycol were added. This two-phase system was heated in an oil bath just below reflux (60° C.) and the solution of compound F (6.07 mmol) in THF (20 ml) was added dropwise via a syringe in a short time period. After that, the mixture was refluxed for two hours, subsequently cooled down, and diethylether (100 ml) and H₂O (50 ml) were added. The organic layer was washed with 1 M HCl (2×50 ml), brine (2×50 ml) and dried (Na₂SO₄). After evaporation, the compound was purified by column chromatography (CH₂Cl₂/hexane=4/1) on silica to yield an oil (2.13 g, 62%) (C₂₉H₂₈O₄S₄).

1,2-Bis(5′-(5-(2-thienyl)-acetic acid)-2′-methylthien-3′-yl)cyclopentene (H)

Compound G (0.81 g, 1.23 mmol) was dissolved in MeOH (20 ml) and THF (20 ml), then 4 M NaOH (2.7 ml) was added. This mixture was stirred for two hours at room temperature. After evaporation of the solvent, H₂O (25 ml) was added and the mixture was acidified in an ice bath with 2 M HCl. A precipitate was formed immediately, which was filtrated over a glass filter. After removal of the residual water by forming an azeotrope with toluene, an off-white solid was obtained (0.28 g, 76%) (C₂₇H₂₄O₄S₄).

1,2-Bis(5′-{N-dodecyl-N′-(4-(2-thienyl)methyl)urea}-2′-methylthien-3′-yl)cyclopentene (I)

Dry CH₂Cl₂ (20 ml) and Et₃N (0.11 ml, 0.76 mmol) were added under a nitrogen atmosphere to compound H (0.204 g, 0.38 mmol). When a solution was obtained, diphenylphosphorylazide (0.16 ml, 0.76 mmol) was added in one time. This mixture was stirred for two hours at room temperature, the temperature was subsequently raised to 50° C., and then stirred for another two hours. Then, dodecylamine (0.17 ml, 0.76 mmol) was added and the mixture was refluxed for two hours and finally stirred at room temperature for 16 hours. The mixture was diluted with diethylether (80 ml) and washed with saturated bicarbonate solution (2×25 ml), H₂O (2×25 ml), 1 M HCl (1×25 ml), brine (2×25 ml) and dried (Na₂SO₄). After evaporation of solvent in vacuo, the obtained solid was submitted to column chromatography (CHCl₃/MeOH=20/1) on silica and was refluxed in CHCl₃/diethylether (excess), and filtrated (G4) to yield a purple solid (36 mg, 10%) (C₅₁H₇₆N₄O₂S₄).

1,2-Bis(5′-(4″-bromophenyl)-2′-methylthien-3′-yl) cyclopentene (J)

1,4-dibromobenzene (3.4 g, 14.4 mmol) was dissolved in THF (12 ml) and after addition of Pd(PPh₃)₄ (0.4 g, 0.3 mmol), the solution was stirred for 15 minutes at room temperature. Then, aqueous Na₂CO₃ (17 ml, 2 M) and six drops of ethylene glycol were added, and the resulting two-phase system was heated in an oil bath until reflux (60° C.). The solution of compound F was added dropwise by a syringe in a few minutes. After addition was complete, the reaction mixture was refluxed for two hours, and then allowed to cool to room temperature. Diethylether (50 ml) and H₂O (50 ml) were added and the organic layer was collected and dried (Na₂SO₄). After evaporation of the solvent, the product was purified by column chromatography (SiO₂, hexane) and gave a yellowish solid (1.30 g, 76%) (C₂₇H₂₂Br₂S₂).

Results

A Gelation Experiment

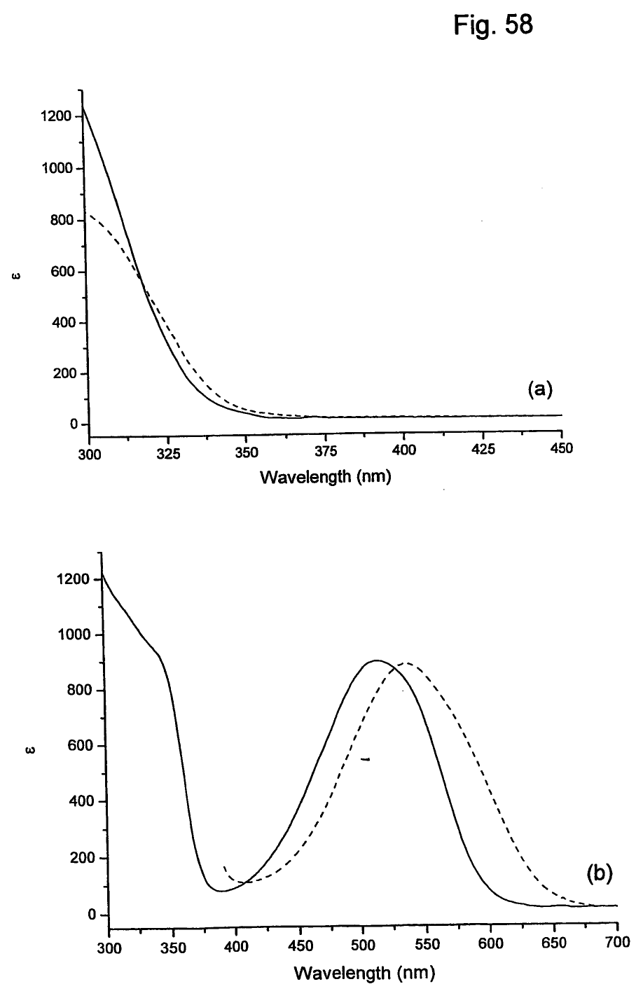

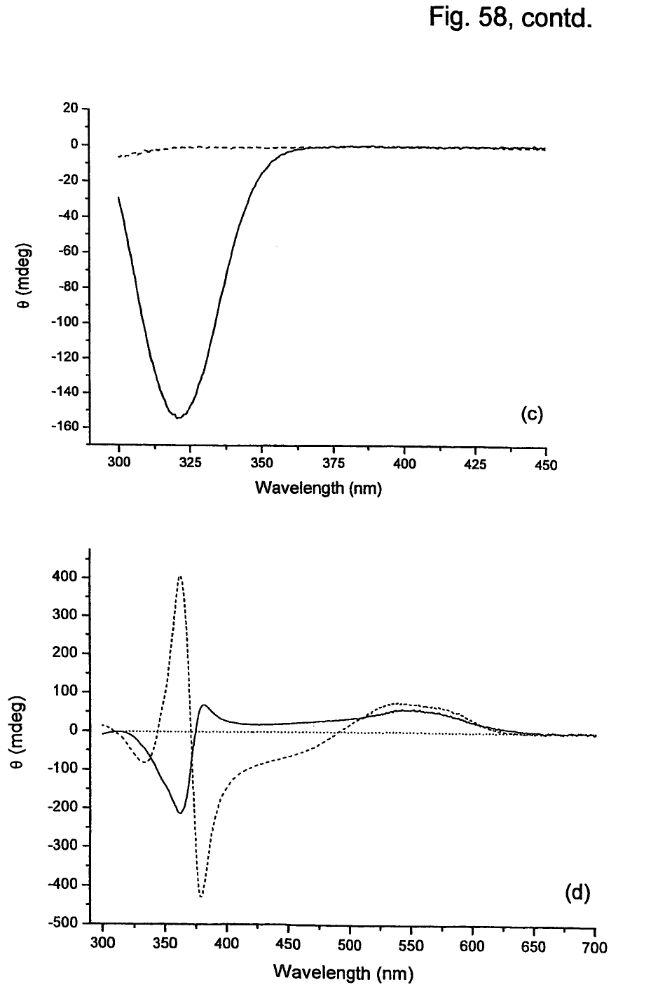

In a typical gelation experiment, a carefully weighed amount of the dithienylcyclopentene derivative under investigation and 1 ml of the solvent are placed in a test tube, which is sealed and then heated until the compound is dissolved. The solution is allowed to cool to room temperature. Gelation was considered to have occurred when a homogeneous solid substance was obtained that exhibited no gravitational flow. Gelation did occur in the following non-limiting cases of solutions of Compound C in 1-phenyloctane, toluene, n-butylether, benzene, tetralin; Compound D in cyclohexane, 1-phenyloctane, toluene, n-butylether, benzene, tetralin; Compound E in hexadecane, cyclohexane, 1-phenyloctane, toluene, n-butylether, benzene, tetralin, 1,4-dioxane, and n-butyl acetate. The gelation process could be followed by UV-vis and CD spectroscopy as depicted in FIG. 58. At low concentrations (0.35 mM), compound D is dissolved in toluene. This solution did not show a CD effect. Increasing the concentration to 1.8 mM causes gelation of the solvent, which is accompanied by a change of the UV-visible spectrum, together with the appearance of a strong CD signal at 323 nm (FIG. 58, panels (a) and (c)). Similar changes in the UV-vis and CD spectrum are observed upon gelation of the 1.8 mM solution of D in toluene by cooling to a temperature below the sol-gel phase transition (from here on, referred to as gel(o)). From a plot of the increase of the CD signal versus the temperature, the sol-gel phase transition temperature of gel(o) was determined to be 29° C. for a 1.8 mM solution of D in toluene.

Photo-Switching of a Gel of Compound D

In a typical photo-switching experiment, a solution (1.8 mM) of Compound D in toluene was prepared by cooling from 70° C. to the desired temperature (typically between 30° C. and 60° C., i.e., above the gel-sol phase transition temperature of the open form of D) in a cuvette of 1 mm path length. This solution was then irradiated at λ₁=313 nm to convert the ring-open form of Compound D to the ring-closed form. By using a 150 W Xe lamp and a 313 nm bandpass filter, the photo-stationary state (PSS) was reached, typically within 10 minutes, which consist of 60 mol % of the closed form and 40 mol % of the open form. During this irradiation process, the solution of D was transformed into a deeply purple colored gel (from here on, referred to as gel(c,I), which could be monitored by UV-vis and CD spectroscopy (FIG. 58, panels (b) and (d)). From a plot of the increase of the CD signal versus the temperature, the sol-gel phase transition temperature of gel(c,I) was determined to be 62° C., showing that the thermal stability has increased by more than 30° C., compared to that of gel(o). It should be noted that dissolving gel(c,I) by heating and subsequent cooling to again a temperature below the gel-sol phase transition, resulted in a CD spectrum different to that of gel(c,I), indicating that gels of the closed form of D can adopt an alternate structure, which is, from here on, referred to as gel(c,II) (see FIG. 58, panel (d)).

Gels of the closed form of D can also be dissolved by converting the closed form to the open form by irradiation with light of wavelength λ₂. Irradiation of gel(c,I) or gel(c,II) with λ₂>450 nm, while keeping the temperature between 30° C. and 60° C., causes dissolution of the gel to give a solution of the open form D, of which the UV-vis and CD spectra are identical to that of the starting solution. This process of gelation-dissolution by alternating irradiations with UV (313 nm) and Vis (>450 nm) light can be repeated many times, showing that the photo-induced gelation process is fully reversible.

In another experiment, a solution (1.8 mM) of Compound D in toluene was prepared by cooling from 70° C. to a temperature below the gel-sol phase transition temperature of the open form (typically between 20° C. and 29° C.) in a cuvette of 1 mm path length. This solution was then irradiated at λ₁=313 nm to convert the ring-open form of Compound D to the ring-closed form, whereas solutions which have been kept in the dark stay colorless and did not form a gel within 1 hour. This clearly shows that gelation of a solution of D can be accelerated by converting the open to the closed form by irradiation with light of wavelength λ₁.

### Example 7-D

### Chemical Substances as a Stimulus for Gel-to-Sol and Sol-to-Gel Transitions

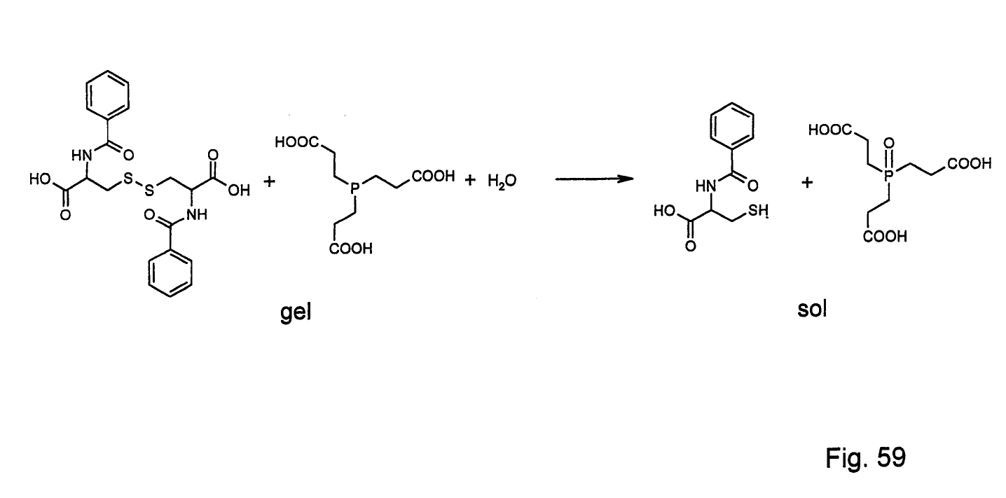

The gel-to-sol transition for a 1 wt % DBC gel (2.2×10⁻⁵ mol; chemical structure of DBC in FIG. 53) was achieved by reducing the disulfide bond of the gelator to a thiol by addition of a 1 mL aqueous solution of tris-(2-carboxy ethyl)phosphine (8.8×10⁻⁵ mol) (FIG. 59). ¹H NMR shows the disappearance of the —CH₂—S—S— signal and the resulting spectrum corresponds to that of benzoyl cysteine, and ³¹P NMR shows the gradual disappearance of the P peak of the phosphine and the appearance of the phosphine oxide peak. Similar results were obtained by using mercaptoethanol, 1,4-dithiothreitol or glutathione instead of tris-(2-carboxy ethyl)phosphine.

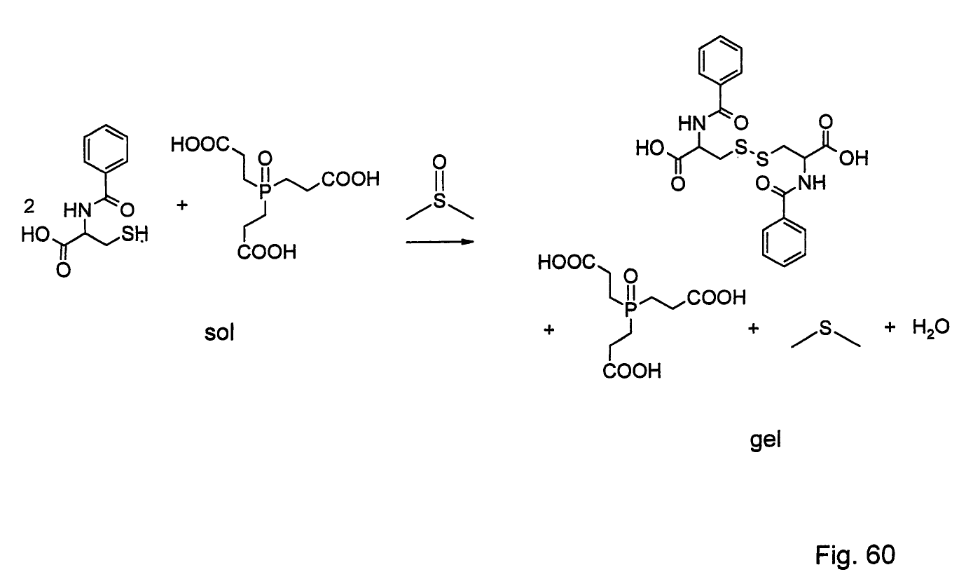

Subsequently, the sol-to-gel transition for this system (FIG. 60), was induced by addition of DMSO (5 μL) to the reaction mixture. DMSO is known to oxidize thiols to disulfides, therefore, the benzoyl cysteine molecules were oxidized to dibenzoyl cystine (DBC) and the formation of a gel was observed. This reaction was also confirmed by ¹H NMR.

### Example 7-E

### Non-Covalent Entrapment in Gels

### Example 7-E1

### Entrapment of Small Molecules/(Model) Drugs

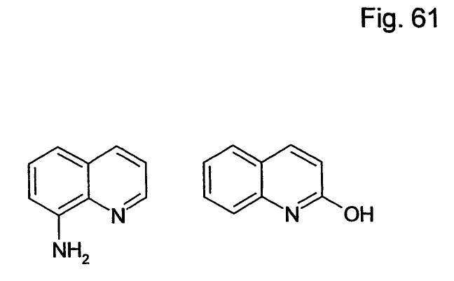

Entrapment of 8-aminoquinoline and 2-hydroxyquinoline (FIG. 61) was achieved by adding a PBS (phosphate-buffered saline) pH 7.4 solution of the quinoline of interest to X mg of DBC (dibenzoyl cystine); X=2, 3.5, 5 or 10, depending on the weight percentage (wt %) of gelator desired. The sample was then heated in a closed vial until complete dissolution of DBC was observed and subsequently allowed to cool to room temperature where gelation of the sample and, thus, entrapment of the quinolines in the gel occured. Entrapment of other small molecules such as cisplatin, (NH₃)₂Pt(Cl)₂, was achieved in a similar manner.

After 24 hours, percentages of 8-aminoquinoline and 2-hydroxyquinoline released from DBC gels of different weight percentages into 1mL of PBS solution at pH 7.4 in contact with the gel, were measured by UV-Vis spectroscopy and are reported in Table J. The initial quinoline concentration in the gel was 0.001M. The error on each percentage is ±4%.

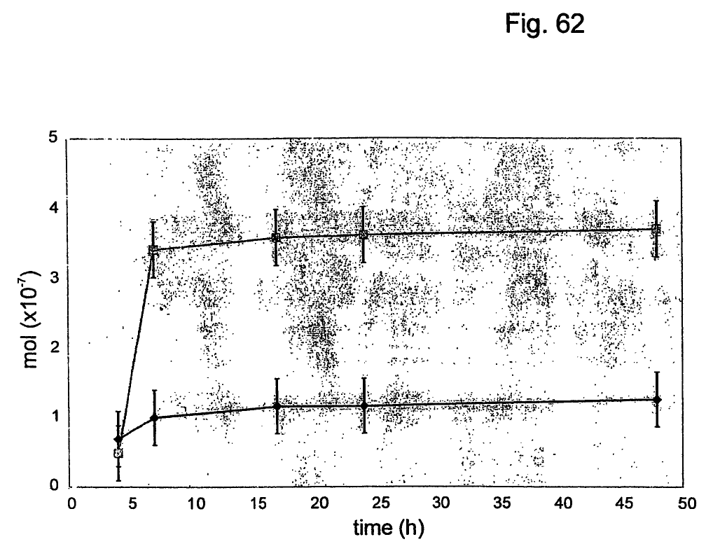

Time-dependent release of 8-aminoquinoline and 2-hydroxyquinoline from 0.2 wt % DBC gels is reported in FIG. 62. The initial quinoline concentration in the gel was 0.001M.

Heating the gels (from 33 to 100° C., depending on the wt % of the gel) and, thus, causing the transition from gel to solution (sol) to occur, resulted in triggered release of the 8-aminoquinoline and 2-hydroxyquinoline from these gels. Moreover, the gel-to-sol transition and, thus, the triggered release of 8-aminoquinoline and 2-hydroxyquinoline was also achieved by increasing the pH of the gels (for DBC, this is approximately 3.8) to above 4 by addition of base, e.g., NaOH 1 M.

### Example 7-E2

### Entrapment of (Polymeric) Micelles

### Entrapment via a pH Change

Gelator molecules 1 and 4 (about 5 mg; chemical structures in FIG. 47) were dissolved in a 0.1 M NaHCO₃ solution (0.25 ml). Surfactant solutions (SDS 9.21 and 27.22 mmol/l, CTAB 0.97 and 4.29 mmol/l, NC₂nC₁₀ lactose 2.91 and 14.15 mmol/l, 0.5 ml) were added, followed by addition of 0.1 M HCl (0.25 ml).

### Entrapment via a Temperature Change

Gelator molecules 2 and 3 (about 5 mg; chemical structures in FIG. 47) were dissolved in surfactant solutions (SDS 4.61 and 13.61 mmol/l, CTAB 0.49 and 1.46 mmol/l, NC₂nC₁₀ lactose 1.46 and 7.08 mmol/l, 1.5 ml) at T=150° C. and subsequently cooled to room temperature.

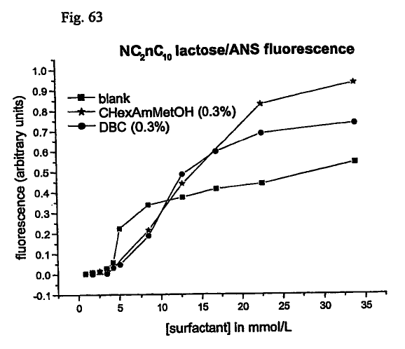

The gelation properties of 1, 2, 4 and DBC in the presence of surfactants and in the presence of polymeric micelles are shown in Table K and Table L, respectively. FIG. 63 shows that micelles are still intact within the gel matrix and that for NC₂nC₁₀ lactose (non-ionic surfactant), the CMC within the gel is identical to the CMC of the surfactant.

### Example 7-E3

### Entrapment of Liposomes

Entrapment of DOPC:chol=80:20 (molar ratio), 200-300 nm liposomes containing calcein in a DBC gel was achieved by adding 1mL of PBS pH 7.4 solution containing 2 mM EDTA, 2 mg DBC and 2.5 μL of the vesicle solution to 90 μL of HCl 1M. Gelation of the whole sample occured immediately and entrapment of the vesicles within the gel was confirmed by fluorescence spectroscopy, which also showed that the vesicles had remained intact since the amount of calcein leakage from the liposomes in the gel was minimal and comparable to that from liposomes in solution. The triggered release of the liposomes from the gel was achieved by increasing the pH of the gels by addition of base, e.g., NaOH 1 M.

### Example 7-F

### Covalent Entrapment in Gels; Use of Enzymatically Cleavable Linkers

Synthesis of Prodrug-Gelling Agent Conjugate

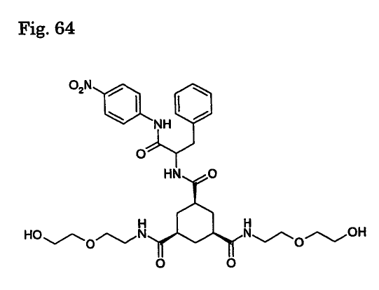

CHex(AmEtOEtOH)₂(AmPhe-pNA) (16) (Chemical Structure in FIG. 64)

a) A solution of cis, cis-1,3,5-cyclohexanetricarboxylic acid (5.40 g, 25 mmol), phenylalanine-p-nitroaniline (1.45 g, 5.0 mmol), and DMT-MM (1.40 g, 5.0 mmol) in methanol (150 mL) was stirred overnight at room temperature, after which the solvent was evaporated and the resultant residue was dissolved in ethyl acetate (200 mL). The solution was washed with 1 N HCl (3×100 mL), brine (100 mL), dried with MgSO₄ and subsequently evaporated to dryness. Precipitation from ethyl acetate/hexanes/methanol gave 0.16 g of what proved to be the disubstituted product. The remaining solution was evaporated to dryness and purified by column chromatography (SiO₂, CH₂Cl₂ going to CH₂Cl₂:MeOH=90:10). Yield: 1.22 g (50.0% based phenylalanine-p-nitroaniline).

b) A solution of the diacid (synthesized under a) (0.48 g, 1.0 mmol), 2-(2-aminoethoxy)-ethanol (0.42 g, 4.0 mmol), and DMT-MM (0.60 g, 2.2 mmol) in methanol (50 mL) was stirred overnight at room temperature, after which the solvent was evaporated to dryness. The crude product was purified by column chromatography (SiO₂, ethyl acetate:ethanol:water=50:30:5 going to 50:30:15). Yield: 0.25 g (38%).

CHex(AmEtOEtOH)₂(AmPhe-pNA) possesses a phenylalanine moiety that can be cleaved at the CO-terminus by a-chymotrypsin, resulting in the release of nitroaniline (a model drug). The prodrug has been mixed with several gelators (neutral: C3-Am-Meth-Am-CH₂CH₂OCH₂CH₂OH as well as ionizable: DBC and C3-Am-Meth-OH) and stabile gels were obtained (0.25 wt % of the prodrug and 0.5 wt % of the gelator). Comparison of the cleavage of this prodrug after layering both the (mixed) gel and solution containing the prodrug with a solution of α-chymotrypsin, showed a strong inhibition of the cleavage in the gel phase. This difference could be observed with the naked eye: after 20 minutes, the solution containing the prodrug turned bright yellow (due to the release of nitroaniline, whereas the gel containing the prodrug turned slightly yellow only after 20 hours.

### Example 7-G

### Encapsulation of Gels in Liposomes (Lipogelosomes)

Lipid mixtures, typically DOPC:cholesterol:DSPE-PEG (70:20:10), are prepared by co-dissolving lipids (Avanti Polar Lipids, Alabaster, Ala.), between 10 and 200 mg in total, in chloroform or ethanol and subsequently removing the solvent by evaporation under vacuum for at least four hours. The dried lipid film is then hydrated, typically in phosphate buffer at pH 7, and warmed to a temperature above the T_{gel} of the gelator to be encapsulated. A solution of the gelator at the same temperature is then added to the lipid solution and mixed for at least one hour at a temperature above the T_{gel} of the gelator.

Subsequently, extrusion of the liposome suspension at a temperature above the T_{gel} of the gelator is carried out to size the liposomes to the desired mean particle diameter. The liposome solution is then rapidly diluted, so as to bring the concentration of the gelator in solution below the critical gelation concentration, and cooled to room temperature to cause gelation of the gelator inside the liposomes.

TABLE A
AcmA-type homologs^{a}

| | | | | | |
|---|---|---|---|---|---|
| | | Acc. | Do- | | |
| Organism | Gene / protein | Number^{b} | MAIN | Sequence | Pi |

| | | | | | | |
|---|---|---|---|---|---|---|
| Agrobacterium | Atu1700 / lipoprotein | AAL42700 | 1 | VTLRPGESIATISNRYGVPEKELLRVNGLK | (SEQ ID NO:22) | 9.69 |
| tumefaciens | | | | TASSAQAGQSILIP | | |
| Agrobacterium | Atu1700 / lipoprotein | AAL42700 | 2 | YKVQPGDSLAKIARANGVSVAALKAANGIS | (SEQ ID NO:23) | 9.99 |
| tumefaciens | | | | NESIRVGQTLAMP | | |
| Anopheles | ebiG8480 / ebiP8480 | EAA01249 | 1 | TYTVKDRDTLTSVAARFDTTPSELTQLNRL | (SEQ ID NO:24) | 6.58 |
| gambiae | | | | ASSFIYSGQQLLVP | | |
| Anopheles | agCG47997 / agCP12171 | EAA01524 | 1 | RHDVERTDTLQGLALKYGCSMEQIRRVNRL | (SEQ ID NO:25) | 9.44 |
| gambiae | | | | LPTDTIFLRPFLMVP | | |
| Anopheles | ENSANGG00000012745 / | EAA10345 | 1 | EAQILPGDTLQAIALRFNCSVGFLVSTKIP | (SEQ ID NO:26) | 9.96 |
| gambiae | ENSANGP00000015234 | | | QLKKLNKIDKDNEIYARNVIRVP | | |
| Aquifex | nlpD1 / lipoprotein | AAC06844 | 1 | YKVKKGDSLWKIAKEYKTSIGKLLELNPKL | (SEQ ID NO:27) | 10.06 |
| aeolicus VF5 | | | | KNRKYLRPGEKICLK | | |
| Aquifex | nlpD1 / lipoprotein | AAC06844 | 2 | YRVKRGDSLIKIAKKFGVSVKETKRVNKLK | (SEQ ID NO:28) | 10.91 |
| aeolicus VF5 | | | | GNRIYVGQKLKIP | | |
| Aquifex | nlpDl / lipoprotein | AAC06844 | 3 | YRVRRGDTLIKIAKRFRTSVKEIKRINRLK | (SEQ ID NO:29) | 12.02 |
| aeolicus VF5 | GNLIRVGQKLKIP | | | | | |
| Arabidopsis | At5g08200 / | AAM98176 | 1 | HRISKFDTLAGVAIKYGVEVADVKKMNNLV | (SEQ ID NO:30) | 9.31 |
| thaliana | hypothetical protein | | | TDLQMFALKSLQIP | | |
| Arabidopsis | At5g23130 / | AAM44993 | 1 | EHRVSKFDTLAGIAIKYGVEVADITKLNGL | (SEQ ID NO:31) | 5.68 |
| thaliana | hypothetical protein | | | VTDLQMFALESLRIP | | |
| Arabidopsis | AT2G23770 / putative | AAC17086 | 1 | TYTIQPNDSYFAIANDTLQGLSTCQALAKQ | (SEQ ID NO:32) | 6.26 |
| thaliana | protein kinase | | | NNVSSQSLFPGMRIVVP | | |
| Arabidopsis | AT2G23770 / putative | AAC17086 | 2 | SYTVVFEDTIAIISDRFGVETSKTLKANEM | (SEQ ID NO:33) | 3.99 |
| thaliana | protein kinase | | | SFENSEVFPFTTILIP | | |
| Arabidopsis | AT2G17120 / predicted | AAL07070 | 1 | EYTIKKDDILSFVATEIFGGLVTYEKISEV | (SEQ ID NO:34) | 4.53 |
| thaliana | GPI-anchored protein | | | NKIPDPNKIEIGQKFWIP | | |
| Arabidopsis | AT2G17120 / predicted | AAL07070 | 2 | AHVVKLGSSLGEIAAQFGTDNTTLAQLNGI | (SEQ ID NO:35) | 4.19 |
| thaliana | GPI-anchored protein | | | IGDSQLLADKPLDVP | | |
| Arabidopsis | At1g21880 / unknown | AAF16531 | 1 | HYKTRPSDNLGSIADSVYGGLVSAEQIQEA | (SEQ ID NO:36) | 4.04 |
| thaliana | protein | | | NSVNDPSLLDVGTSLVIP | | |
| Arabidopsis | At1q21880 / unknown | AAF1G531 | 2 | SYVVKEIDTLVGIARRYSTTITDLMNVNAM | (SEQ ID NO:37) | 4.19 |
| thaliana | protein | | | GAPDVSSGDILAVP | | |
| Arabidopsis | At1g77630 / predicted | AAG51658 | 1 | HYKTRTSDTLGSIADSVYGGLVSPEQIQVA | (SEQ ID NO:38) | 4.32 |
| thaliana | GPI-anchored protein | | | NSETDLSVLDVGTKLVIP | | |
| Arabidopsis | At1g77630 / predicted | AAG51658 | 2 | SYVVRGIDTMAGIAKRFSTSVTDLTNVNAM | (SEQ ID NO:39) | 4.45 |
| thaliana | GPI-anchored protein | | | GAPDINPGDILAVP | | |
| Arabidopsis | At1g55000 / unknown | AAG00879 | 1 | SHRICRGDSVTSLAVKYAVQVMDIKRLNNM | (SEQ ID NO:40) | 9.94 |
| thaliana | protein | | | MSDHGIYSRDRLLIP | | |
| Arabidopsis | At1g51940 / putative | AAF99862 | 1 | SYVAMAGDSVQSLSSRFGVSMDRIEDVNGI | (SEQ ID NO:41) | 3.75 |
| thaliana | protein kinase | | | LNLDNITAGDLLYIP | | |
| Bacillus cereus | cwh / putative cell | CAB69802 | 1 | HTVKKNDTLWGISKQYGVSIQSIKQANNKG | (SEQ ID NO:42) | 9.60 |
| | wall hydrolase | | | NDKTEIGEQL | | |
| Bacillus cereus | cwh / putative cell | CAB69802 | 2 | YQVQPGDSLETIAKRYNVTVQSIKQMNNTV | (SEQ ID NO:43) | 9.31 |
| | wall hydrolase | | | GNKLYTGQHL | | |
| Bacillus cereus | sleL / cortical | BAA92376 | 1 | VTVRSGDSVYSLASKYGSTPDEIVKDNGLN | (SEQ ID NO:44) | 4.44 |
| | fragment-lytic enzyme | | | PAEtLVVGQALIVN | | |
| Bacillus cereus | sleL / cortical | BAA92376 | 2 | YYVQPGDSLYRISQTYNVPLASLAKVNNLS | (SEQ ID NO:45) | 9.23 |
| | fragment-lytic enzyme | | | LKSiLHVGQQLYVP | | |
| Bacillus | BH1600 / unknown | BAB05319 | 1 | FTVSKGDTLWGIAQKHEMSVETLMEINGLN | (SEQ ID NO:46) | 4.37 |
| halodurans | conserved protein | | | DTLIHPGDDLIV | | |
| Bacillus | BH1600 / unknown | BAB05319 | 2 | HVVKTGDTLYRIALNHQLTVEELQANNDLT | (SEQ ID NO:47) | 4.57 |
| halodurans | conserved protein | | | DELIFPGQVLAL | | |
| Bacillus | BH2879 / spore | BAB06598 | 1 | HQVQSGDTLYLLSEQYGVPMEAIKRINERS | (SEQ ID NO:48) | 5.59 |
| halodurans | cortex-lytic enzyme | | | SNTIYRGEQLTI | | |
| Bacillus | BH1221 / BH1221 | BAB04940 | 1 | HIVQKGDTLWKLAKKYGVDFEQLKAANSQL | (SEQ ID NO:49) | 9.53 |
| halodurans | unknown conserved | | | ANPDMIMPGMKIKIP | | |
| | protein | | | | | |
| Bacillus | BH0693 / BH0693 | BAB04412 | 1 | YTVQPGDTLSAIAARFGSTVLEIQRAN1QD | (SEQ ID NO:50) | 4.78 |
| halodurans | unknown conserved | | | PRFIEPNVIFPGWTLVIP | | |
| | protein | | | | | |
| Bacillus | BH0693 / BH0693 | BAB04412 | 2 | YLPVPGDTLFRISQRFSAHFDLIAGVNRLQ | (SEQ ID NO:51) | 6.75 |
| halodurans | unknown conserved | | | DPN1IFVGQLLWVP | | |
| | protein | | | | | |
| Bacillus | BH0693 / BH0693 | BAB04412 | 3 | YEIEVGDTLEGISRRFQIPITKILAANEGR | (SEQ ID NO:52) | 6.28 |
| halodurans | unknown conserved | | | PGFSLDFIFVGFRLLLP | | |
| | protein | | | | | |
| Bacillus | BH1803 / BH1803 | BAB05522 | 1 | HTVVSGDTMWKIAARYQVGVSEIIQANPQV | (SEQ ID NO:53) | 8.44 |
| halodurans | unknown conserved | | | SNPNVIYPGQKL | | |
| | protein | | | | | |
| Bacillus phage | orf 15 / Lysozyme | AAA32288 | 1 | YKVKSGDNLTKIAKKHNTTVATLLKLNPSI | (SEQ ID NO:54) | 10.22 |
| PZA | | | | KDPNMIRVGQTINVT | | |
| Bacillus phage | orf 15 / Lysozyme | AAA32288 | 2 | HKVKSGDTLSKIAVDNKTTVSRLMSLNPEI | (SEQ ID NO:55) | 10.17 |
| PZA | | | | TNPNHIKVGQTIRLS | | |
| Bacillus | X69507 / | CAA49259 | 1 | ILIRPGDSLWYFSDLFKIPLQLLLDSNRNI | (SEQ ID NO:56) | 8.59 |
| sphaericus | endopeptidase I | | | NPQLLQVGQRIQIP | | |
| Bacillus | X69507 / | CAA49259 | 2 | YTITQGDSLWQIAQNKNLPLNAILLVNPEI | (SEQ ID NO:57) | 6.75 |
| sphaericus | endopeptidase I | | | QPSRLHIGQTIQVP | | |
| Bacillus | lytE / LytE | AAC25975 | 1 | IKVKKGDTLWDLSRKYDTTISKIKSENHLR | (SEQ ID NO:58) | 9.52 |
| subtilis | | | | SDIIYVGQTLSIN | | |
| Bacillus | lytE / LytE | AAC25975 | 2 | YKVKSGDSLWKISKKYGMTINELKKLNGLK | (SEQ ID NO:59) | 10.14 |
| subtilis | | | | SDLLRVGQVLKLK | | |
| Bacillus | lytE / LytE | AAC25975 | 3 | YKVKSGDSLSKIASKYGTTVSKLKSLNGLK | (SEQ ID NO:60) | 10.00 |
| subtilis | | | | SDVIYVNQVLKVK | | |
| Bacillus | spoVID / SpoVID | AAA22808 | 1 | CIVQQEDTIERLCERYEITSQQLIRMNSL | (SEQ ID NO:61) | 4.33 |
| subtilis | | | | ALDDELKAGQILYIP | | |
| Bacillus | yaaH / hypothetical | BAA05252 | 1 | MVKQGDTLSAIASQYRTTTNDITETNEIP | (SEQ ID NO:62) | 4.10 |
| subtilis | protein | | | NPDSLVVGQTIVIP | | |
| Bacillus | yaaH / hypothetical | BAA05252 | 2 | YDVKRGDTLTSIARQFNTTAAELARVNRI | (SEQ ID NO:63) | 10.25 |
| subtilis | protein | | | QLNTVLQIGFRLYIP | | |
| Bacillus | yojL / hypothetical | AAC17860 | 1 | IKVKSGDSLWKLSRQYDTTISALKSENKL | (SEQ ID NO:64) | 9.90 |
| subtilis | protein | | | KSTVLYVGQSLKVP | | |
| Bacillus | yojL / hypothetical | AAC17860 | 2 | YTVAYGDSLWMIAKNHKMSVSELKSLNSL | (SEQ ID NO:65) | 9.78 |
| subtilis | protein | | | SSDLIRPGQKLKIK | | |
| Bacillus | yojL / hypothetical | AAC17860 | 3 | YTVKLGDSLWKIANSLNMTVAELKTLNGL | (SEQ ID NO:66) | 9.23 |
| subtilis | protein | | | TSDTLYPKQVLKIG | | |
| Bacillus | yojL / hypothetical | AAC17860 | 4 | YKVKAGDSLWKIANRLGVTVQSIRDKNNL | (SEQ ID NO:67) | 9.82 |
| subtilis | protein | | | SSDVLQIGQVLTIS | | |
| Bacillus | yocH / hypothetical | AAB84474 | 1 | ITVQKGDTLWGISQKNGVNLKDLKEWNKL | (SEQ ID NO:68) | 9.23 |
| subtilis | protein | | | TSDKIIAGEKLTIS | | |
| Bacillus | yocH / hypothetical | AAB84474 | 2 | YTIKAGDTLSKIAQKFGTTVNNLKVWNNL | (SEQ ID NO:69) | 9.60 |
| subtilis | protein | | | SSDMIYAGSTLSVK | | |
| Bacillus | ykvP / hypothetical | CAB13251 | 1 | HHVTPGETLSIIASKYNVSLQQLMELNHF | (SEQ ID NO:70) | 8.40 |
| subtilis | protein | | | KSDQIYAGQIIKIR | | |
| Bacillus | xlyB / amidase | AAB87514 | 1 | YHVKKGDTLSGIAASHGASVKTLQSINHI | (SEQ ID NO:71) | 9.70 |
| subtilis | | | | TDPNHIKIGQVIKLP | | |
| Bacillus | yrbA / hypothetical | CAB75322 | 1 | HIVQKGDSLWKIAEKYGVDVEEVKKLNTQ | (SEQ ID NO:72) | 8.28 |
| subtilis | protein | | | LSNPDLIMPGMKIKVP | | |
| Bacillus | ydhD / hypothetical | BAA19695 | 1 | HIVGPGDSLFSIGRRYGASVDQIRGVNGL | (SEQ ID NO:73) | 5.43 |
| subtilis | protein | | | DETNIVPGQALLIP | | |
| Bacillus | ykuD / hypothetical | CAA10867 | 1 | YQVKQGDTLNSIAADFRISTAALLQANPS | (SEQ ID NO:74) | 5.96 |
| subtilis | protein | | | LQAGLTAGQSIVIP | | |
| Bacillus | xlyA / amidase | AAA22645 | 1 | YVVKQGDTLTSIARAFGVTVAQLQEWNNI | (SEQ ID NO:75) | 4.78 |
| subtilis | | | | EDPN1IRVGQVLIVS | | |
| Bacillus | yhdD / hypothetical | CAA74437 | 1 | IKVKSGDSLWKLAQTYNTSVAALTSANHL | (SEQ ID NO:76) | 9.53 |
| subtilis | protein | | | STTVLSIGQTLTIP | | |
| Bacillus | YhdD / hypothetical | CAA74437 | 2 | YTVKSGDSLWLIANEFKMTVQELKKLNGL | (SEQ ID NO:77) | 9.60 |
| subtilis | protein | | | SSDLIRAGQKLKVS | | |
| Bacillus | yhdD / hypothetical | CAA74437 | 3 | YKVQLGDSLWKIANKVNMSIAELKVLNNL | (SEQ ID NO:78) | 9.70 |
| subtilis | protein | | | KSDTIYVNQVLKTK | | |
| Bacillus | yhdD / hypothetical | CAA74437 | 4 | YTVKSGDSLWKIANNYNLTVQQIRNINNL | (SEQ ID NO:79) | 9.60 |
| subtilis | protein | | | KSDVLYVGQVLKLT | | |
| Bacillus | yhdD / hypothetical | CAA74437 | 5 | YTVKSGDSLWVIAQKFNVTAQQIREKNNL | (SEQ ID NO:80) | 9.70 |
| subtilis | protein | | | KTDVLQVGQKLVIS | | |
| Bacillus | yqbp / hypothetical | BAA12412 | 1 | YTVKKGDTLWDLAGKEYGDSTKWPKIWKV | (SEQ ID NO:81) | 10.73 |
| subtilis | protein | | | NKKAMIKRSKRNIRQPGHWLFPGQKLKIP | | |
| Bacillus | xkdp / homologous to | CAA94038 | 1 | YTVKKGDTLWDIAGRFYGNSTQWRKIWNA | (SEQ ID NO:82) | 11.17 |
| subtilis | yqbP of the skin | | | NKTAMIKRSKRNIRQPGHWLFPGQKLKIP | | |
| | element | | | | | |
| Bacillus | ypbE / hypothetical | AAC83949 | 1 | HTVQKKETLYRISNKYYKSRTGEEKIRAY | (SEQ ID NO:83) | 9.40 |
| subtilis | protein | | | NHLNGNDVYTGQVLDIP | | |
| Bacillus | yneA / hypothetical | CAA97614 | 1 | IEVQQGDTLWSIADQVADTKKINKNDFIE | (SEQ ID NO:84) | 4.03 |
| subtilis | protein | | | WVADKNQLQTSDIQPGDELVIP | | |
| Bacillus | phi-105 / ORF46 | BAA36652 | 1 | YTVKKGDTLSEIAVKTGVSMAKLQAYNCI | (SEQ ID NO:85) | 9.90 |
| subtilis phage | | | | KNANKITVGQVLKLT | | |
| 105 | | | | | | |
| Bacillus | orfl5 / morphogenesis | CAA67646 | 1 | HVVKKGDTLSEIAKKIKTSTKTLLELNPT | (SEQ ID NO:86) | 10.10 |
| subtilis phage | protein | | | IKNPNKIYVGQRINVG | | |
| 103 | | | | | | |
| Bacillus | orfl5 / morphogenesis | CAA67646 | 2 | YRIKRGETLTGIAKKNKTTVSQLMKLNPN | (SEQ ID NO:87) | 10.58 |
| subtilis phage protein | | | IKNANNIYAGQTIRLK | | | |
| 103 | | | | | | |
| Bacillus | XLYA / amidase | AAA22645 | 1 | YVVKQGDTLTSIARAFGVTVAQLQEWNNI | (SEQ ID NO:88) | 4.78 |
| subtilis phage | | | | EDPNLIRVGQVLIVS | | |
| PBSX | | | | | | |
| Bartonella | ORF-401 / NlpD/LppB | AAF80360 | 1 | YIVQSGDTLFSIAQQKGISVESLKVANGM | (SEQ ID NO:89) | 6.12 |
| bacilliformis | homolog | | | GDNAIYICQKLVI | | |
| Borrelia | BB0262 / conserved | AAC66685 | 1 | HKIKPGETLSHVAARYQITSETLISFNEI | (SEQ ID NO:90) | 9.70 |
| Burgdorferi B31 | hypothetical protein | | | KDVRNIKPNSVIKVP | | |
| Borrelia | BB0262 / conserved | AAC66685 | 2 | YIVKKNDSISSIASAYNVPKVDILDSNNL | (SEQ ID NO:91) | 4.78 |
| Burgdorferi B31 | hypothetical protein | | | DNEVLFLGQKLFIP | | |
| Borrelia | BB0625 / putative | AAC66988 | 1 | YKVVKGDTLFSIAIKYKVKVSDLKRINKL | (SEQ ID NO:92) | 10.00 |
| Burgdorferi B31 | amidase | | | NVDNIKAGQILIIP | | |
| Borrelia | BB0625 / putative | AAC66988 | 2 | YTAKEGDTIESISKLVGLSQEEIIAWNDL | (SEQ ID NO:93) | 5.10 |
| Burgdorferi B31 | amidase | | | RSKDLKVGMKLVLT | | |
| Borrelia | BB0625 / putative | AAC66988 | 3 | YMVRKGDSLSKLSQDFDISSKDILKFNFL | (SEQ ID NO:94) | 9.16 |
| Burgdorferi B31 | amidase | | | NDDKLKIGQQLFLK | | |
| Borrelia | BB0625 / putative | AAC66988 | 4 | HYVKRGETLGRIAYIYGVTAKDLVALNGN | (SEQ ID NO:95) | 9.99 |
| Burgdorferi B31 | amidase | | | PAINLKAGSLLNVL | | |
| Borrelia | BB0625 / putative | AAC66988 | 5 | HSVAVGETLYSIARHYGVLIEDLKNWNNL | (SEQ ID NO:96) | 7.02 |
| Burgdorferi B31 | amidase | | | SSIINIMHDQKLKIF | | |
| Borrelia | BB0761 / conserved | AAC67117 | 1 | YKVKKGDTFFKIANKINGWQSGIATINLL | (SEQ ID NO:97) | 9.31 |
| Burgdorferi 531 | hypothetical protein | | | DSPAVSVGQEILIP | | |
| Caenorhabditis | TO1C4.1 / | AAB09177 | 1 | HTIKSGDTCWKIASEASISVQELEGLNSK | (SEQ ID NO:98) | 4.47 |
| elegans | hypothetical protein | | | KSCANLAVGLSEQEFTEMNEELDCNRLAV | | |
| | TO1C4.1 | | | GNEICVS | | |
| Caenorhabditis | TO1C4.1 / | AAB09177 | 2 | KIHVKEGDTCYTIWTSQHLTEKQFMDMNE | (SEQ ID NO:99) | 4.33 |
| elegans | hypothetical protein | | | ELNCGMLEIGNEVCVC | | |
| | TO1C4.1 | | | | | |
| Caenorhabditis | TO1C4.1 / | AAB09177 | 3 | YATVTPGSSCYTISASYGLNLAELQTTYN | (SEQ ID NO:100) | 3.37 |
| elegans | hypothetical protein | | | CDALQVDDTIGVS | | |
| | TO1C4.1 | | | | | |
| Caenorhabditis | TO1C4.1 / | AAB09177 | 4 | RIEILNGDTCGFLENAFQTNNTEMEIANEG | (SEQ ID NO:101) | 4.00 |
| elegans | hypothetical protein | | | VKCDNLPIGRMMCVW | | |
| | TO1C4.1 | | | | | |
| Caenorhabditis | TO1C4.1 / | AAB09177 | 5 | FQMVNQFQSCEDINQRSQISNRKLLELNPT | (SEQ ID NO:102) | 8.00 |
| elegans | hypothetical protein | | | FRCDEMAKYEQICLG | | |
| | TO1C4.1 | | | | | |
| Caenorhabditis | F52E1.13 / | AAL08036 | 1 | DYTITETDTLERVAASHDCTVGELMKLNKM | (SEQ ID NO:103) | 5.67 |
| elegans | hypothetical protein | | | ASRMVFPGQKILVP | | |
| | F52E1.13c | | | | | |
| Caenorhabditis | F43G9.2 / | CAE02105 | 1 | ERKVKNGDTLNKLAIKYQVNVAEIKRVNNM | (SEQ ID NO:104) | 10.00 |
| elegans | hypothetical protein | | | VSEQDFMALSKVKIP | | |
| | F43G9.2 | | | | | |
| Caenorhabditis | F07G11.9 / | AAG24054 | 1 | WTEIKSGDSCWNIASNAKISVERLQQLNEG | (SEQ ID NO:105) | 8.56 |
| elegans | hypothetical protein | | | MKCDKLPLGDKLCLA | | |
| | F07G11.9 | | | | | |
| Caenorhabditis | F07G11.9 / | AAG24054 | 2 | KLKLKAEDTCFKIWSSQKLSERQFLGHNEG | (SEQ ID NO:106) | 7.91 |
| elegans | hypothetical protein | | | MDCDKLKVGKEVCVA | | |
| | F07G11.9 | | | | | |
| Caenorhabditis | F07G11.9 / | AAG24054 | 3 | HKIQKGDTCFKIWTTNKISEKQFRNLNKGL | (SEQ ID NO:107) | 8.94 |
| elegans | hypothetical protein | | | ˜CDKLEIGKEVCIS | | |
| F07G11. 9 | | | | | | |
| Caenorhabditis | F07G11.9 / | AAG24054 | 4 | LKIKEGDTCYNIWTSQKISEQEFNELNKGL | (SEQ ID NO:108) | 4.65 |
| elegans | hypothetical protein | | | DCDKLEIGKEVCVT | | |
| F07G11. 9 | | | | | | |
| Caenorhabditis | F07G11.9 / | AAG24054 | 5 | YRFKKGDTCYKIWTSHKMSEKQFRALNRGI | (SEQ ID NO:109) | 9.46 |
| elegans | hypothetical protein | | | DCDRLVPGKELCVG | | |
| | F07G11.9 | | | | | |
| Caenorhabditis | F07G11.9 / | AAG24054 | 6 | ITVKPGDTCFSTWTSQKNTQQQFMDTNPEL | (SEQ ID NO:110) | 4.39 |
| elegans | hypothetical protein | | | DCDKLEIGKEVCVT | | |
| | F07G11.9 | | | | | |
| Caenorhabditis | F07G11.9 / | AAG24054 | 7 | VKINPGDTCFNIWTSQBNTQQQFMDLNKRL | (SEQ ID NO:111) | 6.22 |
| elegans | hypothetical protein | | | DCDKLEVGKEVCVA | | |
| | F07G11.9 | | | | | |
| Caenorhabditis | F07G11.9 / | AAG24054 | 8 | VQINPGDTCFKIWSAQKLTEQQFHELNKGL | (SEQ ID NO:112) | 4.72 |
| elegans | hypothetical protein | | | DCDRLEVGKEVCIA | | |
| | F07Gll.9 | | | | | |
| Caenorhabditis | F07G11.9 / | AAG24054 | 9 | TEVKEGDTCFKIWSAHKITEQQFMEMI4RG | (SEQ ID NO:113) | 5.16 |
| elegans | hypothetical protein | | | LDCNRLEVGKEVCIV | | |
| | F07G11.9 | | | | | |
| Caenorhabditis | F07G11.9 / | AAG24054 | 10 | IKVKEGDTCFKIWSAQKMTEQQEMEMNRGL | (SEQ ID NO:114) | 7.92 |
| elegans | hypothetical protein | | | DCNKLMVGKEVCVS | | |
| | F07G11.9 | | | | | |
| Caenorhabditis | F07G11.9 / | AAG24054 | 11 | ATITPGNTCFNISVAYGINLTDLQIZTYD | (SEQ ID NO:115) | 4.23 |
| elegans | hypothetiprotein | | | CKALEVGDTICVS | | |
| | F07G11. 9 | | | | | |
| Caenorhabditis | F07G11.9 / | AAG24054 | 12 | IEVIKGDTCWELENAFKTNQTEMERANEG | (SEQ ID NO:116) | 4.77 |
| elegans | hypothetical protein | | | VKCDNLPIGRNMCVW | | |
| | F07G11. 9 | | | | | |
| Caenorhabditis | F07G11.9 / | AAG24054 | 13 | IQNFNQYQSCNQVNQQNQISNRKLMDLNP | (SEQ ID NO:117) | 9.62 |
| elegans | hypothetical protein | | | TFRGDRLQKSEQVSQK | | |
| | F07G11.9 | | | | | |
| Caenorhabditis | B0041.3 / | AAC24252 | 1 | IYQVQTDDTLERIALKHNCSVSSLVRANK | (SEQ ID NO:118) | 10.12 |
| elegans | hypothetical protein | | | LWSPSALFMKQFIRIP | | |
| | F0041.3 | | | | | |
| Campylobacter | Cj0645 / putative | CAB75281 | 1 | YTVKSGDSLYKIAKNYNISVDEIREFNKI | (SEQ ID NO:119) | 9.46 |
| jejuni | secreted | | | AKNHLSINQKLII | | |
| | transglycosylase | | | | | |
| Caulobacter | CC1996 / peptidase, | AAK23971 | 1 | YVVQTGDTMFAIAKRFNVTAAALAEENDL | (SEQ ID NO:120) | 9.31 |
| crescentus | M23/M37 family | | | KSGAAIKKGQKLLL | | |
| Caulobacter | CC1996 / peptidase, | AAK23971 | 2 | YSVQTGDTLGEIAKRFNVSVKALAAENNL | (SEQ ID NO:121) | 10.00 |
| crescentus | M23/M37 family | | | RATASLKKGQKIAL | | |
| Caulobacter | CC1996 / peptidase, | AAK23971 | 3 | HTVKSGDTLTAIARKFDMSVSELAEA | (SEQ ID NO: 122) | 6.76 |
| crescentus | M23/M37 family | | | NKL | | |
| Chlamydia | TC0890 / conserved | AAF39685 | 1 | HVVKQGETLSKIASKYNIPVAELKKLN | (SEQ ID NO:123) | 9.87 |
| muridarum | hypothetical protein | | | KLNSDTIFTDQRIRLP | | |
| Chlamydia | nlpD / Muramidase | AAC68354 | 1 | VIVKKGDFLERIARSNHTTVSALMQLN | (SEQ ID NO:124) | 9.98 |
| trachomatis | | | | DLSSTQLQIGQVLRVP | | |
| Chlamydia | nlpD / Muramidase | AAC68354 | 2 | YVVKEGDSPWAIALSNGIRLDELLKLN | (SEQ ID NO:125) | 9.97 |
| trachomatis | | | | GLDEQKARBLRPGDRLRIR | | |
| Chlamydia | papQ / Invasin repeat | AAC68203 | 1 | HIVKQGETLSKIASKYNIPVVELKKLN | (SEQ ID NO:126) | 9.87 |
| trachomatis | family phosphatase | | | KLNSDTIFTDQRIRLP | | |
| Chlamydomonas | EYE2 / EYE2 | AAE43040 | 1 | YTVQKGETLWDVAVQHGVSMRTIKELN | (SEQ ID NO:127) | 8.34 |
| reinhardtii | | | | KLSGKEP1LKEGQQLLVP | | |
| Chlamydophila | CP0155 / conserved | AAF38036 | 1 | YVVQDGDSLWLIAKRFGIPMDKIIQKN | (SEQ ID NO:128) | 10.00 |
| pneumoniae | hypothetical protein | | | GLNHHRLFPGKVLKLP | | |
| AR39 | | | | | | |
| Chlamydophila | amiB / Ainidase | AAD18918 | 1 | YIVREGDSLSKIAKKYKLSVTELKKIN | (SEQ ID NO:129) | 9.47 |
| pneumoniae | | | | KLDSDAIYAGQRLCLQ | | |
| CWL029 | | | | | | |
| Chlamydophila | nlpD / Muramidase | AAD19040 | 1 | VVVKKGDFLERIARANHTTVAKLMQIN | (SEQ ID NO:130) | 10.17 |
| pneumoniae | | | | DLTTTQLKIGQVIKVP | | |
| CWL029 | | | | | | |
| Chlamydophila | nlpD / Muramidase | AAD19040 | 2 | YIVQEGDSPWTIALRNHIRLDDLLKMN | (SEQ ID NO:131) | 8.38 |
| pneumoniae | | | | DLDEYKARRLKPGDQLRIR | | |
| CWL029 | | | | | | |
| Citrobacter | eae / bacterial | AAA23097 | 1 | YTLKTGESVAQLSKSQGISVPVIWSLN | (SEQ ID NO:132) | 9.08 |
| freundii | adhesin | | | KHLYSSESEMMKASPGQQIILP | | |
| Clostridium | CAC2747 / putative | AAK80693 | 1 | YVVKPGDSVYTIARRFRVTPQSILDAN | (SEQ ID NO:133) | 10.26 |
| acetobutylicum | chitinase | | | NLQNNRLVVGQAIVVP | | |
| Clostridium | CAC2747 / putative | AAK80693 | 2 | YRVRQGDTLWSIARRFRVTPKSITDL | (SEQ ID NO:134) | 11.34 |
| acetobutylicum | chitinase | | | NNIENPSQIQPGLVIRIP | | |
| Clostridium | camb / chitinase | AAC13727 | 1 | YTIQPGDTFWAIAQRRGTTVDVIQSL | (SEQ ID NO:135) | 8.74 |
| acetobutylicum | | | | NPGVVPTRLQVGQVINVP | | |
| Clostridium | camb / chitinase | AAC13727 | 2 | YTIQPGDTFWAIAQRRGTTVDVIQSL | (SEQ ID NO:136) | 8.74 |
| acetobutylicum | | | | NPGVNPARLQVGQVINVP | | |
| Colletotrichum | CIH1 / Glycoprotein | AJ001441 | 1 | THKVKSCESLTTIAEKYDTGICNIAK | (SEQ ID NO:137) | 4.89 |
| lindemuthianum | CIH1 precursor | | | LNNLADPNFIDLNQDLQIP | | |
| Colletotrichum | CIH1 / Glycoprotein | AJ001441 | 2 | YSVVSGDTLTSIAQALQITLQSLKDA | (SEQ ID NO:138) | 5.38 |
| lindemuthianum | CIH1 precursor | | | NPGVVPEHLNVGQKLNVP | | |
| Deinococcus | cwp / putaive cell | AAF10484 | 1 | VRPGQTLYRIALQNGLSVAELQRLNG | (SEQ ID NO:139) | 10.67 |
| radiodurans | wall protein | | | LHSTTIEVGQVLRVT | | |
| Deinococcus | cwp / putaive cell | AAF10484 | 2 | YTVRRGDTLTSIGKFVGLRVEQLQRL | (SEQ ID NO:140) | 11.45 |
| radiodurans | wall protein | | | NGLKGNTIAVGQVLRLT | | |
| Deinococcus | cwp / putaive cell | AAP10484 | 3 | YRVQPGDTLPKIGVKVGLRVEQLRRI | (SEQ ID NO:141) | 10.88 |
| radiodurans | wall protein | | | NGLTGDALQIGQVLRLT | | |
| Deinococcus | cwp / putaive cell | AAF10484 | 4 | YTVRRGDTLTSIGKFVGLP.VEQLQR | (SEQ ID NO:142) | 10.88 |
| radiodurans | wall protein | | | LNGLKDNTIAVGQVLRLT | | |
| Deinococcus | cwp / putaive cell | AAF10484 | 5 | YVVVPGDTLAKIGVKVGLRVEQLQRL | (SEQ ID NO:143) | 9.52 |
| radiodurans | wall protein | | | NGLSGTTIEVGQVLKL | | |
| Deinococcus | DR1749t / | AAF11303 | 1 | YTVKAGDTLSRIAGAYGTDASTLMPM | (SEQ ID NO:144) | 10.26 |
| radiodurans | endopeptidase-related | | | NGLRSTTIQVGQRLQIGSS | | |
| | protein | | | | | |
| Deinococcus | DR0848 / lipoprotein, | AAF10427 | 1 | YTVKKGDTLYSLARGSGLTVDALMRL | (SEQ ID NO:145) | 9.82 |
| radiodurans | putative | | | NGLSTPELRVGQVIKLP | | |
| Deinococcus | DR0848 / | AAF10897 | 1 | LTVQRGDTAYSIARRNGLTVDLLLAY | (SEQ ID NO:146) | 8.59 |
| radiodurans | endopeptidase-related | | | NNLASPDIEVGQVLRLR | | |
| | putative | | | | | |
| Deinococcus | DR1325 / | AAF10897 | 2 | HTAQRGDTIYGLSRMYGVSVDALLAA | (SEQ ID NO:147) | 6.76 |
| radiodurans | endopeptidase-related | | | NTLPRDTKLEVGQVLQLP | | |
| | protein | | | | | |
| Deinococcus | DR1749 / | AAF11303 | 1 | YTVKAGDTLSRIAGAYGTDASTLMB | (SEQ ID NO:148) | 10.26 |
| radiodurans | endopeptidase-related | | | 34NGLRSTTIQVGQRLQIG | | |
| | protein | | | | | |
| Deinococcus | DR2291 / cell wall | AAF11838 | 1 | YRVKPGETLYRIALNAGLSEETVQQA | (SEQ ID NO:149) | 8.44 |
| radiodurans | endopeptidase | | | NPVLRGGHALYAGQMLTI | | |
| | putative | | | | | |
| Deinococcus | DR2291 / cell wall | AAF11838 | 2 | FRVRKGEDLKKLAQRLGVSEGDIRRD | (SEQ ID NO:150) | 11.23 |
| radiodurans | endopeptidase | | | NPQIDRRGSLNAGQVLRLP | | |
| | putative | | | | | |
| Deinococcus | DR2291 / cell wall | AAF11838 | 3 | HRVEIGDTFYSVARRYGINPIALQEY | (SEQ ID NO:151) | 8.50 |
| radiodurans | endopeptidase | | | NPRLAGQTLNVGAVLSLV | | |
| | putative | | | | | |
| Deinococcus | DR0888 / hypothetical | AAF104671 | 1 | YTVKPGDSLSKIAEHYYGDQNEYKKI | (SEQ ID NO:152) | 6.92 |
| radiodurans | protein | | | AHYNNISNPDLIQPGQKL | | |
| Dictyosteliuin | ORF_ID:dd_03106 / | AAM33687 | 1 | LYTVKEKDTLTGISLQFSMPRDVLIQANBL | (SEQ ID NO:153) | 9.81 |
| discoideum | Hypothetical protein | | | LHSEKLLKPGTQLWVY | | |
| Dictyostelium | LYSA /LysA | AAM18799 | 1 | LYTVKEKDTLTGISLQFSMPRDVLIQ | (SEQ ID NO:154) | 9.81 |
| discoideum | (Fragment) | | | ANRLLHSEKLLKPGTQLWVY | | |
| Drosophila | L82 / L82A | AAD28508 | 1 | YTVGNRDTLTSVAARFDTTPSELTHL | (SEQ ID NO:155) | 6.75 |
| melanogaster | | | | NRLNSSFIYPGQQLLVP | | |
| Drosophila | CG12207 / LD22649p | AAK93098 | 1 | RHIVEKTDTLQGIALKYGCTTEQIRR | (SEQ ID NO:156) | 9.19 |
| melanogaster | | | | ANRLFASDSLFLRQFLLVP | | |
| Drosophila | 1 (3) 82Fd / CG32464- | AAF52045 | 1 | SYTVGNRDTLTSVAARFDTTPSELTH | (SEQ ID NO:157) | 7.53 |
| melanogaster | PB | | | LNRLNSSFIyPGQQLLVP | | |
| Drosophila | CG17985 / LD36653P | AAK93296 | 1 | EVKVQEGDTLQALALRFHSSVADIKR | (SEQ ID NO:158) | 9.43 |
| melanogaster | | | | LNKIDRENEIHAHRVIRIP | | |
| Enterococcus | M58002/ muramidase | AAA67325 | 1 | YTVKSGDTLNKIAAQYGVSVANLRSW | (SEQ ID NO:159) | 9.70 |
| faecalis | | | | I4GISGDLIEVGQKLIVK | | |
| Enterococcus | M58002/ muramidase | AAA67325 | 2 | YTVKSGDTLNKIAAQYGVTVANLRSW | (SEQ ID NO:160) | 9.70 |
| faecalis | | | | MGISGDLIFVGQKLIVK | | |
| Enterococcus | M58002/ muramidase | AAA67325 | 3 | YTIKSGDTLNKIAAQYGVSVANLRSW | (SEQ ID NO:161) | 9.70 |
| faecalis | | | | NGISGDLIFAGQKIIVK | | |
| Enterococcus | M58002/ muramidase | AAA67325 | 4 | YTIKSGDTLNKISAQFGVSVANLRSW | (SEQ ID NO:162) | 9.82 |
| faecalis | | | | NGIIcGDLIFAGQTIIVK | | |
| Enterococcus | M58002/ muramidase | AAA67325 | 5 | HTVKSGDSLWGLSMQYGISIQKIKQL | (SEQ ID NO:163) | 9.31 |
| faecalis | | | | NGLSGDTIyIGQTLKVG | | |
| Enterococcus | mur2 / muramidase | P39046 | 1 | YTVKSGDSVWGISHSFGITMAQLIEW | (SEQ ID NO:164) | 9.31 |
| hirae | | | | NNIKNNFIYPGQKLTIK | | |
| Enterococcus | mur2 / muramidase | P39046 | 2 | YTVKSGDSVWKIANDHGISMHQLIEW | (SEQ ID NO:165) | 6.75 |
| hirae | | | | NNIKNNFVYPGQQLVVS | | |
| Enterococcus | mur2 / muramidase | P39046 | 3 | YTVKAGESVWSVSNKFGISMNQLIQW | (SEQ ID NO:166) | 9.88 |
| hirae | | | | NNIKNNFIYPGQKLIVK | | |
| Enterococcus | mur2 / muramidase | P39046 | 4 | YTVKAGESVWGVANKNGISMNQLIEW | (SEQ ID NO:167) | 9.60 |
| hirae | | | | NNIKNNFIYPGQKLIVK | | |
| Enterococcus | mur2 / muramidase | P39046 | 5 | LTVKAGESVWGVANKHHTTHDQLIEW | (SEQ ID NO:168) | 6.93 |
| hirae | | | | NNIKNNFIYPGQEVIVK | | |
| Enterococcus | mur2 / muramidase | P39046 | 6 | YTVKAGESVWGVADSHGITMNQLIEW | (SEQ ID NO:169) | 6.76 |
| hirae | | | | NNIKNNFIYPGQQLIVK | | |
| Escherichia | ORF_f259 / unknown | AAA83046 | 1 | YTVKRGDTLYRISRTTGTSVKELARLNGIS | (SEQ ID NO:170) | 10.11 |
| coli K-12 | protein | | | PPYTIEVGQKLKLG | | |
| Escherichia | dniR / | AAC73316 | 1 | YTVRSGDTLSSIASRLGVSTKDLQQW | (SEQ ID N0:171) | 10.55 |
| coli K12 | transcriptional | | | NKLRGSKLKPGQSLTIG | | |
| | regulator | | | | | |
| Escherichia | dniR / | AAC73316 | 2 | YRVRKGDSLSSIAKRMGVNIKDVMRW | (SEQ ID NO:172) | 10.16 |
| coli K12 | transcriptional | | | NSDTANLQPGDKLTLF | | |
| | regulator | | | | | |
| Escherichia | nlpD / lipoprotein | AAG57849 | 1 | YTVKKGDTLFYIAWTTGNDFRDLAQ | (SEQ ID N0:173) | 8.38 |
| coli 0157:H7 | | | | P.NNIQAPYALNVGQTLQVG | | |
| Escherichia | yebA / Hypothetical | P24204 | 1 | YVVSTGDTLSSILMQYGIDMGDISQ | (SEQ ID NO:174) | 4.36 |
| coli 06 | metalloprotease yebA | | | LAAADKELRNLKIGQQLSWT | | |
| Escherichia | mltD / lytic murein | P23931 | 1 | YTVRSGDTLSSIASRLCVSTKDLQQ | (SEQ ID NO:175) | 10.55 |
| coli 06 | transglycosylase D | | | WNKLRGSKLKPGQSLTIG | | |
| Escherichia | mltD / lytic murein | P23931 | 2 | YRVRKGDSLSSIAKRHGVNIKDVMR | (SEQ ID NO:176) | 10.16 |
| coli 06 | transglycosylase D | | | WNSDTANLQPGDKLTLP | | |
| Fugu rubripes | SINFRUG00000124889 / | n.a.y.^{c} | 1 | VESRDTLNSISLKFDTTPNKLVQLN | (SEQ ID NO:177) | 10.11 |
| | SINFRUP00000131961 | | | KLPSRAVVPCQVLYVP | | |
| Fugu rubripes | SINFRUG00000141170 / | n.a.y.^{c} | 1 | EHRVQPGETLQGLALKYGVSMEQIK | (SEQ ID NO:178) | 10.23 |
| | SINFRUP00000149824 | | | RANRMYTNDSIHLRKSLSIP | | |
| Fugu rubripes | SINFRUG00000141267 / | n.a.y.^{c} | 1 | VGANDSLNSIALNFNITPNKLVQLN | (SEQ ID NO:179) | 10.63 |
| | SINFRUP00000149934 | | | KLFSRSVYPGQKLFVP | | |
| Fugu rubripes | SINFRUG00000151284 / | n.a.y.^{c} | 1 | EREVLDGDTLNKLALQYGCKVADIK | (SEQ ID NO:180) | 6.54 |
| | SINFRUP00000160868 | | | RLNNLMQEQDFYALKSVRIP | | |
| Fugu rubripes | SINFRU000000154956 / | n.a.y.^{c} | 1 | TRDIQEGDTLNSISLQYHCSLADIK | (SEQ ID NO:181) | 5.66 |
| | SINFRUP00000164894 | | | RANNLLTEQDFFALRSVKIP | | |
| Fugu rubripes | SINFRUG00000155347 / | n.a.y.^{c} | 1 | EHRVTDSDTLQGIALKYGVTMEQIK | (SEQ ID NO:182) | 8.52 |
| | SINFRUP0000165314 | | | RANKLFSNDCIFLRNSLNIP | | |
| Haemophilus | amiB / amidase | AAC21744 | 1 | HIVKKGESLGSLSNKYHVKVSDIIK | (SEQ ID NO:183) | 10.10 |
| influenzae | | | | LNQLKRKTLWLNESIKIP | | |
| Haemophilus | amiB / amidase | AAC21744 | 2 | HKVTKNQTLYAISREYNIPVNILLS | (SEQ ID NO:184) | 10.46 |
| influenzae | | | | LNPHLKNGKVITGQKIKLR | | |
| Haemophilus | HI0409 / | AAC220681 | YTVTEGDTLKDVLVLSGLDDSSVQP | (SEQ ID NO:185) | 4.09 | |
| influenzae | hypothetical | | | LIALDPELAHLKAGQQFYWI | | |
| | metalloprotease | | | | | |
| Haemophilus | lppB / Outer | AAC223631 | YKVNKODTMPLIAYLAGIDVKELAA | (SEQ ID NO:186) | 6.21 | |
| influenzae | membrane | | | LNNLSEPNYNLSLGQVLKIS | | |
| | lipoprotein B | | | | | |
| Haemophilus | lppB / Outer | AAA723481 | YKVRKGDTMFLIAYISGMDIKELAT | (SEQ ID NO:187) | 8.31 | |
| somnus | membrane | | | LNMMSEPYHLSIGQVLKIA | | |
| | lipoprotein B | | | | | |
| Helicobacter | HP1572 / DniR | AAD08609 | 1 | HVVLPKETLSSIAKRYQVSISNIQL | (SEQ ID NO:188) | 9.99 |
| pylori 26695 | | | | ANDLKDSNIFIHQRLIIR | | |
| Helicobacter | dniR / regulatory | AAD07052 | 1 | HVVLPKETLSSIAKRYQVSISSIQL | (SEQ ID NO:189) | 9.99 |
| pylon J99 | protein | | | ANNLKDSNIFIHQRLII | | |
| Homo sapiens | AY037156 / | AAK67635 | 1 | EHQLEPGDTLAGLALKYGVTMEQIK | (SEQ ID NO:190) | 9.23 |
| | hypothetical protein | | | RAMRLYTNDSIFLKKTLYIP | | |
| | SB1 45 | | | | | |
| Homo sapiens | MGC35274 / | AAH33515 | 1 | RHVEHRVRAGDTLQGIALKYGVTME | (SEQ ID NO:191) | 10.68 |
| | hypothetical protein | | | QIKRANKLFTNDCIFLKKTLNIPVI | | |
| | MGC35274 | | | | | |
| Homo sapiens | ERAP140 / estrogen | AAM27392 | 1 | EYTAGNQDTLNSIALKFNITPNKLV | (SEQ ID NO:192) | 9.22 |
| | receptor-associated | | | ELNKLFTHTIVPGQVLFVP | | |
| | protein | | | | | |
| Homo sapiens | L0C116068 / MGC:7041 | XP_057302 | 1 | KDIQEGDTLNAIALQYCCTVADIKRVNNLI | (SEQ ID NO:193) | 4.68 |
| | hypothetical protein | | | SDQDFFALBSIKIP | | |
| Homo sapiens | OXR1 / oxidation | AAH32710 | 1 | EYTVESRDSLNSIALKFDTTPNELV | (SEQ ID NO:194) | 4.67 |
| | resistance 1 | | | QLNKLFSRAVVTGQVLYVP | | |
| | hypothetical protein | | | | | |
| Kluyveromyces | ORF2 / put. killer | CAA25334 | 1 | YKVSSGESCSSIAVKYYPLSLNDTENYNKG | (SEQ ID NO:195) | 8.01 |
| lactis | toxin large subunit | | | NYGWKGCSSLQKDYNLCVSDGSAPRPVSNP | | |
| Lactobacillus | lys / lysin | JC5911 | 1 | YTVVSGDSWWKIAQRNGLSMYTLASQNCKS | (SEQ ID NO:196) | 9.78 |
| phage phi-gle | | | | IYSTIYPGNKLIIK | | |
| Lactococcus- | acmD / N- | AAK04639 | 1 | YKVQEGDSLSAIAAQYGTTVDALVSAMSLE | (SEQ ID NO:197) | 3.81 |
| lactis IL1403 | acetylmuramidase (EC | NANDIHVGEVLQVA | | | | |
| | 3.5.1.28) | | | | | |
| Lactococcus | acmD / N- | AAK04639 | 2 | YTVKSGDSLYSIAEQYGMTVSSLMSANGIY | (SEQ ID NO:198) | 3.87 |
| lactis IL1403 | acetylmuramidase (EC | | | DVNSMLQVGQVLQVTV | | |
| | 3.5.1.28) | | | | | |
| Lactococcus | acmD / N- | AAK04639 | 3 | YTIQNGDSIYSIATANGMTADQLAALNGFG | (SEQ ID NO:199) | 4.30 |
| lactis IL1403 | acetylmuramidase (EC | | | INDMIHPGQTIRI | | |
| | 3.5.1.28) | | | | | |
| Lactococcus | TagH / Teichoic acid | AAK05013 | 1 | TYIVQAGDSLSIIAENHGYSVEEIQQVNPG | (SEQ ID NO:200) | 3.84 |
| lactis IL1403 | ABC transporter ATP | | | VDFSVIHPGQEINLP | | |
| | binding protein | | | | | |
| Lactococcus | acmD / N- | AAK04370 | 1 | TYTVKSGDTLWCISQKYGISVAQIQSANNL | (SEQ ID NO:4) | 10.05 |
| lactis IL1403 | acetylmuramidase (EC | | | KSTVIYIGQKLVLT | | |
| | 3.5.1.28) | | | | | |
| Lactococcus | acmD / N- | AAK04370 | 2 | TIKVKSGDTLWGLSVKYKTTIAQLKSWNHL | (SEQ ID NO:5) | 10.37 |
| lactis IL1403 | acetylmurainidase (EC | | | NSDTIFIGQNLIVS | | |
| | 3.5.1.28) | | | | | |
| Lactococcus | acmD / N- | AAK04370 | 3 | IHKVVKGDTLWGLSQKSGSPIASIKAWNHL | (SEQ ID NO:6) | 10.69 |
| lactis IL1403 | acetylmuramidase (EC | | | SSDTILIGQYLRIK | | |
| | 3.5.1.28) | | | | | |
| Lactococcus | acmA / muramidase | AAC33367 | 1 | TYTVKSGDTLWGISQRYGISVAQIQSANNL | (SEQ ID NO:1) | 10.11 |
| lactis MG1363 | | | | KSTIIYIGQKLVLT | | |
| Lactococcus | acmA / muramidase | AAC33367 | 2 | TVKVKSGDTLWALSVKYKTSIAQLKSWNHL | (SEQ ID NO:2) | 10.16 |
| lactis MG1363 | | | | SSDTIYIGQNLIVS | | |
| Lactococcus | acmA / muramidase | AAC33367 | 3 | IHKVVKGDTLWGLSQKSGSPIASIKAWNHL | (SEQ ID NO:3) | 10.69 |
| lactis MG1363 | | | | SSDTILIGQYLRIK | | |
| Lactococcus | lysB / muramidase | AAA20878 | 1 | YIVKQGDTLSGIASNLGTNWQELARQNSLS | (SEQ ID NO:201) | 6.07 |
| lactis phage | | | | NPNMIYSGQVISLT | | |
| LC3 | | | | | | |
| Lactococcus | lysB / muramidase | AAA20878 | 2 | YTVQSGDNLSSIARRLGTTVQSLVSMNGIS | (SEQ ID NO:202) | 8.50 |
| lactis phage | | | | NPNLIYAGQTLNY | | |
| LC3 | | | | | | |
| Lactococcus | lys / Lysin | AAK38070 | 1 | YTVSSGDNLSSIASRLGTTVQSLVSMNGIS | (SEQ ID NO:203) | 5.83 |
| lactis phage | NPNLIYAGQTL | | | | | |
| TP901-1 | | | | | | |
| Lactococcus | tp901-1lys(2) / Lysin | AAK38070 | 2 | YIVKQGDTLSGIASNWGTNWQELARQNSLS | (SEQ ID NO:204) | 8.50 |
| lactis phage | | | | NPNVIYTGQVIRFT | | |
| TP901-1 | | | | | | |
| Lactococcus | orfA / lysin | AAF12705 | 1 | YIVKQGDTLSGIASNWGTNWQELARQNSLS | (SEQ ID NO:205) | 8.50 |
| lactis phaqe | | | | NPNtIYTGQVIRFT | | |
| TPW22 | | | | | | |
| Lactococcus | orfA / lysin | AAF12705 | 2 | YTVRSGDNLSSIASRLGTTVQSLVSMNGIS | (SEQ ID NO:206) | 8.59 |
| lactis phage | | | | NPS1IYAGQTLN | | |
| TPW22 | | | | | | |
| Lactococcus | lys / glycosidase | AAA32615 | 1 | YVVKQGDTLSGIASNWGTNWQELARQNSLS | (SEQ ID NO:207) | 6.07 |
| lactis phage | (muramidase) | | | NPNMIYAGQVISFT | | |
| Tuc2009 | | | | | | |
| Lactococcus | Tuc2 / glycosidase | AAA32615 | 2 | YTVQSGDNLSSIAILLGTTVQSLVSMNGIS | (SEQ ID NO:208) | 3.80 |
| lactis phage | (muramidase) | | | NPNLIYAGQTLNY | | |
| Tuc2009 | | | | | | |
| Lactococcus- | lys / endolysin | AAD02487 | 1 | YTVQSGDTLGAIAAKYGTTYQKLASLNGIG | (SEQ ID NO:209) | 9.17 |
| oenos phage | | | | SPYIIIPGEKLKVS | | |
| 10mc | | | | | | |
| Lactococcus- | lys / endolysin | AAD02487 | 2 | YKVASGDTLSAIASKYGTSVSKLVSLNGLK | (SEQ ID NO:210) | 9.63 |
| oenos phage | | | | NANYIYVGENLKIK | | |
| 10mc | | | | | | |
| Listeria grayi | P60 / adherence and | Q01835 | 1 | VVVASGDTLWGIASKTGTTVDQLKQLNKLD | (SEQ ID NO:211) | 9.40 |
| | invasion protein P60 | | | SDRTVPGQKLTIK | | |
| Listeria qrayi | P60 / adherence and | Q01835 | 2 | YKVKSGDTIWALSVKYGVPVQKLIEWNNLS | (SEQ ID NO:212) | 9.53 |
| | invasion protein P60 | | | SSSIYVGQTIAVK | | |
| Listeria grayi | P60 / adherence and | Q01835 | 3 | YKVQNGDSLGKIASLFKVSVADLTNWNNLN | (SEQ ID NO:213) | 8.34 |
| | invasion protein P60 | | | ATITIYAGQELSVK | | |
| Listeria inocua | P60 / adherence and | Q01836 | 1 | VVVEAGDTLWGIAQSKGTTVDAIKKANNLT | (SEQ ID NO:214) | 8.35 |
| | invasion protein P60 | | | TDKIVPGQKLQVN | | |
| Listeria inocua | P60 / adherence and | Q01836 | 2 | HNVKSGDTIWALSVKYGVSVQDIMSWNHLS | (SEQ ID NO:215) | 9.31 |
| | invasion protein P60 | | | SSSIYVGQKPAIK | | |
| Listeria inocua | lin2838 / N-acetyl- | NP_472166 | 1 | YTVVKGDSLWRIANNHKVTIANLKSWHNLK | (SEQ ID NO:216) | 10.00 |
| | muramidase | | | SDFIYPGQKLKVS | | |
| Listeria inocua | lin2838 / N-acetyl- | NP_472166 | 2 | YTVAKGDSLWRIATNHKVTIANLKSWNNLK | (SEQ ID NO:217) | 10.00 |
| | muramidase | | | SDFIYPGQKLKVS | | |
| Listeria inocua | lin2838 / N-acetyl- | NP_472166 | 3 | YTVAKGDSLWRIATNHKVTIANLKSWNNLK | (SEQ ID NO:218) | 10.00 |
| | muramidase | | | SDFIYPGQKLKVS | | |
| Listeria inocua | lin2838 / N-acetyl- | NP_472166 | 4 | YTVKKGDSLWAISRQYKTTVDNIKAWNKLT | (SEQ ID NO:219) | 10.10 |
| | muramidase | | | SNMIHVGQKLTIK | | |
| Listeria inocua | lin0879 / | NP_470220 | 1 | YKVKSGDTLPGVAKKFDVTVAEIKDWNHLS | (SEQ ID NO:220) | 8.31 |
| | wall-associated | | | SDHLQTGQKLQLT | | |
| | protein precursor | | | | | |
| Listeria | P60 / adherence and | Q01837 | 1 | VVVEAGDTLWGIAQDKGTTVDALKKANNLT | (SEQ ID NO:221) | 6.17 |
| ivanovii | invasion protein P60 | | | SDKIVPGQKLQIT | | |
| Listeria | P60 / adherence and | Q01837 | 2 | YTVKSGDTIWALSSKYGTSVQNIMSWNNLS | (SEQ ID NO:222) | 9.31 |
| ivanovii | invasion protein P60 | | | SSSIYVGQVLAVK | | |
| Listeria | P60 / adherence and | Q01837 | 3 | YTVKSGDTLSKIATTFGTTVSKIKALNGLN | (SEQ ID NO:223) | 9.88 |
| ivanovii | invasion protein P60 | | | SDNLQVGQVLKVK | | |
| Listeria | P60 / adherence and | P21171 | 1 | VVVEAGDTLWGIAQSKGTTVDAIKKANNLT | (SEQ ID NO:224) | 8.35 |
| monocytogenes | invasion protein P60 | | | TDKIVPGQKLQVN | | |
| Listeria | P60 / adherence and | P21171 | 2 | HAVKSGDTIWALSVKYGVSVQDIMSWNNLS | (SEQ ID NO:225) | 9.31 |
| monocytoqenes | invasion protein P60 | | | SSSIYVGQKLAIK | | |
| Listeria | lmo2691 / N-acetyl- | NP_466213 | 1 | YTVVKGDSLWRIANNHKVTVANLKAWNNLK | (SEQ ID NO:226) | 10.00 |
| monocytogenes | muramidase | | | SDFIYPGQKLKVS | | |
| Listeria | lmo2691 / N-acetyl- | NP_466213 | 2 | YTVVKGDSLWRIANNNKVTIANLKAWNNLK | (SEQ ID NO:227) | 10.00 |
| monocytogenes | muramidase | | | SDFIYPGQKLKVS | | |
| Listeria | lmo2691 / N-acetyl- | NP_466213 | 3 | YTVAKGDSLWBIANNNKVTIANLKAWNNL1 | (SEQ ID NO:228) | 10.00 |
| monocytogenes | muramidase | | | cSDFIYPGQKLKVS | | |
| Listeria | lmo2691 / N-acetyl- | NP_466213 | 4 | YTVKKGDSLWAISRQYKTTVDNIKAWNKLT | (SEQ ID NO:229) | 10.10 |
| monocytogenes | muramidase | | | SNMIHVGQKLTIK | | |
| Listeria | lmo2522 /cell wall | NP_466045 | 1 | YKVQDGDSLWKISNENNVSIKQLKEDNNLS | (SEQ ID NO:230) | 4.53 |
| monocytogenes | binding | | | SDIIFPNQTLQVN | | |
| Listeria | lmo2522 /cell wall | NP_466045 | 2 | YTVVACDTLGHIAVDNGVTVNQLKSWNNLS | (SEQ ID NO:231) | 5.30 |
| monocytogenes | binding | | | SDLIIVGQKLSIG | | |
| Listeria | P60 / adherence and | Q01838 | 1 | VVVEAGDTLWGIAQDNGTTVDALKKANKLTT | (SEQ ID NO:232) | 6.17 |
| seeligeri | invasion protein P60 | | | DKIVPGQKLQVT | | |
| Listeria | P60 / adherence and | Q01838 | 2 | HTVKSGDTIWALSVKYGASVQDLMSWNNLSS | (SEQ ID NO:233) | 8.39 |
| seeligeri | invasion protein P60 | | | SSIYVGQNIAVK | | |
| Listeria | P60 / adherence and | Q01838 | 3 | YTVKSGDTLGKIASTFGTTVSKIKALNGLTS | (SEQ ID NO:234) | 9.60 |
| seeligeri | invasion protein P60 | | | DNLQVGDVLKVK | | |
| Listeria | P60 / adherence and | M80348 | 1 | VVVEAGDTLWGIAQSKGTTVDALKKANNLTS | (SEQ ID NO:235) | 8.35 |
| welshimeri | invasion protein P60 | | | DKIVPGQKLQVT | | |
| Listeria | P60 / adherence and | M80348 | 2 | HTVKSGDTIWALSVKYGASVQDLMSWNNLSS | (SEQ ID NO:236) | 9.31 |
| welshimeri | invasion protein P60 | | | SSIYVGQKIAVK | | |
| Listeria | P60 / adherence and | M80348 | 3 | YTVKSGDSLSKIANTFGTSVSKIKALNNLTS | (SEQ ID NO:237) | 9.88 |
| welshimeri | invasion protein P60 | | | DNLQVGTVLKVK | | |
| Macaca | Q9N012 / unknown | AAK67635 | 1 | EHQLEPGDTLAGLALKYGVTMEQIKRANRLY | (SEQ ID NO:238) | 9.23 |
| fascicularis | | | | TNDSIFLKKTLYIP | | |
| Mesorhizobium | mll1077 / lipoprotein | BAB48532 | 1 | YTVQSGDTMSSIARKTGVGVVALKQANGMKD | (SEQ ID NO:239) | 9.82 |
| loti | | | | GLLKIGQTL | | |
| Mus musculus | unknown / unnamed | BAB25902 | 1 | HRVRAGDTLQGIALKYGVTMEQIKRANKLFN | (SEQ ID NO:240) | 10.62 |
| | protein similar to | | | NERIFLKKTLSIP | | |
| | C18 gene | | | | | |
| Mus musculus | ENSMUSG00000042753 / | BAC28427 | 1 | QRELAQEDSLNKLALQYGCKVADIKKANNFIRE | (SEQ ID NO:241) | 9.19 |
| | ENSMUSP00000036686 | | | QDLYALKSIKIP | | |
| Mus musculus | BAC35742 / | XP_127083 | 1 | EHQLEPGDTLAGLALKYGVTMEQIKRTNRLY | (SEQ ID NO:242) | 9.00 |
| | hypothetical protein | | | TNDSIFLKKTLYIP | | |
| | MGC38837 | | | | | |
| Mus musculus | BC003322 / Unknown | AAH03322 | 1 | TKDIQEGDTLNAVALQYCCTVADIKRVNNLI | (SEQ ID NO:243) | 4.67 |
| | (protein for | | | SDQDFFALRSIKIP | | |
| | MGC: 7041) | | | | | |
| Mus musculus | OXR1 / Nucleolar | AAK303658 | 1 | EYTVESRDSLNSIALKFDTTPNELVQLNKLF | (SEQ ID NO:244) | 4.67 |
| | protein C7 | | | SRAVVTGQVLYVP | | |
| Neisseria | NMB1297 / membrane- | AAF41673 | 1 | HRVVEGDTLFNIAKRYNVSVADLIVANNIKG | (SEQ ID NO:245) | 9.99 |
| meningitidis | bound lytic murein | | | NTIQKGQVLRL | | |
| | transglycosylase D | | | | | |
| Neisseria | NMB1483 / lipoprotein | AAF41839 | 1 | HTIVRGDTVYNISKRYHISQDDFRAWNGMTD | (SEQ ID NO:246) | 9.40 |
| meningitidis | NlpD, putative | | | NTLSIGQIVKVK | | |
| Neisseria | NMB01O9 / conserved | AAF40568 | 1 | YTVKQGDTLWGISGKYLYSPWQWGRLWDANr | (SEQ ID NO:247) | 5.41 |
| meningitidis | hypothetical protein | | | DQIHNPD1IYPDQVLV | | |
| Neisseria | NMA0165 / putative | CAB83479 | 1 | YTVKQGDTLWGISGKYLYSPWQWGRLWDANR | (SEQ ID NO:248) | 6.75 |
| meningitidis | periplasmic protein | | | DQIHNPD1IYLDQVL | | |
| Neisseria | NMB1483 / lipoprotein | AAF41839 | 1 | HTIVRGDTVYNISKRYHISQDDFRAWNGMTD | (SEQ ID NO:249) | 9.40 |
| meningitidis | NlpD, putative | | | NTLSIGQIVKVK | | |
| Neisseria | NMA1692 / putative | CAB84920 | 1 | HTIVRGDTVYNISKRYHISQDDERAWNGMTD | (SEQ ID NO:250) | 9.40 |
| meningitidis | membrane peptidase | | | NTLSIGQIVKVK | | |
| Qenococcus oeni | Lys44 / similar to | AAD10705 | 1 | YTVRSGDTLGAIAAKYGTTYQKLASLNGIGS | (SEQ ID NO:251) | 9.52 |
| phage f0g44 | phage encoded lysins | | | PYIIIPGEKLKVS | | |
| Oenococcus oeni | Lys44 / similar to | AAD10705 | 1 | YKVASGDTLSAIASKYGTSVSKLVSLNGLKN | (SEQ ID NO:252) | 9.90 |
| phage f0g45 | phage encoded lysins | | | ANYIYVGQTLRIK | | |
| Oryza sativa | OJ1006F06.19 / | AAN05509 | 1 | IYVVQPQDGLDAIARNVFNAFVTYQEIAAAN | (SEQ ID NO:253) | 4.06 |
| | unknown protein | | | NIPDPNKINVSQTLWIP | | |
| Oryza sativa | OJ1006F06.19 / | AAN05509 | 2 | AYSVGKGENTSAIAAKYGVTESTLLTP.NKI | (SEQ ID NO:254) | 6.64 |
| | unknown protein | | | DDPTKLQMGQILDVP | | |
| Oryza sativa | OSJNBb0024B16.6 / | AAL83617 | 1 | SNTVRRGDTVPGIALKYSIQVTDIKRFNNMM | (SEQ ID NO:255) | 10.43 |
| | hypothetical protein | | | SDHGIYLRERLLIP | | |
| Oryza sativa | B1100D10.27 / | BAB92550 | 1 | VHGVQASETCFSVSQSAGLTQDQFLAFNPNI | (SEQ ID NO:256) | 4.36 |
| | B1100D10.27 protein | | | NCAKVFVGQWVCLD | | |
| Oryza sativa | B1100D10.27 / | BAB92549 | 1 | VHGVEAGETCDSIARRFHAGLGRAPEFRLVS | (SEQ ID NO:257) | 8.07 |
| | B1100D10.27 protein | | | LNPNINCRELFVGQWVCIQ | | |
| Oryza sativa | OSJNBA0026J14.13 / | BAB89226 | 1 | SYAVQDGDTLGNTASLFRSSWKDILDLNPRV | (SEQ ID NO:258) | 4.55 |
| | Protein kinase-like | | | ANPDFIKPGWILFIP | | |
| Oryza sativa | OSJNBA0029C15.5 / | AAL59043 | 1 | LHRVGKLDTLAGTATKYGVEVADIKRLNGLS | (SEQ ID NO:259) | 10.47 |
| | Putative thyinidine | | | TDLQMFAHKTLRIP | | |
| | kinase | | | | | |
| Oryza sativa | BAA82379.1 / putative | BAA82379 | 1 | SHTIRDTGVETYFIIANLTYQGLSTCQALIA | (SEQ ID NO:260) | 5.41 |
| | receptor kinase | | | QNPLHDSRGLVAGDNLTVP | | |
| Oryza sativa | BAA82379.1 / putative | BAA82379 | 2 | TYLVTWGDTVSAIAARFRVDAQEVLDANTLT | (SEQ ID NO:261) | 3.91 |
| | receptor kinase | | | ESSIIYPFTTLLVP | | |
| Pasteurella | PM0903 / unknown | AAK02987 | 1 | HKVKAGENLTGVARKYGVKVNDILALNKLKR | (SEQ ID NO:262) | 10.21 |
| multocida | protein | | | RELWIGETLKIP | | |
| Pasteurella | PM0903 / unknown | AAK02987 | 2 | HTVKKGQTLYAIAREYHIPPNQLLKLNPNLK | (SEQ ID NO:263) | 10.29 |
| muitocida | | | | DGKVLTGQKIKLR | | |
| Pasteurella | PM1614 / unknown | AAK03698 | 1 | YTVRKGDSMYLISYISGLSIKEIAALNNLSE | (SEQ ID NO:264) | 8.30 |
| multocida | protein | | | PYTLATGQVLKLS | | |
| Pichia acaciae | U02596 / killer toxin | AAA20835 | 1 | PYIVQEDDTCVSIASKYPGLTEQDIIDYNSK | (SEQ ID NO:265) | 3.75 |
| | | | | NGDFYGCFNLWEGDKICIS | | |
| Pichia | ORF2 / hypothetical | CAC34265 | 1 | HTNASGESCAFLIEKFTNVETEINLNKWNED | (SEQ ID NO:266) | 4.74 |
| etchellsii | chitinase | | | NPTWYGCSNGHPYVGDKVCVS | | |
| Pichia | ORF2 / hypothetical | CAC34265 | 2 | YYIKSGDTCYDLASKYYLSTEQLESFNEKTY | (SEQ ID NO:267) | 4.51 |
| etchellsii | chitinase | | | GWFCCNNLQLGQAMCLG | | |
| Prevotella | phg / hemagglutinin | AF017417 | 1 | HTVRSNESLYDISQQYGVRLKNIMKANRKIV | (SEQ ID NO:268) | 10.67 |
| intermedia | | | | KRGIKAGDRVVL | | |
| Proteobacterium | AF279106 / predicted | AAG10447 | 1 | YKIQKGDVLSEIAIRFGVTVEEINTENKLNN | (SEQ ID NO:269) | |
| EBAC31A08 | amidase | | | KPIYPGQLIKIN 8.30 | | |
| Pseudomonas | PA3623 / conserved | AAG07011 | 1 | YIVRRGDTLYSIAFRFGWDWKALAARNGIAP | (SEQ ID NO:270) | 9.98 |
| aeruginosa PA01 | hypothetical protein | | | PYTIQVGQAIQFG | | |
| Pseudomonas | mltD / lytic murein | AAG05201 | 1 | YRVRSGDSLHSIANRYRITVAELKSANRLSS | (SEQ ID NO:271) | 11.38 |
| aeruginosa PA01 | transglycosylase D | | | NHLRKGQQL | | |
| Pseudomonas | mltD / lytic murein | AAG05201 | 2 | YKVKNGDSLWQIARNNGVDVNDLKRWNGLDK | (SEQ ID NO:272) | 9.87 |
| aeruginosa PA01 | transglycosylase D | | | HALKVGQTLKL | | |
| Pseudomonas | mltD / lytic murein | AAG05201 | 3 | YKVKQGDSNYLIAKRFNVEMKHLQRWNPRSK | (SEQ ID NO:273) | 11.11 |
| aeruginosa PA01 | transglycosylase D | | | QALKPGQTL | | |
| Pseudomonas | nlpD / lipoprotein | CAB46190 | 1 | YIVKPGDTLFSIAFRYGWDYKELAARNGIPA | (SEQ ID NO:274) | 9.69 |
| putida WCS358 | | | | PYTIRPGQPIRFS | | |
| Rattus | AF078811 / tropic | AAF02216 | 1 | GYTVKSGDTLSAIAAANGVSLANLLSWNNLS | (SEQ ID NO:275) | 8.43 |
| norvegicus | 1808 | | | LQAIIYPGQKLTIQN | | |
| Rattus | AF078811 / tropic | AAF02216 | 2 | SYTVKSGDTLYGIAAKLGTNVQTLLSLNGLQ | (SEQ ID NO:276) | 9.31 |
| norvegicus | 1808 | | | LSSTIYVGQVLKTTG | | |
| Salmonella | nlpD / lipoprotein | AAL21805 | 1 | TVKKGDTLFYIAWITGNDFRDLAQRNSISAP | (SEQ ID NO:277) | 9.12 |
| typhimurium LT2 | | | | YSLNVGQTLQVG | | |
| Sinorhizobium | lppB / lipoprotein | NP_385642 | 1 | IMVRQGDTVTVLARRFGVPEKEILKANGLKS | (SEQ ID NO:278) | 10.26 |
| meliloti | | | | ASQVEPGQRLVIP | | |
| Solanum | eji / Erwinia-induced | AA032065 | 1 | IYTVVSGDPLSHIVTDIFAGLFTVQELQTVN | (SEQ ID NO:279) | 4.06 |
| tuberosum | protein 1 | | | NISNPNLIQPGDKLWIP | | |
| Solanum | eil / Erwinia-induced | AA032065 | 2 | GRLVISGNSIEAIAQQYNVSQETLLRLNGLA | (SEQ ID NO:280) | 4.58 |
| tuberosum | protein 1 | | | SPKELLAGAVLDVP | | |
| Staphylococcus | protA / human serum | AA263251 | 1 | HVVKPGDTVNDIAKANGTTADKIAADNKLAD | (SEQ ID NO:281) | 5.60 |
| aureus | albumin-binding cell | | | KNMIKPGQELVVD | | |
| | surface protein | | | | | |
| Staphylococcus | lytN / autolysin | AAD23962 | 1 | YTVKKGDTLSAIALKYKTTVSNIQNTNNIAN | (SEQ ID NO:282) | 10.00 |
| aureus | homolog | | | PNLIFIGQKLKVP | | |
| Staphylococcus | AJ250906 / autolysin | CAC80837 | 1 | HTVKPGBSVWAISNKYGISIAKLKSLNNLTS | (SEQ ID NO:283) | 9.83 |
| aureus | | | | NLIFPNQVL | | |
| Staphylococcus | AJ250906 / autolysin | CAC80837 | 2 | YTVQAGDSLSLIASKYGTTYQKIMSLNGLNN | (SEQ ID NO:284) | 9.53 |
| aureus | | | | PFIYPGQKLKVT | | |
| Staphylococcus | AJ250906 / autolysin | CAC80837 | 3 | YTVQAGDSLSLIASKYGTTYQNIMRLNGLNN | (SEQ ID NO:285) | 9.31 |
| aureus | | | | FFIYPGQKL | | |
| Staphylococcus | MW0627 / secretory | BAB94492 | 1 | HTVQSGESLWSIAQKYNTSVESIKQNNQLDN | (SEQ ID NO:286) | 5.45 |
| aureus | antigen SsaA | | | NLVFPGQ | | |
| | homologue | | | | | |
| Staphylococcus | MW0627 / secretory | BAB94492 | 2 | HTVQAGESLNIIASRYGVSVDQLMAANNLRG | (SEQ ID NO:287) | 6.75 |
| aureus | antigen SsaA | | | YLIMPNQTL | | |
| | homologue | | | | | |
| Streptococcus | spr0096 / | AAK98900 | 1 | YTVKEGDTLSBIAETHNTTVEKLAENNHIDN | (SEQ ID NO:288) | 4.43 |
| pneumoniae | Hypothetical protein | | | IHLIYVDQELVI | | |
| Synechocystis | nlpD / lipoprotein | BAA18621 | 1 | HQVKEGESLWQISQAFQVDAKAIALANSIST | (SEQ ID NO:289) | 4.50 |
| sp. PCC 6803 | | | | DTELQAGQVLNIP | | |
| Synechocystis | slr0878 / | BAA17599 | 1 | HVVKAGETIDSIAAQYQLVPATLISVNNQLS | (SEQ ID NO:290) | 5.32 |
| sp. PCC 6803 | hypothetical protein | | | SGQVTPGQTILIP | | |
| Thermotoga | TM0409 / conserved | AAD35494 | 1 | YKVQKNDTLYSISLNFGISPSLLLDWNPGLD | (SEQ ID NO:291) | 5.43 |
| maritima | hypothetical protein | | | PHSLRVGQEIVIP | | |
| Thermotoga | TM0409 / conserved | AAD35494 | 2 | YTVKKGDTLDAIAKRFFTTATFIKEANQLKS | (SEQ ID NO:292) | 9.70 |
| maritima | hypothetical protein | | | YTIYAGQKLFIP | | |
| Thermotoga | TM1686 / conserved | AAD36753 | 1 | HVVKRGETLWSIANQYGVRVGDIVLINRLED | (SEQ ID NO:293) | 8.50 |
| maritima | hypothetical protein | | | PDRIVAGQVLKIG | | |
| Treponema | TP0623 / lytic murein | AAC65596 | 1 | HTIRSGDTLYALARRYGLGVDTLKAHNRAHS | (SEQ ID NO:294) | 10.53 |
| pallidum | transglycosylase D | | | ATHLKIGQKLIIP | | |
| Treponema | TP0623 / lytic murein | AAC65596 | 2 | HVVQQGDTLWSLAKRYGVSVENLAEENNLAV | (SEQ ID NO:295) | 4.90 |
| pallidum | transglycosylase D | | | DATLSLGMILKTP | | |
| Treponema | TP0155 / conserved | AAC65145 | 1 | YEVREGDVVGRIAQRYDISQDAIISLNKLRS | (SEQ ID NO:296) | 9.52 |
| pallidum | hypothetical protein | | | TRALQVGQLLKIP | | |
| Treponema | TP0155 / conserved | AAC65145 | 2 | YTVKNGDTFSSIAAAHQISLERLVLLNTPSS | (SEQ ID NO:297) | 8.50 |
| pallidum | hypothetical protein | | | SKBSPPSVRTLVSP | | |
| Treponema | TP0444 / conserved | AAC65431 | 1 | HVIAKGETLPSLSRRYGVPLSALAQANNLAN | (SEQ ID NO:298) | 10.90 |
| pallidum | hypothetical protein | | | VHQLVPGQRIVVP | | |
| Treponema | TP0444 / conserved | AAC65431 | 2 | YTVRRGDTLFSIARNLNCSLAALLAANGISA | (SEQ ID NO:299) | 8.54 |
| pallidum | hypothetical protein | | | AHTIHPGDVLVIP | | |
| Vibrio cholerae | VC0344 / amidase | AAF93517 | 1 | HVVKTGDFLGKLATTYKVSVASIKKENNLKS | (SEQ ID NO:300) | 9.90 |
| | | | | DTLVLGQKLKIT | | |
| Vibrio cholerae | VC0344 / amidase | AAF93517 | 2 | HKVQRGESIGLIANQYGVSVDALKKANNLI˜Z | (SEQ ID NO:301) | 9.82 |
| | | | | SSTISVGQLLTI | | |
| Vibrio cholerae | VC0344 / amidase | AAF93517 | 3 | HKVQRGEFLSKIADQYNVSVDSIRQANQLRT | (SEQ ID NO:302) | 8.50 |
| | | | | DQLLVGQQLII | | |
| Vibrio cholerae | mltD / MltD | AAF95381 | 1 | YKVKSGDTLSTIADKYNTTAKVIKEANQIAS | (SEQ ID NO:303) | 9.52 |
| | | | | NQIRVGSYLFVP | | |
| Vibrio cholerae | mltD / MltD | AAF95381 | 2 | HTVNSGESLWTIAKQYNVPYQSLAKWNGMAP | (SEQ ID NO:304) | 9.87 |
| | | | | KDALRKGQKLVIW | | |
| Vibrio cholerae | mltD / MltD | AAF95381 | 3 | YKVRSGDTLSGIANKFKVKTADIVKWNDLNS | (SEQ ID NO:305) | 9.82 |
| | | | | TQYLKAGQQLKLY | | |
| Volvox carteri | AF058716 / chitinase | AAC13727 | 1 | YTIQPGDTFWAIAQRRGTTVDVIQSLNPGVV | (SEQ ID NO:306) | 8.74 |
| f. nagariensis | | | | PTRLQVGQVINVP | | |
| Volvox carteri | AF058716 / chitinase | AAC13727 | 2 | YTIQPGDTPWAIAQRRGTTVDVIQSLNPGVN | (SEQ ID NO:307) | 8.74 |
| f.naqariensis | | | | PARLQVGQVINVP | | |
| Xylella | XF0925 / hypothetical | AAF83735 | 1 | YTVQKGDTLWGLAKRLFKKPWLWPEIWQANP | (SEQ ID NO:308) | 9.31 |
| fastidiosa | protein | | | QINNPH1IYPGDVISLA | | |
| Xylella | XF0855 / lipoprotein | AAF83665 | 1 | VVVKQGDTLYAISRRTGVAPQDLAAWNRLTA | (SEQ ID NO:309) | 10.16 |
| fastidiosa | XF0855 | | | SKTIYPGQVLRLY | | |

CONSENSUS REPEAT:
hhhlppG-(.)₀₋₂ol.tlut(.)₀₋₂phshsh(.)₀₋₆pplhphN(.)₀₋₂₂hhssstlhs(.)₀₋₂Gpplpls
Grouping of amino acids to classes and the classes abbreviation (the key) used within the consensus
sequence:

| Class | Key | Residues |
|---|---|---|
| alcohol | 0 | S,T |
| aliphatic | l | I,L,V |
| any | . A,C,D,E,F,G,H,I,K,L,M,N,P,Q,R,S,T,V,W,Y | |
| aromatic | a | F,H,W,Y |
| charged | c | D,E,H,K,R |
| hydrophobic | h | A,C,F,G,H,I,K,L,M,R,T,V,W,Y |
| negative | - | D,E |
| polar | p | C,D,E,H,K,N,Q,R,S,T |
| positive | + | H,K,R |
| small | s | A,C,D,G,N,P,S,T,V |
| tiny | u | A,G,S |
| tumlike | t | A,C,D,E,G,H,K,N,Q,R,S,T |
| | (.)₀₋ₙ: | insertion of 0 to n amino acids of any type. |

^{a)}Proteins listed were obtained by homology search using the BLAST program with the repeats of L. lactis AcmA.
^{b)}Genbank or SWISSPROT accession numbers.
^{c)}Not available yet.

TABLE B
Calculated pIs of individual repeat sequences
of the AcmA and AcmD protein anchors.

| | |
|---|---|
| AcmA anchor domain | AcmD anchor domain |

| Repeat | Calculated pI | Repeat | Calculated pI |
|---|---|---|---|
| A1 | 9.75 | D1 | 4.15 |
| A2 | 9.81 | D2 | 3.78 |
| A3 | 10.02 | D3 | 4.15 |
| A1A2A3 | 10.03 | D1D2D3 | 3.85 |

TABLE C
Hybrid protein anchors composed of different AcmA and AcmD repeat
sequences and their calculated pIs.
Composition

| AcmA-repeat sequence | AcmD-repeat sequence | Calculated PI |
|---|---|---|
| A1A2A3 | — | 10.03 |
| A1A2 | D1 | 9.53 |
| A1A2A3 | D1D2D3 | 8.66 |
| A1 | D2 | 8.45 |
| A3 | D1D2 | 7.39 |
| A1A2 | D1D2D3 | 6.08 |
| A3 | D1D2D3 | 5.07 |
| A1 | D1D2D3 | 4.37 |
| — | D1D2D3 | 3.85 |

TABLE D
Adherence of Lactococcus lactis NZ9000 carrying
plasmids pNG304, pNG3041, pCWS1a, pCWS2a, and
pCWS3a with human intestine 407 cells.

| Strain | 407 cells ^{a)} | Bacteria ^{b)} | Mean value |
|---|---|---|---|
| NZ9000(pNG304) | 54 | 255 | 4.7 |
| NZ9000(pNG3041) | 78 | 447 | 5.7 |
| NZ9000(pCWS1a) | 73 | 1342 | 18.4 |
| NZ9000(pCWS2a) | 80 | 2131 | 27.4 |
| NZ9000(pCWS3a) | 64 | 1970 | 30.8 |

^{a)} Number of whole epithelial cells in 10 randomly chosen fields of view in light microscopy
^{b)} Number of lactococcal cells in contact with Henle cells

TABLE E
Growth of mutated MscL strains on agar plates; growth is indicated by
++++ to − corresponding to growth equal to cells
expressing wild-type MscL to an absence of growth, respectively.

| | −IPTG | +IPTG |
|---|---|---|
| WT | ++++ | ++++ |
| G22S | ++++ | ++ |
| S02H | ++++ | ++++ |
| S02H/G22S | ++++ | ++ |
| I03H | ++++ | ++++ |
| I03H/G22S | ++++ | ++ |
| I04H | ++++ | ++++ |
| I04H/G22S | ++++ | + |
| K05H | ++++ | ++++ |
| K05H/G22S | ++++ | +++ |
| E06H | ++++ | ++++ |
| E06/G22S | ++++ | +++ |
| F07H | ++++ | ++++ |
| F07H/G22S | ++++ | ++ |
| R08H | ++++ | ++++ |
| R08H/G22S | ++++ | ++ |
| E09H | ++++ | ++++ |
| E09H/G22S | ++++ | − |
| F10H | ++++ | ++++ |
| F10H/G22S | ++++ | + |
| A11H | ++++ | ++++ |
| A11H/G22S | ++++ | ++ |
| M12H | ++++ | ++++ |
| M12H/G22S | ++++ | ++ |
| R13H | ++++ | +++ |
| R13H/G22S | ++++ | − |

TABLE F
Gating threshold of MscL for WT, G22S and K05H/G22S
measured in spheroplasts by patch clamp. The threshold
for activating (mutant) MscL is shown as the ratio
of their activation pressure to that of MscS.

| | pH 5.85 | pH 7.5 | pH 6* |
|---|---|---|---|
| WT | 1.53 ± 0.185 | 1.56 ± 0.139 | 1.64 ± 0.08 |
| | (n = 5) | (n = 7) | (n = 9) |
| G22S | 1.19 ± 0.07 | 1.139 ± 0.086 | 1.14 ± 0.14 |
| | (n = 6) | (n = 15) | (n = 4) |
| K05H/G22S | 1.148 ± 0.113 | 1.45 ± 0.045 | NA |
| | (n = 6) | (n = 3) | |

*K. Yoshimura, et. al., 1999, Biophys. J. 77: 1960-1972

TABLE G
MTSET-shock assay of WT^{Ll}, G20C^{Ll}, V21C^{Ll}, WT^{Ec}, and
G22C^{Ec} MscL strains.
Growth of pB104 harboring Lac-inducible plasmid with WT^{Ll}, G20C^{Ll},
V21C^{Ll}, WT^{Ec}, and G22C^{Ec} MscL inserts after MTSET-shock (left)
or without MTSET-shock (right) on agar plates with IPTG. Growth
is indicated by ++++ to − corresponding to
growth equal to cells expressing wild-type MscL to an absence
of growth, respectively.

| | +MTSET-shock | −MTSET-shock |
|---|---|---|
| WT L. lactis | ++++ | ++++ |
| G20C L. lactis | − | +++ |
| V21C L. lactis | − | ++++ |
| WT E. coli | ++++ | ++++ |
| G22C E. coli | − | ++++ |

TABLE H
Gelation properties of 5a and 5b

| | | |
|---|---|---|
| Solvent | 5a | 5b |

| | | | | |
|---|---|---|---|---|
| water | g | (5) | g | (10) |
| 1 N NaOH | i | | g | (5) |
| 25% Ammonia | g | (2) | g | (5) |
| 1 N NaHCO₃ | i | | sol | |
| buffer PH 7.9 | g | (10) | g | (10) |
| buffer PH 7.2 | p | | g | (20) |
| buffer PH 6.3 | sol | (p > 20) | sol | |
| buffer PH 5.7 | sol | | sol | |

g = gel;
i = insoluble;
p = precipitate;
sol = soluble.
Values in brackets correspond to the wt % of 5a or 5b used.

TABLE I
Gelation properties of 6

| solvent(s) | state | weight % |
|---|---|---|
| H₂O | insoluble | 0.05 |
| MeOH | soluble | — |
| EtOH | soluble | — |
| DMSO | soluble | — |
| H₂O/MeOH (1:1) | gel | 0.5, 0.25 |
| H₂O/MeOH (4:1) | gel (turbid) | 1.0, 0.5 |
| H₂O/EtOH (1:1) | gel | 1.0, 0.5, 0.25 |
| H₂O/EtOH (2:1) | gel | 0.16 |
| H₂O/EtOH (4:1) | gel (turbid) | 1.0, 0.5 |

TABLE J
Percentages of 8-aminoquinoline and 2-hydroxyquinoline
released from DBC gels of different weight percentages
into 1 mL of PBS solution at pH 7.4 in contact with the
gel, after 24 hours, measured by UV-Vis spectroscopy.

| DBC wt % | 8-aminoquinoline | 2-hydroxyquinoline |
|---|---|---|
| 0.35 | 15 | 38 |
| 0.5 | 12 | 37 |
| 1.0 | 10 | 36 |

TABLE K
Gelation properties of 1, 2, 4 and DBC in the presence
of concentrations of surfactants below and above the critical
micelle concentration (CMC) of the surfactant.

| | | | | | Nonionic | |
|---|---|---|---|---|---|---|
| | anionic | (SDS) | cationic | (CTAB) | (Lactose der.) | |
| | < | > | < | > | < | > |
| | CMC | CMC | CMC | CMC | CMC | CMC |
| ChexAmMetOH | G | G | C | C | G | G |
| 1 | | | | | | |
| ChexAmPheAmR | G | G | G | G | G | G |
| 4 | | | | | | |
| ChexAmPheEsR | G^{a} | G^{a} | G^{a} | G^{a} | G^{a} | G^{a} |
| 2 | | | | | | |
| DBC | G | G | G | C | G | G |
| ChexAmMetOH | G | G | C | C | G | G |
| 1* | | | | | | |
| ChexAmMetOH | G | G | C | C | G | G |
| 1** | | | | | | |
| DBC* | G | G | G | G | G | G |
| DBC** | G | G | G | G | G | G |

G = gel,
C = crystalline,
^{a}= not stable,
*=pH reversible gelation,
**=pH reversible gelation followed by thermoreversible gelation

TABLE L
Gelation properties of 1, 2, 4 and DBC in the presence
of polymeric micelles (Pluronoic P105, BASF)

nonionic
(Pluronic P 105)

| | 5 | 100 |
|---|---|---|
| | mg · ml⁻¹ | mg · ml⁻¹ |
| ChexAmMetOH 1 | p | p |
| ChexAmPheAmR 4 | p | p |
| CHexAmPheEsR 2 | g^{a} | g^{a} |
| DBC | g | g^{a} |
| ChexAmMetOH 1* | g | g |
| ChexAmMetOH 1** | p | p |
| DBC* | g | g |
| DBC** | g | g |

p = precipitate,
g = gel,
^{a}= not stable,
*=pH reversible gelation,
**=pH reversible gelation followed by thermoreversible gelation

1. A delivery vehicle for delivering a substance of interest to a predetermined site, said delivery vehicle comprising:
a substance of interest;
means for inducing availability of at least one compartment of said delivery vehicle towards an exterior of said delivery vehicle; and
targeting means for directing said delivery vehicle to a predetermined site.

2. The delivery vehicle of claim 1, wherein said targeting means comprises an AcmA type protein anchor.

3. The delivery vehicle of claim 2, wherein said AcmA type protein anchor is selected from the group consisting of AcmA, AcmD, homologs thereof, hybrids thereof, mutants thereof, and combinations of any thereof.

4. The delivery vehicle of claim 1, wherein binding of the targeting means to a target is dependent on pH.

5. The delivery vehicle of claim 1, wherein targeting means is capable of binding to a eukaryotic cell.

6. The delivery vehicle of claim 1, wherein the delivery vehicle is a film.

7. The delivery vehicle of claim 1, wherein the delivery vehicle is a membrane.

8. The delivery vehicle of claim 1, wherein the delivery vehicle is a liposome.

9. The delivery vehicle of claim 1, wherein the delivery vehicle comprises an asymmetrical bilayer.

10. The delivery vehicle of claim 7, wherein the membrane is PEG-ylated.

11. The delivery vehicle of claim 10, wherein the membrane comprises cholesterol.

12. The delivery vehicle of claim 7, wherein the membrane further comprises a proteinaceous channel.

13. The delivery vehicle of claim 12, wherein the proteinaceous channel comprises a solute proteinaceous channel.

14. The delivery vehicle of claim 12, wherein the proteinaceous channel comprises a mechanosensitive channel.

15. The delivery vehicle of claim 14, wherein the mechanosensitive channel is of a large conductance channel (MscL) origin or a functional equivalent thereof.

16. The delivery vehicle of claim 1, wherein said means for inducing availability of said at least one compartment is activated by a stimulus selected from the group consisting of radiation; pH; solute concentration; pressure; chemical substances; temperature; enzyme-catalyzed alteration of the delivery vehicle; and combinations of any thereof.

17. The delivery vehicle of claim 16, wherein the radiation is light, sound, a magnetic field, an electrical field, and any combination thereof.

18. The delivery vehicle of claim 1, wherein said delivery vehicle further comprises a gelator having a structure selected from the group of structures consisting of: 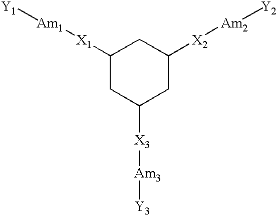
wherein:
X₁=x₂=x₃=—NH—, —C(O)—, —NH—C(O)—, or —C(O)—;
Am₁=Am₂=Am₃=an alpha, beta or gamma amino acid, or an oligopeptide of from 2 to 4 alpha, beta or gamma amino acids;
x₁=y₂=y₃=—OH, —OR, or —NHR;
wherein X₁, X₂ or X₃ is —C(O)— or —NH—C(O)—, Y₁, Y₂, and Y₃ are selected from the group consisting of —C(O)R, —C(O)—NHR, —C(O)—OR, and R;
wherein X₁, X₂ or X₃ is —NH—, R is H or a substituted or unsubstituted, branched, cyclic or straight alkyl, alkenyl or alkynyl group; 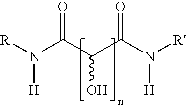
wherein:
n is 3 or 4;
wherein R and R′ represent the same or different substituents selected from the group consisting of substituted or unsubstituted branched groups containing cyclic or linear alkyl, alkenyl, or alkynyl groups having from 1 to 40 carbon atoms; 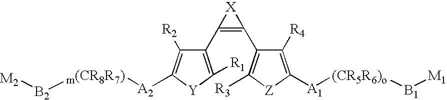
wherein:
X is a moiety selected from the group of moieties consisting of —(CH₂)ₙ₋, —(CF₂)ₙ₋, —C(═O)—O—C(═O)— and —C(═O)—NR—C(═O)—, wherein n is 3 or 4 and R is hydrogen, a (cyclo)alkyl group or an aryl group;
Y and Z each are nitrogen or sulfur;
R₁ and R₃ each are an alkyl group;
R₂ and R₄ each are hydrogen or an alkyl group;
A₁ and A₂ each are absent or are an aryl group;
R₅, R₆, R₇, and R₈ each are hydrogen, an alkyl group or an aryl group;
m and o each are integers selected from the group of integers consisting of 0, 1, 2, 3, and 4;
B₁ and B₂ are each hydrogen bonding moieties; and
M₁ and M₂ each are an aryl group, a (cyclo)alkyl group, or —CR₉R₁₀R₁₁;
wherein R₉, R₁₀ and R₁₁ each are hydrogen, a (cyclo)alkyl group, an aralkyl group or an aryl group; 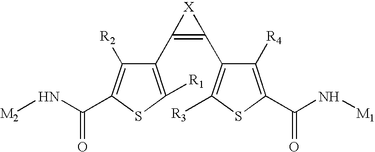
wherein:
R₁ and R₃ are both methyl, and the other symbols have the same meanings as defined above; and
R₂ and R₄ each are hydrogen or a methyl group.

19. The delivery vehicle of claim 18, wherein each amino acid of the alpha, beta or gamma amino acids of Am₁, Am₂ or Am₃ is substituted with a substituted or unsubstituted, branched, cyclic or straight alkyl or alkenyl group which contains an aromatic, ester or ether moiety, or one or more other heteroatoms selected from the group consisting of N, S, O, P and B.

20. The delivery vehicle of claim 19, wherein the substituted branched, cyclic or straight alkyl or alkenyl group does not contain more than 12 carbon atoms.

21. The delivery vehicle of claim 18, wherein each of Am₁, Am₂ and Am₃ contain zero or one substituent.

22. The delivery vehicle of claim 18, wherein the substituted or unsubstituted branched, cyclic or straight chain alkyl, alkenyl or alkynyl group of the Y₁, Y₂, or Y₃ is an aromatic, ester or ether moiety or one or more other heteroatoms and has from 1 to 40 carbon atoms.

23. The delivery vehicle of claim 18, wherein the substituted or unsubstituted branched groups of the R or R′ are aromatic.

24. The delivery vehicle of claim 18, wherein R₂ and R₄ have the same meaning.

25. The delivery vehicle of claim 18, wherein said means for inducing availability of said at least one compartment comprises a viscous material that, upon application of a signal, becomes less viscous.

26. The delivery vehicle of claim 25, wherein said signal comprises light, sound or an electrical field.

27. The delivery vehicle of claim 18, wherein -said means for inducing availability of said at least one compartment comprises an enzyme-cleavable linkage.

28. The delivery vehicle of claim 18, wherein the gelator is contained within a liposome.

29. The delivery vehicle of claim 1, wherein said delivery vehicle comprises a hydrophobin or a functional equivalent thereof.

30. The delivery vehicle of claim 1, wherein the substance of interest is selected from the group consisting of: a nucleic acid; a peptide; a drug; a diagnostic component; and any combination thereof.

31. The delivery vehicle of claim 30, wherein the nucleic acid is RNA or DNA.

32. The delivery vehicle of claim 30, wherein the drug is an organic molecule or a biomolecule.

33. The delivery vehicle of claim 32, wherein the biomolecule is a nucleic acid or a peptide.

34. The delivery vehicle of claim 30, wherein the diagnostic component is a radionuclide, a contrast fluid, an antibody, an antigen or a ligand.

35. A method for delivering a substance of interest to a predetermined site the method comprising:
producing a delivery vehicle comprising:
a substance of interest;
means for inducing availability of at least one compartment of said delivery vehicle towards an exterior of said delivery vehicle;
targeting means for directing said delivery vehicle to a predetermined site;
administering the delivery vehicle to a subject; and
inducing the availability of said substance of interest to an exterior of the delivery vehicle.

36. The method according to claim 35, wherein inducing the availability of said substance of interest to the exterior of the delivery vehicle comprises:
allowing the delivery vehicle to arrive at the predetermined site; and
applying a signal which causes the induction to the predetermined site.

37. The method according to claim 35, wherein the induction takes place by opening a protein channel.

38. The method according to claim 35, wherein the predetermined site is a part of a body of the subject.

39. The method according to claim 38, wherein the part of the body is a joint or a solid tumor.

40. A process for producing a delivery vehicle, the process comprising:
generating a lipid vehicle comprising a proteinaceous channel;
wherein the lipid vehicle further comprises a small hydrophilic molecule.

41. (canceled)


SEQUENCE LISTING (309 sequences)

SEQ ID NO: 1 - protein, 44 aa, Lactococcus lactis MG1363
TYTVKSGDTLWGISQRYGISVAQIQSANNLKSTIIYIGQKLVLT

SEQ ID NO: 2 - protein, 44 aa, Lactococcus lactis MG1363
TVKVKSGDTLWALSVKYKTSIAQLKSWNHLSSDTIYIGQNLIVS

SEQ ID NO: 3 - protein, 44 aa, Lactococcus lactis MG1363
IHKVVKGDTLWGLSQKSGSPIASIKAWNHLSSDTILIGQYLRIK

SEQ ID NO: 4 - protein, 44 aa, Lactococcus lactis IL1430
TYTVKSGDTLWGISQKYGISVAQIQSANNLKSTVIYIGQKLVLT

SEQ ID NO: 5 - protein, 44 aa, Lactococcus lactis IL1430
TIKVKSGDTLWGLSVKYKTTIAQLKSWNHLNSDTIFIGQNLIVS

SEQ ID NO: 6 - protein, 44 aa, Lactococcus lactis IL1430; identical to SEQ ID NO: 3

SEQ ID NO: 7 - protein, 149 aa, Artificial Sequence
  Sequences encompassed in the wider definition of hydrophobin
XCXXXXXXXXXXXXXXXXXXXXXXXXXXXXXXXXXXXXXXXXXXXXXXXXXXCCXXXXXXXXXXXXXXXXXXXXXXXXXXXXXXXXXXXXXXXCXXXXXXXXXXXXXXXXXXXXXXXCXXXXXXXXXCCXXXXXXXXXXXXXXXXXXCX

SEQ ID NO: 8 - protein, 370 aa, Artificial Sequence
  Sequences encompassed in teh wider definition of hyrdrophobin
XCXXXXXXXXXXXXXXXXXXXXXXXXXXXXXXXXXXXXXXXXXXXXXXXXXXCXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXXCXXXXXXXXXXXXXXXXXXXXXXXXXXXXXXXXXXXXXXXXXXXXXXXXXXCXXXXXCXXXXXXXXXXXXXXXXXXXXXXXXXXXXXXXXXXXXXXXXXXXXXXXXXXCX

SEQ ID NO: 9 - DNA, 31 bp, Artificial Sequence
  primer MltDrepeat.fw
ggaagactcaattgctgctgtacagtcgacg

SEQ ID NO: 10 - DNA, 52 bp, artificial Sequence
  primer MltDrepeat2.rev
taataagcttaaaggtctccaattcccaacgtcagcttatcgcctggtttgc

SEQ ID NO: 11 - DNA, 35 bp, Artificial Sequence
  primer cArepeat3.fw
ccgtctccaattcaatctgctgctgcttcaaatcc

SEQ ID NO: 12 - DNA, 58 bp, artificial Sequence
  primer cArepeat3.rev
taataagcttaaaggtctccaattccttttattcgtagatactgaccaattaaaatag

SEQ ID NO: 13 - DNA, 38 bp, artificial Sequence
  primer cDrepeat1.fw
ccgtctccaatttcaggaggaactgctgttacaactag

SEQ ID NO: 14 - DNA, 50 bp, artificial Sequence
  primer cDrepeat3.rev
taataagcttaaaggtctccaattccagcaacttgcaaaacttctcctac

SEQ ID NO: 15 - DNA, 31 bp, artificial Sequence
  primer CWS1
atataaagcttgcaaagtctgaaaacgaagg

SEQ ID NO: 16 - DNA, 35 bp, artificial Sequence
  primer CWS2
ccgtctcaagctcactattcttcacgttgtttccg

SEQ ID NO: 17 - DNA, 30 bp, artificial Sequence
  primer MSA2-1
accatggcaaaaaatgaaagtaaatatagc

SEQ ID NO: 18 - DNA, 48 bp, artificial Sequence
  primer MSA2-4
cggtctctagcttataagcttagaattcgggatgttgctgctccacag

SEQ ID NO: 19 - DNA, 36 bp, artificial Sequence
  primer CWS3
atttaagcttttaccggatgtaagttgaccattacg

SEQ ID NO: 20 - DNA, 35 bp, artificial Sequence
  primer Prt.Myc2
aagatcttctttgaaataagtttttgttccgtgct

SEQ ID NO: 21 - protein, 21 aa, artificial Sequence
  hydrophobic N-terminal domain of the protein anchor cA
KETWWETWWTEWSQPKKKRKV

SEQ ID NO: 22 - protein, 44 aa, Argobacter tumefaciens
VTLRPGESIATISNRYGVPEKELLRVNGLKTASSAQAGQSILIP

SEQ ID NO: 23 - protein, 43 aa, Argobacterium tumefaciens
YKVQPGDSLAKIARANGVSVAALKAANGISNESIRVGQTLAMP

SEQ ID NO: 24 - protein, 44 aa, Anopheles gambiae
TYTVKDRDTLTSVAARFDTTPSELTQLNRLASSFIYSGQQLLVP

SEQ ID NO: 25 - protein, 45 aa, Anopheles gambiae
RHDVERTDTLQGLALKYGCSMEQIRRVNRLLPTDTIFLRPFLMVP

SEQ ID NO: 26 - protein, 53 aa, Anopheles gambiae
EAQILPGDTLQAIALRFNCSVGFLVSTKIPQLKKLNKIDKDNEIYARNVIRVP

SEQ ID NO: 27 - protein, 45 aa, Aquifex aeolicus V5
YKVKKGDSLWKIAKEYKTSIGKLLELNPKLKNRKYLRPGEKICLK

SEQ ID NO: 28 - protein, 43 aa, Aquifex aeolicus VF5
YRVKRGDSLIKIAKKFGVSVKEIKRVNKLKGNRIYVGQKLKIP

SEQ ID NO: 29 - protein, 43 aa, Aquifex aeolicus VF5
YRVRRGDTLIKIAKRFRTSVKEIKRINRLKGNLIRVGQKLKIP

SEQ ID NO: 30 - protein, 44 aa, Arabidopsis thaliana
HRISKFDTLAGVAIKYGVEVADVKKMNNLVTDLQMFALKSLQIP

SEQ ID NO: 31 - protein, 45 aa, Arabidopsis thaliana
EHRVSKFDTLAGIAIKYGVEVADITKLNGLVTDLQMFALESLRIP

SEQ ID NO: 32 - protein, 47 aa, Arabidopsis thaliana
TYTIQPNDSYFAIANDTLQGLSTCQALAKQNNVSSQSLFPGMRIVVP

SEQ ID NO: 33 - protein, 46 aa, Arabidopsis thaliana
SYTVVFEDTIAIISDRFGVETSKTLKANEMSFENSEVFPFTTILIP

SEQ ID NO: 34 - protein, 48 aa, Arabidopsis thaliana
EYTIKKDDILSFVATEIFGGLVTYEKISEVNKIPDPNKIEIGQKFWIP

SEQ ID NO: 35 - protein, 45 aa, Arabidopsis thaliana
AHVVKLGSSLGEIAAQFGTDNTTLAQLNGIIGDSQLLADKPLDVP

SEQ ID NO: 36 - protein, 48 aa, Arabidopsis thaliana
HYKTRPSDNLGSIADSVYGGLVSAEQIQEANSVNDPSLLDVGTSLVIP

SEQ ID NO: 37 - protein, 44 aa, Arabidopsis thaliana
SYVVKEIDTLVGIARRYSTTITDLMNVNAMGAPDVSSGDILAVP

SEQ ID NO: 38 - protein, 48 aa, Arabidopsis thaliana
HYKTRTSDTLGSIADSVYGGLVSPEQIQVANSETDLSVLDVGTKLVIP

SEQ ID NO: 39 - protein, 44 aa, Arabidopsis thaliana
SYVVRGIDTMAGIAKRFSTSVTDLTNVNAMGAPDINPGDILAVP

SEQ ID NO: 40 - protein, 45 aa, Arabidopsis thaliana
SHRICRGDSVTSLAVKYAVQVMDIKRLNNMMSDHGIYSRDRLLIP

SEQ ID NO: 41 - protein, 45 aa, Arabidopsis thaliana
SYVAMAGDSVQSLSSRFGVSMDRIEDVNGILNLDNITAGDLLYIP

SEQ ID NO: 42 - protein, 40 aa, Bacillus cereus
HTVKKNDTLWGISKQYGVSIQSIKQANNKGNDKTFIGEQL

SEQ ID NO: 43 - protein, 40 aa, Bacillus cereus
YQVQPGDSLETIAKRYNVTVQSIKQMNNTVGNKLYTGQHL

SEQ ID NO: 44 - protein, 44 aa, Bacillus cereus
VTVRSGDSVYSLASKYGSTPDEIVKDNGLNPAETLVVGQALIVN

SEQ ID NO: 45 - protein, 44 aa, Bacillus cereus
YYVQPGDSLYRISQTYNVPLASLAKVNNLSLKSILHVGQQLYVP

SEQ ID NO: 46 - protein, 42 aa, Bacillus halodurans
FTVSKGDTLWGIAQKHEMSVETLMEINGLNDTLIHPGDDLIV

SEQ ID NO: 47 - protein, 42 aa, Bacillus holodurans
HVVKTGDTLYRIALNHQLTVEELQANNDLTDELIFPGQVLAL

SEQ ID NO: 48 - protein, 42 aa, Bacillus halodurans
HQVQSGDTLYLLSEQYGVPMEAIKRINERSSNTIYRGEQLTI

SEQ ID NO: 49 - protein, 45 aa, Bacillus halodurans
HIVQKGDTLWKLAKKYGVDFEQLKAANSQLANPDMIMPGMKIKIP

SEQ ID NO: 50 - protein, 48 aa, bacillus halodurans
YTVQPGDTLSAIAARFGSTVLEIQRANLQDPRFIEPNVIFPGWTLVIP

SEQ ID NO: 51 - protein, 44 aa, Bacillus halodurans
YLPVPGDTLFRISQRFSAHFDLIAGVNRLQDPNLIFVGQLLWVP

SEQ ID NO: 52 - protein, 47 aa, Bacillus halodurans
YEIEVGDTLFGISRRFQIPITKILAANEGRPGFSLDFIFVGFRLLLP

SEQ ID NO: 53 - protein, 42 aa, Bacillus halodurans
HTVVSGDTMWKIAARYQVGVSEIIQANPQVSNPNVIYPGQKL

SEQ ID NO: 54 - protein, 45 aa, Bacillus phage PZA
YKVKSGDNLTKIAKKHNTTVATLLKLNPSIKDPNMIRVGQTINVT

SEQ ID NO: 55 - protein, 45 aa, Bacillus phage PZA
HKVKSGDTLSKIAVDNKTTVSRLMSLNPEITNPNHIKVGQTIRLS

SEQ ID NO: 56 - protein, 44 aa, Bacillus sphaericus
ILIRPGDSLWYFSDLFKIPLQLLLDSNRNINPQLLQVGQRIQIP

SEQ ID NO: 57 - protein, 44 aa, Bacillus sphaericus
YTITQGDSLWQIAQNKNLPLNAILLVNPEIQPSRLHIGQTIQVP

SEQ ID NO: 58 - protein, 43 aa, Bacillus subtilis
IKVKKGDTLWDLSRKYDTTISKIKSENHLRSDIIYVGQTLSIN

SEQ ID NO: 59 - protein, 43 aa, Bacillus subtilis
YKVKSGDSLWKISKKYGMTINELKKLNGLKSDLLRVGQVLKLK

SEQ ID NO: 60 - protein, 43 aa, Bacillus subtilis
YKVKSGDSLSKIASKYGTTVSKLKSLNGLKSDVIYVNQVLKVK

SEQ ID NO: 61 - protein, 44 aa, Bacillus subtilis
CIVQQEDTIERLCERYEITSQQLIRMNSLALDDELKAGQILYIP

SEQ ID NO: 62 - protein, 43 aa, Bacillus subtilis
MVKQGDTLSAIASQYRTTTNDITETNEIPNPDSLVVGQTIVIP

SEQ ID NO: 63 - protein, 44 aa, Bacillus subtilis
YDVKRGDTLTSIARQFNTTAAELARVNRIQLNTVLQIGFRLYIP

SEQ ID NO: 64 - protein, 43 aa, Bacillus subtilis
IKVKSGDSLWKLSRQYDTTISALKSENKLKSTVLYVGQSLKVP

SEQ ID NO: 65 - protein, 43 aa, Bacillus subtilis
YTVAYGDSLWMIAKNHKMSVSELKSLNSLSSDLIRPGQKLKIK

SEQ ID NO: 66 - protein, 43 aa, Bacillus subtilis
YTVKLGDSLWKIANSLNMTVAELKTLNGLTSDTLYPKQVLKIG

SEQ ID NO: 67 - protein, 43 aa, Bacillus subtilis
YKVKAGDSLWKIANRLGVTVQSIRDKNNLSSDVLQIGQVLTIS

SEQ ID NO: 68 - protein, 43 aa, Bacillus subtilis
ITVQKGDTLWGISQKNGVNLKDLKEWNKLTSDKIIAGEKLTIS

SEQ ID NO: 69 - protein, 43 aa, Bacillus subtilis
YTIKAGDTLSKIAQKFGTTVNNLKVWNNLSSDMIYAGSTLSVK

SEQ ID NO: 70 - protein, 43 aa, Bacillus subtilis
HHVTPGETLSIIASKYNVSLQQLMELNHFKSDQIYAGQIIKIR

SEQ ID NO: 71 - protein, 44 aa, Bacillus subtilis
YHVKKGDTLSGIAASHGASVKTLQSINHITDPNHIKIGQVIKLP

SEQ ID NO: 72 - protein, 45 aa, Bacillus subtilis
HIVQKGDSLWKIAEKYGVDVEEVKKLNTQLSNPDLIMPGMKIKVP

SEQ ID NO: 73 - protein, 43 aa, Bacillus subtilis
HIVGPGDSLFSIGRRYGASVDQIRGVNGLDETNIVPGQALLIP

SEQ ID NO: 74 - protein, 43 aa, Bacillus subtilis
YQVKQGDTLNSIAADFRISTAALLQANPSLQAGLTAGQSIVIP

SEQ ID NO: 75 - protein, 43 aa, Bacillus subtilis
YVVKQGDTLTSIARAFGVTVAQLQEWNNIEDPNIRVGQVLIVS

SEQ ID NO: 76 - protein, 43 aa, Bacillus subtilis
IKVKSGDSLWKLAQTYNTSVAALTSANHLSTTVLSIGQTLTIP

SEQ ID NO: 77 - protein, 43 aa, Bacillus subtilis
YTVKSGDSLWLIANEFKMTVQELKKLNGLSSDLIRAGQKLKVS

SEQ ID NO: 78 - protein, 43 aa, Bacillus subtilis
YKVQLGDSLWKIANKVNMSIAELKVLNNLKSDTIYVNQVLKTK

SEQ ID NO: 79 - protein, 43 aa, Bacillus subtilis
YTVKSGDSLWKIANNYNLTVQQIRNINNLKSDVLYVGQVLKLT

SEQ ID NO: 80 - protein, 43 aa, Bacillus subtilis
YTVKSGDSLWVIAQKFNVTAQQIREKNNLKTDVLQVGQKLVIS

SEQ ID NO: 81 - protein, 58 aa, Bacillus subtilis
YTVKKGDTLWDLAGKEYGDSTKWPKIWKVNKKAMIKRSKRNIRQPGHWLFPGQKLKIP

SEQ ID NO: 82 - protein, 58 aa, Bacillus subtilis
YTVKKGDTLWDIAGRFYGNSTQWRKIWNANKTAMIKRSKRNIRQPGHWLFPGQKLKIP

SEQ ID NO: 83 - protein, 46 aa, Bacillus subtilis
HTVQKKETLYRISNKYYKSRTGEEKIRAYNHLNGNDVYTGQVLDIP

SEQ ID NO: 84 - protein, 51 aa, Bacillus subtilis
IEVQQGDTLWSIADQVADTKKINKNDFIEWVADKNQLQTSDIQPGDELVIP

SEQ ID NO: 85 - protein, 44 aa, Bacillus subtilis phage 103
YTVKKGDTLSEIAVKTGVSMAKLQAYNGIKNANKITVGQVLKLT

SEQ ID NO: 86 - protein, 45 aa, Bacillus subtilis phage 103
HVVKKGDTLSEIAKKIKTSTKTLLELNPTIKNPNKIYVGQRINVG

SEQ ID NO: 87 - protein, 45 aa, Bacillus subtilis phage 103
YRIKRGETLTGIAKKNKTTVSQLMKLNPNIKNANNIYAGQTIRLK

SEQ ID NO: 88 - protein, 44 aa, Bacillus subtilis phage PBSX
YVVKQGDTLTSIARAFGVTVAQLQEWNNIEDPNLIRVGQVLIVS

SEQ ID NO: 89 - protein, 42 aa, Bartonella bacilliformis
YIVQSGDTLFSIAQQKGISVESLKVANGMGDNAIYIGQKLVI

SEQ ID NO: 90 - protein, 44 aa, Borrelia burgdorferi
HKIKPGETLSHVAARYQITSETLISFNEIKDVRNIKPNSVIKVP

SEQ ID NO: 91 - protein, 43 aa, Borrelia burgdorferi
YIVKKNDSISSIASAYNVPKVDILDSNNLDNEVLFLGQKLFIP

SEQ ID NO: 92 - protein, 43 aa, Borrelia burgdorferi
YKVVKGDTLFSIAIKYKVKVSDLKRINKLNVDNIKAGQILIIP

SEQ ID NO: 93 - protein, 43 aa, Borrelia burgdorferi
YTAKEGDTIESISKLVGLSQEEIIAWNDLRSKDLKVGMKLVLT

SEQ ID NO: 94 - protein, 43 aa, Borrelia burgdorferi
YMVRKGDSLSKLSQDFDISSKDILKFNFLNDDKLKIGQQLFLK

SEQ ID NO: 95 - protein, 43 aa, Borrelia burgdorferi
HYVKRGETLGRIAYIYGVTAKDLVALNGNPAINLKAGSLLNVL

SEQ ID NO: 96 - protein, 44 aa, Borrelia burgdorferi
HSVAVGETLYSIARHYGVLIEDLKNWNNLSSIINIMHDQKLKIF

SEQ ID NO: 97 - protein, 43 aa, Borrelia burgdorferi
YKVKKGDTFFKIANKINGWQSGIATINLLDSPAVSVGQEILIP

SEQ ID NO: 98 - protein, 65 aa, Caenorhabditis elegans
HTIKSGDTCWKIASEASISVQELEGLNSKKSCANLAVGLSEQEFTEMNEELDCNRLAVGNEICVS

SEQ ID NO: 99 - protein, 45 aa, Caenorhabditis elegans
KIHVKEGDTCYTIWTSQHLTEKQFMDMNEELNCGMLEIGNEVCVD

SEQ ID NO: 100 - protein, 42 aa, Caenorhabditis elegans
YATVTPGSSCYTISASYGLNLAELQTTYNCDALQVDDTICVS

SEQ ID NO: 101 - protein, 45 aa, Caenorhabditis elegans
RIEILNGDTCGFLENAFQTNNTEMEIANEGVKCDNLPIGRMMCVW

SEQ ID NO: 102 - protein, 45 aa, Caenorhabditis elegans
FQMVNQFQSCEDINQRSQISNRKLLELNPTFRCDEMAKYEQICLG

SEQ ID NO: 103 - protein, 44 aa, Caenorhabditis elegans
DYTITETDTLERVAASHDCTVGELMKLNKMASRMVFPGQKILVP

SEQ ID NO: 104 - protein, 45 aa, Caenorhabditis elegans
ERKVKNGDTLNKLAIKYQVNVAEIKRVNNMVSEQDFMALSKVKIP

SEQ ID NO: 105 - protein, 45 aa, Caenorhabditis elegans
WTEIKSGDSCWNIASNAKISVERLQQLNEGMKCDKLPLGDKLCLA

SEQ ID NO: 106 - protein, 45 aa, Caenorhabditis elegans
KLKLKAEDTCFKIWSSQKLSERQFLGHNEGMDCDKLKVGKEVCVA

SEQ ID NO: 107 - protein, 43 aa, Caenorhabditis elegans
HKIQKGDTCFKIWTTNKISEKQFRNLNKGLCDKLEIGKEVCIS

SEQ ID NO: 108 - protein, 44 aa, Caenorhabditis elegans
LKIKEGDTCYNIWTSQKISEQEFNELNKGLDCDKLEIGKEVCVT

SEQ ID NO: 109 - protein, 44 aa, Caenorhabditis elegans
YRFKKGDTCYKIWTSHKMSEKQFRALNRGIDCDRLVPGKELCVG

SEQ ID NO: 110 - protein, 44 aa, Caenorhabditis elegans
ITVKPGDTCFSIWTSQKNTQQQFMDINPELDCDKLEIGKEVCVT

SEQ ID NO: 111 - protein, 44 aa, Caenorhabditis elegans
VKINPGDTCFNIWTSQBNTQQQFMDLNKRLDCDKLEVGKEVCVA

SEQ ID NO: 112 - protein, 44 aa, Caenorhabditis elegans
VQINPGDTCFKIWSAQKLTEQQFHELNKGLDCDRLEVGKEVCIA

SEQ ID NO: 113 - protein, 44 aa, Caenorhabditis elegans
TEVKEGDTCFKIWSAHKITEQQFMEMIRGLDCNRLEVGKEVCIV

SEQ ID NO: 114 - protein, 44 aa, Caenorhabditis elegans
IKVKEGDTCFKIWSAQKMTEQQFMEMNRGLDCNKLMVGKEVCVS

SEQ ID NO: 115 - protein, 42 aa, Caenorhabditis elegans
ATITPGNTCFNISVAYGINLTDLQIZTYDCKALEVGDTICVS

SEQ ID NO: 116 - protein, 44 aa, Caenorhabditis elegans
IEVIKGDTCWFLENAFKTNQTEMERANEGVKCDNLPIGRNMCVW

SEQ ID NO: 117 - protein, 45 aa, Caenorhabditis elegans
IQNFNQYQSCNQVNQQNQISNRKLMDLNPTFRCDRLQKSEQVSQK

SEQ ID NO: 118 - protein, 45 aa, Caenorhabditis elegans
IYQVQTDDTLERIALKHNCSVSSLVRANKLWSPSALFMKQFIRIP

SEQ ID NO: 119 - protein, 42 aa, Campylobacter jejuni
YTVKSGDSLYKIAKNYNISVDEIREFNKIAKNHLSINQKLII

SEQ ID NO: 120 - protein, 43 aa, Caulobacter crescentus
YVVQTGDTMFAIAKRFNVTAAALAEENDLKSGAAIKKGQKLLL

SEQ ID NO: 121 - protein, 43 aa, Caulobacter crescentus
YSVQTGDTLGEIAKRFNVSVKALAAENNLRATASLKKGQKIAL

SEQ ID NO: 122 - protein, 29 aa, Caulobacter crescentus
HTVKSGDTLTAIARKFDMSVSELAEANKL

SEQ ID NO: 123 - protein, 43 aa, Chlamydia muridarum
HVVKQGETLSKIASKYNIPVAELKKLNKLNSDTIFTDQRIRLP

SEQ ID NO: 124 - protein, 43 aa, Chlamydia trachomatis
VIVKKGDFLERIARSNHTTVSALMQLNDLSSTQLQIGQVLRVP

SEQ ID NO: 125 - protein, 46 aa, Chlamydia trachomatis
YVVKEGDSPWAIALSNGIRLDELLKLNGLDEQKARBLRPGDRLRIR

SEQ ID NO: 126 - protein, 43 aa, Chlamydia trachomatis
HIVKQGETLSKIASKYNIPVVELKKLNKLNSDTIFTDQRIRLP

SEQ ID NO: 127 - protein, 44 aa, Chlamydomonas reinhardtii
YTVQKGETLWDVAVQHGVSMRTIKELNKLSGKEPLKEGQQLLVP

SEQ ID NO: 128 - protein, 43 aa, Chlamydophila pneumoniae AR39
YVVQDGDSLWLIAKRFGIPMDKIIQKNGLNHHRLFPGKVLKLP

SEQ ID NO: 129 - protein, 43 aa, Chlamydophila pneumoniae CWL029
YIVREGDSLSKIAKKYKLSVTELKKINKLDSDAIYAGQRLCLQ

SEQ ID NO: 130 - protein, 43 aa, Chlamydophila pneumoniae CWL029
VVVKKGDFLERIARANHTTVAKLMQINDLTTTQLKIGQVIKVP

SEQ ID NO: 131 - protein, 46 aa, Chlamydophila pneumoniae CWL029
YIVQEGDSPWTIALRNHIRLDDLLKMNDLDEYKARRLKPGDQLRIR

SEQ ID NO: 132 - protein, 49 aa, Citrobacter freundii
YTLKTGESVAQLSKSQGISVPVIWSLNKHLYSSESEMMKASPGQQIILP

SEQ ID NO: 133 - protein, 43 aa, Clostridium acetobutylicum
YVVKPGDSVYTIARRFRVTPQSILDANNLQNNRLVVGQAIVVP

SEQ ID NO: 134 - protein, 44 aa, Clostridium acetobutylicum
YRVRQGDTLWSIARRFRVTPKSITDLNNIENPSQIQPGLVIRIP

SEQ ID NO: 135 - protein, 44 aa, Clostridium acetobutylicum
YTIQPGDTFWAIAQRRGTTVDVIQSLNPGVVPTRLQVGQVINVP

SEQ ID NO: 136 - protein, 44 aa, Clostridium acetobutylicum
YTIQPGDTFWAIAQRRGTTVDVIQSLNPGVNPARLQVGQVINVP

SEQ ID NO: 137 - protein, 45 aa, Colletotrichum lindemuthianum
THKVKSGESLTTIAEKYDTGICNIAKLNNLADPNFIDLNQDLQIP

SEQ ID NO: 138 - protein, 44 aa, Colletotrichum lindemuthianum
YSVVSGDTLTSIAQALQITLQSLKDANPGVVPEHLNVGQKLNVP

SEQ ID NO: 139 - protein, 41 aa, Deinococcus radiodurans
VRPGQTLYRIALQNGLSVAELQRLNGLHSTTIEVGQVLRVT

SEQ ID NO: 140 - protein, 43 aa, Deinococcus radiodurans
YTVRRGDTLTSIGKFVGLRVEQLQRLNGLKGNTIAVGQVLRLT

SEQ ID NO: 141 - protein, 43 aa, Deinococcus radiodurans
YRVQPGDTLPKIGVKVGLRVEQLRRINGLTGDALQIGQVLRLT

SEQ ID NO: 142 - protein, 43 aa, Deinococcus radiodurans
YTVRRGDTLTSIGKFVGLPVEQLQRLNGLKDNTIAVGQVLRLT

SEQ ID NO: 143 - protein, 42 aa, Deinococcus radiodurans
YVVVPGDTLAKIGVKVGLRVEQLQRLNGLSGTTIEVGQVLKL

SEQ ID NO: 144 - protein, 45 aa, Deinococcus radiodurans
YTVKAGDTLSRIAGAYGTDASTLMPMNGLRSTTIQVGQRLQIGSS

SEQ ID NO: 145 - protein, 43 aa, Deinococcus radiodurans
YTVKKGDTLYSLARGSGLTVDALMRLNGLSTPELRVGQVIKLP

SEQ ID NO: 146 - protein, 43 aa, Deinococcus radiodurans
LTVQRGDTAYSIARRNGLTVDLLLAYNNLASPDIEVGQVLRLR

SEQ ID NO: 147 - protein, 44 aa, Deinococcus radiodurans
HTAQRGDTIYGLSRMYGVSVDALLAANTLPRDTKLEVGQVLQLP

SEQ ID NO: 148 - protein, 42 aa, Deinococcus radiodurans
YTVKAGDTLSRIAGAYGTDASTLMBNGLRSTTIQVGQRLQIG

SEQ ID NO: 149 - protein, 44 aa, Deinococcus radiodurans
YRVKPGETLYRIALNAGLSEETVQQANPVLRGGHALYAGQMLTI

SEQ ID NO: 150 - protein, 45 aa, Deinococcus radiodurans
FRVRKGEDLKKLAQRLGVSEGDIRRDNPQIDRRGSLNAGQVLRLP

SEQ ID NO: 151 - protein, 44 aa, Deinococcus radiodurans
HRVEIGDTFYSVARRYGINPIALQEYNPRLAGQTLNVGAVLSLV

SEQ ID NO: 152 - protein, 44 aa, Deinococcus radiodurans
YTVKPGDSLSKIAEHYYGDQNEYKKIAHYNNISNPDLIQPGQKL

SEQ ID NO: 153 - protein, 46 aa, Dictyostelium discoideum
LYTVKEKDTLTGISLQFSMPRDVLIQANBLLHSEKLLKPGTQLWVY

SEQ ID NO: 154 - protein, 46 aa, Dictyostelium discoideum
LYTVKEKDTLTGISLQFSMPRDVLIQANRLLHSEKLLKPGTQLWVY

SEQ ID NO: 155 - protein, 43 aa, Drosophila melanogaster
YTVGNRDTLTSVAARFDTTPSELTHLNRLNSSFIYPGQQLLVP

SEQ ID NO: 156 - protein, 45 aa, Drosophila melanogaster
RHIVEKTDTLQGIALKYGCTTEQIRRANRLFASDSLFLRQFLLVP

SEQ ID NO: 157 - protein, 44 aa, Drosophila melanogaster
SYTVGNRDTLTSVAARFDTTPSELTHLNRLNSSFIYPGQQLLVP

SEQ ID NO: 158 - protein, 45 aa, Drosophila melanogaster
EVKVQEGDTLQALALRFHSSVADIKRLNKIDRENEIHAHRVIRIP

SEQ ID NO: 159 - protein, 43 aa, Enterococcus faecalis
YTVKSGDTLNKIAAQYGVSVANLRSWIGISGDLIFVGQKLIVK

SEQ ID NO: 160 - protein, 43 aa, Enterococcus faecalis
YTVKSGDTLNKIAAQYGVTVANLRSWMGISGDLIFVGQKLIVK

SEQ ID NO: 161 - protein, 43 aa, Enterococcus faecalis
YTIKSGDTLNKIAAQYGVSVANLRSWNGISGDLIFAGQKIIVK

SEQ ID NO: 162 - protein, 44 aa, Enterococcus faecalis
YTIKSGDTLNKISAQFGVSVANLRSWNGIICGDLIFAGQTIIVK

SEQ ID NO: 163 - protein, 43 aa, Enterococcus faecalis
HTVKSGDSLWGLSMQYGISIQKIKQLNGLSGDTIYIGQTLKVG

SEQ ID NO: 164 - protein, 43 aa, Enterococcus hirae
YTVKSGDSVWGISHSFGITMAQLIEWNNIKNNFIYPGQKLTIK

SEQ ID NO: 165 - protein, 43 aa, Enterococcus hirae
YTVKSGDSVWKIANDHGISMHQLIEWNNIKNNFVYPGQQLVVS

SEQ ID NO: 166 - protein, 43 aa, Enterococcus hirae
YTVKAGESVWSVSNKFGISMNQLIQWNNIKNNFIYPGQKLIVK

SEQ ID NO: 167 - protein, 43 aa, Enterococcus hirae
YTVKAGESVWGVANKNGISMNQLIEWNNIKNNFIYPGQKLIVK

SEQ ID NO: 168 - protein, 43 aa, Enterococcus hirae
LTVKAGESVWGVANKHHITHDQLIEWNNIKNNFIYPGQEVIVK

SEQ ID NO: 169 - protein, 43 aa, Enterococcus hirae
YTVKAGESVWGVADSHGITMNQLIEWNNIKNNFIYPGQQLIVK

SEQ ID NO: 170 - protein, 44 aa, Escherichia coli K12
YTVKRGDTLYRISRTTGTSVKELARLNGISPPYTIEVGQKLKLG

SEQ ID NO: 171 - protein, 43 aa, Escherichia coli K12
YTVRSGDTLSSIASRLGVSTKDLQQWNKLRGSKLKPGQSLTIG

SEQ ID NO: 172 - protein, 42 aa, Escherichia coli
YRVRKGDSLSSIAKRMGVNIKDVMRWNSDTANLQPGDKLTLF

SEQ ID NO: 173 - protein, 44 aa, Escherichia coli O157H7
YTVKKGDTLFYIAWITGNDFRDLAQPNNIQAPYALNVGQTLQVG

SEQ ID NO: 174 - protein, 45 aa, Escherichia coli O6
YVVSTGDTLSSILMQYGIDMGDISQLAAADKELRNLKIGQQLSWT

SEQ ID NO: 175 - protein, 43 aa, Escherichia coli O6; identical to SEQ ID NO: 171

SEQ ID NO: 176 - protein, 42 aa, Escherichia coli O6
YRVRKGDSLSSIAKRHGVNIKDVMRWNSDTANLQPGDKLTLF

SEQ ID NO: 177 - protein, 41 aa, Fugu rubripes
VESRDTLNSISLKFDTTPNKLVQLNKLFSRAVVPGQVLYVP

SEQ ID NO: 178 - protein, 45 aa, Fugu rubripes
EHRVQPGETLQGLALKYGVSMEQIKRANRMYTNDSIHLRKSLSIP

SEQ ID NO: 179 - protein, 41 aa, Fugu rubripes
VGANDSLNSIALNFNITPNKLVQLNKLFSRSVYPGQKLFVP

SEQ ID NO: 180 - protein, 45 aa, Fugu rubripes
EREVLDGDTLNKLALQYGCKVADIKRLNNLMQEQDFYALKSVRIP

SEQ ID NO: 181 - protein, 45 aa, Fugu rubripes
TRDIQEGDTLNSISLQYHCSLADIKRANNLLTEQDFFALRSVKIP

SEQ ID NO: 182 - protein, 45 aa, Fugu rubripes
EHRVTDSDTLQGIALKYGVTMEQIKRANKLFSNDCIFLRNSLNIP

SEQ ID NO: 183 - protein, 43 aa, Haemophilus influenzae
HIVKKGESLGSLSNKYHVKVSDIIKLNQLKRKTLWLNESIKIP

SEQ ID NO: 184 - protein, 44 aa, Haemophilus influenzae
HKVTKNQTLYAISREYNIPVNILLSLNPHLKNGKVITGQKIKLR

SEQ ID NO: 185 - protein, 45 aa, Haemophilus influenzae
YTVTEGDTLKDVLVLSGLDDSSVQPLIALDPELAHLKAGQQFYWI

SEQ ID NO: 186 - protein, 45 aa, Haemophilus influenzae
YKVNKGDTMFLIAYLAGIDVKELAALNNLSEPNYNLSLGQVLKIS

SEQ ID NO: 187 - protein, 44 aa, Haemophilus somnus
YKVRKGDTMFLIAYISGMDIKELATLNMMSEPYHLSIGQVLKIA

SEQ ID NO: 188 - protein, 43 aa, Helicobacter pylori 26695
HVVLPKETLSSIAKRYQVSISNIQLANDLKDSNIFIHQRLIIR

SEQ ID NO: 189 - protein, 42 aa, Helicobacter pylori J99
HVVLPKETLSSIAKRYQVSISSIQLANNLKDSNIFIHQRLII

SEQ ID NO: 190 - protein, 45 aa, Homo sapiens
EHQLEPGDTLAGLALKYGVTMEQIKRAMRLYTNDSIFLKKTLYIP

SEQ ID NO: 191 - protein, 50 aa, Homo sapiens
RHVEHRVRAGDTLQGIALKYGVTMEQIKRANKLFTNDCIFLKKTLNIPVI

SEQ ID NO: 192 - protein, 44 aa, Homo sapiens
EYTAGNQDTLNSIALKFNITPNKLVELNKLFTHTIVPGQVLFVP

SEQ ID NO: 193 - protein, 44 aa, Homo sapiens
KDIQEGDTLNAIALQYCCTVADIKRVNNLISDQDFFALBSIKIP

SEQ ID NO: 194 - protein, 44 aa, Homo sapiens
EYTVESRDSLNSIALKFDTTPNELVQLNKLFSRAVVTGQVLYVP

SEQ ID NO: 195 - protein, 60 aa, Kluyveromyces lactis
YKVSSGESCSSIAVKYYPLSLNDIENYNKGNYGWKGCSSLQKDYNLCVSDGSAPRPVSNP

SEQ ID NO: 196 - protein, 44 aa, Lactobacillus phage phi-gle
YTVVSGDSWWKIAQRNGLSMYTLASQNGKSIYSTIYPGNKLIIK

SEQ ID NO: 197 - protein, 44 aa, Lactococcus lactis IL1430
YKVQEGDSLSAIAAQYGTTVDALVSAMSLENANDIHVGEVLQVA

SEQ ID NO: 198 - protein, 46 aa, Lactococcus lactis IL1430
YTVKSGDSLYSIAEQYGMTVSSLMSANGIYDVNSMLQVGQVLQVTV

SEQ ID NO: 199 - protein, 43 aa, Lactococcus lactis IL403
YTIQNGDSIYSIATANGMTADQLAALNGFGINDMIHPGQTIRI

SEQ ID NO: 200 - protein, 45 aa, Lactococcus lactis IL403
TYIVQAGDSLSIIAENHGYSVEEIQQVNPGVDFSVIHPGQEINLP

SEQ ID NO: 201 - protein, 44 aa, Lactococcus lactis phage LC3
YIVKQGDTLSGIASNLGTNWQELARQNSLSNPNMIYSGQVISLT

SEQ ID NO: 202 - protein, 43 aa, Lactococcus lactis phage LC3
YTVQSGDNLSSIARRLGTTVQSLVSMNGISNPNLIYAGQTLNY

SEQ ID NO: 203 - protein, 41 aa, Lactococcus lactis phage TP901-1
YTVSSGDNLSSIASRLGTTVQSLVSMNGISNPNLIYAGQTL

SEQ ID NO: 204 - protein, 44 aa, Lactococcus lactis phage TP901-1
YIVKQGDTLSGIASNWGTNWQELARQNSLSNPNVIYTGQVIRFT

SEQ ID NO: 205 - protein, 44 aa, Lactococcus lactis phage TPW22
YIVKQGDTLSGIASNWGTNWQELARQNSLSNPNTIYTGQVIRFT

SEQ ID NO: 206 - protein, 42 aa, Lactococcus lactis phage TPW22
YTVRSGDNLSSIASRLGTTVQSLVSMNGISNPSLIYAGQTLN

SEQ ID NO: 207 - protein, 44 aa, Lactococcus lactis phage Tuc2009
YVVKQGDTLSGIASNWGTNWQELARQNSLSNPNMIYAGQVISFT

SEQ ID NO: 208 - protein, 43 aa, Lactococcus lactis phage Tuc2009
YTVQSGDNLSSIAILLGTTVQSLVSMNGISNPNLIYAGQTLNY

SEQ ID NO: 209 - protein, 44 aa, Lactococcus oenos phage 10mcx
YTVQSGDTLGAIAAKYGTTYQKLASLNGIGSPYIIIPGEKLKVS

SEQ ID NO: 210 - protein, 44 aa, Lactococcus oenos phage 10mc
YKVASGDTLSAIASKYGTSVSKLVSLNGLKNANYIYVGENLKIK

SEQ ID NO: 211 - protein, 43 aa, Listeria grayi
VVVASGDTLWGIASKTGTTVDQLKQLNKLDSDRIVPGQKLTIK

SEQ ID NO: 212 - protein, 43 aa, Listeria grayi
YKVKSGDTIWALSVKYGVPVQKLIEWNNLSSSSIYVGQTIAVK

SEQ ID NO: 213 - protein, 44 aa, Listeria grayi
YKVQNGDSLGKIASLFKVSVADLTNWNNLNATITIYAGQELSVK

SEQ ID NO: 214 - protein, 43 aa, Listeria innocua
VVVEAGDTLWGIAQSKGTTVDAIKKANNLTTDKIVPGQKLQVN

SEQ ID NO: 215 - protein, 43 aa, Listeria innocua
HNVKSGDTIWALSVKYGVSVQDIMSWNHLSSSSIYVGQKPAIK

SEQ ID NO: 216 - protein, 43 aa, Listeria innocua
YTVVKGDSLWRIANNHKVTIANLKSWHNLKSDFIYPGQKLKVS

SEQ ID NO: 217 - protein, 43 aa, Listeria innocua
YTVAKGDSLWRIATNHKVTIANLKSWNNLKSDFIYPGQKLKVS

SEQ ID NO: 218 - protein, 43 aa, Listeria innocua; identical to SEQ ID NO: 217

SEQ ID NO: 219 - protein, 43 aa, Listeria innocua
YTVKKGDSLWAISRQYKTTVDNIKAWNKLTSNMIHVGQKLTIK

SEQ ID NO: 220 - protein, 43 aa, Listeria innocua
YKVKSGDTLPGVAKKFDVTVAEIKDWNHLSSDHLQTGQKLQLT

SEQ ID NO: 221 - protein, 43 aa, Listeria ivanovii
VVVEAGDTLWGIAQDKGTTVDALKKANNLTSDKIVPGQKLQIT

SEQ ID NO: 222 - protein, 43 aa, Listeria ivanovii
YTVKSGDTIWALSSKYGTSVQNIMSWNNLSSSSIYVGQVLAVK

SEQ ID NO: 223 - protein, 43 aa, Listeria ivanovii
YTVKSGDTLSKIATTFGTTVSKIKALNGLNSDNLQVGQVLKVK

SEQ ID NO: 224 - protein, 43 aa, Listeria monocytogenes; identical to SEQ ID NO: 214

SEQ ID NO: 225 - protein, 43 aa, Listeria monocytogenes
HAVKSGDTIWALSVKYGVSVQDIMSWNNLSSSSIYVGQKLAIK

SEQ ID NO: 226 - protein, 43 aa, Listeria monocytogenes
YTVVKGDSLWRIANNHKVTVANLKAWNNLKSDFIYPGQKLKVS

SEQ ID NO: 227 - protein, 43 aa, Listeria monocytogenes
YTVVKGDSLWRIANNNKVTIANLKAWNNLKSDFIYPGQKLKVS

SEQ ID NO: 228 - protein, 43 aa, Listeria monocytogenes
YTVAKGDSLWBIANNNKVTIANLKAWNNLCSDFIYPGQKLKVS

SEQ ID NO: 229 - protein, 43 aa, Listeria monocytogenes; identical to SEQ ID NO: 219

SEQ ID NO: 230 - protein, 43 aa, Listeria monocytogenes
YKVQDGDSLWKISNENNVSIKQLKEDNNLSSDIIFPNQTLQVN

SEQ ID NO: 231 - protein, 43 aa, Listeria monocytogenes
YTVVAGDTLGHIAVDNGVTVNQLKSWNNLSSDLIIVGQKLSIG

SEQ ID NO: 232 - protein, 43 aa, Listeria seeligeri
VVVEAGDTLWGIAQDNGTTVDALKKANKLTTDKIVPGQKLQVT

SEQ ID NO: 233 - protein, 43 aa, Listeria seeligeri
HTVKSGDTIWALSVKYGASVQDLMSWNNLSSSSIYVGQNIAVK

SEQ ID NO: 234 - protein, 43 aa, Listeria seeligeri
YTVKSGDTLGKIASTFGTTVSKIKALNGLTSDNLQVGDVLKVK

SEQ ID NO: 235 - protein, 43 aa, Listeria welshimeri
VVVEAGDTLWGIAQSKGTTVDALKKANNLTSDKIVPGQKLQVT

SEQ ID NO: 236 - protein, 43 aa, Listeria welshimeri
HTVKSGDTIWALSVKYGASVQDLMSWNNLSSSSIYVGQKIAVK

SEQ ID NO: 237 - protein, 43 aa, Listeria welshimeri
YTVKSGDSLSKIANTFGTSVSKIKALNNLTSDNLQVGTVLKVK

SEQ ID NO: 238 - protein, 45 aa, Macaca fascicularis
EHQLEPGDTLAGLALKYGVTMEQIKRANRLYTNDSIFLKKTLYIP

SEQ ID NO: 239 - protein, 40 aa, Mesorhizobium loti
YTVQSGDTMSSIARKTGVGVVALKQANGMKDGLLKIGQTL

SEQ ID NO: 240 - protein, 44 aa, Mus musculus
HRVRAGDTLQGIALKYGVTMEQIKRANKLFNNERIFLKKTLSIP

SEQ ID NO: 241 - protein, 45 aa, Mus musculus
QRELAQEDSLNKLALQYGCKVADIKKANNFIREQDLYALKSIKIP

SEQ ID NO: 242 - protein, 45 aa, Mus musculus
EHQLEPGDTLAGLALKYGVTMEQIKRTNRLYTNDSIFLKKTLYIP

SEQ ID NO: 243 - protein, 45 aa, Mus musculus
TKDIQEGDTLNAVALQYCCTVADIKRVNNLISDQDFFALRSIKIP

SEQ ID NO: 244 - protein, 44 aa, Mus musculus; identical to SEQ ID NO: 194

SEQ ID NO: 245 - protein, 42 aa, Neisseria meningitidis
HRVVEGDTLFNIAKRYNVSVADLIVANNIKGNTIQKGQVLRL

SEQ ID NO: 246 - protein, 43 aa, Neisseria meningitidis
HTIVRGDTVYNISKRYHISQDDFRAWNGMTDNTLSIGQIVKVK

SEQ ID NO: 247 - protein, 47 aa, Neisseria meningitidis
YTVKQGDTLWGISGKYLYSPWQWGRLWDANRDQIHNPDLIYPDQVLV

SEQ ID NO: 248 - protein, 46 aa, Neisseria meningitidis
YTVKQGDTLWGISGKYLYSPWQWGRLWDANRDQIHNPDLIYLDQVL

SEQ ID NO: 249 - protein, 43 aa, Neisseria meningitidis; identical to SEQ ID NO: 246

SEQ ID NO: 250 - protein, 43 aa, Neisseria meningitidis
HTIVRGDTVYNISKRYHISQDDERAWNGMTDNTLSIGQIVKVK

SEQ ID NO: 251 - protein, 44 aa, Oenococcus oeni phage f0g45
YTVRSGDTLGAIAAKYGTTYQKLASLNGIGSPYIIIPGEKLKVS

SEQ ID NO: 252 - protein, 44 aa, Oenococcus oeni phage f0g45
YKVASGDTLSAIASKYGTSVSKLVSLNGLKNANYIYVGQTLRIK

SEQ ID NO: 253 - protein, 48 aa, Oryza sativa
IYVVQPQDGLDAIARNVFNAFVTYQEIAAANNIPDPNKINVSQTLWIP

SEQ ID NO: 254 - protein, 45 aa, Oryza sativa
AYSVGKGENTSAIAAKYGVTESTLLTPNKIDDPTKLQMGQILDVP

SEQ ID NO: 255 - protein, 45 aa, Oryza sativa
SNTVRRGDTVPGIALKYSIQVTDIKRFNNMMSDHGIYLRERLLIP

SEQ ID NO: 256 - protein, 45 aa, Oryza sativa
VHGVQASETCFSVSQSAGLTQDQFLAFNPNINCAKVFVGQWVCLD

SEQ ID NO: 257 - protein, 50 aa, Oryza sativa
VHGVEAGETCDSIARRFHAGLGRAPFFRLVSLNPNINCRELFVGQWVCIQ

SEQ ID NO: 258 - protein, 46 aa, Oryza sativa
SYAVQDGDTLGNIASLFRSSWKDILDLNPRVANPDFIKPGWILFIP

SEQ ID NO: 259 - protein, 45 aa, Oryza sativa
LHRVGKLDTLAGIAIKYGVEVADIKRLNGLSTDLQMFAHKTLRIP

SEQ ID NO: 260 - protein, 50 aa, Oryza sativa
SHTIRDTGVETYFIIANLTYQGLSTCQALIAQNPLHDSRGLVAGDNLTVP

SEQ ID NO: 261 - protein, 45 aa, Oryza sativa
TYLVTWGDTVSAIAARFRVDAQEVLDANTLTESSIIYPFTTLLVP

SEQ ID NO: 262 - protein, 43 aa, Pasteurella multocida
HKVKAGENLTGVARKYGVKVNDILALNKLKRRELWIGETLKIP

SEQ ID NO: 263 - protein, 44 aa, Pasteurella multocida
HTVKKGQTLYAIAREYHIPPNQLLKLNPNLKDGKVLTGQKIKLR

SEQ ID NO: 264 - protein, 44 aa, Pasteurella multocida
YTVRKGDSMYLISYISGLSIKEIAALNNLSEPYTLATGQVLKLS

SEQ ID NO: 265 - protein, 50 aa, Pichia acaciae
FYIVQEDDTCVSIASKYPGLTEQDIIDYNSKNGDFYGCFNLWEGDKICIS

SEQ ID NO: 266 - protein, 52 aa, Pichia etchellsii
HTNASGESCAFLIEKFTNVETEINLNKWNEDNPTWYGCSNGHPYVGDKVCVS

SEQ ID NO: 267 - protein, 48 aa, Pichia etchellsii
YYIKSGDTCYDLASKYYLSTEQLESFNEKTYGWFCCNNLQLGQAMCLG

SEQ ID NO: 268 - protein, 43 aa, Prevotella intermedia
HTVRSNESLYDISQQYGVRLKNIMKANRKIVKRGIKAGDRVVL

SEQ ID NO: 269 - protein, 43 aa, Proteobacterium EBAC31A08
YKIQKGDVLSEIAIRFGVTVEEINTENKLNNKPIYPGQLIKIN

SEQ ID NO: 270 - protein, 44 aa, Pseudomonas aeruginosa PA01
YIVRRGDTLYSIAFRFGWDWKALAARNGIAPPYTIQVGQAIQFG

SEQ ID NO: 271 - protein, 40 aa, Pseudomonas aeruginosa PA01
YRVRSGDSLHSIANRYRITVAELKSANRLSSNHLRKGQQL

SEQ ID NO: 272 - protein, 42 aa, Pseudomonas aeruginosa PA01
YKVKNGDSLWQIARNNGVDVNDLKRWNGLDKHALKVGQTLKL

SEQ ID NO: 273 - protein, 40 aa, Pseudomonas aeruginosa
YKVKQGDSNYLIAKRFNVEMKHLQRWNPRSKQALKPGQTL

SEQ ID NO: 274 - protein, 44 aa, Pseudomonas putida WCS358
YIVKPGDTLFSIAFRYGWDYKELAARNGIPAPYTIRPGQPIRFS

SEQ ID NO: 275 - protein, 46 aa, Rattus norvegicus
GYTVKSGDTLSAIAAANGVSLANLLSWNNLSLQAIIYPGQKLTIQN

SEQ ID NO: 276 - protein, 46 aa, Rattus norvegicus
SYTVKSGDTLYGIAAKLGTNVQTLLSLNGLQLSSTIYVGQVLKTTG

SEQ ID NO: 277 - protein, 43 aa, Salmonella typhimurium LT2
TVKKGDTLFYIAWITGNDFRDLAQRNSISAPYSLNVGQTLQVG

SEQ ID NO: 278 - protein, 44 aa, Sinorhizobium meliloti
IMVRQGDTVTVLARRFGVPEKEILKANGLKSASQVEPGQRLVIP

SEQ ID NO: 279 - protein, 48 aa, Solanum tuberosum
IYTVVSGDFLSHIVTDIFAGLFTVQELQTVNNISNPNLIQPGDKLWIP

SEQ ID NO: 280 - protein, 45 aa, Solanum tuberosum
GRLVISGNSIEAIAQQYNVSQETLLRLNGLASPKELLAGAVLDVP

SEQ ID NO: 281 - protein, 44 aa, Staphylococcus aureus
HVVKPGDTVNDIAKANGTTADKIAADNKLADKNMIKPGQELVVD

SEQ ID NO: 282 - protein, 44 aa, Staphylococcus aureus
YTVKKGDTLSAIALKYKTTVSNIQNTNNIANPNLIFIGQKLKVP

SEQ ID NO: 283 - protein, 40 aa, Staphylococcus aureus
HTVKPGBSVWAISNKYGISIAKLKSLNNLTSNLIFPNQVL

SEQ ID NO: 284 - protein, 43 aa, Staphylococcus aureus
YTVQAGDSLSLIASKYGTTYQKIMSLNGLNNFFIYPGQKLKVT

SEQ ID NO: 285 - protein, 40 aa, Staphylococcus aureus
YTVQAGDSLSLIASKYGTTYQNIMRLNGLNNFFIYPGQKL

SEQ ID NO: 286 - protein, 38 aa, Staphylococcus aureus
HTVQSGESLWSIAQKYNTSVESIKQNNQLDNNLVFPGQ

SEQ ID NO: 287 - protein, 40 aa, Staphylococcus aureus
HTVQAGESLNIIASRYGVSVDQLMAANNLRGYLIMPNQTL

SEQ ID NO: 288 - protein, 43 aa, Streptococcus pneumoniae
YTVKEGDTLSBIAETHNTTVEKLAENNHIDNIHLIYVDQELVI

SEQ ID NO: 289 - protein, 44 aa, Synechococcus sp. PCC 6803
HQVKEGESLWQISQAFQVDAKAIALANSISTDTELQAGQVLNIP

SEQ ID NO: 290 - protein, 44 aa, Synechococcus sp. PCC 6803
HVVKAGETIDSIAAQYQLVPATLISVNNQLSSGQVTPGQTILIP

SEQ ID NO: 291 - protein, 44 aa, Thermotoga maritima
YKVQKNDTLYSISLNFGISPSLLLDWNPGLDPHSLRVGQEIVIP

SEQ ID NO: 292 - protein, 43 aa, Thermotoga maritima
YTVKKGDTLDAIAKRFFTTATFIKEANQLKSYTIYAGQKLFIP

SEQ ID NO: 293 - protein, 44 aa, Thermotoga maritima
HVVKRGETLWSIANQYGVRVGDIVLINRLEDPDRIVAGQVLKIG

SEQ ID NO: 294 - protein, 44 aa, Treponema pallidum
HTIRSGDTLYALARRYGLGVDTLKAHNRAHSATHLKIGQKLIIP

SEQ ID NO: 295 - protein, 44 aa, Treponema pallidum
HVVQQGDTLWSLAKRYGVSVENLAEENNLAVDATLSLGMILKTP

SEQ ID NO: 296 - protein, 44 aa, Treponema pallidum
YEVREGDVVGRIAQRYDISQDAIISLNKLRSTRALQVGQLLKIP

SEQ ID NO: 297 - protein, 45 aa, Treponema pallidum
YTVKNGDTFSSIAAAHQISLERLVLLNTPSSSKBSPPSVRTLVSP

SEQ ID NO: 298 - protein, 44 aa, Treponema pallidum
HVIAKGETLFSLSRRYGVPLSALAQANNLANVHQLVPGQRIVVP

SEQ ID NO: 299 - protein, 44 aa, Treponema pallidum
YTVRRGDTLFSIARNLNCSLAALLAANGISAAHTIHPGDVLVIP

SEQ ID NO: 300 - protein, 43 aa, Vibrio cholerae
HVVKTGDFLGKLATTYKVSVASIKKENNLKSDTLVLGQKLKIT

SEQ ID NO: 301 - protein, 43 aa, Vibrio cholerae
HKVQRGESIGLIANQYGVSVDALKKANNLIZSSTISVGQLLTI

SEQ ID NO: 302 - protein, 42 aa, Vibrio cholerae
HKVQRGEFLSKIADQYNVSVDSIRQANQLRTDQLLVGQQLII

SEQ ID NO: 303 - protein, 43 aa, Vibrio cholerae
YKVKSGDTLSTIADKYNTTAKVIKEANQIASNQIRVGSYLFVP

SEQ ID NO: 304 - protein, 44 aa, Vibrio cholerae
HTVNSGESLWTIAKQYNVPYQSLAKWNGMAPKDALRKGQKLVIW

SEQ ID NO: 305 - protein, 44 aa, Vibrio cholerae
YKVRSGDTLSGIANKFKVKTADIVKWNDLNSTQYLKAGQQLKLY

SEQ ID NO: 306 - protein, 44 aa, Volvox carteri f. nagariesnsis; identical to SEQ ID NO: 135

SEQ ID NO: 307 - protein, 44 aa, Volvox carteri f. nagariensis; identical to SEQ ID NO: 136

SEQ ID NO: 308 - protein, 47 aa, Xylella fastidiosa
YTVQKGDTLWGLAKRLFKKPWLWPEIWQANPQINNPHIYPGDVISLA

SEQ ID NO: 309 - protein, 44 aa, Xylella fastidiosa
VVVKQGDTLYAISRRTGVAPQDLAAWNRLTASKTIYPGQVLRLY